(19) United States
(12) Patent Application Publication
(10) Pub. No.: US 2025/0228976 A1
(43) Pub. Date: Jul. 17, 2025

(54) AAV CAPSIDS THAT ENABLE CNS-WIDE GENE DELIVERY THROUGH INTERACTIONS WITH THE TRANSFERRIN RECEPTOR

(71) Applicant: The Broad Institute, Inc., Cambridge, MA (US)

(72) Inventors: Benjamin Deverman, Cambridge, MA (US); Qin Huang, Cambridge, MA (US); Ken Chan, Cambridge, MA (US); Andrew Barry, Cambridge, MA (US); Fatmaelzahraa Sobhy Abdelmouty Eid, Cambridge, MA (US)

(21) Appl. No.: 19/020,432

(22) Filed: Jan. 14, 2025

Related U.S. Application Data
(63) Continuation of application No. PCT/US2023/070285, filed on Jul. 14, 2023.
(60) Provisional application No. 63/368,470, filed on Jul. 14, 2022.
(60) Provisional application No. 63/432,336, filed on Dec. 13, 2022.

(51) Int. Cl. A61K 48/00; C07K 14/005; C12N 15/10; C12N 15/86

(52) U.S. Cl. CPC A61K 48/0058; C07K 14/005; C12N 15/1082; C12N 15/86; C12N 2750/14122; C12N 2750/14143; C12N 2750/14145

(57) ABSTRACT

An engineered AAV capsid is provided, in which at least one protein on the capsid is modified to include a n-mer motif, which promotes transduction of the capsid into the central nervous system (CNS) through interaction with the Transferrin receptor. Further embodiments provide a vector system comprising one or more vectors encoding AAV capsids and a method of delivering cargo to the CNS. The method comprises administering, in vivo or in vitro, a AAV capsid according to embodiments described herein and the AVV capsid comprises one or more cargo molecules.

### CROSS-REFERENCE TO RELATED APPLICATIONS

This application is a continuation application of PCT/US2023/070285, filed Jul. 14, 2023, which claims the benefit of U.S. Provisional Application No. 63/432,336, filed Dec. 13, 2022 and U.S. Provisional Application No. 63/368,470 filed Jul. 14, 2022. The entire contents of the above-identified applications are hereby fully incorporated herein by reference.

### STATEMENT REGARDING FEDERALLY SPONSORED RESEARCH

This invention was made with government support under Grant Nos. NS111689 and MH120096 awarded by the National Institutes of Health. The government has certain rights in the invention.

### REFERENCE TO AN ELECTRONIC SEQUENCE LISTING

Reference is made to the electronic sequence listing (“BROD-5515US_ST26.xml”; Size is 51,570,009 bytes, created on Feb. 19, 2025) is herein incorporated by reference in its entirety.

### REFERENCE TO ELECTRONIC TABLES

Reference is made to electronic Tables 1-20 and 22 filed herewith in the United States Patent and Trademark Office. Reference is also made to Tables 1-13 filed on Jul. 14, 2022, in the United States Patent and Trademark Office, and assigned Ser. No. 63/368,470. The Table are herein incorporated by reference in their entirety.

LENGTHY TABLES
The patent application contains a lengthy table section. A copy of the table is available in electronic form from the USPTO web site (https://seqdata.uspto.gov/?pageRequest=docDetail&DocID=US20250228976A1). An electronic copy of the table will also be available from the USPTO upon request and payment of the fee set forth in 37 CFR 1.19(b)(3).

### TECHNICAL FIELD

The subject matter disclosed herein relates generally to enhancing transduction of an engineered AA capsid into the central nervous system (CNS) through interaction with the Transferrin receptor. In particular examples described herein, at least one protein on the capsid is modified to include an n-mer motif. Particular examples relate to a vector system having one or more vectors encoding AAV capsids and a method of delivering cargo to the CNS, in which an AAV capsid according to the examples described herein is administered in vivo or in vitro, and the AAV capsid comprises one or more cargo molecules.

### BACKGROUND

The development of gene therapies for neurodevelopmental and neurological disorders has been constrained by the inability to efficiently deliver genes throughout the CNS. Several studies have reported engineered AAV9 capsids, most notably the AAV-PHP.B family, that are capable of highly effective gene transfer throughout the CNS after intravenous administration in adult mice. To date however, none of the engineered AAV capsids that cross the blood-brain barrier (BBB) and transduce the mouse brain with high efficiency have been shown to exhibit their enhanced CNS tropism in primates. In this work, Applicants take a mechanism-first approach and engineer AAV capsids that interact with the Transferrin Receptor (TFRC).

Citation or identification of any document in this application is not an admission that such a document is available as prior art to the present invention.

### SUMMARY

In one aspect, provided herein is a composition comprises a targeting moiety effective to increase transduction of central nervous system tissues (CNS) via binding to a transferrin receptor (TFRC), optionally further comprising a cargo coupled to or otherwise associated with the targeting moiety. In addition, one aspect provided herein is of a vector system comprising one or more vectors encoding a targeting moiety effective to increase transduction of central nervous system tissues (CNS). Additional embodiments provided herein comprise polypeptides or particles encoded or produced by a vector system herein or a cell comprising the composition, vector, polynucleotide or particle provided herein. In one aspect, provided herein is a method of delivering one or more cargos to the CNS by administering a composition provided herein.

In an example embodiment, the targeting moiety binds to the extracellular domain of TFRC. In an example embodiment, the targeting moiety binds to one or more of the apical, helical, and/or protease-like domain of the extracellular domain. In an example embodiment, the targeting moiety binds to the apical domain.

In an example embodiment, the targeting moiety comprises a n-mer motif, the n-mer motif comprising or consisting of an amino acid sequence of X₁-X₂-X₃-X₄-X₅-X₆-X₇ wherein: X₁ comprises of Y, M, F, and L; X₂ comprises of S, H, T, and A; X₃ comprises of K and R; X₄ comprises of A, G, I, L, M, N, Q, S, T, V and H; X₅ comprises of N, G, A, L, M, Q, S, and T; X₆ comprises of A, T, H, N, F, I, P, L, Y, G, S, V, D, E, M, and Q; and X₇ comprises of D and N. In an example embodiment, X₁ comprises of Y, M, and L; X₂ comprises of S, H, T, and A; X₃ comprises of K and R; X₄ comprises of A, G, I, L, M, N, Q, S, T, and V; X₅ comprises N; X₆ comprises of A, T, H, N, F, I, P, L, and Y; and X₇ comprises of D and N; or X₁ comprises of Y, M, and L; X₂ comprises of S, H, T, and A; X₃ comprises of K and R; X₄ comprises of A, G, I, L, M, N, Q, S, T, and V; X₅ comprises G; X₆ comprises of A, G, F, H, I, L, N, P, S, T, V, and Y; and X₇ comprises of D and N; or X₁ comprises of L and Y; X₂ comprises of A, H, and S; X₃ comprises of K and R; X₄ comprises of A, G, H, I, L, M, N, Q, S, T, and V; X₅ comprises G; X₆ comprises P; and X₇ comprises D; or X₁ comprises of L and Y; X₂ comprises of A, H, and S; X₃ comprises of K and R; X₄ comprises of A, G, H, I, L, M, N, Q, S, T, and V; X₅ comprises G; X₆ comprises P; and X₇ comprises N; or X₁ comprises Y; X₂ comprises S; X₃ comprises K; X₄ comprises of A, I, L, M, N, Q, S, T, and V; X₅ comprises G; X₆ comprises of X; and X₇ comprises Y, P, T, Q, V, F, L, H, S, A, E, D, I, and M; or X₁ comprises of L and Y; X₂ comprises S; X₃ comprises of R and K; X₄ comprises of V, I, T, L, and A; X₅ comprises of S and A; X₆ comprises of P, R, Y, F, H, I, K, and W; and X₇ comprises D; or X₁ comprises of F, L, M, and Y; X₂ comprises H; X₃ comprises of K and R; X₄ comprises of A, L, and M; X₅ comprises of A, G, L, M, N, Q, S, and T; X₆ comprises of A, D, E, F, H, I, L, M, N, Q, P, S, T, V, and Y; and X₇ comprises of D and N; or X₁ comprises of L, M, and Y; X₂ comprises H; X₃ comprises of K and R; X₄ comprises of A, L, and M; X₅ comprises of A, G, L, M, N, Q, S, and T; X₆ comprises of A, D, E, F, H, I, L, M, N, Q, P, S, T, V, and Y; and X₇ comprises N; or X₁ comprises of L, M, and Y; X₂ comprises H; X₃ comprises of K and R; X₄ comprises of A, L, and M; X₅ comprises of A, G, L, M, N, Q, S, and T; X₆ comprises of A, D, E, F, H, I, L, M, N, Q, P, S, T, V, and Y; and X₇ comprises D; or X₁ comprises of L, M, and Y; X₂ comprises H; X₃ comprises of K and R; X₄ comprises L; X₅ comprises of S, Q, G, T, N, and L; X₆ comprises of P, T, V, I, Q, L, and A; and X₇ comprises D; or X₁ comprises F; X₂ comprises S; X₃ comprises R; X₄ comprises L; X₅ comprises G; X₆ comprises of A, H, N, L, V, S, P, and T; and X₇ comprises N; or X₁ comprises F; X₂ comprises A; X₃ comprises R; X₄ comprises of T, S, and N; X₅ comprises G; X₆ comprises of Y, F, H, P, and A; and X₇ comprises N; or X₁ comprises F; X₂ comprises H; X₃ comprises of K and R; X₄ comprises L; X₅ comprises G; X₆ comprises of I, P, and S; and X₇ comprises of N and D. In an example embodiment, the n-mer motif is selected from the group consisting of LHRLGPN (SEQ ID NO: 36834), YSRIGPN (SEQ ID NO: 14632), LHRLGPN (SEQ ID NO: 36834), LHRLGPD (SEQ ID NO: 36413), LHRAGPD (SEQ ID NO: 36894), YSRIGPD (SEQ ID NO: 38223), LSRIGPD (SEQ ID NO: 36274), LARSGPD (SEQ ID NO: 18035), YSRNSDN (SEQ ID NO: 16626), LHKAGPN (SEQ ID NO: 36305), LSRIGPN (SEQ ID NO: 36347), LAKSGPN (SEQ ID NO: 36287), YARNGPN (SEQ ID NO: 14048) and YSRNSDN (SEQ ID NO: 16626). In an example embodiment, the n-mer motif is YSRIGPN (SEQ ID NO: 14632).

In an example embodiment, the targeting moiety comprises a n-mer motif, the n-mer motif comprising or consisting of an amino acid sequence of X₁-X₂-X₃-X₄-X₅-X₆-X₇ wherein: X₁ comprises of Y or L; X₂ comprises H; X₃ comprises A; X₄ comprises of K, R, N, and A; X₅ comprises of G, Q, L, and S; X₆ comprises of P, L, I, N, D, and T; and X₇ comprises N. In the example embodiment, the targeting moiety comprises a n-mer motif, the n-mer motif comprising or consisting of an amino acid sequence of X₁-X₂-X₃-X₄-X₅-X₆-X₇ wherein: X₁ comprises of A, F, H, I, L, N, P, R, S, T, V, and Y; X₂ comprises of A, D, E, F, G, H, I, K, L, M, N, Q, R, S, T, W, and Y; X₃ comprises S; X₄ comprises of S and T; X₅ comprises N; X₆ comprises G; and X₇ comprises of I, R, and V. In an example embodiment, X₁ comprises of F, L, and Y; X₂ comprises of D, E, H, N, Q, S, and T; X₃ comprises S; X₄ comprises of S and T; X₅ comprises N; X₆ comprises G; and X₇ comprises of I and V; or X₁ comprises of V, P, I, S, T, H, and A; X₂ comprises of A, D, E, F, G, H, I, K, L, M, N, Q, R, S, T, and Y; X₃ comprises of S; X₄ comprises of S and T; X₅ comprises N; X₆ comprises G; and X₇ comprises of I and V; or X₁ comprises of A, F, I, L, P, S, T, V, and Y; X₂ comprises of D, E, N, Q, S, and T; X₃ comprises S; X₄ comprises of T and S; X₅ comprises N; X₆ comprises G; and X₇ comprises R; or X₁ comprises R; X₂ comprises of E, D, Q, and T; X₃ comprises S; X₄ comprises of S and T; X₅ comprises N; X₆ comprises G; and X₇ comprises of I and V. In an example embodiment, the n-mer motif is selected from the group consisting of FRSTNGV (SEQ ID NO: 16070), VESTNGR (SEQ ID NO: 36431), VDSTNGV (SEQ ID NO: 12206), VQSTNGV (SEQ ID NO: 36423), VSSTNGV (SEQ ID NO: 12333), TESTNGR (SEQ ID NO: 17558), VQSTNGI (SEQ ID NO: 11292) and FVSTNGV (SEQ ID NO: 11162).

In an example embodiment, the wherein the targeting moiety comprises a n-mer motif, the n-mer motif comprising or consisting of an amino acid sequence of X₁-X₂-X₃-X₄-X₅-X₆-X₇ wherein: X₁ comprises of R and T; X₂ comprises of T, L, M, S, G, D, N, E, R, K, Y, and W; X₃ comprises of G, E, D, I, F, H, S, A, M, P, V, Y, W, Q, and T; X₄ comprises of D, T, E, H, N, and G; X₅ comprises of A, V, S, T, and D; X₆ comprises of Y, F, P and A; and X₇ comprises of A and P. In an example embodiment, X₁ comprises R; X₂ comprises of T, M, L, and S; X₃ comprises of Y, S, A, M, I, F, and P; X₄ comprises D; X₅ comprises of A, V, S, and T; X₆ comprises of Y and F; and X₇ comprises P; or X₁ comprises R; X₂ comprises of T, M, L, and S; X₃ comprises of Y, S, A, M, I, F, and P; X₄ comprises D; X₅ comprises of A, V, S, and T; X₆ comprises of Y and F; and X₇ comprises A; or X₁ comprises R; X₂ comprises of G, T, D, S, N, and E; X₃ comprises of E, D, P, S, and G; X₄ comprises of D, T, E, H, and N; X₅ comprises of V, A, and T; X₆ comprises of Y and F; and X₇ comprises P; or X₁ comprises R; X₂ comprises of G, L, T, D, and S; X₃ comprises of D, P, S, and G; X₄ comprises of D, E, H, and N; X₅ comprises of V and T; X₆ comprises of Y and F; and X₇ comprises P; or X₁ comprises T; X₂ comprises of R, K, Y, and W; X₃ comprises of E, W, Y, Q, S, and T; X₄ comprises G; X₅ comprises D; X₆ comprises of P and A; and X₇ comprises of A and P. In an example embodiment, the n-mer motif is selected from the group consisting of RGEDVYP (SEQ ID NO: 36864), RLEDVFP (SEQ ID NO: 36264), RTYDSYP (SEQ ID NO: 37938), RTYDAYP (SEQ ID NO: 38571), RTYDSFP (SEQ ID NO: 37806), RTETVYP (SEQ ID NO: 36486), RTETVFP (SEQ ID NO: 36389), and RTEHVFP (SEQ ID NO: 36603).

In an example embodiment, the targeting moiety comprises a n-mer motif, the n-mer motif comprising or consisting of an amino acid sequence of X₁-X₂-X₃-X₄-X₅-X₆-X₇ wherein: X₁ comprises L; X₂ comprises C; X₃ comprises of K and R; X₄ comprises P; X₅ comprises C; X₆ comprises of L, S, D, A, N, Q, H, P, and V; and X₇ comprises of E, T, G, A, D, N, and S. In an example embodiment, the n-mer motif is LCKPCLD (SEQ ID NO: 36437) or LCKPCPT (SEQ ID NO: 36438). In an example embodiment, the targeting moiety comprises a n-mer motif, the n-mer motif comprising or consisting of an amino acid sequence of X₁-X₂-X₃-X₄-X₅-X₆-X₇ wherein: X₁ comprises of Y, and F; X₂ comprises of W, F, and Y; X₃ comprises of S, T, H, A and Q; X₄ comprises G; X₅ comprises of I, T, V, Q, M, H, K, and R; X₆ comprises of I, P, H, L, M, A, Q, T, V, K, and R; and X₇ comprises of A, S, D, E, and N. In an example embodiment, X₁ comprises of Y and F; X₂ comprises of W, F, and Y; X₃ comprises of T and S; X₄ comprises G; X₅ comprises of I, T, V, Q, M, and H; X₆ comprises of I, P, H, L, M, A, Q, T, and V; and X₇ comprises of A, S, D, and E; or X₁ comprises of Y and F; X₂ comprises of W, F, and Y; X₃ comprises of T and S; X₄ comprises G; X₅ comprises of I, T, V, Q, M, and H; X₆ comprises of K and R; and X₇ comprises of A, S, D, and E; or X₁ comprises of Y and F; X₂ comprises of W, F, and Y; X₃ comprises of T and S; X₄ comprises G; X₅ comprises of K and R; X₆ comprises of I, P, H, L, M, A, Q, T, and V; and X₇ comprises of A, S, D, and E; or X₁ comprises Y; X₂ comprises F; X₃ comprises T; X₄ comprises G; X₅ comprises of K, R, Q, M, H, and I; X₆ comprises of T, R, H, K, V, and L; and X₇ comprises E; or X₁ comprises Y; X₂ comprises F; X₃ comprises of T, S, H, and A; X₄ comprises G; X₅ comprises of K, R, and T; X₆ comprises of I, P, H, L, M, A, Q, and T; and X₇ comprises of D and N; or X₁ comprises Y; X₂ comprises W; X₃ comprises T; X₄ comprises G; X₅ comprises of K, M, V, and T; X₆ comprises of P, V, I, H, Q, T, M, and L; and X₇ comprises of E and D; or X₁ comprises of Y and F; X₂ comprises F; X₃ comprises of S, H, A, and Q; X₄ comprises G; X₅ comprises of K, Q, and R; X₆ comprises of I, V, L, K, H, R, Q, and M; and X₇ comprises E;

In the example embodiment, the targeting moiety comprises a n-mer motif, the n-mer motif comprising or consisting of an amino acid sequence of X₁-X₂-X₃-X₄-X₅-X₆-X₇ wherein: X₁ comprises of K, R, S, G, N, T, M, Q, V, D, I, and E; X₂ comprises of D, S, N, M, L, G, P, E, and A; X₃ comprises of E, D, G, S, A, R, Q, T, P, and N; X₄ comprises of F, Y, T, V, S, N, A, G, and H; X₅ comprises of T, K, S, R, V, and H; X₆ comprises of T, S, G, V, A, K, R, N, D, E, and H; and X₇ comprises of F, W, and Y. In an example embodiment, X₁ comprises of K and R; X₂ comprises of D, S, and N; X₃ comprises E; X₄ comprises F; X₅ comprises of T, K, S, R, and V; X₆ comprises of T, S, G, and V; and X₇ comprises of F, W, and Y; or X₁ comprises of K and R; X₂ comprises D; X₃ comprises D; X₄ comprises of F and Y; X₅ comprises of T, S, V, and H; X₆ comprises of T, S, G, V, and A; and X₇ comprises of F, W, and Y; or X₁ comprises of S, R, G, N, T, M, and Q; X₂ comprises D; X₃ comprises G; X₄ comprises of T, V, S, N, and Y; X₅ comprises S; X₆ comprises of K and R; and X₇ comprises W; or X₁ comprises of R, V, D, I, Q, and K; X₂ comprises of M, L, and G; X₃ comprises of S, E, A, R, and Q; X₄ comprises D; X₅ comprises R; X₆ comprises of T, A, S, G, K, and N; and X₇ comprises W; or X₁ comprises of D, I, E, Q, V, S, and K; X₂ comprises of L, M, G, and P; X₃ comprises of E, A, S, D, Q, and T; X₄ comprises of S and A; X₅ comprises R; X₆ comprises of D, S, E, T, G, and A; and X₇ comprises W; or X₁ comprises G; X₂ comprises of E, S, P, G, and A; X₃ comprises of D, E, P, and N; X₄ comprises of G, H, T, S, and N; X₅ comprises V; X₆ comprises of R, K, and S; and X₇ comprises of W and Y; or X₁ comprises R; X₂ comprises E; X₃ comprises of D, E, P, and N; X₄ comprises of G, H, T, S, and N; X₅ comprises V; X₆ comprises of R, K, and S; and X₇ comprises of W and Y; or X₁ comprises G; X₂ comprises of G and S; X₃ comprises of G, E, S, A, P, and D; X₄ comprises of T, G, and S; X₅ comprises S; X₆ comprises of S, T, H, K, R, A, and N; and X₇ comprises W. In an example embodiment, the n-mer motif is selected from the group consisting of KDEFTTF (SEQ ID NO: 36308), KDDFTTY (SEQ ID NO: 36336), RDEFTTY (SEQ ID NO: 36615), KDEFSTY (SEQ ID NO: 36390), RDEFTSF (SEQ ID NO: 36701), and REDHVSW (SEQ ID NO: 37067).

In an example embodiment, the targeting moiety comprises a n-mer motif, the n-mer motif comprising or consisting of an amino acid sequence of X₁-X₂-X₃-X₄-X₅-X₆-X₇ wherein: X₁ comprises of V, I, R, N and D; X₂ comprises of A, G, and S; X₃ comprises of L, T, S, H, and G; X₄ comprises of K, R, and E; X₅ comprises G; X₆ comprises of W, R, A, and I; and X₇ comprises of D and G. In an example embodiment the n-mer motif is selected from the group consisting of IALKGWD (SEQ ID NO: 36248), NALEGRD (SEQ ID NO: 36407), VALEGRD (SEQ ID NO: 36604), and VALKGWD (SEQ ID NO: 17701). In an example embodiment the targeting moiety comprises a n-mer motif, the n-mer motif comprising or consisting of an amino acid sequence of X₁-X₂-X₃-X₄-X₅-X₆-X₇ wherein: X₁ comprises of L, M, and W; X₂ comprises of F, R, W, K, T, and Y; X₃ comprises of D and S; X₄ comprises G; X₅ comprises T; X₆ comprises of P, G, S, N, A, and R; and X₇ comprises of A, P, S, and Y.

In an example embodiment, the targeting moiety comprises a n-mer motif, the n-mer motif comprising or consisting of an amino acid sequence of X₁-X₂-X₃-X₄-X₅-X₆-X₇ wherein: X₁ comprises of P, N, and K; X₂ comprises of Y and F; X₃ comprises of A; X₄ comprises of R and K; X₅ comprises of S; X₆ comprises of P, V, A, R, I, L, S, E; and X₇ comprises of E, D, M, and L.

In an example embodiment, a composition comprising a targeting moiety effective to increase transduction of CNS tissues comprises a n-mer motif, the n-mer motif comprising or consisting of an amino acid sequence of Z₁-X₁-Z₂-X₂-X₃-X₄-X₅ wherein Z₁ comprises Y, F, or L, Z₂ comprises S, R, or K, X₁-X₅ are independently selected amino acids. In an example embodiment, X₁ optionally comprises of A, S, or H; X₂ optionally comprises of S, T, L, or I; X₃ optionally comprises N or G; X₄ optionally comprises G; X₅ optionally comprises of N, D, I, V, or R. In an example embodiment, the n-mer motif is selected from the group consisting of YSRIGPN (SEQ ID NO: 14632), YSRLNMN (SEQ ID NO: 14301), YSRLNKD (SEQ ID NO: 16577), and YHRLSNN (SEQ ID NO: 16636). In an example embodiment, the targeting moiety comprises a n-mer motif, the n-mer motif comprising or consisting of an amino acid sequence of is X₁-H-X₂-L-X₃-X₄-X₅ wherein X₁-X₅ are independently selected amino acids. In an example embodiment, the n-mer motif is VHRLQDK (SEQ ID NO: 16602) or LHALSHN (SEQ ID NO: 16608).

In an example embodiment, the n-mer motif is PSATNGV (SEQ ID NO: 20486), QVSTNGI (SEQ ID NO: 16021), SYSSNGV (SEQ ID NO: 16234), HQSSNGV (SEQ ID NO: 15978), VGSINGI (SEQ ID NO: 16200), AMSTNGR (SEQ ID NO: 16000), SASTNGV (SEQ ID NO: 16127), YMSTNGV (SEQ ID NO: 16042), YYSSNGV (SEQ ID NO: 16206), VHSTNGI (SEQ ID NO: 16134), PLSTNGV (SEQ ID NO: 16233), VYSTNGI (SEQ ID NO: 16059), IISTNGV (SEQ ID NO: 16054), RSVSSNGV (SEQ ID NO: 20502), YKSSNGV (SEQ ID NO: 16123), FRSTNGV (SEQ ID NO: 16070, and/or FVSTNGV (SEQ ID NO: 11162). In an example embodiment, the n-mer is selected from any one of the amino acid sequences of Tables 1-22, or any combination thereof. In an example embodiment, the n-mer motif is selected from the amino acid sequences of SEQ ID NO: 10952-20481 and 36241-42428. In an example embodiment, the targeting moiety is part of a viral capsid protein, including an AAV capsid.

In an example embodiment, the targeting moiety is inserted or substituted in loop IV, loop VIII, or both of an AAV capsid protein. In an example embodiment, the targeting moiety is IPFSRVNPDT (SEQ ID NO: 20285), LGFARTGAAD (SEQ ID NO: 20274), LGFTKSSGSD (SEQ ID NO: 20270), LRYSKTQGES (SEQ ID NO: 20266), SPYARSSAGV (SEQ ID NO: 20271), VGWSRLDLTT (SEQ ID NO: 20262). In an example embodiment, the n-mer is inserted between two amino acids of the one or more capsid proteins such that the n-mer is external to the AAV capsid when the protein comprising the n-mer is incorporated into the AAV capsid. In an example embodiment, the viral capsid protein is an AAV viral capsid protein. In an example embodiment, the n-mer is inserted between amino acids 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10. In an example embodiment, the targeting moiety is inserted between two consecutive amino acids within amino acids 451-460 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10. In an example embodiment, the capsid protein is VP1, VP2, VP3, or a combination thereof.

In an example embodiment, the cargo is a polynucleotide, one or more polypeptides, a ribonucleoprotein complex. In an example embodiment, the polynucleotide encodes one or more polypeptides and/or a RNAi oligonucleotide. In an example embodiment, the polynucleotide encodes one or more polypeptides. In an example embodiment, the one or more polypeptides comprise an enzyme or an antibody, including therapeutically useful enzyme or antibody (or antigen-binding form thereof). In an example embodiment, the polynucleotide encodes a CRISPR-Cas system. In an exemplary embodiment, the cargo is a recombinant AAV genome that is incorporated into a capsid comprising the n-mer and the recombinant AAV genome encodes a therapeutic protein or nucleic acid, including being operably linked to an appropriate regulatory sequence that directs expression of the protein or nucleic acid in the target tissue. In an example embodiment, the polynucleotide is operably linked to a regulatory sequences that promotes expression in the CNS.

In one aspect, A viral capsid or viral particle, comprising any composition as described herein. In an example embodiment, the viral capsid or viral particle further comprises a recombinant viral genome, wherein the recombinant viral genome encodes a therapeutic protein or nucleic acid, control polypeptide or nucleic acid, and/or selectable marker polypeptide or nucleic acid. In an example embodiment, the therapeutic protein or nucleic acid, control polypeptide or nucleic acid, and/or selectable marker polypeptide or nucleic acid is operably linked to a regulatory sequence that promotes expression in the CNS. In an example embodiment, the viral capsid or viral particle is an AAV viral capsid or AAV viral particle. In an example embodiment, the recombinant viral genome is a recombinant AAV viral genome. In an example embodiment, the targeting moiety is inserted between amino acids of 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10.

In one aspect, provided herein is a composition comprising one or more vectors encoding a targeting moiety effective to increase transduction of central nervous system tissues (CNS). In an example embodiment, a vector system comprising one or more vectors, wherein at least one of the one or more vectors encodes a targeting moiety effective to increase transduction of central nervous system tissues (CNS) via binding to a transferrin receptor (TFRC), and optionally wherein at least one of the one or more vectors encodes a recombinant AAV genome comprising a transgene encoding a protein or a polypeptide Also provided is a composition comprising a polypeptide or viral capsid comprising the targeting moiety, e.g., the n-mer that is effective to increase transduction of CNS tissues as described herein optionally further comprising a cargo, such as a recombinant AAV genome encoding a therapeutic protein or nucleic acid incorporated within, coupled to or otherwise associated with the targeting moiety containing polypeptide. In an example embodiment, the targeting moiety binds to the extracellular domain of TFRC. In an example embodiment, the targeting moiety binds to one or more of the apical, helical, and/or protease-like domain of the extracellular domain. In an example embodiment, the targeting moiety binds the apical domain. In an example embodiment, the targeting moiety comprises any n-mer motif described herein. In an example embodiment, the targeting moiety comprises a n-mer motif, the n-mer motif comprising or consisting of an amino acid sequence of Z₁-X₁-Z₂-X₂-X₃-X₄-X₅ wherein Z₁ comprises of Y, F, and L, Z₂ comprises of S, R, and K, X₁-X₅ are independently selected amino acids. In an example embodiment, X₁ optionally comprises of A, S, or H; X₂ optionally comprises of S, T, L, or I; X₃ optionally comprises N or G; X₄ optionally comprises G; X₅ optionally comprises of N, D, I, V, or R. In an example embodiment, the n-mer motif is selected from the group consisting of YSRIGPN (SEQ ID NO: 14632), YSRLNMN (SEQ ID NO: 14301), YSRLNKD (SEQ ID NO: 16577), and YHRLSNN (SEQ ID NO: 16636).

In an example embodiment, the targeting moiety encoded by a vector system or incorporated into a viral vector, including an AAV capsid, comprises a n-mer motif, the n-mer motif comprising or consisting of an amino acid sequence of is X₁-H-X₂-L-X₃-X₄-X₅ wherein X₁-X₅ are independently selected amino acids. In an example embodiment, the n-mer motif is VHRLQDK (SEQ ID NO: 16602) or LHALSHN (SEQ ID NO: 16608). In an example embodiment, the n-mer motif is comprises PSATNGV (SEQ ID NO: 20486), QVSTNGI (SEQ ID NO: 16021), SYSSNGV (SEQ ID NO: 16234), HQSSNGV (SEQ ID NO: 15978), VGSINGI (SEQ ID NO: 16199), AMSTNGR (SEQ ID NO: 16000), SASTNGV (SEQ ID NO: 16127), YMSTNGV (SEQ ID NO: 16042), YYSSNGV (SEQ ID NO: 16206), VHSTNGI (SEQ ID NO: 16134), PLSTNGV (SEQ ID NO: 16233), VYSTNGI (SEQ ID NO: 16059), IISTNGV (SEQ ID NO: 16054), RSVSSNGV (SEQ ID NO: 20502), YKSSNGV (SEQ ID NO: 16123), FRSTNGV (SEQ ID NO: 16070), and/or FVSTNGV (SEQ ID NO: 11162). In an example embodiment, provided herein are vectors or recombinant polypeptides, including, engineered AAV capsids wherein the n-mer is selected from any one as listed in any of Tables 1-22, or any combination thereof. In an example embodiment, provided herein are vectors wherein the n-mer motif is selected from SEQ ID NOS: 10952-20481 and 36241-42428. In an example embodiment, provided herein are vectors which encode the targeting moiety is part of a viral capsid protein.

In an example embodiment, the targeting moiety is inserted or substituted in loop IV and/or loop VIII of an AAV capsid protein. In an example embodiment, the targeting moiety is IPFSRVNPDT (SEQ ID NO: 20285), LGFARTGAAD (SEQ ID NO: 20274), LGFTKSSGSD (SEQ ID NO: 20270), LRYSKTQGES (SEQ ID NO: 20266), SPYARSSAGV (SEQ ID NO: 20271), VGWSRLDLTT (SEQ ID NO: 20262). In an example embodiment, provided herein are vectors wherein the n-mer is inserted between two amino acids of the one or more capsid proteins such that the n-mer is external to the AAV capsid. In an example embodiment, provided herein are vectors wherein the viral capsid protein is an AAV viral capsid protein. In an example embodiment, provided herein are vectors wherein the n-mer is inserted between amino acids 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10. In an example embodiment, the targeting moiety is inserted between two amino acids within amino acids 451-460 of a capsid protein of AAV9, or in an analogous position of a capsid protein from AAV1, AAV2, AAV3, AAV4, AAV5, AAV6, AAV7, AAV8, AAVrh8, AAV rh. 74, or AAV rh. 10. In an example embodiment, provided herein are vectors wherein the capsid protein is VP1, VP2, VP3, or a combination thereof.

In an example embodiment, provided herein are vectors wherein the cargo is a polynucleotide, one or more polypeptides, a ribonucleoprotein complex. In an example embodiment, provided herein are vectors wherein the polynucleotide encodes one or more polypeptides and/or a RNAi oligonucleotide. In an example embodiment, provided herein are vectors wherein the polynucleotide encodes one or more polypeptides. In an example embodiment, provided herein are vectors wherein the one or more polypeptides comprise enzymes or antibodies. In an example embodiment, the polynucleotide encodes a CRISPR-Cas system. In an example embodiment, the polynucleotide is operably linked to a regulatory sequence that promotes expression in the CNS.

In one aspect, provided herein is a composition comprising a polypeptide encoded or produced by the vector systems described herein. In an example embodiment, the polypide is a capsid protein, optionally an AAV capsid polypeptide. In one aspect, provided herein is a composition comprising a particle produced by the vector systems described herein. In an example embodiment, the particle is a viral particle, optionally an AAV particle. In one aspect, provided herein is a composition comprising a cell comprising the composition, vector, polypeptide, or particle described herein.

In one aspect, provided herein is a method of delivering one or more cargos to the CNS, comprising: administering, in vivo or in vitro, the engineered AAV capsid as described herein, or the vector as described herein. In an example embodiment, provided herein a method of delivery wherein the cargo is a recombinant AAV genome which encodes an RNAi oligonucleotide, a polynucleotide encoding a polypeptide, or a polypeptide, optionally operably linked to regulatory sequences that promote expression in the target tissue (such as the CNS). In an example embodiment, provided herein a method of delivery wherein the polypeptide includes an enzyme or antibody. In an example embodiment, provided herein a method of delivery wherein the cargo encodes a Cas polypeptide, a guide molecule, or both. In an example embodiment, provided herein a method of delivery wherein the cargo encodes a nuclease or a nucleic acid component of a RNA-guided nuclease. In an example embodiment, provided herein a method of delivery wherein the cargo is one or more polynucleotides encoding the nuclease and nucleic acid component of a RNA-guided nuclease.

In one aspect, a method of creating humanized transgenic non-human animals comprising: delivering to one or more cells of a non-human animal a vector system or a recombinant viral particle comprising a recombinant viral genome, wherein the vector system or recombinant viral genome encodes a human transferrin polypeptide, wherein the encoded human transferrin polypeptide is under the control of a tissue-specific promoter or miRNA binding element that has selective activity in the desired cell, tissue, or organ.

In example embodiments, the one or more cells are endothelial cells. In an example embodiment, the one or more cells are CNS cells. In example embodiments, the one or more cells are of the CNS vasculature, lungs, kidneys, liver, or any combination thereof. In example embodiments, the endothelial cells are of the CNS vasculature. In an example embodiment, the recombinant viral particle, optionally an AAV viral particle, comprises a capsid polypeptide, optionally an AAV capsid polypeptide, wherein the capsid polypeptide comprises a CNS specific n-mer motif. In an example embodiment, the CNS-specific n-mer motif comprises X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2. In an example embodiment, the CNS-specific n-mer motif comprises or consists of NNSTRGG (SEQ ID NO: 42429), GNSARNI (SEQ ID NO: 42430), and GNSVRDF (SEQ ID NO: 42431). In an example embodiment, the transgenic non-human animal is a rodent, optionally a mouse.

In one aspect, provided herein is a humanized transgenic non-human animal comprising: one or more cells expressing a human transferrin polypeptide, optionally wherein the one or more cells are CNS cells. In an example embodiment, the transgenic non-human animal is a rodent, optionally a mouse. A humanized transgenic non-human animal produced by any method described herein. In an example embodiment, the humanized non-human animal has a suppressed immune system.

In one aspect, provided herein is a method of screening n-mer motifs capable of conferring transduction of central nervous system (CNS) tissues via binding to a transferrin receptor (TFRC) in humanized transgenic non-human animals comprising: introducing one or more compositions comprising a candidate n-mer motif to humanized non-human transgenic animal described herein; and detecting binding of the compositions that bind to a transferrin receptor (TFRC) and/or detecting transduction or uptake by one or more CNS cells of the humanized transgenic non-human animal. In an example embodiment, the candidate n-mer motif comprises or consists of X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2. In an example embodiment, the composition is a viral particle comprising one or more capsid proteins each comprising the candidate n-mer motif.

In an example embodiment, the viral particle is an AAV viral particle and the one or more capsid proteins is an AAV capsid protein, optionally wherein the candidate n-mer motif is inserted in between amino acids 588-589 of an AAV9 capsid polypeptide or in an analogous position of a capsid protein from AAV1, AAV2, AAV3, AAV4, AAV5, AAV6, AAV7, AAV8, AAVrh8, AAV rh.74, or AAV rh. 10. In an example embodiment, at least one of the one or more compositions furthers comprises a cargo. In an example embodiment, the cargo is or encodes a therapeutic nucleic acid or polypeptide, a selectable marker, or a control polypeptide or nucleic acid.

In one aspect, a method of in vivo modeling comprising introducing a second vector system capable of targeting a transgenic polypeptide in a transgenic non-human animal as described herein. In example embodiments, the non-human animal has a suppressed immune system.

The method of screening vector systems capable of transduction of central nervous system (CNS) tissues via binding to a transferrin receptor (TFRC) in humanized transgenic non-human animals comprising: (a) introducing a plurality of vector systems to one or more humanized transgenic non-human animal and (b) detecting the vector systems that bind to a transferrin receptor (TFRC).

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Structural model of AAV9 highlighting the regions modified. The model highlights the loop IV residues where substitution mutagenesis was performed in some examples (black) and the residues (AA588-589) between which loop VIII insertions were made (dark gray) in other examples. The K449R mutation is shown in green.

FIG. 2A-2B—Capsids that bind human TFRC are capable of dramatically enhanced binding and transduction of CHO cells expressing human TFRC. The indicated AAVs packaging an AAV-CAG-GFP-2A-Luciferase-WPRE-pA genome were administered to control CHO cells or CHO cells expressing human TFRC (hTFRC) at 10K vg/cell. (A). The graph shows binding to the indicated CHO cells with the indicated AAV. (B) The graph shows the transduction efficiency normalized to AAV for the indicated AAV. Transduction was measured 24 hours after virus administration with a luciferase reporter assay.

FIG. 3—Surface human TFRC protein levels on hCMEC/D3 cells with a lentivirus expressing TFRC. Cells transduced with the indicated MOI of VSV-G pseudotyped lentivirus expressing human TFRC were incubated at 4C with an antibody that binds TFRC. The cells were then fixed without membrane permeabilization and immunostained with anti-TFRC (ThermoFisher, cat #14-0719-82) to assess the levels of cell surface exposed TFRC. Images show immunostaining and cell nuclei.

FIG. 4—TFRC binding capsids more efficiently transduce hCMEC/D3 cells, and binding and transduction of hCMEC/D3 cells are further increased by elevating TFRC expression by lentiviral delivery of TFRC. The indicated AAVs were administered to both control and human TFRC expressing hCMEC/D3 cells at 5,000 vg/cell. Binding and transduction were assessed as described in FIG. 2.

FIG. 5A-5B—The enhanced transduction of hCMEC/D3 cells by AAV-BI19 relative to AAV9 is blocked by a human TFRC antibody. The AAV9 or AAV-BI19 were used to package AAV-CAG-GFP-2A-Luciferase-WPRE-pA genome. The indicated AAVs were administered to hCMEC/D3 cells at 2,000K vg/cell in the absence or presence of the indicated concentration of monoclonal anti-TFRC antibody. Transduction (A) was assessed 24 hours after virus administration, and binding (B) was assessed at 1 hour.

FIG. 6—Expression of human TFRC in mouse brain endothelial cells in vivo increases CNS transduction by AAV-BI19. (Left) The schematic shows the experimental design. In the 2-step transduction assay, step one is the delivery of the target receptor gene to brain endothelial cells in NSG mice using intravenous administration of AAV-BI30 (Krolak et al. Nature Cardiovascular Research 2022). In this experiment, the target receptor is human TFRC or mouse LY6A, which is used as a positive control (LY6A is required for efficient entry of AAV-PHP.eB to the CNS and is deficient in NSG mice. 7 Days later, the second virus (either AAV-BI19:CAG-mScarlet, which binds hTFRC, or AAV-PHP.eB:CAG-mScarlet, which binds LY6A) or is delivered. Delivering AAV-BI19 to animals that received the non-target receptor, AAV-BI30:LY6A (upper right), and AAV-PHP.eB to animals that received hTFRC serve as negative controls (lower left). CNS transduction by AAV-BI19 is enhanced in animals that previously received the AAV-BI19 receptor hTFRC (lower right). Images show transduction in the brains of mice 3 weeks after delivery of the second virus. All AAVs were delivered at 1e11 vg/animal.

FIG. 7—Representative SDS-PAGE showing Fc-control (Fc-ctrl), human TFRC-Fc (hTFRC), mouse TFRC-Fc (mTFRC), and marmoset TFRC-Fc (marTFRC). The indicated proteins were produced in HEK293 cells and pulled down with ProA-magnetic beads. The proteins were then separated by SDS-PAGE and stained for total protein using SyPro Ruby.

FIG. 8A-8B—Clustering analysis of 7-mer sequences that bind human TFRC. AAV 7-mer insertions found to interact with human TFRC were found to form distinct sequence clusters. (A) Sequences were one-hot encoded, projected with UMAP, and clustered with a Gaussian mixture model (k=30). (B) Cluster sequence logos show the AA frequency in each cluster, the cluster number (k) and the number (n) of sequences per cluster. A list of sequences in each cluster is provided in Table 12.

FIG. 9—Individual binding to CHO cells stably expressing TFRC.

FIG. 10—The enhanced transduction of hCMEC/D3 cells by BI-19 relative to AAV9 is blocked by an anti-human TFRC antibody.

FIG. 11—Overexpression of TFRC on mouse brain vasculature in vivo increases BI-19 transduction in the CNS.

FIG. 12A-12B—BI19 is actively transported across a human brain vascular layer in vitro. hCMEC cells were cultured until confluent on a Transwell insert. A pool of viruses was added to the top chamber, and the fraction of the vector found in the bottom chamber was assessed at the indicated time by qPCR. Virus was added to cells at 37 C or at 4 C. The 4 C control demonstrates the amount of the viral particles that enter the bottom chamber without being actively transported. (B) The graphs provide the fraction of AAV genomes found in the bottom chamber at 37C divided by the fraction of the AAV genomes found in the bottom chamber at 4 C.

FIG. 13A-13C—Expression of human TFRC in mouse brain endothelial cells in vivo increases CNS transduction by AAV-BI19. (A) The schematic shows the experimental design. In the 2-step transduction assay, step one is the delivery of the target receptor gene to brain endothelial cells in NSG mice using intravenous administration of AAV-BI30 (Krolak et al. Nature Cardiovascular Research 2022). In this experiment, the target receptor is human TFRC or mouse LY6A, which is used as a positive control (LY6A is required for efficient entry of AAV-PHP.eB to the CNS and is deficient in NSG mice). (B) Images show sagittal sections of the mouse brain transduced by AAV-PHP.eB (left) or AAV-BI19 (right) in mice made to express mouse LY6A or human TFRC through delivery of the genes encoding these proteins by AAV-BI30. AAV-BI30:CAG-human TFRC or AAV-BI30:CAG-mouse LY6A vectors were administered intravenously to adult NSG mice at 1e11 vg/animal. 28 days later, the second virus, either AAV-BI19, which binds hTFRC or AAV-PHP.eB, which binds mouse LY6A, was delivered at a dose of 5e11 vg/animal. Both vectors packaged the same AAV genome (AAV-CAG-NLS-mScarlet-2A-Luciferase-pA). CNS transduction by AAV-BI19 is enhanced in animals that previously received the AAV-BI19 receptor hTFRC (lower right). Delivering AAV-BI19 to animals that received the non-target receptor, AAV-BI30: LY6A (upper right), and AAV-PHP.eB to animals that received hTFRC serve as negative controls (lower left). Images show transduction (assessed by imaging native mScarlet fluorescence) in the brains of mice three weeks after delivery of AAV-PHP.eB or AAV-BI19.

FIG. 14A-14D—AAV-BI19 more efficiently crosses the BBB and transduces neurons throughout the brain in mice where the extracellular domain of human TFRC was knocked into the mouse Tfrc gene. (A-B) 8e11 vg/mouse of AAV-BI19 or AAV9 packaging CAG-NLS-mScarlet-2A-Luciferase-pA was systemically administered (injection into the retro-orbital sinus) to adult huTFRC KI mice (B-hTFRC, CytoBiogen). Expression was assessed seven days after AAV administration. (A-B) The images show matched exposures of native nuclear mScarlet expression in whole sagittal brain sections from homozygous B-hTFRC mice after transduction with AAV-BI19 (A) or AAV9 (B). (C-D) AAV-BI19 (C) or AAV9 (D) transduction of neurons (NeuN immunostaining) in the brain of huTFRC KI mice. The mScarlet exposures in C and D were not matched. The mScarlet exposures in D were longer in order to show transduction by AAV9.

FIG. 15—AAV-BI19 transduces human brain endothelial cells in the presence of human holo-Transferrin. Cells from the hCMEC human brain endothelial cell line were preincubated with or without human Transferrin at the indicated concentration and then transduced by AAV-BI19 or AAV9 packaging CAG-GFP-2A-Luciferase (5000 vg/cell) and assessed for transduction 24 hours later by measuring Luciferase activity. The graph shows transduction normalized relative to AAV9 in the absence of human Transferrin.

FIG. 16A-16E—AAV9 can be reprogrammed to bind human TfR1. (16A) AAV9-based 7-mer NNK capsid libraries (random 7-mer insertion between residues 588-589 in VP1) were screened for selective binding of huTfR1 as an Fc fusion protein or when transiently overexpressed on HEK293 and CHO cells. (16B) (SEQ ID NOS: 42456-42459) Four huTfR1-binding variants that shared a common motif were identified. Each variant was individually produced to carry a CAG-NLS-mScarlet-p2A-Luc-WPRE construct. They exhibited enhanced species-specific binding (16C) and transduction (16D) of CHO cells stably expressing TFRC from human but not macaque, marmoset, or mouse. For the binding assay, each cell line was seeded at 30,000 cells per well and, 24 hours later, each well was subjected to a media change with the fresh media that contained each AAV9 variant carrying CAG-NLS-mScarlet-P2A-Luciferase-SV40-WPRE at 3,000 vg/cell. The cells were maintained at 4° C. with gentle rocking for one hour and washes with PBS. Then, DNA was extracted and subjected to qPCR targeting mScarlet. For the transduction assay, each cell line was seeded at 100,000 cells per well and, 24 hours later, each well was subjected to a media change with the fresh media that contained each AAV9 variant carrying CAG-NLS-mScarlet-P2A-Luciferase-SV40-WPRE at 3,000 vg/cell. The cells were maintained at 37° C. with 5% CO₂ for 24 hours. Then, transduction was measured using the britelite Plus reporter gene assay system. A one-way ANOVA test was performed to assess significant differences; **** indicates P<0.0001. (16E) Characterization of the binding kinetics between AAV-BI19 and TfR1 in the presence or absence of Holo-Tf. Fitting of Bio-Layer Interferometry (BLI) traces of immobilized AAV-BI19 capsids incubated at 30° C. with 6.25-200 nM of Peptidisc-reconstituted huTfR1 showed a strong binding between the variant and the receptor (top left), whereas AAV9 capsids demonstrated no significant association with huTfR1 (top right). The effect of Holo-Transferrin (Holo-Tf) on AAV-BI19 binding was tested by incubation of AAV-BI19 capsids with 6.25-200 nM of Peptidisc-reconstituted huTfR1 in the presence of a molar excess of 200 nM Holo-Tf (bottom left). AAV-BI19 binds to huTfR1-in the presence of Holo-Tf. AAV9 showed no significant binding to huTfR1 regardless of the presence of Holo-Tf (bottom right).

FIG. 17A-17B—TfR1 expression in CHO cells. Applicants established stable CHO cell lines expressing human, macaque, marmoset, or mouse TFRC via random lentiviral integration of each exogenous construct under the control of an EF-1 alpha promoter. (17A) RNA was extracted from each cell line, reverse transcribed, and the cDNA was subjected to qPCR using species-specific primers (Table 23). (17B) Each cell line was immunostained with anti-TfR1 antibodies: abcam ab84036, polyclonal, targeting the N-terminal (mostly intracellular) domain of huTfR1, or OKT9, monoclonal, targeting the apical domain of huTfR1.

FIG. 18A-18D—TfR1-targeted capsids transduce human brain endothelial cells via interactions with huTfR1. The TfR1-targeted variants exhibited enhanced binding (18A) and transduction (18B) of human brain microvascular endothelial cells (hBMVEC) and hCMEC/D3 cells. For the binding assay, hBMVEC or hCMEC/D3 cells were seeded at 15,000 or 30,000 cells per well, respectively, and, 24 hours later, each well was subjected to a media change with the fresh media that contained each AAV9 variant carrying CAG-NLS-mScarlet-P2A-Luciferase-SV40-WPRE at 12,000 or 6,000 vg/cell for hBMVEC or hCMEC/D3 cells, respectively. The cells were maintained at 4° C. with gentle rocking for one hour. Then, DNA was extracted and subjected to qPCR targeting mScarlet. For the transduction assay, hBMVEC or hCMEC/D3 cells were seeded at 5,000 or 10,000 cells per well, respectively, and, 24 hours later, each well was subjected to a media change with the fresh media that contained each AAV9 variant carrying CAG-NLS-mScarlet-P2A-Luciferase-SV40-WPRE at 12,000 or 6,000 vg/cell for hBMVEC or hCMEC/D3 cells, respectively. Transduction was measured using the britelite Plus reporter gene assay system 24 hours after AAV addition. A two-tailed t-test was performed to assess significant differences; ****, ***, **, and * indicate P<0.0001, <0.001, <0.01, and <0.05 respectively. (18C) AAV-BI19 transduction of hCMEC/D3 cells was inhibited by the OKT9 antibody that targets the huTfR1 apical domain but not an antibody that targets the Transferrin binding domain of huTfR1. The hCMEC/D3 cells were seeded at 7,500 cells per well. Two days later, 100 μL of media with 3e8 vg/mL of the indicated AAV and the specified concentration of OKT9 or R&D AF2474 antibody was transferred to each well. Transduction was measured 24 hours later using the britelite Plus reporter gene assay system. A two-way ANOVA test was performed to assess significant differences; **** indicates P<0.0001. (18D) AAV-BI19 is actively transported across an endothelial cell barrier. Transwell inserts were seeded with hCMEC cells and grown to confluence. Barcoded AAV-BI19, AAV9, and AAV2 were pooled and applied to the upper chamber of the transwell at 25,000 vg/cell/AAV in media heated to 37° C. or chilled to 4° C. as indicated. The amount of virus transported across the transwell at three hours at the indicated temperatures was quantified by qPCR and normalized to the corresponding starting titer. Data is represented as four replicates±SEM. An unpaired t-test was performed to assess significant differences; * indicates P<0.05 and ns indicates no statistical difference. A dashed line is drawn at 10,000 vg, below which there is reduced ability to accurately quantify the amount of virus.

FIG. 19A-19D—AAV-BI19 binding to the TfR1 apical domain depends on Y247 and T248 residues present in the human protein but not macaque or mouse. (19A) (SEQ ID NOS: 42460-42462) Alignment of the human, macaque, and marmoset TfR1 apical domain amino acid sequences from residue 200-380. The differences from the human sequence are highlighted in gray. Amino acid differences between human and macaque proteins are highlighted in four groups (colored outlines, 19A) and on Alphafold2 structural models (19B). (19C) CHO cells were transiently transfected to express the wild type human TfR1 (huTfR1) or variants with the indicated substitutions where the human residue was replaced by the corresponding macaque residue or (19D) the wild type macaque TfR1 (macTfR1) or a variant with D247Y and S248T substitutions (n=4 replicate transfections per plasmid±SEM shown). Cells were maintained at 37° C. with 5% CO₂. At 24 hours post-transfection, AAV-BI19 or AAV9 encoding ssCAG-NLS-mScarlet-P2A-Lucierase-pA was applied at 10,000 vg/cell. After 24 hours, viral transduction was measured using the britelite Plus reporter gene assay system. For (19C), a one-way ANOVA was performed to determine significant differences in transduction among the groups using the wild type huTfR1 as the control. To account for multiple comparisons, a Dunnett's correction was applied. Only those comparisons with a P≤0.05 are reported with an asterisk (* or **** indicate P≤0.05 or ≤0.0001, respectively). For (19D), an unpaired two-tailed t-test was performed between the two groups (** indicates P≤0.01).

FIG. 20—TfR1 expression in B-hTFR1 mice. RNA was extracted from the cortex and spinal cord regions of C57BL/6J or B-hTFR1 mice that had been treated with either AAV9 or AAV-BI19. The RNA was reversed transcribed and the cDNA was subjected to qPCR using primers spanning exon 1 and exon 2 of the mouse Tfrc gene (Table 23). Note that, in B-hTFR1 mice, exons 4-19 of the mouse Tfrc gene that encode the extracellular region have been replaced by the corresponding human exons. TfR1 expression was not found to be significantly different between C57BL/6J and B-hTFR1 mice (two-way ANOVA).

FIG. 21A-21I—AAV-BI19 efficiently delivers genes to the CNS in knock-in mice expressing the huTfR1 extracellular domain. AAV-BI19 or AAV9 encoding ssCAG-NLS-mScarlet-P2A-Luciferase-pA was intravenously injected into adult female C57BL/6J or C57BL/6-Tfr1tm1TFR1/Bcgen (B-hTFR1) mice at 5×10¹¹ vg/mouse. After three weeks, the (21A) biodistribution of vector genomes per mouse genome, (21B) mScarlet transcript levels relative to AAV9 in C57BL/6J mice, and (21C) luciferase activity relative to AAV9 in C57BL/6J were measured within different organs. For AAV9 in C57BL/6J and AAV-BI19 in B-hTFR1, n=4 mice per group, ±SEM shown. For AAV9 in B-hTFR1 and AAV-BI19 in C57BL/6J n=3 mice per group, ±SEM shown. A two-way ANOVA was performed to determine significant differences within organs among the four groups of mice with AAV-BI19 in B-hTFR1 as the main comparison group and using a Bonferroni multiple comparison correction; ****, ***, **, and * indicate P≤0.0001, P≤0.001, P≤0.01, and P≤0.05, respectively, between AAV-BI19 in B-hTFR1 and each of the other groups of mice. Representative (21D) whole brain and (21E) spinal cord images of the C57BL/6J and B-hTFR1 mice treated with AAV-BI19 or AAV9 encoding ssCAG-NLS-mScarlet-P2A-Luciferase-pA are shown at a dose of 5e11 vg per mouse. Native mScarlet fluorescence was assessed three weeks post injection. Images show the cells transduced by AAV-BI19 overlaid with (21F) NeuN⁺ neurons or (21G) SOX9⁺ stained cells in the cortex, thalamus, and striatum of B-hTFR1 mice. The percentage of (21H) NeuN⁺ neurons or (21I) SOX9⁺ astrocytes that expressed mScarlet in the cortex, striatum, and thalamus were assessed (n=3 mice for AAV9 in B-hTFR1 and AAV-BI19 in C57BL/6J mice, n=4 mice for AAV9 in C57BL/6J and AAV-BI19 in B-hTFR1 mice; error bars indicate±SEM). A two-way ANOVA was performed to determine significant differences in transduction within specific brain regions among the four groups of mice across the two capsids with AAV-BI19 in B-hTFR1 as the main comparison group and using a Bonferroni multiple comparison correction. The **** indicates P≤0.0001 between AAV-BI19 in B-hTFR1 and each of the three other groups of mice (AAV9 in C57BL/6J, AAV9 in B-hTFR1, and AAV-BI19 in C57BL/6J).

FIG. 22A-22B—AAV-BI19 exhibits similar transduction to AAV9 in the liver and dorsal root ganglion in B-hTFR1 mice. ssAAV-BI19:CAG-NLS-mScarlet-P2A-Lucierase-pA or ssAAV9:CAG-NLS-mScarlet-P2A-Luciferase-pA was intravenously injected into adult female C57BL/6J or B-hTFR1 mice at a dose of 5×10¹¹ vg/mouse. At three weeks post-injection, native fluorescence was assessed in the (22A) liver and (22B) dorsal root ganglion.

FIG. 23—Expression of huTfR1 in mouse brain endothelial cells in vivo increases CNS transduction by AAV-BI19. In the 2-step transduction assay, step one is the delivery of the target receptor gene to brain endothelial cells in NSG mice using intravenous administration of AAV-BI30 (Krolak, T., et al. (2022). A high-efficiency AAV for endothelial cell transduction throughout the central nervous system. Nature Cardiovascular Research 1, 389-400). In this experiment, the target receptors were human TfR1 (huTfR1, encoded by the TFRC gene) or mouse LY6A, which was used as a positive control as LY6A is required for the efficient entry of AAV-PHP.eB into the CNS and is deficient in NSG mice. AAV-BI30 was systemically administered at 1e11 vg/mouse to deliver AAV-CAG-hTFRC-WPRE-3x-miR122-pA (top) or AAV-CAG-LY6A-WPRE-3x-miR122-pA (bottom). At 28 days post-injection, the second virus (5e11 vg/mL of either AAV-BI19:CAG-mScarlet, which binds huTfR1 (left), or AAV-PHP.eB:CAG-mScarlet, which binds LY6A (right) was systemically administered. This experiment was performed with three mice per condition. Representative images show the AAV transduction in sagittal mouse brain sections of mice three weeks after the delivery of the second virus. CNS transduction by AAV-PHP.eB and AAV-BI19 was enhanced in the animals that were previously treated with AAV-BI30: LY6A (bottom right) or AAV-BI30: hTFRC (top left), respectively. AAV-BI19 did not exhibit enhanced CNS transduction in the animals that had received the non-target receptor, AAV-BI30: LY6A (bottom left). As a negative control, AAV-PHP.eB also did not exhibit enhanced CNS transduction in the animals that had received AAV-BI30: hTFRC (top right).

FIG. 24—The vast majority of capsids with 7-mer sequences conforming to the motif families and genera defined in Table 21 bind human TFR1-Fc fusion proteins. For each family/genus, the percentage of sequences contained within the motif that were measured in the library is provided within the x-axis label.

FIG. 25—Extensive functional characterization of capsids that bind human TFR1. Capsids were selected based on human TFR1-Fc binding and enhanced transduction activity in one or more of the following in vivo assays: transduction of the B-hTFR1 KI mouse brain or spinal cord transduction or the 2-step assay with BI30:CAG-humanTFRC. The capsids with enhanced in vivo tropisms in human TFRC expressing mice, were then assessed for additional functions relevant to the selection of top capsid candidates. Note the vast majority of selected sequences showed elevated enrichment relative to AAV9 for the following assays: B-hTFR1 KI (brain and spinal cord), 2-step hTFRC brain, human TFR1-Fc binding, binding to CHO cells expressing human TFRC (CHO-hTFRC), and binding to hCMEC and hCMEC-hTFRC cells. In contrast these capsids were not positively enriched for transduction of control mice (2-step mice transduced with AAV-BI30-CAG-LY6A). Left, AAV was encoded into the library with 5 nucleotide sequences at 1×, 10×, 100× copies relative to other capsid sequences in the library to facilitate accurate comparisons in assays where AAV9 at 1× performs poorly, e.g., in vivo CNS transduction.

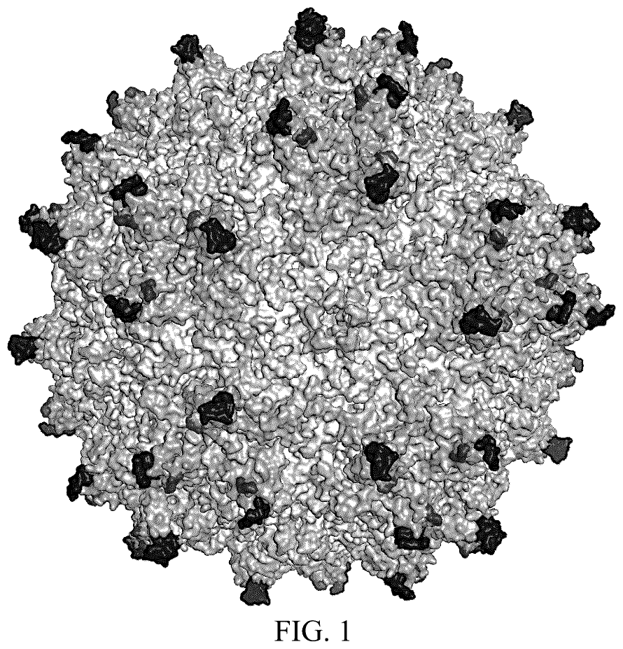

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment”, “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide targeting moieties which promote transduction into the CNS through interaction with the Transferrin receptor. These targeting moieties may be incorporated into particles, such as viral capsid delivery particles, to confer tropism on the delivery particles and promote transduction of CNS. Example CNS tissue include brain and spinal cord tissue. Example CNS cell types include neurons and glial cells. Further embodiments disclosed herein provide a vector system comprising one or more vectors encoding AAV capsids according to embodiments described herein. Accordingly, embodiments disclosed herein provide compositions capable of delivering cargos with enhanced selectivity and efficiency to the CNS vasculature. Embodiments disclosed herein also provide vector systems for the generation and loading of such delivery particles with a cargo. Likewise, embodiments disclosed herein provide methods for use of such compositions to target CNS endothelial cells, in vitro and in vivo, with implications for both therapeutic and research purposes.

Additional feature and advantages of the aforementioned embodiments are further described below.

### Targeting Moieties and Compositions Thereof

In example embodiments, compositions are provided herein comprising a targeting moiety with an enhanced tropism for endothelial cells of the CNS. A target moiety with an enhanced tropism for endothelial cells of the CNS promotes, increases, or otherwise improves binding to, and in some cases, transduction of the CNS as compared to a natural or wild-type target moiety. This targeting moiety may be coupled directly to a cargo to be delivered such as an oligonucleotide or polypeptide. Alternatively, the targeting molecule may be incorporated into a delivery particle to confer tropism for endothelial cells of the CNS on the delivery particle. A non-limiting example of delivery particle is a viral capsid particle. In such embodiments, the targeting moiety may be incorporated into a viral capsid polypeptide such that the targeting moiety is incorporated into the assembled viral capsid. However, other particle delivery systems where the targeting moiety may be incorporated or attached, for example on exosomes or liposomes, are also envisioned and encompassed as alternative embodiments herein.

In a preferred embodiment, provided herein is a composition comprising a targeting moiety effective to increase transduction of central nervous system (CNS) tissues via binding to a transferrin receptor (TFRC), optionally further comprising a cargo coupled to or otherwise associated with the targeting moiety. A target moiety with an increased transduction promotes, enhances, or otherwise improves binding to, and in some cases, transduction of the CNS as compared to a natural or wild-type target moiety. In an example embodiment, the targeting moiety binds to the extracellular domain of TFRC. In an example embodiment, the targeting moiety binds to one or more of the apical, helical, and/or protease-like domain of the extracellular domain. In an example embodiment, the targeting moiety binds to the apical domain. In an example embodiment, the n-mer is an animo acid sequence of length n. The length of the n-mer may be any necessary length to transduce the CNS. In example embodiments, the n-mer is 1, 2, 3, 4, 5, 6, 7, 8, 9, 10, 11, or 12 amino acids in length. In an example embodiment, the n-mer motif has a length of at least 7 amino acids. In an example embodiment, a composition comprising a targeting moiety effective to increase transduction of CNS tissues comprises a n-mer motif, the n-mer motif comprising or consisting of an amino acid sequence of Z₁-X₁-Z₂-X₂-X₃-X₄-X₅ wherein Z₁ is Y, F, or L, Z₂ is S, R, or K, X₁-X₅ are independently selected amino acids. In an example embodiment, X₁ optionally comprises of A, S, or H; X₂ optionally comprises of S, T, L, or I; X₃ optionally comprises N or G; X₄ optionally comprises G; X₅ optionally comprises of N, D, I, V, or R. In an example embodiment, the targeting moiety comprises a n-mer motif, the n-mer motif comprising or consisting of an amino acid sequence of is X₁-H-X₂-L-X₃-X₄-X₅ wherein X₁-X₅ are independently selected amino acids.

In example embodiments, the n-mer can be used to increase transduction in target cells i.e., CNS cells and tissues. The increase in transduction efficiency (which may correspond to the tropism efficiency) of the n-mer to a cell may be compared to a composition that does not contain the targeting moiety for example inclusion of one or more targeting moieties in a composition can result in an increase in transduction and or transduction efficiency by 10%, 20%, 30%, 40%, 50%, 60% 70% 80% 90% a 100% or more. In an exemplary embodiment the increase in transduction and or transduction efficiency is one and a half fold, two-fold, three-fold, four-fold, five-fold, six-fold, seven-fold, eight-fold, nine-fold, ten-fold, 20-fold, 30-fold, 40-fold, 50-fold, 60-fold, 70-fold, 80-fold or more relative to a composition lacking the n-mer. In one embodiment the transduction and/or transduction efficiency is increased or enhanced in endothelial cells, in one embodiment increase in endothelial cells of the vasculature, for example, the central nervous system vasculature. In embodiments, the transduction and/or transduction efficiency is increased or enhanced in cells of the central nervous system. In embodiments, the transduction and/or transduction efficiency is increased or enhanced in neurons and glial cells. In an embodiment, the composition comprising a n-mer is selective to a target cell as compared to other cell types and/or other virus particles. As used herein, ‘selective’ and ‘cell-selective’ refers to preferential targeting for cells as compared to other cell types. Preferably, the targeting moiety is selective for a desired target (e.g. cell, organ, system e.g. CNS tissues) or set of targets by at least 2:1, 3:1, 4:1, 5:1, 6:1 7:1, 8:1, 9:1. 10:1 or more; or 10%, 20%, 30%, 40%, 50%, 60%, 70%, 75% 80%, 85% 90% or more, relative to other targets or cells (e.g. CNS). In an example embodiment, the composition comprising a targeting moiety described herein can have an increased uptake, delivery rate, transduction rate, efficiency, amount, or a combination thereof in a target cell (e.g., endothelial cells across the CNS e.g., brain endothelium, arterio-venous axis in brain, retina, and spinal cord vasculature) as compared to other cells types (e.g. muscle cells) and/or other virus particles (e.g., AAVs not containing the targeting moiety) and other compositions that do not contain the cell-selective n-mer motif of the present invention.

In an example embodiment, the n-mer motif is selected from the group consisting of YSRIGPN (SEQ ID NO: 14632), YSRLNMN (SEQ ID NO: 14301), YSRLNKD (SEQ ID NO: 16577), and YHRLSNN (SEQ ID NO: 16636). In an example embodiment, the n-mer motif is VHRLQDK (SEQ ID NO: 16602) or LHALSHN (SEQ ID NO: 16608). In an example embodiment, the n-mer motif is comprises PSATNGV (SEQ ID NO: 20486), QVSTNGI (SEQ ID NO: 16021), SYSSNGV (SEQ ID NO: 16234), HQSSNGV (SEQ ID NO: 15978), VGSINGI (SEQ ID NO: 16199), AMSTNGR (SEQ ID NO: 16000), SASTNGV (SEQ ID NO: 16127), YMSTNGV (SEQ ID NO: 16042), YYSSNGV (SEQ ID NO: 16206), VHSTNGI (SEQ ID NO: 16134), PLSTNGV (SEQ ID NO: 16233), VYSTNGI (SEQ ID NO: 16059), IISTNGV (SEQ ID NO: 16054), RSVSSNGV (SEQ ID NO: 20502), YKSSNGV (SEQ ID NO: 16123), FRSTNGV (SEQ ID NO: 16070, and/or FVSTNGV (SEQ ID NO: 11162). In an example embodiment, the n-mer is selected from any of the amino acid sequences in Tables 1-13, or any combination thereof. In an example embodiment, the n-mer motif is selected from a peptide having an amino acid sequence of one of SEQ ID NO:10952-20481. In an example embodiment, the targeting moiety is part of (e.g., inserted between consecutive amino acids of) a viral capsid protein, including an AAV capsid protein.

### Transferrin Receptor (TFRC) Binding

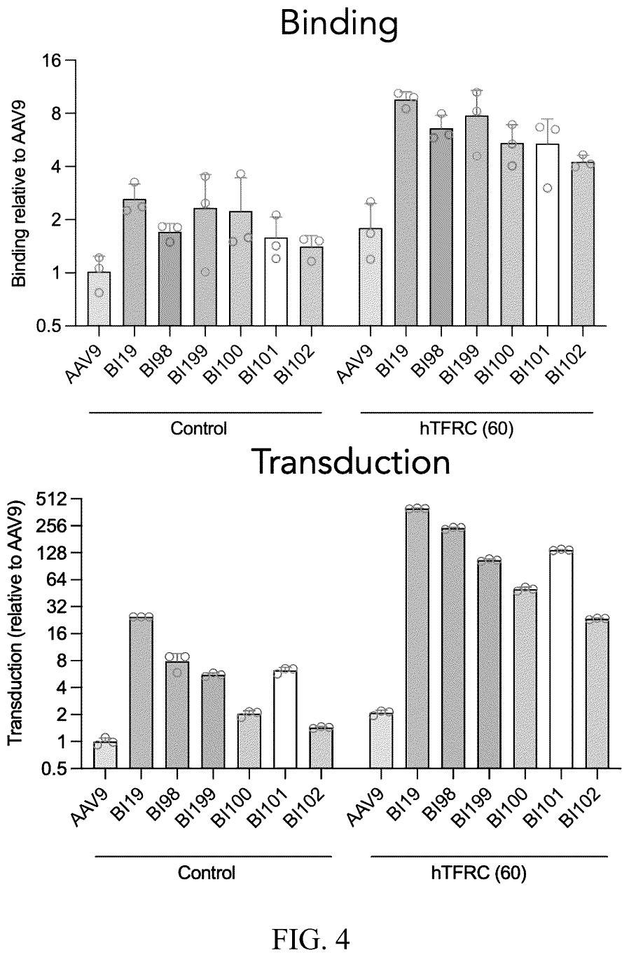

In a preferred embodiment, the targeting moiety binds to a Transferrin receptor. The TFRC (i.e., TfR1 protein encoded by the TFRC gene, or CD71) comprises of two types of receptors: TFRC1 (or cluster of differentiation 71 (CD71)) and TFRC2. TFRC2 is less common than TFRC1 and is predominantly expressed in hepatocytes. TFRC1 binds transferrin (TF) with high affinity and is commonly expressed. TFRC1 is a type II transmembrane glycoprotein of around 90 kDa and comprises of around 760 amino acids. TFRC1 is typically found as a dimer linked by disulfide bonds on the cell surface, see FIG. 4.

The TFRC1 domain contains an extracellular C-terminal domain (around 671 amino acids) and comprises of the TF binding site. The extracellular C-terminal domain comprises of three subdomains: apical, helical, and protease-like domain, see FIG. 4. Furthermore, the extracellular C-terminal domain comprises of three N-linked glycosylation sites at asparagine residues 251, 317, and 727 and one O-linked glycosylation site at threonine 104, which contribute to adequate function of the receptor. TFRC1 additionally comprises of a transmembrane domain (around 29 amino acids), and an intracellular N-terminal domain (around 61 amino acids). Alternative to the delivery of iron through TF with TFRC1, uptake of iron can also occur through H-ferritin binding through the apical domain. See also Candelaria, P. V.; et al. Antibodies Targeting the Transferrin Receptor 1 (TfR1) as Direct Anti-Cancer Agents. Frontiers in Immunology, 2021, 12, hereby incorporated by reference.

In an example embodiment, the targeting moiety binds to the extracellular domain of TFRC. In an example embodiment, the targeting moiety binds to one or more of the apical, helical, and/or protease-like domain. In an example embodiment, the targeting moiety binds to the apical domain.

### Engineered Viral Capsids

Described herein are various embodiments of engineered viral capsids, such as adeno-associated virus (AAV) capsids, that can be engineered to confer cell-selective tropism, such as CNS tissue- and cell-specific tropism, to an engineered viral particle. Engineered viral capsids can be lentiviral, retroviral, adenoviral, or AAV capsids. The engineered capsids can be included in an engineered virus particle (e.g., an engineered lentiviral, retroviral, adenoviral, or AAV virus particle), and can confer cell-selective tropism to the engineered viral particle. The engineered viral capsids described herein can include one or more engineered viral capsid proteins described herein. The engineered viral capsids described herein can include one or more engineered viral capsid proteins described herein that can contain one or more target moiety as described elsewhere herein.

The engineered viral capsids can be variants of wild-type viral capsid. For example, in some embodiments, the engineered AAV capsids can be variants of wild-type AAV capsids. In some embodiments, the wild-type AAV capsids can be composed of VP1, VP2, VP3 capsid proteins or a combination thereof. In other words, the engineered AAV capsids can include one or more variants of a wild-type VP1, wild-type VP2, and/or wild-type VP3 capsid proteins. In some embodiments, the serotype of the reference wild-type AAV capsid can be AAV-1, AAV-2, AAV-3, AAV-4, AAV-5, AAV-6, AAV-8, AAV-9 or any combination thereof. In some embodiments, the serotype of the wild-type AAV capsid can be AAV-9. The engineered AAV capsids can have a different tropism than that of the reference wild-type AAV capsid.

In some embodiments, the target moiety is incorporated into a viral protein, such as a capsid protein, including but not limited to lentiviral, adenoviral, AAV, bacteriophage, retroviral proteins. In some embodiments, the target moiety is located between two amino acids of the viral protein such that the target moiety is external (i.e., is presented on the surface of) to a viral capsid. In an example embodiment, the target moiety disclosed herein can be inserted between two consecutive amino acids in the wild-type viral protein (VP) (or capsid protein), including in regions that are surface exposed when incorporated into a viral capsid. In some embodiments, the target moiety can be inserted between two consecutive amino acids in a variable amino acid region in a viral capsid protein.

In some embodiments, the target moiety can be inserted between two consecutive amino acids in a variable amino acid region in an AAV capsid protein. The core of each wild-type AAV viral protein contains an eight-stranded beta-barrel motif (betaB to betaI) and an alpha-helix (alphaA) that are conserved in autonomous parvovirus capsids (see e.g., DiMattia et al. 2012. J. Virol. 86 (12): 6947-6958). Structural variable regions (VRs), also referred to as “loops”, occur in the surface loops that connect the beta-strands, which cluster to produce local variations in the capsid surface. AAVs have 12 variable regions (also referred to as hypervariable regions) (see e.g., Weitzman and Linden. 2011. “Adeno-Associated Virus Biology.” In Snyder, R. O., Moullier, P. (eds.) Totowa, NJ: Humana Press). In one example embodiment, one or more target moiety can be inserted between two amino acids in one or more of the 12 variable regions in the wild-type AVV capsid proteins. In one example embodiment, the one or more target moieties can be each be inserted between two amino acids in VR-I, VR-II, VR-III, VR-IV, VR-V, VR-VI, VR-VII, VR-III, VR-IX, VR-X, VR-XI, VR-XII, or a combination thereof. In an example embodiment, the targeting moiety is inserted or substituted in loop IV and/or loop VIII. In an example embodiment, the targeting moiety is IPFSRVNPDT (SEQ ID NO: 20285), LGFARTGAAD (SEQ ID NO: 20274), LGFTKSSGSD (SEQ ID NO: 20270), LRYSKTQGES (SEQ ID NO: 20266), SPYARSSAGV (SEQ ID NO: 20271), VGWSRLDLTT (SEQ ID NO: 20262).

In one example embodiment, the engineered capsid is a modified AAV1 capsid and can have a target moiety motif inserted after or a neighbor of amino acid 590 (i.e., between amino acid 590 and 591). In one example embodiment, the engineered capsid is a modified AAV3 capsid and can have a target moiety motif inserted after or a neighbor of amino acid 586. In one example embodiment, the engineered capsid is a modified AAV4 capsid and can have a target moiety motif inserted after or a neighbor of amino acid 586. In one example embodiment, the engineered capsid is a modified AAV5 capsid and can have a target moiety motif inserted after or a neighbor of amino acid 575. In one example embodiment, the engineered capsid is a modified AAV6 capsid and can have a target moiety inserted at or a neighbor of amino acid 585 and optionally Y705-731, T492V, K531E. In one example embodiment, the engineered capsid is a modified AAV8 capsid and can have a target moiety inserted after or a neighbor of amino acid 585 and 590. In one example embodiment, the engineered capsid is a modified AAV9 capsid and can have a target moiety inserted in between amino acid 588 and 589. (Büning, H.; Srivastava, A. Capsid Modifications for Targeting and Improving the Efficacy of AAV Vectors. Molecular Therapy-Methods & Clinical Development 2019, 12, 248-265). In one example embodiment, the engineered capsid can have a 7-mer motif inserted between amino acids 588 and 589 of an AAV9 viral protein. SEQ ID NO: 20506 is a reference AAV9 capsid sequence for at least referencing the insertion sites discussed above. In an example embodiment, the engineered capsid can have a 7-mer motif inserted between two consecutive amino acids within amino acids 451-460 of a capsid protein of AAV9 viral protein. SEQ ID NO: 20506 is a reference AAV9 capsid sequence for at least referencing the insertion sites discussed above. It will be appreciated that target moieties can be inserted in analogous positions in AAV viral proteins of other serotypes, such as but not limited to, AAV1, AAV2, AAV3, AAV4, AAV5, AAV6, AAV7, AAV8, AAV9, AAV rh.74, AAV rh. 10 capsid polypeptide. In some embodiments as previously discussed, the target moiety(s) can be inserted between any two contiguous amino acids within the AAV viral protein and in some embodiments the insertion is made in a variable region.

In one example embodiment, the first 1, 2, 3, or 4 amino acids of a target moiety can replace 1, 2, 3, or 4 amino acids of a polypeptide into which it is inserted and preceding the insertion site. Using an AAV as another non-limiting example, one or more of the target moieties can be inserted into e.g., an AAV9 capsid polypeptide between amino acids 588 and 589 and the insert can replace amino acids 586, 587, and 588 such that the amino acid immediately preceding the target moiety after insertion is residue 585. It will be appreciated that this principle can apply in any other insertion context and is not necessarily limited to insertion between residues 588 and 589 of an AAV9 capsid or equivalent position in another AAV capsid. It will further be appreciated that in some embodiments, no amino acids in the polypeptide into which the target moiety is inserted are replaced by the target moiety. In an example embodiment, the AAV capsid protein is selected from SEQ ID NO: 20506.

In some embodiments, in addition to the n-mer motif(s) the targeting moiety can include a polypeptide, a polynucleotide, a lipid, a polymer, a sugar, or a combination thereof.

The engineered viral capsid and/or capsid proteins can be encoded by one or more engineered viral capsid polynucleotides. In some embodiments, the engineered viral capsid polynucleotide is an engineered AAV capsid polynucleotide, engineered lentiviral capsid polynucleotide, engineered retroviral capsid polynucleotide, or engineered adenovirus capsid polynucleotide. In some embodiments, an engineered viral capsid polynucleotide (e.g., an engineered AAV capsid polynucleotide, engineered lentiviral capsid polynucleotide, engineered retroviral capsid polynucleotide, or engineered adenovirus capsid polynucleotide) can include a 3′ polyadenylation signal. The polyadenylation signal can be an SV40 polyadenylation signal.

In some embodiments, the engineered polynucleotide can be included in a polynucleotide that is configured to express the engineered capsid in a host cell system for production of viral particles. The host cell system may also include a construct that expresses a recombinant viral genome that comprises a transgene encoding a polypeptide or nucleic acid operably linked to one or more regulatory sequences that promote expression of the transgene in a target cell, including a recombinant AAV genome where the transgene and regulatory sequences are flanked by AAV ITR sequences.

In some embodiments, the engineered AAV capsid encoding polynucleotide can be included in a polynucleotide that is configured to be an express the engineered capsid in a host cell system for production of AAV viral particles. The host cell system may also include a construct that expresses a recombinant AAV viral genome that comprises a transgene encoding a polypeptide or nucleic acid operably linked to one or more regulatory sequences that promote expression of the transgene in a target cell, including a recombinant AAV genome where the transgene and regulatory sequences are flanked by AAV ITR sequences. In some embodiments, the engineered AAV capsid encoding polynucleotide can be operably coupled to a poly adenylation tail. In some embodiments, the poly adenylation tail can be an SV40 poly adenylation tail. In some embodiments, the AAV capsid encoding polynucleotide can be operably coupled to a promoter. In some embodiments, the regulatory sequence that regulates expression of the transgene is a promoter and can be a tissue specific promoter. In some embodiments, the tissue specific promoter is specific for muscle (e.g., cardiac, skeletal, and/or smooth muscle), neurons and supporting cells (e.g., astrocytes, glial cells, Schwann cells, etc.), fat, spleen, liver, kidney, immune cells, spinal fluid cells, synovial fluid cells, skin cells, cartilage, tendons, connective tissue, bone, pancreas, adrenal gland, blood cell, bone marrow cells, placenta, endothelial cells, and combinations thereof. In some embodiments, the promoter can be a constitutive promoter. Suitable tissue specific promoters and constitutive promoters are discussed elsewhere herein and are generally known in the art and can be commercially available. Suitable neuronal tissue/cell specific promoters include, but are not limited to, GFAP promoter (astrocytes), SYN1 promoter (neurons), and NSE/RU5′ (mature neurons).

### Further Capsid Modifications

In one example embodiment, the viral capsid protein may comprise one or more mutations relative to wild type. In an example embodiment, the one or more mutations comprise a K449R substitution in a capsid polypeptide of AAV 9 20507, or a substitution in an analogous position of a capsid polypeptide from AAV1, AAV2, AAV3, AAV4, AAV5, AAV6, AAV7, AAV8, AAVrh8, AAV rh.74, or AAV rh. 10. In an example embodiment, the K449R substituted AAV capsid is selected from SEQ ID NO: 20507.

In example embodiments, the viral capsid protein may comprise additional targeting motifs that are in addition to the n-mer motifs of the present disclosure. Without being bound by theory the additional targeting moieties can be antibodies or fragments thereof. In some embodiments, the additional targeting moiety can be any molecule or composition capable of recognizing, binding, attaching to, or otherwise interacting with a binding partner that can be present on the surface of a target cell. Binding partners include, but are not limited to nucleic acids, proteins, peptides, sugars, fats, or any combination thereof or any other molecule or molecules that are present on the surface of a target cell. In some embodiments, the binding partner is unique to a cell type or cell state or a to a group of related cell types or cell states. In some embodiments, the binding partner is a receptor, channel, or other complex present on the surface of a target cell. These additional targeting moieties can be used to target, e.g., specific cell types or cell states within those the set of target cells targeted by the n-mer motif. As used herein, “cell state” is used to describe transient elements of a cell's identity. Cell state can be thought of as the transient characteristic profile or phenotype of a cell. Cell states arise transiently during time-dependent processes, either in a temporal progression that is unidirectional (e.g., during differentiation, or following an environmental stimulus) or in a state vacillation that is not necessarily unidirectional and in which the cell may return to the origin state. Vacillating processes can be oscillatory (e.g., cell-cycle or circadian rhythm) or can transition between states with no predefined order (e.g., due to stochastic, or environmentally controlled, molecular events). These time-dependent processes may occur transiently within a stable cell type (as in a transient environmental response), or may lead to a new, distinct type (as in differentiation). See e.g., Wagner et al., 2016. Nat Biotechnol. 34 (11): 1145-1160.

In some embodiments, the additional targeting moiety is or includes a peptide or a polypeptide. In some embodiments, the additional targeting moiety is or includes an antibody or fragment thereof. Exemplary antibodies and fragments thereof are described in greater detail elsewhere herein, see e.g., discussion on exemplary cargos. In some embodiments, the additional targeting moiety is or includes an aptamer. In some embodiments, the additional targeting moiety is or includes a small molecule. In some embodiments, the additional targeting moiety is or includes a nucleic acid (e.g., DNA or RNA). In some embodiments, the additional targeting moiety is or includes a receptor. In some embodiments, the additional targeting moiety is or includes a receptor ligand. In some embodiments, the additional targeting moiety is or includes a carbohydrate (e.g., a sugar). In some embodiments, the additional targeting moiety is or includes a lipid. In some embodiments, the additional targeting moiety is an engineered protein scaffold. In some embodiments, the additional targeting moiety is an affibody. In some embodiments, the additional targeting moiety is an antibody mimetic. In some embodiments, the additional targeting moiety is an engineered binding protein, such as a designed ankyrin repeat proteins (DARPins) (see e.g., Plückthun et al., Annu. Rev. Pharmacol. Toxicol. (2015) 55 (1): 489-511), avimers (Silverman et al., Nat. Biotechnol. (2005) 23 (12): 1556-1561 and Jeong et al. Nat. Biotechnol. (2005) 23 (12): 1493-1494), or affibodies (see e.g., Nord et al., Nat. Biotechnol. (1997) 15 (8): 772-777). In example embodiments, the additional targeting moiety is a receptor ligand or binding protein. In some embodiments, the additional targeting moiety is attached or otherwise coupled to the capsid surface. In some embodiments, the additional targeting moiety is encoded by a vector that produces a capsid of the present invention described herein.

### Engineered Vectors and Vector Systems

Also provided herein are vectors and vector systems that can contain one or more of the engineered polynucleotides described herein that can encode one or more of the target moieties of the present invention, including but not limited to engineered viral polynucleotides (e.g., polynucleotides encoding engineered AAV capsid proteins). In a preferred embodiment, provided herein is a vector system comprising one or more vectors encoding a targeting moiety effective to increase transduction of central nervous system tissues (CNS), optionally further comprising a cargo coupled to or otherwise associated with the targeting moiety or further comprising a construct which encodes the cargo, including a recombinant viral genome comprising a transgene. In preferred embodiment, the targeting moiety encoded in the vector system binds to Transferrin Receptor (TFRC). As used in this context, engineered viral capsid polynucleotides refers to any one or more of the polynucleotides described herein capable of encoding an engineered viral capsid as described elsewhere herein and/or polynucleotide(s) capable of encoding one or more engineered viral capsid proteins described elsewhere herein. Further, where the vector includes an engineered viral capsid polynucleotide described herein, the vector can also be referred to and considered an engineered vector or system thereof although not specifically noted as such. In embodiments, the vector can contain one or more polynucleotides encoding one or more elements of an engineered viral capsid described herein. The vectors and systems thereof can be useful in producing bacterial, fungal, yeast, plant cells, animal cells, and transgenic animals that can express one or more components of the engineered viral capsid, particle, or other compositions described herein. Within the scope of this disclosure are vectors containing one or more of the polynucleotide sequences described herein. One or more of the polynucleotides that are part of the engineered viral capsid and system thereof described herein can be included in a vector or vector system.

In example embodiments, a vector used in the production of the rAAVs disclosed herein comprises a rep gene and cap gene). The rep gene typically encodes Rep78, Rep68, Rep52 and Rep40 from a single ORF. These replication factors aid AAV genome replication and virion assembly. The cap gene typically encodes the three capsid proteins (i.e., virion protein 1 (VP1), VP2 and VP3) from a single ORF as well. In addition, the three capsid proteins are regulated by transcription from a start codon (ACG) and alternative splicing. The cap gene also encodes, from an in-frameshifted ORF, an assembly-activating protein (AAP). The AAP is essential for capsid assembly.

In some embodiments, the vector can include an engineered viral (e.g., AAV) capsid polynucleotide having a 3′ polyadenylation signal. In some embodiments, 3′ polyadenylation is an SV40 polyadenylation signal. In some embodiments the vector does not have splice regulatory elements. In some embodiments, the vector includes one or more minimal splice regulatory elements. In some embodiments, the vector can further include a modified splice regulatory element, wherein the modification inactivates the splice regulatory element. In some embodiments, the modified splice regulatory element is a polynucleotide sequence sufficient to induce splicing, between a rep protein polynucleotide and the engineered viral (e.g., AAV) capsid protein variant polynucleotide. In some embodiments, the polynucleotide sequence can be sufficient to induce splicing is a splice acceptor or a splice donor. In some embodiments, the viral (e.g., AAV) capsid polynucleotide is an engineered viral (e.g., AAV) capsid polynucleotide as described elsewhere herein. It some embodiments, the vector does not include one or more minimal splice regulatory elements, modified splice regulatory agent, splice acceptor, and/or splice donor.

The vectors and/or vector systems can be used, for example, to express one or more of the engineered viral (e.g., AAV) capsid and/or other polynucleotides in a cell, such as a producer cell, to produce engineered viral (e.g., AAV) particles and/or other compositions (e.g., polypeptides, particles, etc.) containing an engineered viral (e.g., AAV) capsid or other composition containing an n-mer motif of the present invention described elsewhere herein. Other uses for the vectors and vector systems described herein are also within the scope of this disclosure. In general, and throughout this specification, the term is a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell).

In some embodiments, the vector can be a bicistronic vector. In some embodiments, a bicistronic vector can be used for one or more elements of the engineered viral (e.g., AAV) capsid system described herein. In some embodiments, expression of elements of the engineered viral (e.g., AAV) capsid system described herein can be driven by a suitable constitutive or tissue specific promoter. Where the element of the engineered viral (e.g., AAV) capsid system is an RNA, its expression can be driven by a Pol III promoter, such as a U6 promoter. In some embodiments, the two are combined.

### Cell-Based Vector Amplification and Expression

Vectors can be designed for expression of one or more elements of the engineered viral (e.g., AAV) capsid system or other compositions containing an n-mer motif of the present invention described herein (e.g., nucleic acid transcripts, proteins, enzymes, and combinations thereof) in a suitable host cell. In an example embodiment, a composition comprising a targeting moiety effective to increase transduction of CNS tissues comprises a n-mer motif, the n-mer motif comprising or consisting of an amino acid sequence of Z₁-X₁-Z₂-X₂-X₃-X₄-X₅ wherein Z₁ is Y, F, or L, Z₂ is S, R, or K, X₁-X₅ are independently selected amino acids. In an example embodiment, X₁ optionally comprises of A, S, or H; X₂ optionally comprises of S, T, L, or I; X₃ optionally comprises N or G; X₄ optionally comprises G; X₅ optionally comprises of N, D, I, V, or R. The n-mer motif may be selected from the group consisting of YSRIGPN (SEQ ID NO: 14632), YSRLNMN (SEQ ID NO: 14301), YSRLNKD (SEQ ID NO: 16577), and YHRLSNN (SEQ ID NO: 16636). In an example embodiment, the targeting moiety comprises a n-mer motif, the n-mer motif comprising or consisting of an amino acid sequence of is X₁-H-X₂-L-X₃-X₄-X₅ wherein X₁-X₅ are independently selected amino acids. In an example embodiment, the n-mer motif is VHRLQDK (SEQ ID NO: 16602) or LHALSHN (SEQ ID NO: 16608). In an example embodiment, the n-mer motif is comprises PSATNGV (SEQ ID NO: 20486), QVSTNGI (SEQ ID NO: 16021), SYSSNGV (SEQ ID NO: 16234), HQSSNGV (SEQ ID NO: 15978), VGSINGI (SEQ ID NO: 16200), AMSTNGR (SEQ ID NO: 16000), SASTNGV (SEQ ID NO: 16127), YMSTNGV (SEQ ID NO: 16042), YYSSNGV (SEQ ID NO: 16206), VHSTNGI (SEQ ID NO: 16134), PLSTNGV (SEQ ID NO: 16233), VYSTNGI (SEQ ID NO: 16059), IISTNGV (SEQ ID NO: 16054), RSVSSNGV (SEQ ID NO: 20502), YKSSNGV (SEQ ID NO: 16123), FRSTNGV (SEQ ID NO: 16070, and/or FVSTNGV (SEQ ID NO: 11162).

In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (In Vitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9 (11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329:840) and pMT2PC (Kaufman, et al., 1987. EMBO J. 6:187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to a transgene in a recombinant genome packaged by the engineered AAV capsid system so as to drive expression of the one or more elements of the transgene delivered by the viral vector as described herein in a tissue specific manner.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more elements of an engineered viral (e.g., AAV) capsid system or other composition containing an n-mer motif described herein are introduced into a host cell such that expression of the elements of the engineered delivery system described herein direct formation of an engineered viral (e.g., AAV) capsid system or other composition containing an n-mer motif described herein (including but not limited to an engineered gene transfer agent particle, which is described in greater detail elsewhere herein). For example, different elements of the engineered viral (e.g., AAV) capsid system or other composition containing an n-mer motif described herein can each be operably linked to separate regulatory elements on separate vectors. RNA(s) of different elements of the engineered delivery system described herein can be delivered to an animal or mammal or cell thereof to produce an animal or mammal or cell thereof that constitutively or inducibly or conditionally expresses different elements of the engineered viral (e.g., AAV) capsid system or other composition containing an n-mer motif described herein that incorporates one or more elements of the engineered viral (e.g., AAV) capsid system or other composition containing an n-mer motif described herein or contains one or more cells that incorporates and/or expresses one or more elements of the engineered viral (e.g., AAV) capsid system or other composition containing an n-mer motif described herein.

In some embodiments, two or more of the elements expressed from the same or different regulatory element(s) can be combined in a single vector, with one or more additional vectors providing any components of the system not included in the first vector. Engineered polynucleotides of the present invention that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more engineered viral (e.g., AAV) capsid proteins or other composition containing an n-mer motif described herein, embedded within one or more intron sequences (e.g., each in a different intron, two or more in at least one intron, or all in a single intron). In some embodiments, the engineered polynucleotides of the present invention (including but not limited to engineered viral polynucleotides) can be operably linked to and expressed from the same promoter.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In embodiments, the polynucleotides and/or vectors thereof described herein (including, but not limited to, the engineered AAV capsid polynucleotides of the present invention) can include one or more regulatory elements that can be operatively linked to the polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, brain), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981).

In some embodiments, the regulatory sequence can be a regulatory sequence described in U.S. Pat. No. 7,776,321, U.S. Pat. Pub. No. 2011/0027239, and PCT publication WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally-and/or spatially-regulated manner, and includes tissue-specific, tissue-preferred and inducible promoters. In some embodiments, the regulated promoter is a tissue specific promoter as previously discussed elsewhere herein. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTn1), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer114), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are discussed elsewhere herein and can be generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

In some embodiments, the vector or system thereof can include one or more elements capable of translocating and/or expressing an engineered polynucleotide of the present invention (e.g., an engineered viral (e.g., AAV) capsid polynucleotide) to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc.

### Selectable Markers and Tags

One or more of the engineered polynucleotides of the present invention (e.g., an engineered viral (e.g., AAV) capsid polynucleotide) can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered polynucleotide of the present invention (e.g., an engineered viral (e.g., AAV) capsid polynucleotide) such that the selectable marker polypeptide, when translated, is inserted between two amino acids between the N- and C-terminus of an engineered polypeptide (e.g., the engineered AAV capsid polypeptide) or at the N- and/or C-terminus of the engineered polypeptide (e.g., an engineered AAV capsid polypeptide). In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the engineered AAV capsid system described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly (NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered AAV capsid system or other compositions and/or systems described herein via suitable linker, such as a glycine or glycine serine linkers as short as GS or GG up to (GGGGG) 3 (SEQ ID NO: 20482) or (GGGGS) 3 (SEQ ID NO: 20483). Other suitable linkers are described elsewhere herein.

The vector or vector system can include one or more polynucleotides encoding one or more n-mers. In some embodiments, the n-mer encoding polynucleotides can be included in the vector or vector system, such as a viral vector system, such that they are expressed within and/or on the virus particle(s) produced such that the virus particles can be targeted to specific cells, tissues, organs, etc. In some embodiments, the n-mer encoding polynucleotides can be included in the vector or vector system such that the engineered polynucleotide(s) of the present invention (e.g., an engineered viral (e.g., AAV) capsid polynucleotide(s)) and/or products expressed therefrom include the n-mer and can be targeted to specific cells, tissues, organs, etc. In some embodiments, such as non-viral carriers, the n-mer can be attached to the carrier (e.g., polymer, lipid, inorganic molecule etc.) and can be capable of targeting the carrier and any attached or associated engineered polynucleotide(s) of the present invention, the engineered polypeptides, or other compositions of the present invention described herein, to specific cells, tissues, organs, etc. In some embodiments, the specific cells are CNS cells.

### Cell-Free Vector and Polynucleotide Expression

In some embodiments, the polynucleotide(s) encoding an n-mer motif of the present invention can be expressed from a vector or suitable polynucleotide in a cell-free in vitro system. In some embodiments, the polynucleotide encoding one or more features of the engineered AAV capsid system can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding an n-mer motif of the present invention and/or other polynucleotides described herein or the transgene contained within the recombinant AAV genome can be codon optimized. In some embodiments, polynucleotides of the engineered AAV capsid system described herein can be codon optimized. In some embodiments, one or more polynucleotides contained in a vector (“vector polynucleotides”) described herein that are in addition to an optionally codon optimized polynucleotide encoding an n-mer motif, including but not limited to, embodiments of the engineered AAV capsid system described herein, can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.or.jp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at www.yeastgenome.org/community/codon_usage.shtml, or Codon selection in yeast, Bennetzen and Hall, J Biol Chem. 1982 Mar. 25; 257(6): 3026-31. As to codon usage in plants including algae, reference is made to Codon usage in higher plants, green algae, and cyanobacteria, Campbell and Gowri, Plant Physiol. 1990 January; 92 (1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46 (4): 449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some embodiments, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an engineered capsid polynucleotide (e.g., an engineered AAV capsid polynucleotide) or other composition of the present invention described herein and can be capable of ferrying the polynucleotide to a cell and/or expressing the polynucleotide. It will be appreciated that this does not exclude the inclusion of a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid or polynucleotide molecule or composition that be attached to or otherwise interact with a polynucleotide to be delivered, such as an engineered AAV capsid polynucleotide of the present invention.

### Naked Polynucleotides

In some embodiments one or more engineered AAV capsid polynucleotides or other polynucleotides of the present invention described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered AAV capsid polynucleotides or other polynucleotides of the present invention described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered AAV capsid polynucleotide(s) or other polynucleotides of the present invention. In some embodiments, the naked polynucleotide can contain other nucleic acids and/or polynucleotides in addition to the engineered AAV capsid polynucleotide(s) or other polynucleotides of the present invention described elsewhere herein. The naked polynucleotides can include one or more elements of a transposon system. Transposons and system thereof are described in greater detail elsewhere herein.

### Non-Viral Polynucleotide Vectors

In some embodiments, one or more of the engineered AAV capsid polynucleotides or other polynucleotides of the present invention can be included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In embodiments, the S/MAR sequence is located downstream of an actively transcribed polynucleotide (e.g., one or more engineered AAV capsid polynucleotides or other polynucleotides or molecules of the present invention) included in the non-viral polynucleotide vector. In some embodiments, the S/MAR can be a S/MAR from the beta-interferon gene cluster. See e.g., Verghese et al. 2014. Nucleic Acid Res. 42: e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments, a non-viral polynucleotide transposon vector system can include a first polynucleotide vector that contains the engineered AAV capsid polynucleotide(s) or other polynucleotides, or molecules of the present invention described herein flanked on 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered AAV capsid polynucleotide(s) or other polynucleotides or molecules of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered AAV capsid polynucleotide(s) or other polynucleotides or molecules of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include Sleeping Beauty transposon system (Tc1/mariner superfamily) (see e.g., Ivics et al. 1997. Cell. 91 (4): 501-510), piggyBac (piggyBac superfamily) (see e.g., Li et al. 2013 110(25): E2279-E2287 and Yusa et al. 2011. PNAS. 108 (4): 1531-1536), Tol2 (superfamily hAT), Frog Prince (Tc1/mariner superfamily) (see e.g., Miskey et al. 2003 Nucleic Acid Res. 31 (23): 6873-6881) and variants thereof.

### Chemical Carriers

In some embodiments, the engineered AAV capsid polynucleotide(s) or other polynucleotides or other molecules of the present invention described herein can be coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the engineered AAV capsid polynucleotide(s) of the present invention), (2) those capable of targeting specific cells, (3) those capable of increasing delivery of the polynucleotide or other molecules (such as the engineered AAV capsid polynucleotide(s)) of the present invention to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the compositions (including particles, polypeptides, polynucleotides, and other compositions described herein) present invention described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticulo endothelial system. In some embodiments, the inorganic particles can be optimized to protect an entrapped molecule from degradation. The suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., super-magnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as an engineered AAV capsid polynucleotide of the present invention). In some embodiments, chemical non-viral carrier systems can include a polynucleotide (such as the engineered AAV capsid polynucleotide(s)) or other composition or molecule of the present invention) and a lipid (such as a cationic lipid). These are also referred to in the art as lipoplexes. Other embodiments of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g., the engineered AAV capsid polynucleotide(s) of the present invention). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the engineered AAV capsid polynucleotides of the present invention), polymethacrylate, and combinations thereof.

In some embodiments, the non-viral carrier can be configured to release an engineered delivery system polynucleotide that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the engineered AAV capsid polynucleotides or other compositions of the present invention describe herein and an environmental triggering agent response element, and optionally a triggering agent. In some embodiments, the particle can include a polymer that can be selected from the group of polymethacrylates and polyacrylates. In some embodiments, the non-viral particle can include one or more embodiments of the compositions microparticles described in US Pat. Pubs. 20150232883 and 20050123596, whose techniques and compositions can be adapted for use in the present invention.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the engineered AAV capsid polynucleotide(s) of the present invention). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an engineered AAV capsid polynucleotide, cargo, or other composition or molecule of the present invention, into a virus particle and producing said virus particle when used alone or with one or more other viral vectors (such as in a viral vector system). Viral vectors and systems thereof can be used for producing viral particles for delivery of and/or expression and/or generation of one or more compositions of the present invention described herein (including, but not limited to, any viral particle and associated cargo). The viral vector can be part of a viral vector system involving multiple vectors. In some embodiments, systems incorporating multiple viral vectors can increase the safety of these systems. Suitable viral vectors can include adenoviral-based vectors, adeno associated vectors, helper-dependent adenoviral (HdAd) vectors, hybrid adenoviral vectors, and the like. Other embodiments of viral vectors and viral particles produce therefrom are described elsewhere herein. In some embodiments, the viral vectors are configured to produce replication incompetent viral particles for improved safety of these systems.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2, 5, or 9. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355: 1911-1912; Lai et al. 2002. DNA Cell. Biol. 21:895-913; Flotte et al., 1996. Hum. Gene. Ther. 7: 1145-1159; and Kay et al. 2000. Nat. Genet. 24: 257-261. The vector can encode the engineered AAV capsids, said capsids forming adenoviral particles.

In some embodiments the vector can be a helper-dependent adenoviral vector or system thereof. These are also referred to in the field as “gutless” or “gutted” vectors and are a modified generation of adenoviral vectors (see e.g., Thrasher et al. 2006. Nature. 443:E5-7). In embodiments of the helper-dependent adenoviral vector system one vector (the helper) can contain all the viral genes required for replication but contains a conditional gene defect in the packaging domain. The second vector of the system can contain only the ends of the viral genome, one or more engineered AAV capsid polynucleotides, and the native packaging recognition signal, which can allow selective packaged release from the cells (see e.g., Cideciyan et al. 2009. N Engl J Med. 361:725-727). Helper-dependent Adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19 (4): 443-452; Alba et al. 2005. Gene Ther. 12:18-S27; Croyle et al. 2005. Gene Ther. 12: 579-587; Amalfitano et al. 1998. J. Virol. 72:926-933; and Morral et al. 1999. PNAS. 96:12816-12821). The techniques and vectors described in these publications can be adapted for inclusion and delivery of the engineered AAV capsid polynucleotides described herein. In some embodiments, the polynucleotide to be delivered via the viral particle produced from a helper-dependent adenoviral vector or system thereof can be up to about 38 kb. Thus, in some embodiments, a adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95: 820-828; Morral et al. 1998. Hum. Gene Ther. 9: 2709-2716; Kubo and Mitani. 2003. J. Virol. 77 (5): 2964-2971; Zhang et al. 2013. PloS One. 8(10) e76771; and Cooney et al. 2015. Mol. Ther. 23 (4): 667-674), whose techniques and vectors described therein can be modified and adapted for use in the engineered AAV capsid system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the engineered AAV capsid system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered AAV capsid system of the present invention.

### Adeno Associated Vectors

In an embodiment, the engineered vector or system thereof can be an adeno-associated vector (AAV). See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer than adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. The AAV vector or system thereof can include one or more engineered capsid polynucleotides described herein.

The AAV vector or system thereof can be operably linked to a regulatory sequence, said regulatory sequence encoding one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the promoter can be a tissue specific promoter as previously discussed. In some embodiments, the tissue specific promoter can drive expression of an engineered capsid AAV capsid polynucleotide described herein.

The AAV vector or system thereof can include one or more polynucleotides that can encode one or more capsid proteins, such as the engineered AAV capsid proteins described elsewhere herein. The engineered capsid proteins can be capable of assembling into a protein shell (an engineered capsid) of the AAV virus particle. The engineered capsid can have a cell-, tissue-, and/or organ-specific tropism.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5, AAV-9 or any combination thereof. One can select the AAV of the AAV with regard to the cells to be targeted; e.g., one can select AAV serotypes 1, 2, 5, 9 or a hybrid capsid AAV-1, AAV-2, AAV-5, AAV-9 or any combination thereof for targeting brain and/or neuronal cells; and one can select AAV-4 for targeting cardiac tissue; and one can select AAV-8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. See also Srivastava. 2017. Curr. Opin. Virol. 21:75-80.

It will be appreciated that while the different serotypes can provide some level of cell, tissue, and/or organ specificity, each serotype still is multi-tropic and thus can result in tissue-toxicity if using that serotype to target a tissue that the serotype is less efficient in transducing. Thus, in addition to achieving some tissue targeting capacity via selecting an AAV of a particular serotype, it will be appreciated that the tropism of the AAV serotype can be modified by an engineered AAV capsid described herein. As described elsewhere herein, variants of wild-type AAV of any serotype can be generated via a method described herein and determined to have a particular cell-specific tropism, which can be the same or different as that of the reference wild-type AAV serotype. In some embodiments, the cell, tissue, and/or specificity of the wild-type serotype can be enhanced (e.g., made more selective or specific for a particular cell type that the serotype is already biased towards). For example, wild-type AAV-9 is biased towards muscle and brain in humans (see e.g., Srivastava. 2017. Curr. Opin. Virol. 21:75-80.) By including an engineered AAV capsid and/or capsid protein variant of wild-type AAV-9 as described herein, the bias for e.g., brain can be reduced or eliminated and/or the septicity increased such that the brain specificity appears reduced in comparison, thus enhancing the specificity for the muscle as compared to the wild-type AAV-9. As previously mentioned, inclusion of an engineered capsid and/or capsid protein variant of a wild-type AAV serotype can have a different tropism than the wild-type reference AAV serotype. For example, an engineered AAV capsid and/or capsid protein variant of AAV-9 can have specificity for a tissue other than muscle or brain in humans.

In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed below, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5. It will be appreciated that wild-type hybrid AAV particles suffer the same specificity issues as with the non-hybrid wild-type serotypes previously discussed.

Advantages achieved by the wild-type based hybrid AAV systems can be combined with the increased and customizable cell-specificity that can be achieved with the engineered AAV capsids can be combined by generating a hybrid AAV that can include an engineered AAV capsid described elsewhere herein. It will be appreciated that hybrid AAVs can contain an engineered AAV capsid containing a genome with elements from a different serotype than the reference wild-type serotype that the engineered AAV capsid is a variant of. For example, a hybrid AAV can be produced that includes an engineered AAV capsid that is a variant of an AAV-9 serotype that is used to package a genome that contains components (e.g., AAV2 ITRs) from an AAV-2 serotype. As with wild-type based hybrid AAVs previously discussed, the tropism of the resulting AAV particle will be that of the engineered AAV capsid.

A tabulation of certain wild-type AAV serotypes as to these cells can be found in Grimm, D. et al, J. Virol. 82:5887-5911 (2008) reproduced below as Table A. Further tropism details can be found in Srivastava. 2017. Curr. Opin. Virol. 21:75-80 as previously discussed.

TABLE A

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In example embodiments, the AAV vector or system thereof is AAV rh.74 or AAV rh.10.

In example embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., a transgene encoding a therapeutic protein or nucleic acid of interest)).

### Vectors Encoding the Transgene

In one example embodiment, the vector encoding the transgene (also referred to as “an artificial genome”) comprises the transgene to be delivered flanked on either side by AAV ITRs. Only ˜145 bp AAV ITRs are required for recombinant AAV (rAAV) propagation because they participate in vector production, induce transgene expression, and ensure continual cell transduction. Accordingly, ˜96% of the AAV genome can be removed for gene therapy. For example, the rep and cap genes can be substituted for the expression cassette containing a promoter (such as those described herein), a therapeutic transgene (for example, IDS) and a poly(A) tail forms the essence of all AAV vectors.

In example embodiments, additional modifications may be implemented to further increase the efficacy of the AAV. For example, the AAV ITRs may be modified to increase the expression of the rAAV vector upon transduction, which may allow the transgene to be expressed without second-strand DNA synthesis; the promoter may be modified to increase transcription; and the codons in the transgene may be engineered to modify mRNA production and/or translation.

In example embodiments, the ITRs are modified to overcome second-strand synthesis after infection. The AAV transduction rate is restricted by the synthesis of dsDNA from the single-stranded AAV genome. ITRs initiate second-strand synthesis. In an example embodiment, modified ITRs are no longer suitable substrates for the Rep68 and Rep78 proteins. As a result, the terminal resolution of replication is obviated and specific self-complementary AAV (scAAV) replication intermediates are produced. The scAAV intermediates comprise plus and minus strands of DNA fused by the modified ITRs encapsulated into the virion shell. Wild-type AAVs package either a single plus-strand or minus-strand DNA. The modified scAAV intermediates are delivered to the nucleus, these plus and minus strands instantaneously anneal to form dsDNA.

In example embodiments, the cis-elements are optimized for targeted delivery. The cis-elements are optimized because the packaging capacity of AAVs is restricted. In an example embodiment, small cis-elements replace long promoter sequences for the delivery of large therapeutic transgenes (e.g., 4.4-4.5 kbs).

In example embodiments, several strategies may be used to deliver transgenes using AAV vectors. Example approach 1 takes advantage of an AAV genome concatemerized via the homologous recombination of ITR sequences. In this approach, transgene cassettes may be split into two or more vectors, which are then delivered to the same cells. After the virus is uncoated, an intact transgene is formed by the homologous recombination between the two or more fragments.

In example approach 2, truncated transgene fragments of different lengths are packaged into different AAV virions at undefined locations on the vector genome. Either homologous recombination of the overlapping regions of the different AAV vector genomes or annealing of different AAV vector genomes at complementary regions via single-stranded templates produces the transgene cassette. In example embodiments, overlapping fragments may be added to the end of the individual AAV vectors to encourage homologous recombination.

In example approach 3, a hybrid dual-vector incorporates an overlapping region with intron splice sites in the split vector transgenes. Approach 3 uses concatemerization activity of AAV genomes to bring independent AAV vector genomes together. Recombination (for example, the starting vectors are segregated into two halves each carrying 5′ and 3′ splicing elements, respectively), and splicing provide the appropriate transgene protein. This strategy may increase the expression of full functional protein.

In example approach 4, an AAV genome is cross-packaged into the capsids of other parvoviruses thus creating chimeric vectors. In example approach 5, intein-mediated protein trans-splicing is used. Intein catalyzes protein splicing thereby causing the ligation of two polypeptides via trans-splicing (this approach is similar to intron-mediated RNA splicing). Multiple AAV vectors are delivered to the same cells. Each of the AAV vectors encode one of the fragments of target proteins, the fragments are flanked by short split inteins. The full-length protein forms after protein trans-splicing. See e.g., Li, C., Samulski, R. J. Engineering adeno-associated virus vectors for gene therapy. Nat Rev Genet 21, 255-272 (2020), herein incorporated by reference.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein. AAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more elements of an engineered AAV capsid system described herein are as used in the foregoing documents, such as International Patent Application Publication WO 2014/093622 (PCT/US2013/074667) and are discussed in greater detail herein.

Virus Particle Production from Viral Vectors

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered AAV capsid polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., a transgene encoding a therapeutic protein or nucleic acid operably linked to a regulatory element that promotes expression in the target tissue) between 2 ITRs; (2) a vector that carries the AAV Rep-Cap encoding polynucleotide, including the engineered capsid protein described herein; and helper polynucleotides. One of skill in the art will appreciate various methods and variations thereof that are both helper and -helper free and as well as the different advantages of each system.

The engineered AAV vectors and systems thereof described herein can be produced by any of these methods.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., engineered AAV capsid system transcripts, proteins, enzymes, mutant forms thereof, fusion proteins thereof, etc.), and virus particles (such as from viral vectors and systems thereof).

AAV capsids prepared from one or more engineered AAV capsid polynucleotides can be used to deliver a recombinant AAV genome encoding a therapeutic protein or nucleic acid of interest. Alternatively, adenovirus or other plasmid or viral vector types as previously described, can be used, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it does not integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of engineered AAV capsid system components (e.g., polynucleotides encoding engineered AAV capsid and/or capsid proteins) to cells via particles. The term “particle” as used herein, refers to any suitable sized particles for delivery of the engineered AAV capsid system components described herein. Suitable sizes include macro-, micro-, and nano-sized particles. In some embodiments, any of the of the engineered AAV capsid system components (e.g., polypeptides, polynucleotides, vectors, and combinations thereof described herein) can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in embodiments, particle delivery can also be advantageous for other engineered capsid system molecules and formulations described elsewhere herein.

### Engineered Virus Particles Including an Engineered Viral (e.g., AAV) Capsid

Also described herein are engineered virus particles (also referred to here and elsewhere herein as “engineered viral particles” that can contain an engineered viral capsid (e.g., AAV capsid, referred to as “engineered AAV particles”) as described in detail elsewhere herein. It will be appreciated that the engineered AAV particles can be adenovirus-based particles, helper adenovirus-based particles, AAV-based particles, or hybrid adenovirus-based particles that contain at least one engineered AAV capsid proteins as previously described. An engineered AAV capsid is one that that contains one or more engineered AAV capsid proteins as are described elsewhere herein. In some embodiments, the engineered AAV particles can include 1-60 engineered AAV capsid proteins described herein. In some embodiments, the engineered AAV particles can contain 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 engineered capsid proteins. In some embodiments, the engineered AAV particles can contain 0-59 wild-type AAV capsid proteins. In some embodiments, the engineered AAV particles can contain 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, or 59 wild-type AAV capsid proteins. The engineered AAV particles can thus include one or more n-mer motifs as is previously described.

The engineered AAV particle can include one or more cargo polynucleotides. Cargo polynucleotides are discussed in greater detail elsewhere herein. Methods of making the engineered AAV particles from viral and non-viral vectors are described elsewhere herein. Formulations containing the engineered virus particles are described elsewhere herein.

### Example Cargos

The n-mers can be coupled to or otherwise associated with a cargo. Cargos can include any molecule that is capable of being coupled to or associated with the n-mers described herein. Cargos can include, without limitation, nucleotides, oligonucleotides, polynucleotides, amino acids, peptides, polypeptides, riboproteins, lipids, sugars, pharmaceutically active agents (e.g., drugs, imaging and other diagnostic agents, and the like), chemical compounds, and combinations thereof. In some embodiments, the cargo is DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatoires, anti-histamines, anti-infectives, radiation sensitizers, chemotherapeutics, radioactive compounds, imaging agents, and combinations thereof. In embodiments, the cargo is a recombinant AAV genome comprising a transgene, for example, encoding a therapeutic protein or nucleic acid, operably linked to regulatory sequences that direct expression of the therapeutic protein or nucleic acid in a target tissue, flanked by AAV ITR sequences.

In some embodiments, the cargo is capable of treating or preventing a neurological disease or disorder, details of which are described herein.

In some embodiments, the cargo is a morpholino, a peptide-linked morpholino, an antisense oligonucleotide, a PMO, a therapeutic transgene, a polynucleotide encoding a therapeutic polypeptide or peptide, a PPMO, one or more peptides, one or more polynucleotides encoding a CRISPR-Cas protein, a guide RNA, or both, a ribonucleoprotein, wherein the ribonucleoprotein comprises a CRISPR-Cas system molecule, a therapeutic transgene RNA, or other gene modifying or therapeutic RNA and/or protein, or any combination thereof.

In some embodiments, one or more n-mers described herein is directly attached to the cargo. In some embodiments, one or more n-mers described herein is indirectly coupled to the cargo, such as via a linker molecule. In some embodiments, one or more one or more n-mers described herein is coupled to associated with a polypeptide or other particle that is coupled to, attached to, encapsulates, and/or contains a cargo.

Exemplary particles include, without limitation, viral particles (e.g., viral capsids, which is inclusive of bacteriophage capsids), polysomes, liposomes, nanoparticles, microparticles, exosomes, micelles, and the like. The term “nanoparticle” as used herein includes a nanoscale deposit of a homogenous or heterogeneous material. Nanoparticles may be regular or irregular in shape and may be formed from a plurality of co-deposited particles that form a composite nanoscale particle. Nanoparticles may be generally spherical in shape or have a composite shape formed from a plurality of co-deposited generally spherical particles. Exemplary shapes for the nanoparticles include, but are not limited to, spherical, rod, elliptical, cylindrical, disc, and the like. In some embodiments, the nanoparticles have a substantially spherical shape.

### Cargo Polynucleotides

Cargos are also described elsewhere herein. In some embodiments, the cargo is a cargo polynucleotide that can be packaged into an engineered viral particle and subsequently delivered to a cell. In some embodiments, delivery is cell selective, e.g., neurons and glial cells of the central nervous system. In some embodiments, the one or more cargo polynucleotides are part of the engineered viral (e.g., AAV) genome of the viral (e.g., AAV) system and packaged within the engineered capsid containing a targeting moiety of the present invention. The cargo polynucleotides can be packaged into an engineered viral (e.g., AAV) particle, which can be delivered to, e.g., a cell. In some embodiments, the cargo polynucleotide can be capable of modifying a polynucleotide (e.g., gene or transcript) of a cell to which it is delivered. As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. Polynucleotide, gene, transcript, etc. modification includes all genetic engineering techniques including, but not limited to, gene editing as well as conventional recombinational gene modification techniques (e.g., whole or partial gene insertion, deletion, and mutagenesis (e.g., insertional and deletional mutagenesis) techniques.

In example embodiments, the cargo molecule is a polynucleotide that is or can encode a vaccine. In example embodiments, the cargo molecule is a polynucleotide encoding an antibody.

### Interference RNAs

In certain example embodiments, the one or more polynucleotides may encode one or more interference RNAs. Interference RNAs are RNA molecules capable of suppressing gene expressions. Example types of interference RNAs include small interfering RNA (siRNA), microRNA (miRNA), and short hairpin RNA (shRNA).

In certain example embodiments, the interference RNA may be a siRNAs. Small interfering RNA (siRNA) molecules are capable of inhibiting target gene expression by interfering RNA. siRNAs may be chemically synthesized, or may be obtained by in vitro transcription, or may be synthesized in vivo in target cell. siRNAs may comprise double-stranded RNA from 15 to 40 nucleotides in length and can contain a protuberant region 3′ and/or 5′ from 1 to 6 nucleotides in length. Length of protuberant region is independent from total length of siRNA molecule. siRNAs may act by post-transcriptional degradation or silencing of target messenger. In some cases, the exogenous polynucleotides encode shRNAs. In shRNAs, the antiparallel strands that form siRNA are connected by a loop or hairpin region.

The interference RNA (e.g., siRNA) may suppress expression of genes to promote long term survival and functionality of cells after transplanted to a subject. In some examples, the interference RNAs suppress genes in TGFβ pathway, e.g., TGFβ, TGFβ receptors, and SMAD proteins. In some examples, the interference RNAs suppress genes in colony-stimulating factor 1 (CSF1) pathway, e.g., CSF1 and CSF1 receptors. In certain embodiments, the one or more interference RNAs suppress genes in both the CSF1 pathway and the TGFβ pathway. TGFβ pathway genes may comprise one or more of ACVR1, ACVR1C, ACVR2A, ACVR2B, ACVRL1, AMH, AMHR2, BMP2, BMP4, BMP5, BMP6, BMP7, BMP8A, BMP8B, BMPR1A, BMPR1B, BMPR2, CDKN2B, CHRD, COMP, CREBBP, CUL1, DCN, E2F4, E2F5, EP300, FST, GDF5, GDF6, GDF7, ID1, ID2, ID3, ID4, IFNG, INHBA, INHBB, INHBC, INHBE, LEFTY1, LEFTY2, LOC728622, LTBP1, MAPK1, MAPK3, MYC, NODAL, NOG, PITX2, PPP2CA, PPP2CB, PPP2R1A, PPP2RIB, RBL1, RBL2, RBX1, RHOA, ROCK1, ROCK2, RPS6KB1, RPS6KB2, SKP1, SMAD1, SMAD2, SMAD3, SMAD4, SMAD5, SMAD6, SMAD7, SMAD9, SMURF1, SMURF2, SP1, TFDP1, TGFB1, TGFB2, TGFB3, TGFBR1, TGFBR2, THBS1, THBS2, THBS3, THBS4, TNF, ZFYVE16, and/or ZFYVE9.

In some embodiments, the cargo polynucleotide is an RNAi molecule, antisense molecule, and/or a gene silencing oligonucleotide or a polynucleotide that encodes an RNAi molecule, antisense molecule, and/or gene silencing oligonucleotide.

As used herein, “gene silencing oligonucleotide” refers to any oligonucleotide that can alone or with other gene silencing oligonucleotides utilize a cell's endogenous mechanisms, molecules, proteins, enzymes, and/or other cell machinery or exogenous molecule, agent, protein, enzyme, and/or polynucleotide to cause a global or specific reduction or elimination in gene expression, RNA level(s), RNA translation, RNA transcription, that can lead to a reduction or effective loss of a protein expression and/or function of a non-coding RNA as compared to wild-type or a suitable control. This is synonymous with the phrase “gene knockdown” Reduction in gene expression, RNA level(s), RNA translation, RNA transcription, and/or protein expression can range from about 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, to 1% or less reduction. “Gene silencing oligonucleotides” include, but are not limited to, any antisense oligonucleotide, ribozyme, any oligonucleotide (single or double stranded) used to stimulate the RNA interference (RNAi) pathway in a cell (collectively RNAi oligonucleotides), small interfering RNA (siRNA), microRNA, and short-hairpin RNA (shRNA). Commercially available programs and tools are available to design the nucleotide sequence of gene silencing oligonucleotides for a desired gene, based on the gene sequence and other information available to one of ordinary skill in the art.

In some embodiments, a cargo polynucleotide, such as an encoding polynucleotide, is flanked by at least a retroelement polypeptide encoding polynucleotide 3′ UTR or portion thereof, such as the proximal region of about 500 base pairs of 3′ UTR. In some embodiments a cargo polynucleotide, such as an encoding polynucleotide, is flanked by a (e.g., endogenous or engineered) retroelement polypeptide (such as a retroviral gag protein or gag homolog) 5′ UTR. In some embodiments a cargo polynucleotide, such as an encoding polynucleotide, is flanked by an (e.g., endogenous or engineered) retroelement polypeptide encoding polynucleotide 5′ and 3′ UTR. In some embodiments, the flanking retroelement polypeptide encoding polynucleotide UTR(s) are from PNMA, Arc, PEG10 or other Sushi Class polypeptide. In some embodiments, the inclusion of 3′ UTR, 5′UTR, or both can increase packaging and/or delivery of the cargo that they flank. These and other packaging elements are described in greater detail elsewhere herein.

### Gene Modification Cargo Polynucleotides

In some embodiments, the cargo molecule can be a polynucleotide or polypeptide or polynucleotide encoding a polypeptide that can alone or when delivered as part of a system, whether or not delivered with other components of the system, operate to modify the genome, epigenome, and/or transcriptome of a cell to which it is delivered. Such systems include, but are not limited to, CRISPR-Cas systems. Other gene modification systems, e.g., TALENs, Zinc Finger nucleases, Cre-Lox, morpholinos, etc. are other non-limiting examples of gene modification systems whose one or more components can be delivered by the engineered viral (e.g., AAV) particles described herein.

In some embodiments, the cargo molecule is or encodes a gene editing system or component thereof. In some embodiments, the cargo molecule is or encodes a CRISPR-Cas system molecule or a component thereof. In some embodiments, the cargo molecule is a polynucleotide that encodes one or more components of a gene modification system (such as a CRISPR-Cas system). In some embodiments the cargo molecule is or encodes a gRNA. CRISPR-Cas system as used herein is intended to encompass by Class 1 and Class 2 CRISPR-Cas systems and derivatives of CRISPR-Cas systems such as base editors, prime editors, and CRISPR-associated transposases (CAST) systems.

In some embodiments, the cargo molecule can be a polynucleotide or polypeptide or polynucleotide encoding a polypeptide that can alone or when delivered as part of a system, whether or not delivered with other components of the system, operate to modify the genome, epigenome, and/or transcriptome of a cell to which it is delivered, is such that it treats or prevents a disease, a disorder, or a symptom thereof of a neurologic disease or disorder, and/or viruses (such as single stranded RNA viruses). In some embodiments, the cargo molecule, whether or not delivered with other components of the system, operates to modify the genome, epigenome, and/or transcriptome of a cell to which it is delivered, is such that it treats or prevents a neurological disease or disorder described further herein.

In some embodiments, the cargo molecule, whether or not delivered with other components of the system, operates to modify the genome, epigenome, and/or transcriptome of a cell to which it is delivered, is such that can modify the GAA gene, such as any of those described in US Pat. App. Pub. 20190284555, the contents of which are incorporated by reference as if expressed in their entirety herein and can be adapted for use with the present invention.

In some embodiments, the cargo molecule is or encodes an antisense oligomer or RNA molecule, such as those described in U.S. Pat. App. Pub. US20160251398, US20150267202, and US20180216111, the contents of which are incorporated by reference as if expressed in their entirety herein and can be adapted for use with the present invention.

In some embodiments, the cargo molecule can be a peptide-oligomer, conjugate as described in e.g., International Patent Application Publication WO2017106304A1, the contents of which are incorporated by reference as if expressed in their entirety herein and can be adapted for use with the present invention.

An embodiment of the invention encompasses methods of modifying a genomic locus of interest to change gene expression in a cell by introducing into the cell any of the compositions described herein.

An embodiment of the invention is that the above elements are comprised in a single composition or comprised in individual compositions. These compositions may advantageously be applied to a host to elicit a functional effect on the genomic level.

### Polypeptides

In certain example embodiments, the cargo molecule may one or more polypeptides or may be a nucleic acid encoding a polypeptide. The polypeptide may be a full-length protein or a functional fragment or functional domain thereof, that is a fragment or domain that maintains the desired functionality of the full-length protein. As used within this section “protein” is meant to refer to full-length proteins and functional fragments and domains thereof. A wide array of polypeptides may be delivered using the engineered delivery vesicles described herein, including but not limited to, secretory proteins, immunomodulatory proteins, anti-fibrotic proteins, proteins that promote tissue regeneration and/or transplant survival functions, hormones, anti-microbial proteins, anti-fibrillating polypeptides, and antibodies. The one or more polypeptides may also comprise combinations of the aforementioned example classes of polypeptides. It will be appreciated that any of the polypeptides described herein can also be delivered via the engineered delivery vesicles and systems described herein via delivery of the corresponding encoding polynucleotide.

### Antibodies

In certain embodiments, the one or more polypeptides may comprise one or more antibodies. The term “antibody” is used interchangeably with the term “immunoglobulin” herein, and includes intact antibodies, fragments of antibodies, e.g., Fab, F(ab′)2 fragments, scFva, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic′ treatment of an intact or complete antibody or antibody chain. Fragments can also be obtained by recombinant means. Exemplary fragments include Fab, Fab′, F(ab′)2, Fabc, Fd, dAb, V_{HH} and scF and/or Fv fragments. As used herein, a preparation of antibody protein “having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

In example embodiments, the antibody is a fragment or portion thereof. In an example embodiment, the antibody is an epitope binding protein or portion thereof. The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

In some embodiments, the cargo or antibody is an antibody fragment or portion. In some embodiments, the cargo is an epitope binding protein. Examples of portions of antibodies or epitope-binding proteins encompassed by the present definition include: (i) the Fab fragment, having V_{L}, C_{L}, V_{H} and C_{H}1 domains; (ii) the Fab′ fragment, which′ is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii)′isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain oligopeptides, form a pair of “antigen binding regions” (Zapata et al., Protein Eng. 8(10):1057-62 (1995); and U.S. Pat. No. 5,641,870).

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

In some embodiments, the antibody is a single-chain antibody (scFvs). The term “single-chain variable fragment”, as used herein refers to a fusion protein containing the variable region(s) of the heavy (V_{H}) and light (V_{L}) of an immunoglobulin that are connected via a linker peptide. The linker peptide typically ranges from about 10 to about 25 amino acids. The linker can be flexible and can contain one or more glycine residues for flexibility. The linker can contain one or more serine or threonine residues to increase or modify solubility. The V_{H} and light (V_{L}) can be linked via the linker in any order. In some embodiments, N terminus of the V_{H} and is coupled, via a linker, C terminus of the (V_{L}). In some embodiments, C terminus of the V_{H} and is coupled, via a linker, N terminus of the (V_{L}). In some embodiments, the scFV is a bivalent or trivalent scFvs. In some embodiments bitrivalent or trivalent scFvs are bi or trispecific, menaing that they can target 2 or 3, respectively, different epitopes. See also e.g., Hollinger, Philipp; Prospero, T; Winter, G (July 1993). “Diabodies”: small bivalent and bispecific antibody fragments”. Proceedings of the National Academy of Sciences of the United States of America. 90 (14): 6444-8; incq, S; Bosman, F; Buyse, M A; Degrieck, R; Celis, L; De Boer, M; Van Doorsselaere, V; Sablon, E (2001). “Expression and purification of monospecific and bispecific recombinant antibody fragments derived from antibodies that block the CD80/CD86-CD28 costimulatory pathway”. Protein Expression and Purification. 22 (1): 11-24. doi: 10.1006/prep.2001.1417; Le Gall, F.; Kipriyanov, SM; Moldenhauer, G; Little, M (1999). “Di-, tri- and tetrameric single chain Fv antibody fragments against human CD19: effect of valency on cell binding”. FEBS Letters. 453 (1): 164-168. doi: 10.1016/S0014-5793(99)00713-9; Huston, J. S.; Levinson, D.; Mudgett-Hunter, M.; Tai, M. S.; Novotný, J.; Margolies, M. N.; Crea, R. (1988). “Protein engineering of antibody binding sites: recovery of specific activity in an anti-digoxin single-chain Fv analogue produced in Escherichia coli”. Proceedings of the National Academy of Sciences of the United States of America. 85 (16): 5879-5883; de Graaf et al., Methods Mol Biol. 2002; 178:379-87. doi: 10.1385/1-59259-240-6:379; Zhou, H. X., J Mol Biol. 2003 May 23; 329(1):1-8. doi: 10.1016/s0022-2836(03)00372-3; Bird and Walker. Trends Biotechnol. 1991 April; 9 (4): 132-7. doi: 10.1016/0167-7799(91)90044-I; Wörn et al., J Mol Biol. 2001 Feb. 2; 305(5):989-1010. doi: 10.1006/jmbi.2000.4265.

As used herein, “heavy chain antibody,” “V_{HH}” or “single-domain antibodies” (sdAbs) refers to an antibody which is composed only of two heavy chains and lacks the two light chains usually found in antibodies (see, e.g., Henry and Mackenzie, Antigen recognition by single-domain antibodies: structural latitudes and constraints. MAbs. 2018 August-September; 10 (6): 815-826). VHH can refer to an antibody or VHH domain. Single-domain antibodies (sdAb) are also referred to as a “nanobody”, which is defined herein as an antibody fragment composed of a single monomeric variable antibody domain. As used herein “VHH” is used interchangeably with “nanobody.” The ˜12-15 kDa variable domains of these antibodies (VHHs and VNARs) can be produced recombinantly and can recognize antigen in the absence of the remainder of the antibody heavy chain. In common antibodies, the antigen binding region consists of the variable domains of the heavy and light chains (VH and VL). Heavy-chain antibodies can bind antigens despite having only VH domains. In certain embodiments, the heavy chain antibody is an antibody derived from cartilaginous fishes (immunoglobulin new antigen receptor (IgNAR)) or camelid ungulates. Non-limiting examples of camelids include dromedaries, camels, llamas and alpacas.

It is intended that the term “antibody” encompass any Ig class or any Ig subclass (e.g., the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, 1 gM antibodies exist in pentameric f-rm, and IgA antibodies exist in monomeric, dimeric or multimeric form.

The term “IgG subclass” refers to the four subclasses of immunoglobulin class IgG-IgG1, IgG2, IgG3, and IgG4 that have “been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 or 4 peptide loops) stabilized, for example, by β pleated sheet and/or intrachain disulfide bond. Domains are further referred to herein as “constant” or “variable”, based on the relative lack of sequence variation within the domains of various class members in the case of “constant” domain, or the significant variation within the domains of various class members in the case of a “variable” domain. Antibody or polypeptide “domains” are often referred to interchangeably in the antibody or polypeptide “regions”. The “constant” domains of an antibody light chain are referred to interchangeably as “light chain constant regions”, “light chain constant domains”, “CL” regions or “CL” domains.” The “constant” domains of an antibody heavy chain are referred to interchangeably as “heavy chain constant regions”, “heavy chain constant domains”, “CH” regions or “CH” domains.” The “variable” domains of an antibody light chain are referred to interchangeably as “light chain variable regions”, “light chain variable domains”, “VL” regions or “VL” domains.” The “variable” domains of an antibody heavy chain are referred to interchangeably as “heavy chain constant regions”, “heavy chain constant domains”, “VH” regions or “VH” domains.

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from non-immunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on small polypeptides of 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar” Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant cross reactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly cross react with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein. “Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92 (6):1981-1988 (1998); Chen et al., Cancer Res. 58 (16):3668-3678 (1998); Harrop et al., J. Immunol. 161 (4): 1786-1794 (1998); Zhu et al., Cancer Res. 58 (15):3209-3214 (1998); Yoon et al., J. Immunol. 160 (7):3170-3179 (1998); Prat et al., J. Cell. Sci. III (Pt2):237-247 (1998); Pitard et al., J. Immunol. Methods 205 (2): 177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

### Engineered Cells and Organisms Expressing Said Engineered AAV Capsids

Described herein are engineered cells that can include one or more of the engineered AAV capsid polynucleotides, polypeptides, vectors, and/or vector systems. In some embodiments, one or more of the engineered AAV capsid polynucleotides can be expressed in the engineered cells. In some embodiments, the engineered cells can be capable of producing engineered AAV capsid proteins and/or engineered AAV capsid particles that are described elsewhere herein. Also described herein are modified or engineered organisms that can include one or more engineered cells described herein. The engineered cells can be engineered to express a cargo molecule (e.g., a cargo polynucleotide) dependently or independently of an engineered AAV capsid polynucleotide as described elsewhere herein, e.g., packaged within an engineered AAV capsid as described herein.

A wide variety of animals, plants, algae, fungi, yeast, etc. and animal, plant, algae, fungus, yeast cell or tissue systems may be engineered to express one or more nucleic acid constructs of the engineered AAV capsid system described herein using various transformation methods mentioned elsewhere herein. This can produce organisms that can produce engineered AAV capsid particles, such as for production purposes, engineered AAV capsid design and/or generation, and/or model organisms. In some embodiments, the polynucleotide(s) encoding one or more components of the engineered AAV capsid system described herein can be stably or transiently incorporated into one or more cells of a plant, animal, algae, fungus, and/or yeast or tissue system. In some embodiments, one or more of engineered AAV capsid system polynucleotides are genomically incorporated into one or more cells of a plant, animal, algae, fungus, and/or yeast or tissue system. Further embodiments of the modified organisms and systems are described elsewhere herein. In some embodiments, one or more components of the engineered AAV capsid system described herein are expressed in one or more cells of the plant, animal, algae, fungus, yeast, or tissue systems.

### Engineered Cells

Described herein are various embodiments of engineered cells that can include one or more of the engineered AAV capsid system polynucleotides, polypeptides, vectors, and/or vector systems described elsewhere herein. In some embodiments, the cells can express one or more of the engineered AAV capsid polynucleotides and can produce one or more engineered AAV capsid particles, which are described in greater detail herein. Such cells are also referred to herein as “producer cells”. It will be appreciated that these engineered cells are different from “modified cells” described elsewhere herein in that the modified cells are not necessarily producer cells unless they include one or more of the engineered AAV capsid polynucleotides, engineered AAV capsid vectors or other vectors described herein that render the cells capable of producing an engineered AAV capsid particle. Modified cells can be recipient cells of an engineered AAV capsid particles and can, in some embodiments, be modified by the engineered AAV capsid particle(s) and/or a cargo polynucleotide delivered to the recipient cell. Modified cells are discussed in greater detail elsewhere herein. The term modification can be used in connection with modification of a cell that is not dependent on being a recipient cell. For example, isolated cells can be modified prior to receiving an engineered AAV capsid molecule.

In an embodiment, the invention provides a non-human eukaryotic organism; for example, a multicellular eukaryotic organism, including a eukaryotic host cell containing one or more components of an engineered delivery system described herein according to any of the described embodiments. In other embodiments, the invention provides a eukaryotic organism; preferably a multicellular eukaryotic organism, comprising a eukaryotic host cell containing one or more components of an engineered delivery system described herein according to any of the described embodiments. In some embodiments, the organism is a host of AAV.

In particular embodiments, the plants, algae, fungi, yeast, etc., cells or parts obtained are transgenic plants, comprising an exogenous DNA sequence incorporated into the genome of all or part of the cells.

The engineered cell can be a prokaryotic cell. The prokaryotic cell can be bacterial cell. The prokaryotic cell can be an archaea cell. The bacterial cell can be any suitable bacterial cell. Suitable bacterial cells can be from the genus Escherichia, Bacillus, Lactobacillus, Rhodococcus, Rodhobacter, Synechococcus, Synechoystis, Pseudomonas, Psedoaltermonas, Stenotrophamonas, and Streptomyces Suitable bacterial cells include, but are not limited to Escherichia coli cells, Caulobacter crescentus cells, Rodhobacter sphaeroides cells, Psedoaltermonas haloplanktis cells. Suitable strains of bacterial include, but are not limited to BL21(DE3), DL21(DE3)-pLysS, BL21 Star-pLysS, BL21-SI, BL21-AI, Tuner, Tuner pLysS, Origami, Origami B pLysS, Rosetta, Rosetta pLysS, Rosetta-gami-pLysS, BL21 CodonPlus, AD494, BL2trxB, HMS174, NovaBlue (DE3), BLR, C41 (DE3), C43 (DE3), Lemo21 (DE3), Shuffle T7, ArcticExpress and ArticExpress (DE3).

The engineered cell can be a eukaryotic cell. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments the engineered cell can be a cell line. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, the engineered cell is a muscle cell (e.g. cardiac muscle, skeletal muscle, and/or smooth muscle), bone cell, blood cell, immune cell (including but not limited to B cells, macrophages, T-cells, CAR-T cells, and the like), kidney cells, bladder cells, lung cells, heart cells, liver cells, brain cells, neurons, skin cells, stomach cells, neuronal support cells, intestinal cells, epithelial cells, endothelial cells, stem or other progenitor cells, adrenal gland cells, cartilage cells, and combinations thereof.

In some embodiments, the engineered cell can be a fungus cell. As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains can include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

In some embodiments, the engineered cell is a cell obtained from a subject. In some embodiments, the subject is a healthy or non-diseased subject. In some embodiments, the subject is a subject with a desired physiological and/or biological characteristic such that when a engineered AAV capsid particle is produced it can package one or more cargo polynucleotides that can be related to the desired physiological and/or biological characteristic and/or capable of modifying the desired physiological and/or biological characteristic. Thus, the cargo polynucleotides of the produced engineered AAV capsid particle can be capable of transferring the desired characteristic to a recipient cell. In some embodiments, the cargo polynucleotides are capable of modifying a polynucleotide of the engineered cell such that the engineered cell has a desired physiological and/or biological characteristic.

In some embodiments, a cell transfected with one or more vectors described herein is used to establish a new cell line comprising one or more vector-derived sequences.

The engineered cells can be used to produce engineered viral (e.g., AAV) capsid polynucleotides, vectors, and/or particles. In some embodiments, the engineered viral (e.g., AAV) capsid polynucleotides, vectors, and/or particles are produced, harvested, and/or delivered to a subject in need thereof. In some embodiments, the engineered cells are delivered to a subject. Other uses for the engineered cells are described elsewhere herein. In some embodiments, the engineered cells can be included in formulations and/or kits described elsewhere herein.

The engineered cells can be stored short-term or long-term for use at a later time. Suitable storage methods are generally known in the art. Further, methods of restoring the stored cells for use (such as thawing, reconstitution, and otherwise stimulating metabolism in the engineered cell after storage) at a later time are also generally known in the art.

### Formulations

The compositions, polynucleotides, polypeptides, particles, cells, vector systems and combinations thereof described herein can be contained in a formulation, such as a pharmaceutical formulation. In some embodiments, the formulations can be used to generate polypeptides and other particles that include one or more CNS-specific n-mers described herein. In some embodiments, the formulations can be delivered to a subject in need thereof. In some embodiments, component(s) of the engineered AAV capsid system, engineered cells, engineered AAV capsid particles, and/or combinations thereof described herein can be included in a formulation that can be delivered to a subject or a cell. In some embodiments, the formulation is a pharmaceutical formulation. One or more of the polypeptides, polynucleotides, vectors, cells, and combinations thereof described herein can be provided to a subject in need thereof or a cell alone or as an active ingredient, such as in a pharmaceutical formulation. As such, also described herein are pharmaceutical formulations containing an amount of one or more of the polypeptides, polynucleotides, vectors, cells, or combinations thereof described herein. In some embodiments, the pharmaceutical formulation can contain an effective amount of the one or more of the polypeptides, polynucleotides, vectors, cells, and combinations thereof described herein. The pharmaceutical formulations described herein can be administered to a subject in need thereof or a cell.

In some embodiments, the amount of the one or more of the polypeptides, polynucleotides, vectors, cells, virus particles, nanoparticles, other delivery particles, and combinations thereof described herein contained in the pharmaceutical formulation can range from about 1 μg/kg to about 10 mg/kg based upon the bodyweight of the subject in need thereof or average bodyweight of the specific patient population to which the pharmaceutical formulation can be administered. The amount of the one or more of the polypeptides, polynucleotides, vectors, cells, and combinations thereof described herein in the pharmaceutical formulation can range from about 1 μg to about 10 g, from about 10 nL to about 10 ml. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells. In embodiments where the pharmaceutical formulation contains one or more cells, the amount can range from about 1 cell to 1×10², 1×10³, 1×10⁴, 1×10⁵, 1×10⁶, 1×10⁷, 1×10⁸, 1×10⁹, 1×10¹⁰ or more cells per nL, μL, mL, or L.

In embodiments, were engineered AAV capsid particles are included in the formulation, the formulation can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles. In some embodiments, the formulation can be 0.1 to 100 mL in volume and can contain 1 to 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, or 1×10²⁰ transducing units (TU)/mL of the engineered AAV capsid particles.

### Pharmaceutically Acceptable Carriers and Auxiliary Ingredients and Agents

In embodiments, the pharmaceutical formulation containing an amount of one or more of the polypeptides, polynucleotides, vectors, cells, virus particles, nanoparticles, other delivery particles, and combinations thereof described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition.

In addition to an amount of one or more of the polypeptides, polynucleotides, vectors, cells, engineered AAV capsid particles, nanoparticles, other delivery particles, and combinations thereof described herein, the pharmaceutical formulation can also include an effective amount of an auxiliary active agent, including but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, and combinations thereof.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone Cortisol). Suitable immunomodulators include, but are not limited to, prednisone, azathioprine, 6-MP, cyclosporine, tacrolimus, methotrexate, interleukins (e.g., IL-2, IL-7, and IL-12), cytokines (e.g., interferons (e.g., IFN-α, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammatoires (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), mebicar, fabomotizole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammatoires (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammatoires (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives)

Suitable anti-histamines include, but are not limited to, H1-receptor antagonists (e.g., acrivastine, azelastine, bilastine, brompheniramine, buclizine, bromodiphenhydramine, carbinoxamine, cetirizine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, parconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proguanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, boceprevir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, cefazoline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, ceftizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, Fosfomycin, metronidazole, aztreonam, bacitracin, penicillin (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, gatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfisoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, Fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, Bacillus Calmette-Guerin (BCG), temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid.

In embodiments where there is an auxiliary active agent contained in the pharmaceutical formulation in addition to the one or more of the polypeptides, polynucleotides, CRISPR-Cas complexes, vectors, cells, virus particles, nanoparticles, other delivery particles, and combinations thereof described herein, amount, such as an effective amount, of the auxiliary active agent will vary depending on the auxiliary active agent. In some embodiments, the amount of the auxiliary active agent ranges from 0.001 micrograms to about 1 milligram. In other embodiments, the amount of the auxiliary active agent ranges from about 0.01 IU to about 1000 IU. In further embodiments, the amount of the auxiliary active agent ranges from 0.001 mL to about 1 mL. In yet other embodiments, the amount of the auxiliary active agent ranges from about 1% w/w to about 50% w/w of the total pharmaceutical formulation. In additional embodiments, the amount of the auxiliary active agent ranges from about 1% v/v to about 50% v/v of the total pharmaceutical formulation. In still other embodiments, the amount of the auxiliary active agent ranges from about 1% w/v to about 50% w/v of the total pharmaceutical formulation.

### Dosage Forms

In some embodiments, the pharmaceutical formulations described herein may be in a dosage form. The dosage forms can be adapted for administration by any appropriate route. Appropriate routes include, but are not limited to, rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art.

Dosage forms adapted for parenteral administration and/or adapted for any type of injection (e.g. intravenous, intraperitoneal, subcutaneous, intramuscular, intradermal, intraosseous, epidural, intracardiac, intraarticular, intracavernous, gingival, subgingival, intrathecal, intravitreal, intracerebral, and intracerebroventricular) can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and resuspended in a sterile carrier to reconstitute the dose prior to administration. Extemporaneous injection solutions and suspensions can be prepared in some embodiments, from sterile powders, granules, and tablets. See e.g., Glascock, J. J., et al. Delivery of Therapeutic Agents Through Intracerebroventricular (ICV) and Intravenous (IV) Injection in Mice. J. Vis. Exp. (56), e2968 and Foley C P, et al. Intra-arterial delivery of AAV vectors to the mouse brain after mannitol mediated blood brain barrier disruption. J Control Release. 2014 Dec. 28; 196:71-78.

The dosage form can also be prepared to prolong or sustain the release of any ingredient. In some embodiments, the one or more of the polypeptides, polynucleotides, vectors, cells, and combinations thereof described herein can be the ingredient whose release is delayed. In other embodiments, the release of an optionally included auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995).

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, the one or more of the polypeptides, polynucleotides, vectors, cells, and combinations thereof described herein can be formulated with a paraffinic or water-miscible ointment base. In some embodiments, the active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, the one or more of the polypeptides, polynucleotides, vectors, cells, and combinations thereof described herein is contained in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient (e.g., the one or more of the polypeptides, polynucleotides, vectors, cells, and combinations thereof described herein and/or auxiliary active agent), which may be generated by various types of metered dose pressurized aerosols, nebulizers, or insufflators.

In some embodiments, the dosage forms can be aerosol formulations suitable for administration by inhalation. In some of these embodiments, the aerosol formulation can contain a solution or fine suspension of the one or more of the polypeptides, polynucleotides, vectors, cells, and combinations thereof described herein and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal, or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of one or more of the polypeptides, polynucleotides, vectors, cells, and combinations thereof described herein. In further embodiments, the aerosol formulation can also contain co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time.

For some dosage forms suitable and/or adapted for inhaled administration, the pharmaceutical formulation is a dry powder inhalable formulation. In addition to the one or more of the polypeptides, polynucleotides, vectors, cells, and combinations thereof described herein, an auxiliary active ingredient, and/or pharmaceutically acceptable salt thereof, such a dosage form can contain a powder base such as lactose, glucose, trehalose, mannitol, and/or starch. In some of these embodiments, the one or more of the polypeptides, polynucleotides, vectors, cells, and combinations thereof described herein is in a particle-size reduced form. In further embodiments, a performance modifier, such as L-leucine or another amino acid, cellobiose octaacetate, and/or metals salts of stearic acid, such as magnesium or calcium stearate.

In some embodiments, the aerosol dosage forms can be arranged so that each metered dose of aerosol contains a predetermined amount of an active ingredient, such as the one or more of the one or more of the polypeptides, polynucleotides, vectors, cells, and combinations thereof described herein.

Dosage forms adapted for ocular administration can include aqueous and/or nonaqueous sterile solutions that can optionally be adapted for injection, and which can optionally contain anti-oxidants, buffers, bacteriostats, solutes that render the composition isotonic with the eye or fluid contained therein or around the eye of the subject, and aqueous and nonaqueous sterile suspensions, which can include suspending agents and thickening agents.

For some embodiments, the dosage form contains a predetermined amount of the one or more of the polypeptides, polynucleotides, vectors, cells, and combinations thereof described herein per unit dose. In some embodiments, the predetermined amount of the Such unit doses may therefore be administered once or more than once a day. Such pharmaceutical formulations may be prepared by any of the methods well known in the art.

### Effective Amounts

In some embodiments, the amount of the primary active agent and/or optional secondary agent can be an effective amount, least effective amount, and/or therapeutically effective amount. As used herein, “effective amount”, “effective concentration”, and/or the like refers to the amount, concentration, etc. of the primary and/or optional secondary agent included in the pharmaceutical formulation that achieve one or more therapeutic effects or desired effect. As used herein, “least effective”, “least effective concentration”, and/or the like amount refers to the lowest amount, concentration, etc. of the primary and/or optional secondary agent that achieves the one or more therapeutic or other desired effects. As used herein, “therapeutically effective amount”, “therapeutically effective concentration” and/or the like refers to the amount, concentration, etc. of the primary and/or optional secondary agent included in the pharmaceutical formulation that achieves one or more therapeutic effects. In some embodiments, the one or more therapeutic effects comprise transducing the CNS.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, L, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Kits

Also described herein are kits that contain one or more of the one or more of the compositions, polypeptides, polynucleotides, vectors, cells, or other components described herein and combinations thereof and pharmaceutical formulations described herein. In embodiments, one or more of the polypeptides, polynucleotides, vectors, cells, and combinations thereof described herein can be presented as a combination kit. As used herein, the terms “combination kit” or “kit of parts” refers to the compounds, or formulations and additional components that are used to package, screen, test, sell, market, deliver, and/or administer the combination of elements or a single element, such as the active ingredient, contained therein. Such additional components include but are not limited to, packaging, syringes, blister packages, bottles, and the like. The combination kit can contain one or more of the components (e.g., one or more of the one or more of the polypeptides, polynucleotides, vectors, cells, and combinations thereof) or formulation thereof can be provided in a single formulation (e.g., a liquid, lyophilized powder, etc.), or in separate formulations. The separate components or formulations can be contained in a single package or in separate packages within the kit. The kit can also include instructions in a tangible medium of expression that can contain information and/or directions regarding the content of the components and/or formulations contained therein, safety information regarding the content of the components(s) and/or formulation(s) contained therein, information regarding the amounts, dosages, indications for use, screening methods, component design recommendations and/or information, recommended treatment regimen(s) for the components(s) and/or formulations contained therein. As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory drive or CD-ROM or on a server that can be accessed by a user via, e.g., a web interface.

In one embodiment, the invention provides a kit comprising one or more of the components described herein. In some embodiments, the kit comprises a vector system and instructions for using the kit. In some embodiments, the vector system includes a regulatory element operably linked to one or more engineered polynucleotides, such as those containing a selective n-mer, as described elsewhere herein and, optionally, a cargo molecule, which can optionally be operably linked to a regulatory element. The one or more engineered polynucleotides such as those containing a selective n-mer, as described elsewhere herein and, can be included on the same or different vectors as the cargo molecule in embodiments containing a cargo molecule within the kit.

In some embodiments, the kit comprises a vector system and instructions for using the kit. In some embodiments, the vector system comprises (a) a first regulatory element operably linked to a direct repeat sequence and one or more insertion sites for inserting one or more guide sequences up-or downstream (whichever applicable) of the direct repeat sequence, wherein when expressed, the guide sequence directs sequence-specific binding of a Cas9 CRISPR complex to a target sequence in a eukaryotic cell, wherein the Cas9 CRISPR complex comprises a Cas9 enzyme complexed with the guide sequence that is hybridized to the target sequence; and/or (b) a second regulatory element operably linked to an enzyme-coding sequence encoding said Cas9 enzyme comprising a nuclear localization sequence. Where applicable, a tracr sequence may also be provided. In some embodiments, the kit comprises components (a) and (b) located on the same or different vectors of the system. In some embodiments, component (a) further comprises two or more guide sequences operably linked to the first regulatory element, wherein when expressed, each of the two or more guide sequences direct sequence specific binding of a CRISPR complex to a different target sequence in a eukaryotic cell. In some embodiments, the Cas9 enzyme comprises one or more nuclear localization sequences of sufficient strength to drive accumulation of said CRISPR enzyme in a detectable amount in the nucleus of a eukaryotic cell. In some embodiments, the CRISPR enzyme is a type V or VI CRISPR system enzyme. In some embodiments, the CRISPR enzyme is a Cas9 enzyme. In some embodiments, the Cas9 enzyme is derived from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, or Porphyromonas macacae Cas9 (e.g., modified to have or be associated with at least one DD), and may include further alteration or mutation of the Cas9, and can be a chimeric Cas9. In some embodiments, the DD-CRISPR enzyme is codon-optimized for expression in a eukaryotic cell. In some embodiments, the DD-CRISPR enzyme directs cleavage of one or two strands at the location of the target sequence. In some embodiments, the DD-CRISPR enzyme lacks or substantially DNA strand cleavage activity (e.g., no more than 5% nuclease activity as compared with a wild-type enzyme or enzyme not having the mutation or alteration that decreases nuclease activity). In some embodiments, the first regulatory element is a polymerase III promoter. In some embodiments, the second regulatory element is a polymerase II promoter. In some embodiments, the guide sequence is at least 16, 17, 18, 19, 20, 25 nucleotides, or between 16-30, or between 16-25, or between 16-20 nucleotides in length.

### Methods of Use for Delivery of Cargo to CNS

The compositions including one or more of the cell-selective targeting moieties, engineered AAV capsid system polynucleotides, polypeptides, vector(s), engineered cells, engineered AAV capsid particles can be used generally to package and/or deliver one or more cargos to neuron and glial cells of the CNS. In some embodiments, delivery is done in cell-selective manner based upon the selectivity of the targeting moiety. In some embodiments this is conferred by the tropism of the engineered AAV capsid, which can be influenced at least in part by the inclusion of one or n-mer motifs described elsewhere herein. In some embodiments, compositions including one or more of the CNS targeting moieties, including engineered AAV capsid particles where the capsids incorporate the targeting moiety, can be administered to a subject or a cell, tissue, and/or organ and facilitate the transfer and/or integration of the cargo to the recipient cell. In other embodiments, engineered cells capable of producing compositions, such as polypeptides and other particles (e.g., engineered AAV capsids and viral particles), containing one or more of the targeting moieties can be generated from the polynucleotides, vectors, and vector systems etc., described herein. This includes without limitation, the engineered AAV capsid system molecules (e.g., polynucleotides, vectors, and vector systems, etc.). In some embodiments, the polynucleotides, vectors, and vector systems etc., described herein capable of generating the compositions, such as polypeptides and other particles (e.g., engineered AAV capsids and viral particles), containing one or more of the targeting moieties can be delivered to a cell or tissue, in vivo, ex vivo, or in vitro. In some embodiments, when delivered to a subject, the composition can transform a subject's cell in vivo or ex vivo to produce an engineered cell that can be capable of making a composition described herein that contains one or more of the cell-selective targeting moieties described herein, including but not limited to the engineered AAV capsid particles, which can be released from the engineered cell and deliver cargo molecule(s) to a recipient cell in vivo or produce personalized engineered compositions (e.g., AAV capsid particles) for reintroduction into the subject from which the recipient cell was obtained.

In some embodiments, an engineered cell can be delivered to a subject, where it can release produced compositions of the present invention (including but not limited to engineered AAV capsid particles) such that they can then deliver a cargo (e.g., a cargo polynucleotide(s)) to a recipient cell. These general processes can be used in a variety of ways to treat and/or prevent disease or a symptom thereof in a subject, generate model cells, generate modified organisms, provide cell selection and screening assays, in bioproduction, and in other various applications.

In some embodiments, the compositions, such as polypeptides and other particles (e.g., engineered AAV capsids and viral particles), containing one or more of the targeting moieties can be delivered to neural cells of the CNS. In another example embodiment, the compositions containing one or more targeting moieties can be delivered to glial cells of the CNS.

In some embodiments, the engineered AAV capsid polynucleotides, vectors, and systems thereof can be used to generate engineered AAV capsid variant libraries that can be mined for variants with a desired cell-selectivity. The description provided herein as supported by the various Examples can demonstrate that one having a desired cell-selectivity in mind could utilize the present invention as described herein to obtain a capsid with the desired cell-selectivity.

### Methods of Treatment

Provided herein are methods for treating a disease or disorder, the method comprising administering to a subject in need thereof, a composition as disclosed herein to cells of the CNS. In an aspect, the compositions used in methods disclosed herein are capable of increasing transduction of neurons and/or glial cells of the CNS, allowing for delivery of cargo and therapeutics directly to such cell types. In embodiments, a method is disclosed wherein the cargo is one or more polypeptides.

### Diseases or Disorders

In embodiments, a method is disclosed wherein the disease or disorder is a cancer, neurological disorder, or infection.

In an embodiment, methods of treatment comprise administering a composition as detailed herein to a subject in need thereof. In an example embodiment, the cancer is a neuroepithelial cancer. In an embodiment, the cancer is a neuroepithelial tumor, for example, Astrocytic tumors, e.g., Diffuse Astrocytoma (fibrillary, protoplasmic, gemistocytic, mixed), Anaplastic (malignant) astrocytoma, Glioblastoma (giant cell, gliosarcoma variants), Pilocytic astrocytoma, Pleomorphic xanthoastrocytoma, or Subependymal giant cell astrocytoma; Oligodendroglial tumors, e.g., Oligodendroglioma, Anaplastic (malignant) Oligodendroglioma, Ependymal tumors, Ependymoma (cellular, papillary, clear cell, tanycytic), Anaplastic (malignant) ependymoma, ependymoma, Subependymoma; Mixed tumors, e.g., Oligoastrocytomaor Anaplastic (malignant) oligoastrocytoma; Choroid plexus tumors, e.g., Choroid Plexus papilloma or Choroid Plexus carcinoma; Neuronal and mixed neuronal-glial tumors, e.g., Gangliocytoma, Gangloglioma, Dysembryoplastic neuroepithelial tumor (DNET), Dysplastic gangliocytoma of the cerebellum (Lhermitte-Duclos), Desmoplastic infantile astrocytoma/ganglioglioma, Central neurocytoma, Anaplastic ganglioglioma, Cerebellar liponeurocytoma, Paraganglioma of the filum terminale; Pineal tumors, e.g., Pineocytoma, Pineoblastoma, Pineal parenchymal tumor of intermediate differentiation; Embryonal tumors, e.g., Medulloblastoma (desmoplastic, large cell, melanotic, medullomyoblastoma), Medulloepithelioma, Supratentorial primitive neuroectodermal tumors, PNETs such as Neuroblastoma, Ganglioneuroblastoma, Ependymoblastoma, or Atypical teratoid/rhabdoid tumor; Neuroblastic tumors, e.g., Olfactory (esthesioneuroblastoma), Olfactory neuroepithelioma, Neuroblastomas of the adrenal gland and sympathetic nervous system; Glial tumors of uncertain etiology, e.g., Astroblastoma, Gliomatosis cerebri, Chordoid glioma of the third ventricle.

In an embodiment, the cancer is a primary cancer metastasized to brain or other region of the central nervous system.

In an example embodiment, the neurological disorder is caused by a neurodegenerative or a neurodevelopmental disease. Example neurodegenerative diseases include, but are not limited to, Alzheimer's disease and other memory disorders, amyotrophic lateral sclerosis (ALS), ataxia, Huntington's disease, Parkinson's disease, motor neuron disease, multiple system atrophy, progressive supranuclear palsy. Examples of neurodevelopmental diseases include, but are not limited to, attention-deficit/hyperactivity disorder (ADHD), autism, learning disabilities, intellectual disability (also known as mental retardation), conduct disorders, cerebral palsy, speech and language disorders, Tourette syndrome, Schizophrenia, Fragile X syndrome, and impairments in vision and hearing.

### Non-Human Transgenic Animals

In one aspect, as provided herein a method of creating humanized transgenic non-human animals comprising: delivering to one or more cells of a non-human animal a vector system or a recombinant viral particle comprising a recombinant viral genome, wherein: the vector system or recombinant viral genome encodes a human transferrin polypeptide, wherein the encoded human transferrin polypeptide is under the control of a tissue-specific promoter or miRNA binding element that has selective activity in the desired cell, tissue, or organ. In an example embodiment, the one or more cells are endothelial cells. In an example embodiment, the one or more cells are CNS cells. In an example embodiment, the one or more cells are of the CNS vasculature, lungs, kidneys, liver, or any combination thereof. In an example embodiment, the endothelial cells are of the CNS vasculature. In an example embodiment, the recombinant viral particle, optionally an AAV viral particle, comprises a capsid polypeptide, optionally an AAV capsid polypeptide, wherein the capsid polypeptide comprises a CNS specific n-mer motif. In an example embodiment, the CNS-specific n-mer motif comprises X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2. In an example embodiment, the CNS-specific n-mer motif comprises or consists of NNSTRGG (SEQ ID NO: 42429), GNSARNI (SEQ ID NO: 42430), and GNSVRDF (SEQ ID NO: 42431). In an example embodiment, the transgenic non-human animal is a rodent, optionally a mouse.

In one aspect, provided herein a humanized transgenic non-human animal comprising: one or more cells expressing a human transferrin polypeptide, optionally wherein the one or more cells are CNS cells. In an example embodiment, the transgenic non-human animal is a rodent, optionally a mouse. In one aspect, provided herein a humanized transgenic non-human animal produced by the methods described herein. In an example embodiment, the humanized non-human animal has a suppressed immune system.

It will be appreciated that in the present methods, where the non-human transgenic organism is multicellular, e.g., an animal or plant, the modification may occur ex vivo or in vitro, for instance in a cell culture and in some instances not in vivo. In other embodiments, it may occur in vivo. In an aspect, the invention provides a method of modifying an organism or a non-human organism by manipulation of a target sequence in a genomic locus of interest comprising: Delivering, e.g., via particle(s) or nanoparticle(s) or vector(s) (e.g., viral vector, e.g., AAV, adenovirus, lentivirus) a non-naturally occurring or engineered composition.

A single cell or a population of cells may preferably be modified ex vivo and then re-introduced, e.g., transplanted to make transgenic organisms that express TFRC in certain cells. The invention in some embodiments comprehends a method of modifying a eukaryote, such as a transgenic eukaryote comprising delivering, e.g., via vector(s) and/or particle(s) and/or nanoparticles a non-naturally occurring or engineered composition. The system may comprise one, two, three or four different vectors; and the system may comprise one, two, three or four different nanoparticle complex(es) delivering the component(s) of the system. Components I, II, III and IV may thus be located on one, two, three or four different vectors, and may be delivered by one, two, three or four different particle or nanoparticle complex(es) or AAVs or components I, II, III and IV can be located on same or different vector(s)/particle(s)/nanoparticle(s), with all combinations of locations envisaged. And complexes that target the CNS or CNS tissue or CNS cells are advantageous.

In some embodiments, vectors are delivered to the eukaryotic cell in a transgenic eukaryote. In some embodiments, said modifying takes place in said eukaryotic cell in a cell culture. In one aspect, the invention provides a method of generating a model eukaryotic cell or a model transgenic eukaryote comprising one or more human proteins.

In one aspect, the present invention provides a transgenic eukaryote, e.g., mouse. In one aspect, the present invention provides a constitutive transgenic eukaryote, e.g., mouse line obtained by crossing of the transgenic mouse with another mouse line. In certain embodiments, progeny (or progenies) derived from said transgenic eukaryote, e.g., mouse line, may be successfully bred over at least five generations without exhibiting increased levels of genome instability or cellular toxicity. In one aspect, the present invention provides a method for simultaneously introducing multiple mutations ex vivo in a tissue, organ or a cell line (of the CNS), or in vivo in the same. It can be appreciated that using the novel targeting moiety tools disclosed herein, may produce a transgenic non-human eukaryote, e.g., animal model with multiple mutations in any number of loci can be envisioned and are within the scope of the present invention. It will be appreciated that such a transgenic non-human eukaryote, e.g., animal model provides a valuable tool for research purposes, e.g., viral transduction, and opens the door for developing and testing new therapeutic interventions for targeting specific tissue involving mutations at multiple loci. Such uses are within the scope of the present invention.

The eukaryotic cell can comprise a constitutive promoter, or a tissue specific promoter, or an inducible promoter; and, the eukaryotic cell can be part of a non-human transgenic eukaryote, e.g., a non-human mammal, primate, rodent, mouse, rat, rabbit, canine, dog, cow, bovine, sheep, ovine, goat, pig, fowl, poultry, chicken, fish, insect or arthropod; advantageously a mouse. The isolated eukaryotic cell or the non-human transgenic eukaryote can express an additional protein or enzyme, such as TfR1; and, the expression of TfR1 can be driven by coding therefor functionally or operatively linked to a constitutive promoter, or a tissue specific promoter, or an inducible promoter.

The eukaryotic cell can be a mammalian cell, e.g., a mouse cell, such as a mouse cell that is part of a transgenic mouse having cells that express TfR1.

Transgenic non-human eukaryotic organisms, e.g., animals are also provided in an aspect of practice of the instant invention. Preferred examples include animals comprising TfR1, in terms of polynucleotides encoding TfR1 or the protein itself. In certain aspects, the invention involves a constitutive or conditional or inducible TfR1 non-human eukaryotic organism, such as an animal, e.g., a primate, rodent, e.g., mouse, rat and rabbit, are preferred; and can include a canine or dog, livestock (cow/bovine, sheep/ovine, goat or pig), fish, fowl or poultry, e.g., chicken, and an insect or arthropod, with it mentioned that it is advantageous if the animal is a model as to a human or animal protein, cell, or tissue, as use of the non-human eukaryotic organisms in condition modeling, e.g., via inducing a plurality, are preferred. To generate transgenic mice with the constructs, as exemplified herein one may inject pure, linear DNA into the pronucleus of a zygote from a pseudo pregnant female, e.g. a CB56 female. Founders may then be identified, genotyped, and backcrossed to CB57 mice. The constructs may then be cloned and optionally verified, for instance by Sanger sequencing. Knock ins are envisaged (alone or in combination

Accordingly, the invention involves a non-human eukaryote, animal, mammal, primate, rodent, etc. or cell thereof or tissue thereof that may be used as a model. For example, a method of the invention may be used to create a non-human eukaryote, e.g., an animal, mammal, primate, rodent or cell that comprises a modification in one or more nucleic acid sequences associated or correlated with a cell or tissue of such (such as the CNS). The cell may be in vivo or ex vivo in the cases of multicellular organisms. In the instance where the cell is in cultured, a cell line may be established if appropriate culturing conditions are met and preferably if the cell is suitably adapted for this purpose (for instance a stem cell). Hence, cell lines are also envisaged.

In an aspect the invention can involve cells, e.g., non-human eukaryotic, e.g., animal, such as mammal, e.g., primate, rodent, mouse, rat, rabbit, etc., or even human cells, transformed to contain TfR1, e.g., such cells as to which a vector that contains nucleic acid molecule(s) encoding a TfR1, e.g., with nucleic acid(s) encoding a promoter and at least one NLS, advantageously two or more NLSs, or such cells that have had their genome altered, e.g., through the vector being an integrating virus or through such cells being stem cells or cells that give rise to a cell line or a living organism (but wherein such an organism is advantageously non-human), that contains and expresses nucleic acid molecule(s) encoding TfR1. Such cells are then transplanted into or onto an animal suitable for expressing a cell or tissue. The cells proliferate on or in the non-human eukaryote, e.g., animal model. The non-human eukaryote, e.g., animal model, having the proliferated heterologous transplanted TfR1-containing cells, is then administered RNA(s) or vector(s), e.g., AAV, adenovirus, lentivirus containing or providing RNA(s), e.g., under the control of a promoter such as a U6 promoter and/or particle(s) and/or nanoparticle(s). The non-human eukaryote, e.g., animal model can then be used for testing, e.g., as to potential therapy and/or putative treatment via a possibly pharmaceutically active compound. The administering can be at or to or for body delivery to the proliferated heterologous transplanted TfR1-containing cells, e.g., direct injection at or near such proliferated heterologous transplanted TfR1-containing cells, or injection or other administration in such a way that the RNA(s) are delivered into the proliferated heterologous transplanted TfR1-containing cells, e.g., injection into the bloodstream whereby bodily functions transport to the proliferated heterologous transplanted TfR1-containing cells. In an aspect of the invention, barcoding techniques of WO/2013/138585 A1 can be adapted or integrated into the practice of the invention. WO/2013/138585 A1 provides methods for simultaneously determining the effect of a test condition on viability or proliferation of each of a plurality of genetically heterogeneous cell types. The number of living cells in the sample after exposure to the test condition as compared to the reference number of cells indicates the effect of the test condition on viability or proliferation of each cell type. WO/2013/138585 A1 also provides methods for simultaneously determining the effect of a test condition on viability or proliferation of each of a plurality of genetically heterogeneous cell types. Referenced patent applications are hereby incorporated by reference.

The practice of the present invention employs, unless otherwise indicated, conventional techniques for generation of genetically modified mice. See Marten H. Hofker and Jan van Deursen, TRANSGENIC MOUSE METHODS AND PROTOCOLS, 2nd edition (2011).

### Screening and Cell Selection

In one aspect, as provided herein a method of screening n-mer motifs capable of conferring transduction of central nervous system (CNS) tissues via binding to a transferrin receptor (TFRC) in humanized transgenic non-human animals comprising: introducing one or more compositions comprising a candidate n-mer motif to humanized non-human transgenic animal of any one of claims Error! Reference source not found.-Error! Reference source not found.; and detecting binding of the compositions that bind to a transferrin receptor (TFRC) and/or detecting transduction or uptake by one or more CNS cells of the humanized transgenic non-human animal. In an example embodiment, the candidate n-mer motif comprises or consists of X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2. In an example embodiment, the composition is a viral particle comprising one or more capsid proteins each comprising the candidate n-mer motif. In an example embodiment, the viral particle is an AAV viral particle and the one or more capsid proteins is an AAV capsid protein, optionally wherein the candidate n-mer motif is inserted in between amino acids 588-589 of an AAV9 capsid polypeptide or in an analogous position of a capsid protein from AAV1, AAV2, AAV3, AAV4, AAV5, AAV6, AAV7, AAV8, AAVrh8, AAV rh.74, or AAV rh. 10. In an example embodiment, at least one of the one or more compositions furthers comprises a cargo. In an example embodiment, the cargo is or encodes a therapeutic nucleic acid or polypeptide, a selectable marker, or a control polypeptide or nucleic acid.

In one aspect, a method of screening targeting moieties, including incorporated within an AAV, capable of transduction of central nervous system (CNS) tissues via binding to a transferrin receptor (TFRC) in humanized transgenic non-human animals comprising: (a) introducing a plurality of vector systems to one or more humanized transgenic non-human animal that express the human TFRC and (b) detecting the targeting moiety that binds to a transferrin receptor (TFRC).

The engineered AAV capsid system vectors, engineered cells, and/or engineered AAV capsid particles described herein can be used in a screening assay and/or cell selection assay. The engineered delivery system vectors, engineered cells, and/or engineered AAV capsid particles can be delivered to a subject and/or cell. In some embodiments, the cell is a eukaryotic cell. The cell can be in vitro, ex vivo, in situ, or in vivo. The engineered AAV capsid system molecules, vectors, engineered cells, and/or engineered AAV capsid particles described herein can introduce an exogenous molecule or compound to subject or cell to which they are delivered. The presence of an exogenous molecule or compound can be detected which can allow for identification of a cell and/or attribute thereof. In some embodiments, the delivered molecules or particles can impart a gene or other nucleotide modification (e.g., mutations, gene or polynucleotide insertion and/or deletion, etc.). In some embodiments the nucleotide modification can be detected in a cell by sequencing. In some embodiments, the nucleotide modification can result in a physiological and/or biological modification to the cell that results in a detectable phenotypic change in the cell, which can allow for detection, identification, and/or selection of the cell. In some embodiments, the phenotypic change can be cell death, such as embodiments where binding of a CRISPR complex to a target polynucleotide results in cell death. Embodiments of the invention allow for selection of specific cells without requiring a selection marker or a two-step process that may include a counter-selection system. The cell(s) may be prokaryotic or eukaryotic cells.

In one embodiment the invention provides for a method of selecting one or more cell(s) by introducing one or more mutations in a gene in the one or more cell(s), the method comprising: introducing one or more vectors, which can include one or more engineered delivery system molecules or vectors described elsewhere herein, into the cell(s), wherein the one or more vectors can include a CRISPR enzyme and/or drive expression of one or more of: a guide sequence linked to a tracr mate sequence, a tracr sequence, and an editing template; or other polynucleotide to be inserted into the cell and/or genome thereof; wherein, for example that which is being expressed is within and expressed in vivo by the CRISPR enzyme and/or the editing template, when included, comprises the one or more mutations that abolish CRISPR enzyme cleavage; allowing homologous recombination of the editing template with the target polynucleotide in the cell(s) to be selected; allowing a CRISPR complex to bind to a target polynucleotide to effect cleavage of the target polynucleotide within said gene, wherein the CRISPR complex comprises the CRISPR enzyme complexed with (1) the guide sequence that is hybridized to the target sequence within the target polynucleotide, and (2) the tracr mate sequence that is hybridized to the tracr sequence, wherein binding of the CRISPR complex to the target polynucleotide induces cell death, thereby allowing one or more cell(s) in which one or more mutations have been introduced to be selected. In a preferred embodiment, the CRISPR enzyme is a Cas protein. In another embodiment of the invention the cell to be selected may be a eukaryotic cell.

The screening methods involving the engineered AAV capsid system molecules, vectors, engineered cells, and/or engineered AAV capsid particles, including but not limited to those that deliver one more CRISPR-Cas system molecules to cell, can be used in detection methods such as fluorescence in situ hybridization (FISH). In some embodiments, one or more components of an engineered CRISPR-Cas system that includes a catalytically inactive Cas protein, can be delivered by an engineered AAV capsid system molecule, engineered cell, and/or engineered AAV capsid particle described elsewhere herein to a cell and used in a FISH method. The CRISPR-Cas system can include an inactivated Cas protein (dCas) (e.g., a dCas9), which lacks the ability to produce DNA double-strand breaks may be fused with a marker, such as fluorescent protein, such as the enhanced green fluorescent protein (eEGFP) and co-expressed with small guide RNAs to target pericentric, centric and teleomeric repeats in vivo. The dCas system can be used to visualize both repetitive sequences and individual genes in the human genome. Such new applications of labelled dCas, dCas CRISPR-Cas systems, engineered AAV capsid system molecules, engineered cells, and/or engineered AAV capsid particles can be used in imaging cells and studying the functional nuclear architecture, especially in cases with a small nucleus volume or complex 3-D structures. (Chen B, Gilbert L A, Cimini B A, Schnitzbauer J, Zhang W, Li G W, Park J, Blackburn E H, Weissman J S, Qi L S, Huang B. 2013. Dynamic imaging of genomic loci in living human cells by an optimized CRISPR/Cas system. Cell 155(7):1479-91. doi: 10.1016/j.cell.2013.12.001., the teachings of which can be applied and/or adapted to the CRISPR systems described herein. A similar approach involving a polynucleotide fused to a marker (e.g., a fluorescent marker) can be delivered to a cell via an engineered AAV capsid system molecule, vector, engineered cell, and/or engineered AAV capsid particle described herein and integrated into the genome of the cell and/or otherwise interact with a region of the genome of a cell for FISH analysis.

Similar approaches for studying other cell organelles and other cell structures can be accomplished by delivering to the cell (e.g., via an engineered delivery AAV capsid molecule, engineered cell, and/or engineered AAV capsid particle described herein) one or more molecules fused to a marker (such as a fluorescent marker), wherein the molecules fused to the marker are capable of targeting one or more cell structures. By analyzing the presence of the markers, one can identify and/or image specific cell structures.

In some embodiments, the engineered AAV capsid system molecules and/or engineered AAV capsid particles can be used in a screening assay inside or outside of a cell. In some embodiments, the screening assay can include delivering a CRISPR-Cas cargo molecule(s) via an engineered AAV capsid particle.

Use of the present system in screening is also provided by the present invention, e.g., gain of function screens. Cells which are artificially forced to overexpress a gene are able to down regulate the gene over time (re-establishing equilibrium) e.g., by negative feedback loops. By the time the screen starts the unregulated gene might be reduced again. Other screening assays are discussed elsewhere herein.

In an embodiment, the invention provides a cell from or of an in vitro method of delivery, wherein the method comprises contacting the delivery system with a cell, optionally a eukaryotic cell, whereby there is delivery into the cell of constituents of the delivery system, and optionally obtaining data or results from the contacting, and transmitting the data or results.

In an embodiment, the invention provides a cell from or of an in vitro method of delivery, wherein the method comprises contacting the delivery system with a cell, optionally a eukaryotic cell, whereby there is delivery into the cell of constituents of the delivery system, and optionally obtaining data or results from the contacting, and transmitting the data or results; and wherein the cell product is altered compared to the cell not contacted with the delivery system, for example altered from that which would have been wild type of the cell but for the contacting. In an embodiment, the cell product is non-human or animal. In some embodiments, the cell product is human.

In some embodiments, a host cell is transiently or non-transiently transfected with one or more vectors described herein. In some embodiments, a cell is transfected as it naturally occurs in a subject optionally to be reintroduced therein. In some embodiments, a cell that is transfected is taken from a subject. In some embodiments, the cell obtained from or is derived from cells taken from a subject, such as a cell line. Delivery mechanisms and techniques of the engineered AAV capsid system, engineered AAV capsid particles are described elsewhere herein.

In some embodiments it is envisaged to introduce the engineered AAV capsid system molecule(s) and/or engineered AAV capsid particle(s) directly to the host cell. For instance, the engineered AAV capsid system molecule(s) can be delivered together with one or more cargo molecules to be packaged into an engineered AAV capsid particle.

The invention is further described in the following examples, which do not limit the scope of the invention described in the claims.

### EXAMPLES

### Example 1—Screening for 7-Mer Modified AAV Capsids that Bind Selectively to Cells Expressing Human TFRC (hTFRC)

A library of capsids with a 7-mer insertion between AA588 and 589 of AAV9 K449R was screened for capsids that bind to HEK293 or CHO cells transiently transfected with human TFRC (hTFRC) more efficiently than cells expressing a control protein (GFP). Sequences that bind more efficiently to cells expressing hTFRC were identified by NGS. Analysis of these sequences identified several motifs present in a subset of sequences: [YFL][AS][RK]X[NG]X[N] (SEQ ID NO: 20484) and XXS[ST]NG[IVR] (SEQ ID NO: 20485), where X=any amino acid.

Applicants generated a second round library comprising sequences identified in the cell-based screen for hTFRC binding and all of the sequences conforming to the above two motifs using oligo pool synthesis. The oligo pool was used to generate a capsid library. The second round library was screened on HEK293 cells with or without transient expression of hTFRC (5,000 vg/cell) as performed in the first round (Table 1). In addition, the second round library was screened to identify capsids that more efficiently transduced (1) hCMEC/D3 cells, which express endogenous hTFRC, (2) mouse brain microvascular endothelial cells (BMVECs) (Table 2) as well as (3) mixed culture spheroids consisting of hCMEC/D3 cells, and primary human astrocytes and pericytes (Cho et al, Nature Communications 2017).

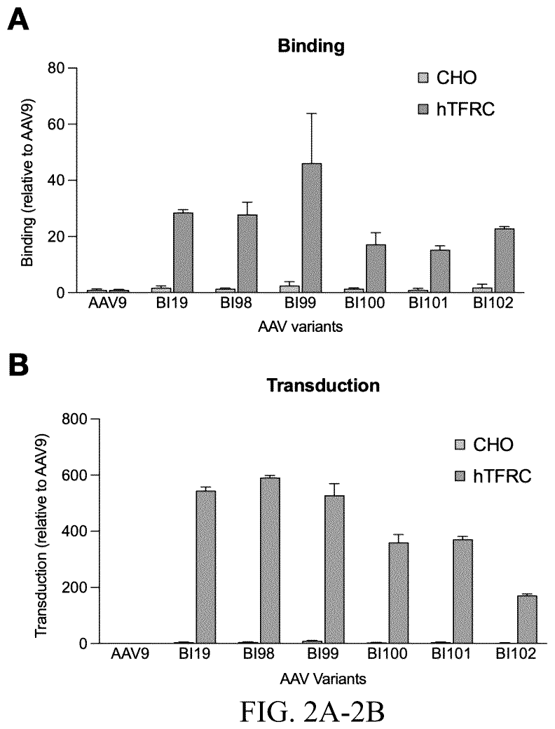

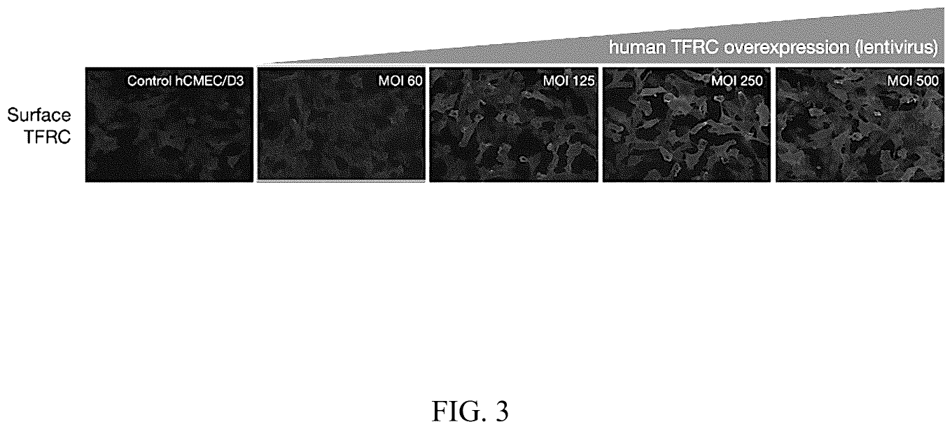

Applicants chose several novel TFRC binding caspids for further study: AAV-BI19: YSRIGPN (SEQ ID NO: 14632); AAV-B198: YSRLNMN (SEQ ID NO: 14301); AAV-BI99: YSRLNKD (SEQ ID NO: 16577); AAV-BI100: VHRLQDK (SEQ ID NO: 16602); AAV-BI101: YHRLSNN (SEQ ID NO: 16636); and AAV-BI102: LHALSHN (SEQ ID NO: 16608). Applicants used these capsids to package a reporter transgene and transduced CHO cells or CHO cells expressing human TFRC (FIG. 2), as well as hCMEC cells or hCMEC cells overexpressing human TFRC (FIG. 3). In both cell types, each of the TFRC binding capsids showed enhanced ability to bind and transduce cells expressing human TFRC relative to AAV9. Notably, the enhanced transduction by these engineered capsids was absent in CHO cells, which lack expression of human TFRC. In contrast, the engineered AAVs each showed enhanced transduction of unmodified hCMEC cells relative to AAV9, which is fitting with the known expression of human TFRC in these cells. The role of TFRC for the enhanced hCMEC transduction of these capsid variants is supported by the finding that transduction was further enhanced in cells with greater cell surface exposed TFRC (FIGS. 3 and 4).

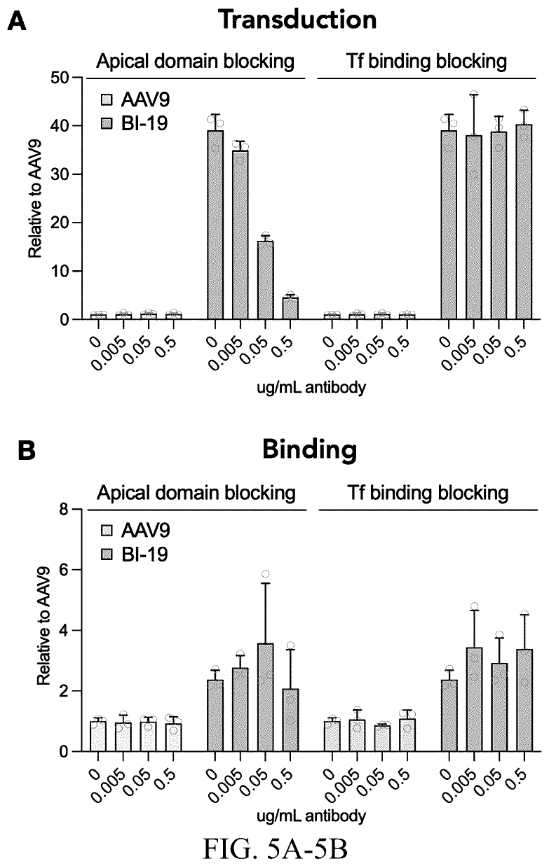

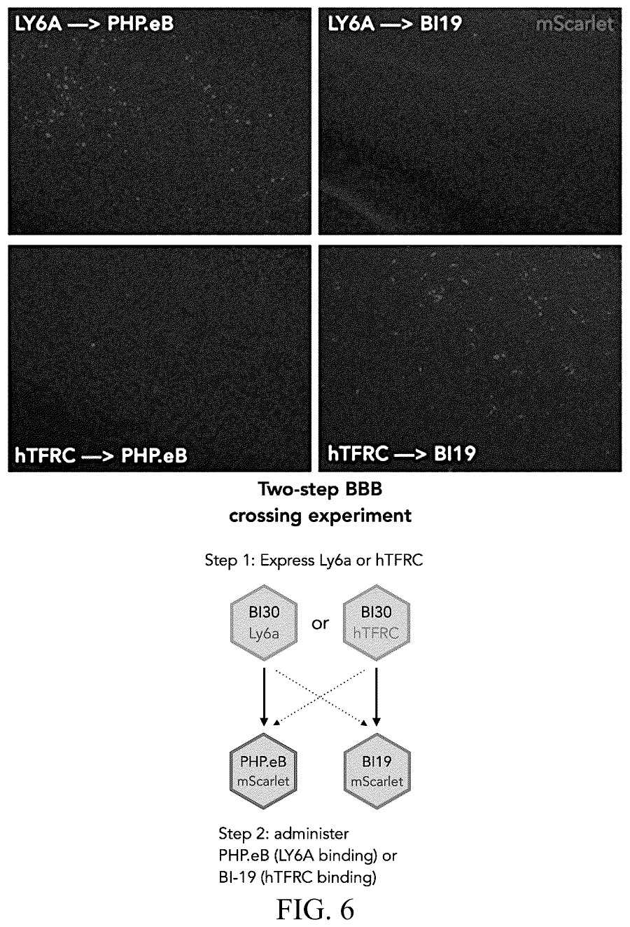

To assess whether the enhanced transduction of hCMEC cells by AAV-BI19 requires endogenous TFRC, Applicants attempted to block the interaction of the AAV with TFRC using several anti-TFRC antibodies: OKT9 (Thermo) and AF2474 (R&D Systems). Applicants found that OKT9, which binds to the TFRC apical domain (Ferrero et al J. of Virology https://doi.org/10.1128/JVI.01868-20) blocked transduction by AAV-BI19 in a dose-dependent manner (FIG. 5). In contrast, AF2474, which blocks binding of Transferrin to TFRC, did not inhibit transduction by AAV-BI19. Together these results suggest that the enhanced tropism of AAV-BI19 for human brain endothelial cells is mediated through binding to the human TFRC apical domain.

### Example 2—Screening for 7-Mer Modified AAV Capsids that Bind Selectively to hTFRC-Fc

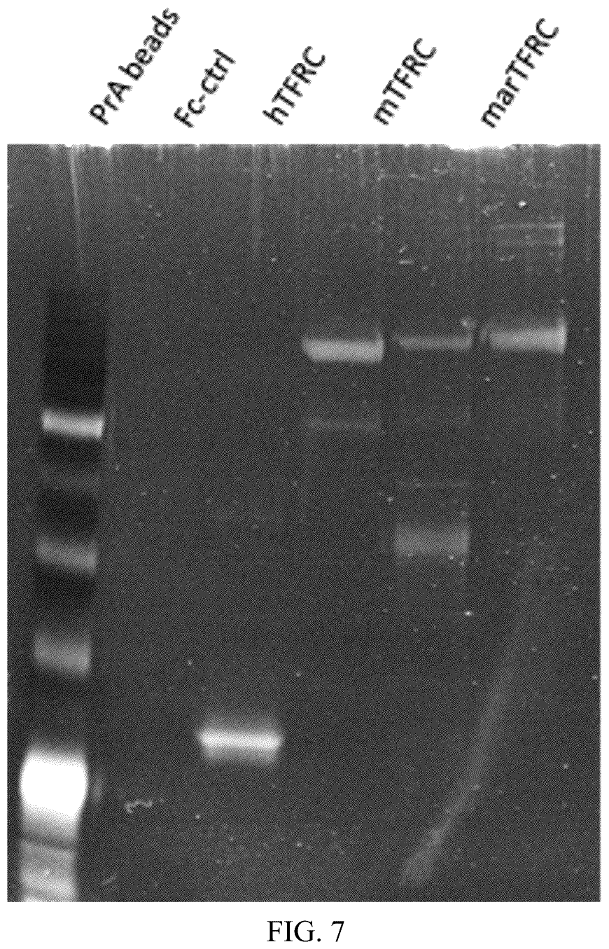

In a second experiment, Applicants screened a separately produced AAV9 K499R 7-mer 588 insertion library for variants capable of selectively binding hTFRC when produced as an Fc fusion protein. To perform this assay, Applicants expressed the human or marmoset TFRC-Fc fusions in HEK293 cells and pulled down the Fc fusion proteins from the cell culture media using ProA-magentic beads (FIG. 7).

The recovered 7-mers found to bind hTFRC-Fc were synthesized as an oligo pool and used to generate a second-round library. Applicants screened the second-round library for variants that bind hTFRC using the same hTFRC-Fc assay (Table 4), variants that bind to CHO cells stably expressing hTFRC (Table 5), variants that bind and transduce CHO cells expressing hTFRC (Table 6), and variants that bind HEK293 cells overexpressing hTFRC (Table 7). A subset of sequences bound HEK293 cells expressing either human TFRC or marmoset TFRC (Table 8).

### Example 3—Multiple Regions of the AAV Capsid can be Engineered to Enable TFRC Binding

To determine whether other regions of the AAV capsid could be engineered to bind to TFRC, Applicants generated libraries where a stretch of 10 AA in loop IV of AAV9 were substituted with a random string of AAs of seven or 10 AAs in length. Applicants also repeated the screen using a new loop VIII 7-mer insertion library as a positive control. Applicants screened these libraries for capsids that gained the ability to bind selectively to human TFRC (Tabel 9 and Table 10) or human TFRC where the apical domain was replaced with the corresponding domain from mouse TFRC (Table 11). Notably, several of the 10 AA sequences recovered in the loop IV substitution library (IPFSRVNPDT (SEQ ID NO: 20285), LGFARTGAAD (SEQ ID NO: 20274), LGFTKSSGSD (SEQ ID NO: 20270), LRYSKTQGES (SEQ ID NO: 20266), SPYARSSAGV (SEQ ID NO: 20271), and VGWSRLDLTT (SEQ ID NO: 20262)) contain the partial [YWFL][AST][RK] (SEQ ID NO: 20492) motif like that observed in human TFRC binding 7-mer insertions at loop VIII, suggesting that this binding motif is functional in multiple structural contexts.

### Example 4—Toward a Systematic Mapping of the Relationship Between AAV 7-Mer Sequence and hTFRC Binding

Applicants have generated a novel type of library (Fit4Function) composed of AAV capsids that uniformly samples only the high production fitness sequence space (International Patent Application WO 2021222636 A1). When used to screen for novel functions (e.g., new receptor binding), the Fit4Function libraries can generate highly reproducible quantitative data suitable for training ML models that predict the function of previously untested sequences within the theoretical sequence space. Applicants screened a Fit4Function library for capsids that selectively bind to TFRC-Fc. Binding was measured at both pH7.4 and pH5.5 and is reported in Table 12. Notably, even within this smaller library (240K unique sequences), several examples of sequences conforming to previously identified motifs were identified (e.g., YAKGGSN (SEQ ID NO: 11535) and YSKSGPG (SEQ ID NO: 20422), [YFL][AS][RK]X[NG]X[N] (SEQ ID NO: 20484); VKSSNGV (SEQ ID NO: 11671), XXS[ST]NG[IVR] (SEQ ID NO: 20485)).

### Clustering and Motif Analyses

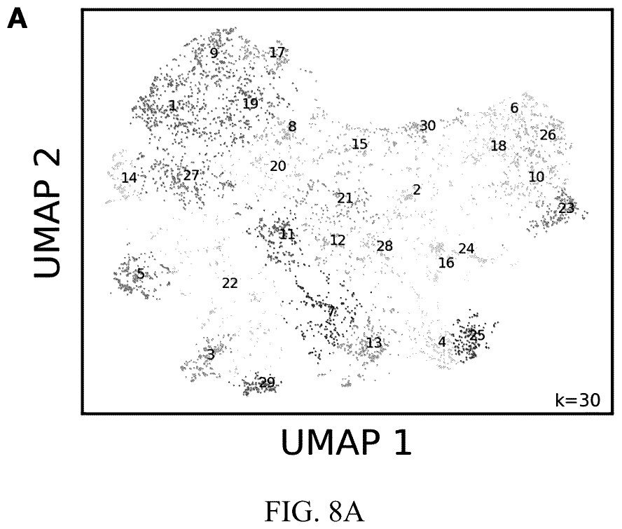

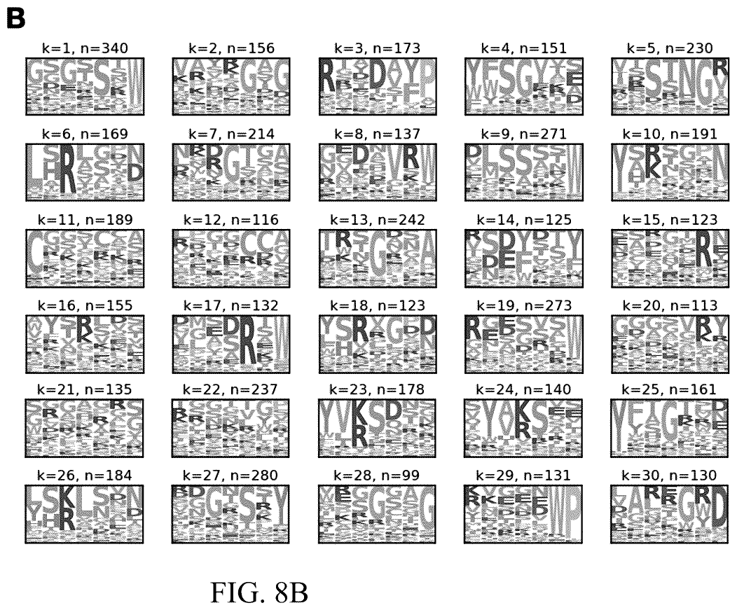

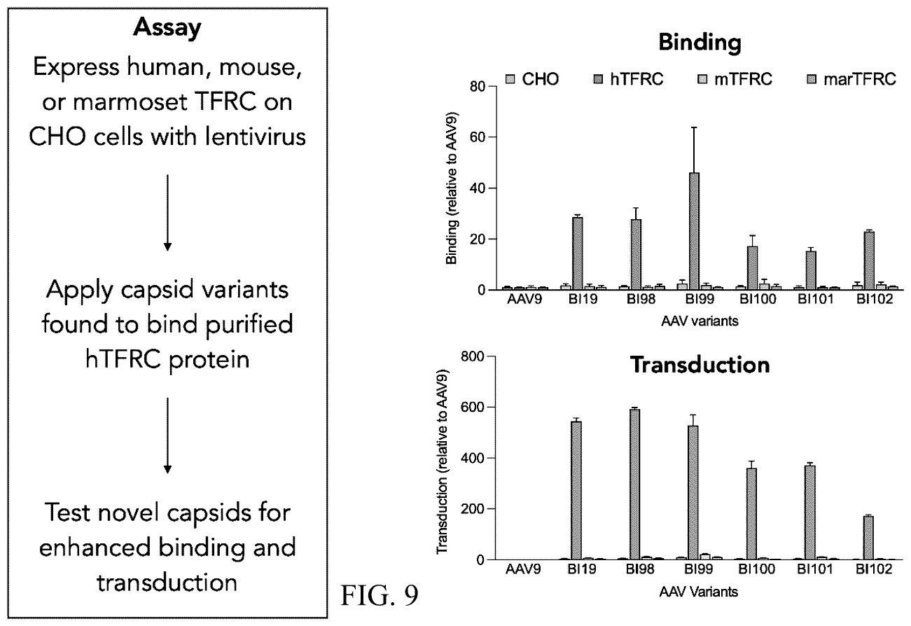

Applicants compiled sequences from all experiments run using human TFRC and the 588 site 7-mer libraries and performed clustering using UMAP. Sequences generated by mutagenesis of the motifs [YFL][AS][RK]X[NG]X[N] (SEQ ID NO: 20484) and XXS[ST]NG[IVR] (SEQ ID NO: 20485) were not included in the clustering analysis (FIG. 8 and Table 13) but were considered when defining core sequence motifs shown below.

### Motif Analysis

X₁X₂S[TS]NGX₃ (SEQ ID NO: 20487), where X₁ is any residue other than D, K, R; X₂ is any residue other than P; and X₃ is a V, I, or R.

X₁X₂X₃X₄X₅X₆X₇, where X₁ and X₂ are aromatic residues (F, W, or Y); X₃ is an S, T, A, H, X₄ is a G, N, H; X₄ is any residue; X₅ is any residue; X₆ is any residue; and X₇ is D, E, A, S, N, H, or P.

X₁CX₂X₃CX₄X₅, where X₁ is L,P,R,A,H,E,S,N, or W; X₂ is G,P,S,T,K,Y,R; X3 is P,K,D,E,L,G, or S; X₄ is any residue, and X5 is any residue.

FAKX₁X₂X₃X₄, where X1 is any residue, X2 is a G or an N, X3 is any residue, and X4 is preferably a N or a D.

FARX₁X₂X₃X₄ where X1 is any residue, X2 is a G or an N, X3 is any residue, and X4 is preferably a N or a D.

FSKX₁X₂X₃X₄ where X1 is any residue, X2 is a G or an N, X3 is any residue, and X4 is preferably a N or a D.

FSRX₁X₂X₃X₄ where X1 is any residue, X2 is a G or an N, X3 is any residue, and X4 is preferably a N or a D.

LAKX₁X₂X₃X₄ where X1 is any residue, X2 is a G or an N, X3 is any residue, and X4 is preferably a N or a D.

LARX₁X₂X₃X₄ where X1 is any residue, X2 is a G or an N, X3 is any residue, and X4 is preferably a N or a D.

LHKX₁X₂X₃X₄ where X1 is any residue, X2 is a G or an N, X3 is any residue, and X4 is preferably a N or a D.

LHRX₁X₂X₃X₄ where X1 is any residue, X2 is a G or an N, X3 is any residue, and X4 is preferably a N or a D.

LSKX₁X₂X₃X₄ where X1 is any residue, X2 is a G or an N, X3 is any residue, and X4 is preferably a N or a D.

LSRX₁X₂X₃X₄ where X1 is any residue, X2 is a G or an N, X3 is any residue, and X4 is preferably a N or a D.

YAKX₁X₂X₃X₄ where X1 is any residue, X2 is a G or an N, X3 is any residue, and X4 is preferably a N or a D.

YARX₁X₂X₃X₄ where X1 is any residue, X2 is a G or an N, X3 is any residue, and X4 is preferably a N or a D.

YHKX₁X₂X₃X₄ where X1 is any residue, X2 is a G or an N, X3 is any residue, and X4 is preferably a N or a D.

YHRX₁X₂X₃X₄ where X1 is any residue, X2 is a G or an N, X3 is any residue, and X4 is preferably a N or a D.

YMKX₁X₂X₃X₄ where X1 is any residue, X2 is a G or an N, X3 is any residue, and X4 is preferably a N or a D.

YMRX₁X₂X₃X₄ where X1 is any residue, X2 is a G or an N, X3 is any residue, and X4 is preferably a N or a D.

YSKX₁X₂X₃X₄ where X1 is any residue, X2 is a G or an N, X3 is any residue, and X4 is preferably a N or a D.

YVKSX₁X₂X₃ (SEQ ID NO: 20488), where X1 is a D, E, Q, N, or A; X2 is N, V, T, P, G, S, M, or A; and X3 is a C, G, I, A, N, R, K, S, or T.

YVRSX₁X₂X₃ (SEQ ID NO: 20504), where X1 is a D, E, Q, N, or A; X2 is N, V, T, P, G, S, M, or A; and X3 is a C, G, I, A, N, R, K, S, or T.

YVKTX₁X₂X₃ (SEQ ID NO: 20489), where X1 is a D, E, Q, N, or A; X2 is N, V, T, P, G, S, M, or A; and X3 is a C, G, I, A, N, R, K, S, or T.

YVRTX₁X₂X₃ (SEQ ID NO: 20490), where X1 is a D, E, Q, N, or A; X2 is N, V, T, P, G, S, M, or A; and X3 is a C, G, I, A, N, R, K, S, or T.

X₁X₂X₃X₄X₅WP, where X₁ is a R, K, F, I, L, T, Y; and X₂, X₃ and X₄ are any residue; and X₅ is D or E.

RX₁X₂X₃X₄X₅P, where X₁ and X₂ are any residue; X₃ is D; X₄ is Y, F, V, A, T, S, I, L; and X₄ is Y, F, V, or M.

RX₁X₂DX₃YP (SEQ ID NO: 20491), where X1 and X are any residues; and X3 is A, V, F, T, or S.

X₁YAKSX₂X₃ (SEQ ID NO: 20501), where X1 is an S, Y, D, N, or A; X2 is an L, Y, V, A, Q, or E; and X₃ is an L, A, P, D, N, or I.

X₁KSSX₂X₃W (SEQ ID NO: 20503), where X1 is V, P, D, S, L; X2 is N, G, V, S, H, A, or R; X3 is V, N, S, H, D, or R.

### Tables

Table 1. Capsids that demonstrate preferential binding to hTFRC expressed in HEK293 cells. The second round 7-mer 588 insertion library was screened for binding HEK293 cells transiently transfected with hTFRC (HEK-hTFRC) or control (GFP) HEK293 (HEK-Control). The table provides enrichment scores for each sequence, where enrichment is the log 2 assay reads per million (RPM)/RPM in the virus library before screening. “−”<=0; “+”>0 to <=1; “++”>1 to <=2; “+++”>2.

Table 2. Capsids that transduce hCMECs in vitro. Capsids with the indicated 21-mer NT sequences and 7-mer AA sequences inserted between AA588 and 589 of AAV9 K449R were evaluated for their ability to transduce hCMEC/D3 and mouse brain microvascular endothelial cells (mBMVEC). The table provides enrichment scores for each sequence, where enrichment is the log 2 assay reads per million (RPM)/RPM in the virus library before screening. “−”<=0; “+”>0 to <=1; “++”>1 to <=2; “+++”>2.

Table 3. Capsids that transduce multicellular BBB model cultures containing endothelial cells, astrocytes, and pericytes. The table provides enrichment scores for each sequence, where enrichment is the log 2 assay reads per million (RPM)/RPM in the virus library before screening. “−”<=0; “+”>0 to <=1; “++”>1 to <=4; “+++”>4.

Table 4. Capsids that selectively bind human TFRC-Fc fusion protein. The second round 7-mer 588 insertion library was screened for binding to hTFRC-Fc fusion proteins in a pull-down assay. The table provides enrichment scores for each sequence, where enrichment is the log 2 assay reads per million (RPM)/RPM in the virus library before screening. “−”<=0; “+”>0 to <=1; “++”>1 to <=2; “+++”>2.

Table 5. Capsids that selectively bind human TFRC-Fc fusion protein and bind CHO cells expressing TFRC. The second round 7-mer 588 insertion library was screened for capsids that bind CHO cells stably expressing human TFRC (hTFRC), untransduced control CHO cells (Untransduced), or control transduced CHO cells (Lenti-control). The table provides enrichment scores for each sequence, where enrichment is the log 2 assay reads per million (RPM)/RPM in the virus library before screening. “−”<=0; “+”>0 to <=1; “++”>1 to <=5; “+++” >5.

Table 6. Capsids that selectively bind human TFRC-Fc fusion protein and bind and transduce CHO cells expressing TFRC. The second round 7-mer 588 insertion library was screened for capsids that bind and transduce CHO cells stably expressing human TFRC (hTFRC), untransduced control CHO cells (Untransduced), or control transduced CHO cells (Lenti-control). The table provides enrichment scores for each sequence, where enrichment is the log 2 assay reads per million (RPM)/RPM in the virus library before screening. “−”<=0; “+”>0 to <=1; “++”>1 to <=5; “+++”>5.

Table 7. Capsids that selectively bind human TFRC-Fc fusion protein and bind HEK cells expressing TFRC. The second round 7-mer 588 insertion library was screened for capsids that bind HEK293 cells transiently expressing human TFRC (HEK-hTFRC) or control transduced HEK293 cells (HEK-GFP). The table provides enrichment scores for each sequence, where enrichment is the log 2 assay reads per million (RPM)/RPM in the virus library before screening. “−”<=0; “+”>0 to <=1; “+”>1 to <=2; “+”>2.

Table 8. Capsids that selectively bind human TFRC-Fc fusion protein and bind marmoset TFRC-Fc fusions. The second round 7-mer 588 insertion library was screened for capsids that bind human and marmoset TFRC-Fc fusion proteins (hTFRC and marTFRC, respectively). The capsids were also evaluated for their ability to bind HEK293 cells transiently expressing human TFRC (HEK-hTFRC) or control transduced HEK293 cells (HEK-GFP). The table provides enrichment scores for each sequence, where enrichment is the log 2 assay reads per million (RPM)/RPM in the virus library before screening. “−”<=0; “+”>0 to <=1; “++”>1 to <=23; “+++”>2.

Table 9. Capsids that selectively bind human and marmoset TFRC-Fc fusion proteins. A 7-mer 588 insertion library was screened for binding to human or marmoset TFRC-Fc fusion proteins (hTFRC and marTFRC, respectively). Binding was performed at pH7.4. Washes were performed at pH7.4 or at pH5.5, as indicated. The table provides enrichment scores for each sequence, where enrichment is the log 2 assay reads per million (RPM)/RPM in the virus library before screening. “−”<=0; “+”>0 to <=1; “++”>1 to <=5; “+++”>5.

Table 10. Capsids that selectively bind human TFRC-Fc fusion protein. A 7-mer or 10-mer loop IV substitution library was screened for binding to human TFRC-Fc fusion proteins. Binding was performed at pH7.4. Washes were performed at pH7.4 or at pH5.5, as indicated. The table provides enrichment scores for each sequence, where enrichment is the log 2 assay reads per million (RPM)/RPM in the virus library before screening. “−”<=0; “+”>0 to <=1; “++”>1 to <=3; “+++”>3.

Table 11. Capsids that selectively bind human TFRC-Fc fusion protein where the apical domain was replaced by the corresponding region of mouse TFRC. A 7-mer or 10-mer loop IV substitution library was screened for binding to human TFRC-Fc fusion proteins. Binding was performed at pH7.4. Washes were performed at pH7.4 or at pH5.5, as indicated. The table provides enrichment scores for each sequence, where enrichment is the log 2 assay reads per million (RPM)/RPM in the virus library before screening. “−”<=0; “+”>0 to <=2; “+”>2 to <=4; “+++”>4.

Table 12. Capsids that selectively bind human TFRC-Fc fusion protein. A 7-mer 588 insertion Fit4Function library was screened for binding to human TFRC-Fc fusion protein. Binding was performed at pH7.4 or pH5.5, as indicated. The table provides enrichment scores for each sequence, where enrichment is the log 2 assay reads per million (RPM)/RPM in the virus library before screening. “−”<=0; “+”>0 to <=2; “+”>2 to <=4; “+”>4.

Table 13. The table provides sequence listing of 7-mers that impart TFRC binding when inserted with AAV9 between AA 588-589 and their cluster assignment (FIG. 7). The table provides the AA SEQ ID, AA sequence, UMAP coordinates, and cluster number.

TABLE B
Individual capsid AA sequences TFRC.

| | |
|---|---|
| >AAV9 | MAADGYLPDWLEDNLSEGIREWWALKPGAPQPKANQQHQDN |
| | ARGLVLPGYKYLGPGNGLDKGEPVNAADAAALEHDKAYDQQ |
| | LKAGDNPYLKYNHADAEFQERLKEDTSFGGNLGRAVFQAKKR |
| | LLEPLGLVEEAAKTAPGKKRPVEQSPQEPDSSAGIGKSGAQPAK |
| | KRLNFGQTGDTESVPDPQPIGEPPAAPSGVGSLTMASGGGAPV |
| | ADNNEGADGVGSSSGNWHCDSQWLGDRVITTSTRTWALPTYN |
| | NHLYKQISNSTSGGSSNDNAYFGYSTPWGYFDFNRFHCHFSPR |
| | DWQRLINNNWGFRPKRLNFKLFNIQVKEVTDNNGVKTIANNLT |
| | STVQVFTDSDYQLPYVLGSAHEGCLPPFPADVFMIPQYGYLTLN |
| | DGSQAVGRSSFYCLEYFPSQMLRTGNNFQFSYEFENVPFHSSYA |
| | HSQSLDRLMNPLIDQYLYYLSKTINGSGQNQQTLKFSVAGPSN |
| | MAVQGRNYIPGPSYRQQRVSTTVTQNNNSEFAWPGASSWALN |
| | GRNSLMNPGPAMASHKEGEDRFFPLSGSLIFGKQGTGRDNVDA |
| | DKVMITNEEEIKTTNPVATESYGQVATNHQSAQAQAQTGWVQ |
| | NQGILPGMVWQDRDVYLQGPIWAKIPHTDGNFHPSPLMGGFG |
| | MKHPPPQILIKNTPVPADPPTAFNKDKLNSFITQYSTGQVSVEIE |
| | WELQKENSKRWNPEIQYTSNYYKSNNVEFAVNTEGVYSEPRPI |
| | GTRYLTRNL* (SEQ ID NO: 20506) |
| >AAV9 K449R | MAADGYLPDWLEDNLSEGIREWWALKPGAPQPKANQQHQDN |
| | ARGLVLPGYKYLGPGNGLDKGEPVNAADAAALEHDKAYDQQ |
| | LKAGDNPYLKYNHADAEFQERLKEDTSFGGNLGRAVFQAKKR |
| | LLEPLGLVEEAAKTAPGKKRPVEQSPQEPDSSAGIGKSGAQPAK |
| | KRLNFGQTGDTESVPDPQPIGEPPAAPSGVGSLTMASGGGAPV |
| | ADNNEGADGVGSSSGNWHCDSQWLGDRVITTSTRTWALPTYN |
| | NHLYKQISNSTSGGSSNDNAYFGYSTPWGYFDFNRFHCHFSPR |
| | DWQRLINNNWGFRPKRLNFKLFNIQVKEVTDNNGVKTIANNLT |
| | STVQVFTDSDYQLPYVLGSAHEGCLPPFPADVFMIPQYGYLTLN |
| | DGSQAVGRSSFYCLEYFPSQMLRTGNNFQFSYEFENVPFHSSYA |
| | HSQSLDRLMNPLIDQYLYYLSRTINGSGQNQQTLKFSVAGPSN |
| | MAVQGRNYIPGPSYRQQRVSTTVTQNNNSEFAWPGASSWALN |
| | GRNSLMNPGPAMASHKEGEDRFFPLSGSLIFGKQGTGRDNVDA |
| | DKVMITNEEEIKTTNPVATESYGQVATNHQSAQAQAQTGWVQ |
| | NQGILPGMVWQDRDVYLQGPIWAKIPHTDGNFHPSPLMGGFG |
| | MKHPPPQILIKNTPVPADPPTAFNKDKLNSFITQYSTGQVSVEIE |
| | WELQKENSKRWNPEIQYTSNYYKSNNVEFAVNTEGVYSEPRPI |
| | GTRYLTRNL* (SEQ ID NO: 20507) |
| >AAV-BI19 | MAADGYLPDWLEDNLSEGIREWWALKPGAPQPKANQQHQDN |
| | ARGLVLPGYKYLGPGNGLDKGEPVNAADAAALEHDKAYDQQ |
| | LKAGDNPYLKYNHADAEFQERLKEDTSFGGNLGRAVFQAKKR |
| | LLEPLGLVEEAAKTAPGKKRPVEQSPQEPDSSAGIGKSGAQPAK |
| | KRLNFGQTGDTESVPDPQPIGEPPAAPSGVGSLTMASGGGAPV |
| | ADNNEGADGVGSSSGNWHCDSQWLGDRVITTSTRTWALPTYN |
| | NHLYKQISNSTSGGSSNDNAYFGYSTPWGYFDFNRFHCHFSPR |
| | DWQRLINNNWGFRPKRLNFKLFNIQVKEVTDNNGVKTIANNLT |
| | STVQVFTDSDYQLPYVLGSAHEGCLPPFPADVFMIPQYGYLTLN |
| | DGSQAVGRSSFYCLEYFPSQMLRTGNNFQFSYEFENVPFHSSYA |
| | HSQSLDRLMNPLIDQYLYYLSRTINGSGQNQQTLKFSVAGPSN |
| | MAVQGRNYIPGPSYRQQRVSTTVTQNNNSEFAWPGASSWALN |
| | GRNSLMNPGPAMASHKEGEDRFFPLSGSLIFGKQGTGRDNVDA |
| | DKVMITNEEEIKTTNPVATESYGQVATNHQSAQYSRIGPNAQA |
| | QTGWVQNQGILPGMVWQDRDVYLQGPIWAKIPHTDGNFHPSP |
| | LMGGFGMKHPPPQILIKNTPVPADPPTAFNKDKLNSFITQYSTG |
| | QVSVEIEWELQKENSKRWNPEIQYTSNYYKSNNVEFAVNTEGV |
| | YSEPRPIGTRYLTRNL* (SEQ ID NO: 20508) |
| >AAV-BI98 | MAADGYLPDWLEDNLSEGIREWWALKPGAPQPKANQQHQDN |
| | ARGLVLPGYKYLGPGNGLDKGEPVNAADAAALEHDKAYDQQ |
| | LKAGDNPYLKYNHADAEFQERLKEDTSFGGNLGRAVFQAKKR |
| | LLEPLGLVEEAAKTAPGKKRPVEQSPQEPDSSAGIGKSGAQPAK |
| | KRLNFGQTGDTESVPDPQPIGEPPAAPSGVGSLTMASGGGAPV |
| | ADNNEGADGVGSSSGNWHCDSQWLGDRVITTSTRTWALPTYN |
| | NHLYKQISNSTSGGSSNDNAYFGYSTPWGYFDFNRFHCHFSPR |
| | DWQRLINNNWGFRPKRLNFKLFNIQVKEVTDNNGVKTIANNLT |
| | STVQVFTDSDYQLPYVLGSAHEGCLPPFPADVFMIPQYGYLTLN |
| | DGSQAVGRSSFYCLEYFPSQMLRTGNNFQFSYEFENVPFHSSYA |
| | HSQSLDRLMNPLIDQYLYYLSRTINGSGQNQQTLKFSVAGPSN |
| | MAVQGRNYIPGPSYRQQRVSTTVTQNNNSEFAWPGASSWALN |
| | GRNSLMNPGPAMASHKEGEDRFFPLSGSLIFGKQGTGRDNVDA |
| | DKVMITNEEEIKTTNPVATESYGQVATNHQSAQYSRLNMNAQ |
| | AQTGWVQNQGILPGMVWQDRDVYLQGPIWAKIPHTDGNFHPS |
| | PLMGGFGMKHPPPQILIKNTPVPADPPTAFNKDKLNSFITQYST |
| | GQVSVEIEWELQKENSKRWNPEIQYTSNYYKSNNVEFAVNTEG |
| | VYSEPRPIGTRYLTRNL* (SEQ ID NO: 20509) |
| >AAV-BI99 | MAADGYLPDWLEDNLSEGIREWWALKPGAPQPKANQQHQDN |
| | ARGLVLPGYKYLGPGNGLDKGEPVNAADAAALEHDKAYDQQ |
| | LKAGDNPYLKYNHADAEFQERLKEDTSFGGNLGRAVFQAKKR |
| | LLEPLGLVEEAAKTAPGKKRPVEQSPQEPDSSAGIGKSGAQPAK |
| | KRLNFGQTGDTESVPDPQPIGEPPAAPSGVGSLTMASGGGAPV |
| | ADNNEGADGVGSSSGNWHCDSQWLGDRVITTSTRTWALPTYN |
| | NHLYKQISNSTSGGSSNDNAYFGYSTPWGYFDFNRFHCHFSPR |
| | DWQRLINNNWGFRPKRLNFKLFNIQVKEVTDNNGVKTIANNLT |
| | STVQVFTDSDYQLPYVLGSAHEGCLPPFPADVFMIPQYGYLTLN |
| | DGSQAVGRSSFYCLEYFPSQMLRTGNNFQFSYEFENVPFHSSYA |
| | HSQSLDRLMNPLIDQYLYYLSRTINGSGQNQQTLKFSVAGPSN |
| | MAVQGRNYIPGPSYRQQRVSTTVTQNNNSEFAWPGASSWALN |
| | GRNSLMNPGPAMASHKEGEDRFFPLSGSLIFGKQGTGRDNVDA |
| | DKVMITNEEEIKTTNPVATESYGQVATNHQSAQYSRLNKDAQ |
| | AQTGWVQNQGILPGMVWQDRDVYLQGPIWAKIPHTDGNFHPS |
| | PLMGGFGMKHPPPQILIKNTPVPADPPTAFNKDKLNSFITQYST |
| | GQVSVEIEWELQKENSKRWNPEIQYTSNYYKSNNVEFAVNTEG |
| | VYSEPRPIGTRYLTRNL* (SEQ ID NO: 20510) |
| >AAV-BI100 | MAADGYLPDWLEDNLSEGIREWWALKPGAPQPKANQQHQDN |
| | ARGLVLPGYKYLGPGNGLDKGEPVNAADAAALEHDKAYDQQ |
| | LKAGDNPYLKYNHADAEFQERLKEDTSFGGNLGRAVFQAKKR |
| | LLEPLGLVEEAAKTAPGKKRPVEQSPQEPDSSAGIGKSGAQPAK |
| | KRLNFGQTGDTESVPDPQPIGEPPAAPSGVGSLTMASGGGAPV |
| | ADNNEGADGVGSSSGNWHCDSQWLGDRVITTSTRTWALPTYN |
| | NHLYKQISNSTSGGSSNDNAYFGYSTPWGYFDFNRFHCHFSPR |
| | DWQRLINNNWGFRPKRLNFKLFNIQVKEVTDNNGVKTIANNLT |
| | STVQVFTDSDYQLPYVLGSAHEGCLPPFPADVFMIPQYGYLTLN |
| | DGSQAVGRSSFYCLEYFPSQMLRTGNNFQFSYEFENVPFHSSYA |
| | HSQSLDRLMNPLIDQYLYYLSRTINGSGQNQQTLKFSVAGPSN |
| | MAVQGRNYIPGPSYRQQRVSTTVTQNNNSEFAWPGASSWALN |
| | GRNSLMNPGPAMASHKEGEDRFFPLSGSLIFGKQGTGRDNVDA |
| | DKVMITNEEEIKTTNPVATESYGQVATNHQSAQVHRLODKAQ |
| | AQTGWVQNQGILPGMVWQDRDVYLQGPIWAKIPHTDGNFHPS |
| | PLMGGFGMKHPPPQILIKNTPVPADPPTAFNKDKLNSFITQYST |
| | GQVSVEIEWELQKENSKRWNPEIQYTSNYYKSNNVEFAVNTEG |
| | VYSEPRPIGTRYLTRNL* (SEQ ID NO: 20511) |
| >AAV-BI101 | MAADGYLPDWLEDNLSEGIREWWALKPGAPQPKANQQHQDN |
| | ARGLVLPGYKYLGPGNGLDKGEPVNAADAAALEHDKAYDQQ |
| | LKAGDNPYLKYNHADAEFQERLKEDTSFGGNLGRAVFQAKKR |
| | LLEPLGLVEEAAKTAPGKKRPVEQSPQEPDSSAGIGKSGAQPAK |
| | KRLNFGQTGDTESVPDPQPIGEPPAAPSGVGSLTMASGGGAPV |
| | ADNNEGADGVGSSSGNWHCDSQWLGDRVITTSTRTWALPTYN |
| | NHLYKQISNSTSGGSSNDNAYFGYSTPWGYFDFNRFHCHFSPR |
| | DWQRLINNNWGFRPKRLNFKLFNIQVKEVTDNNGVKTIANNLT |
| | STVQVFTDSDYQLPYVLGSAHEGCLPPFPADVFMIPQYGYLTLN |
| | DGSQAVGRSSFYCLEYFPSQMLRTGNNFQFSYEFENVPFHSSYA |
| | HSQSLDRLMNPLIDQYLYYLSRTINGSGQNQQTLKFSVAGPSN |
| | MAVQGRNYIPGPSYRQQRVSTTVTQNNNSEFAWPGASSWALN |
| | GRNSLMNPGPAMASHKEGEDRFFPLSGSLIFGKQGTGRDNVDA |
| | DKVMITNEEEIKTTNPVATESYGQVATNHQSAQYHRLSNNAQ |
| | AQTGWVQNQGILPGMVWQDRDVYLQGPIWAKIPHTDGNFHPS |
| | PLMGGFGMKHPPPQILIKNTPVPADPPTAFNKDKLNSFITQYST |
| | GQVSVEIEWELQKENSKRWNPEIQYTSNYYKSNNVEFAVNTEG |
| | VYSEPRPIGTRYLTRNL* (SEQ ID NO: 20512) |
| >AAV-BI102 | MAADGYLPDWLEDNLSEGIREWWALKPGAPQPKANQQHQDN |
| | ARGLVLPGYKYLGPGNGLDKGEPVNAADAAALEHDKAYDQQ |
| | LKAGDNPYLKYNHADAEFQERLKEDTSFGGNLGRAVFQAKKR |
| | LLEPLGLVEEAAKTAPGKKRPVEQSPQEPDSSAGIGKSGAQPAK |
| | KRLNFGQTGDTESVPDPQPIGEPPAAPSGVGSLTMASGGGAPV |
| | ADNNEGADGVGSSSGNWHCDSQWLGDRVITTSTRTWALPTYN |
| | NHLYKQISNSTSGGSSNDNAYFGYSTPWGYFDFNRFHCHFSPR |
| | DWQRLINNNWGFRPKRLNFKLFNIQVKEVTDNNGVKTIANNLT |
| | STVQVFTDSDYQLPYVLGSAHEGCLPPFPADVFMIPQYGYLTLN |
| | DGSQAVGRSSFYCLEYFPSQMLRTGNNFQFSYEFENVPFHSSYA |
| | HSQSLDRLMNPLIDQYLYYLSRTINGSGQNQQTLKFSVAGPSN |
| | MAVQGRNYIPGPSYRQQRVSTTVTQNNNSEFAWPGASSWALN |
| | GRNSLMNPGPAMASHKEGEDRFFPLSGSLIFGKQGTGRDNVDA |
| | DKVMITNEEEIKTTNPVATESYGQVATNHQSAQLHALSHNAQ |
| | AQTGWVQNQGILPGMVWQDRDVYLQGPIWAKIPHTDGNFHPS |
| | PLMGGFGMKHPPPQILIKNTPVPADPPTAFNKDKLNSFITQYST |
| | GQVSVEIEWELQKENSKRWNPEIQYTSNYYKSNNVEFAVNTEG |
| | VYSEPRPIGTRYLTRNL* (SEQ ID NO: 20513) |

### Methods for Examples 1-4

### Plasmids

The AAV9, AAV-BI18, BI19, BI98-BI102 and AAV-BI159-165 Rep-Cap plasmids were generated by gene synthesis (GenScript). The CAG-WPRE-hGH pA backbone was obtained from Viviana Gradinaru through Addgene (#99122). GFP, GFP-2A-luciferase, and mScarlet cDNAs were synthesized as gBlocks (IDT) or synthesized and cloned (GenScript).

Full length TFRC cDNA expression and lentiviral plasmids were cloned by inserting the open reading frames of TFRC (human: NM_003234.4; mouse TFRC: NM_011638.4; marmoset TFRC: NM_001301847.1) into the lentiviral backbone pLenti-EF-FH-TAZ-ires-blast (Addgene #52083) with EcoRI/SalI sites.

For Fc fusion proteins, the coding sequence of the extracellular regions of TFRC (human: 89-760aa; mouse: 89-763aa; marmoset: 89-760aa) were amplified by PCR and inserted into pCMV6-XL4 FLAG-NGRN-Fc (Addgene #115773) with EcoRV and Xbal sites. Signal peptide H7 (ATGGAGTTTGGGCTGAGCTGGGTTTTCCTCGTTGCTCTTTTTAGAGGTGTCCAGTGT (SEQ ID NO: 20493)) was introduced to N terminal of TFRC sequences to direct protein secretion. hTFRC-mApical construct was made by replacing the human TFRC apical domain (AA188-384) by mouse TFRC apical domain (AA 190-386).

### Cell Lines and Primary Cultures

HEK293T/17 (CRL-11268), Pro5 (CRL-1781) and CHO-K1 (CCL-61) were obtained from ATCC. mBMVEC cells (C57-6023) and hBMVECs cells (H-6023) were obtained from Cell Biologics and cultured as directed by the manufacturer. hCMEC/D3 cells were from Millipore-Sigma (SCC066).

### Expression and Purification of Fc-Tagged Protein in HEK293-FT Cells

26 million HEK293-FT cells were seated per 150 mm plate the day before transfection. The next day, complete media was changed to Pro293TMa-CDMTM media with two brief rinses with Pro293TMa-CDMTM media to remove serum. Cells were transduced with PEI and 40 ug DNA per plate a few hours after media change. The media was replaced 18 hours after transfection. At the second day post-transfection, cell supernatants containing secreted recombinant protein were passed through a 0.45-mm pore size filter and purified on Protein A-Sepharose. 200 ul Protein A-Sepharose were incubated with 100 ml cell culture supernatant overnight at 4C with shaking. The next day, the beads were collected and washed 3 times with 10 ml of PBS, and the proteins were eluted in 200 ul of 100 mM glycine (pH2.7). Then 1/10 volume of 1M Tris (pH8.8) was added to the eluted protein fractions to neutralize the pH.

### Lentivirus Production

Lentivirus was produced with a third-generation lentivirus system by cotransfection of three packaging plasmids (pMDLg-RRE, pRSV-Rev and pVSV-G) and vector plasmid encoding hTFRC at a ratio of 4:1:1:6 in HEK cells. Lentivirus was harvested 3 days after transfection, filtered with 0.45 um PES to remove the cell debris.

HEK293 and CHO stable cell line generation

CHO cells were seeded 150,000/well in 12 well plates and transduced with lentivirus containing media for 48 hrs, then transferred to 10 cm dishes for selection with 10 ug/mL blasticidin for about a week. Once all cells had died in the control plate, the selected CHO cells were maintained in media with 1 ug/mL blasticidin. The expression of hTFRC was validated with immunostaining with OKT9 antibody from ThermoFisher (Catalog #14-0719-82).

### Library Cloning

The oligo pool or NNK PCR products were assembled into the RNA expression plasmid with previously described methods in Deverman et al. Nature Biotechnology 2016. For generating the 7-mer 588 site insertion capsid libraries, the hand-mixed (IDT) primer 5′-GTATTCCTTGGTTTTGAACCCAACCGGTCTGCGCCTGTGCMNNMNNMNNMNNMNN MNNMNNTTGGGCACTCTGGTGGTTTGTG-3′ (SEQ ID NO: 20494) primer was used with forward primer 5′-CACTCATCGACCAATACTTGTACTATCTCT (SEQ ID NO: 20495) to PCR amplify a modified AAV9 template (K449R) 10 ng pUC57-wtAAV9-X/A plasmid (as in Deverman et al Nature Biotechnology 2016) using Q5 Hot Start High-Fidelity 2× Master Mix (NEB, M0494S) following the manufacturer's protocol.

To generate the loop IV libraries the forward primers (10merNNK) 5′-ATCGACCAATACTTGTACTATCTCTCTAGAACTNNKNNKNNKNNKNNKNNKNNKNN KNNKNNKCTAAAATTCAGTGTGGCCGGACC (SEQ ID NO: 20496) or (7merNNK) 5′-ATCGACCAATACTTGTACTATCTCTCTAGAACTNNKNNKNNKNNKNNKNNKNNKCT AAAATTCAGTGTGGCCGGACC (SEQ ID NO: 20497) were used with the reverse primer 5-TTGTCCTTGTTGAAGGCCGTTGGAG (SEQ ID NO: 20498) to amplify the same modified AAV9 template (K449) as described above.

To assemble an oligonucleotide Library Synthesis (OLS) Pool (Agilent) into an AAV genome, 5 pM of the OLS pool was used as an initial reverse primer along with 0.5 μM of the forward primer to amplified and extended 10 ng of DNA plasmid containing a fragment of AAV9 (pUC57-wtAAV9-X/A) for 5 cycles using Q5® High-Fidelity 2× Master Mix (NEB #M0492S) following the manufacturer's protocol. After the 5-cycle amplification and extension of the oligo pool, the reactions were spiked with 0.5 μM of primer 5′-GTATTCCTTGGTTTTGAACCCAACCG (SEQ ID NO: 20499) (588 site insertion libraries) or 5′-CACTCATCGACCAATACTTGTACTATCTCT (SEQ ID NO: 20495) (loop IV libraries) and amplified for an additional 25 cycles. The PCR product was then purified using Agencourt AMPure XP SPRI paramagnetic beads (Beckman Coulter #A63880) or column purified using a Zymo Research DNA Clean & Concentrator-5 kit (Zymo Research #D4013) following the manufacturer's protocol.

All library PCR products were cleaned up using AMPure XP beads (Beckman, A63881) following the manufacturer's protocol. The NNK PCR insert was assembled into a linearized mRNA selection vector (AAV9-CMV-Express, described below) with NEBuilder HiFi DNA Assembly Master Mix (NEB, E2621L). Afterwards, Quick CIP (NEB, M0508S) was spiked into the reaction and incubated at 37° C. for 30 min to dephosphorylate unincorporated dNTPs. Finally, T5 Exonuclease (NEB M0663S) was added to the reaction mixture and incubated at 37° C. for 30 min to remove unassembled products. The final assembled product was cleaned up using AMPure XP beads (Beckman, A63881) following the manufacturer's protocol quantified using the Qubit dsDNA HS Assay Kit (Thermo Fisher Scientific, Q32851).

### Synthetic Round 2 Library Design

The library oligo pool consisted of 7-mer insertion sequences recovered from the Round 1 binding assays. In addition, the Experiment 1, round 2 library contained all possible sequences conforming to the following 7-mer motifs [FLY][AS][KR]X[GN]XN (SEQ ID NO: 20500) and XXS[ST]NG[VIR] (SEQ ID NO: 20505), where X is any residue. In experiment 2, the round 2 library comprised only those sequences recovered through binding to Fc-target proteins including TFRC, control sequences including those containing stop codons (to aid in the assessment of cross packaging), and WT and other reference sequences. All sequences were encoded by two distinct nucleotide sequences designed to serve as biological replicates. In Experiment 4, the F4F “Hammerhead” library was generated as described in Published Patent Application US20210403946 A1.

### RNA Selection Plasmids

The mRNA selection vector (AAV9-CMV-Express) was designed to enrich for functional AAV capsid sequences by recovering capsid mRNA from transduced cells. AAV9-CMV-Express uses a ubiquitous CMV enhancer and AAV5 p41 gene regulatory elements to drive AAV9 Cap expression. The AAV9-CMV-Express plasmid was constructed by cloning the following elements into an AAV genome plasmid in the listed order: a cytomegalovirus (CMV) enhancer-promoter, a synthetic intron and the AAV5 P41 promoter along with 3′ end of the AAV2 Rep gene, which includes the splice donor sequences for the capsid RNA. The capsid gene splice donor sequence in AAV2 Rep was modified from CAGGTACCA to a consensus donor sequence CAGGTAAGT. The AAV9 capsid gene sequence was synthesized with nucleotide changes at 1,344, 1,346 and 1,347 (which introduces a K449R mutation) and at 1,782 (which is a silent mutation) to introduce restriction enzyme recognition sites for NNK library PCR insert fragment cloning. The AAV2 polyadenylation sequence was replaced with a simian virus 40 (SV40) late polyadenylation signal. A previously described cap-deficient Rep-AAP AAV helper plasmid1 was supplied in trans to generate virus with the AAV9-CMV-Express vector.

### Virus Production and Purification

Recombinant AAVs were generated following a previously published protocol (Challis et al 2019) with minor modifications as described below. HEK293T/17 cells (ATCC, CRL-11268) were seeded at 22 million cells per 15-cm plate the day before transfection and grown in DMEM with GlutaMAX (Gibco, 10569010) supplemented with 5% FBS and 1× non-essential amino acid solution (NEAA) (Gibco, 11140050). The next day, the cells were triple transfected with 39.93 ug of total plasmid DNA encoding rep-cap, pHelper and an ITR-flanked transgene at a plasmid ratio of 4:2:1, respectively, using polyethylenimine (PEI) MAX (Polyscience, 24765-1) at a DNA: PEI ratio of 1:3.5. Twenty hours after transfection, the medium was changed to fresh DMEM with GlutaMAX supplemented with 5% FBS and 1× NEAA. Seventy-two hours after transfection, cells were scraped and pelleted at 2,000 RCF for 10 min. The pellets were resuspended in 7 ml of Salt Active Nuclease (SAN) digestion buffer (500 mM NaCl, 40 mM Tris-base, 10 mM MgCl2, SAN enzyme (ArcticZymes, 70920-202) at 100 U/ml) for every ten plates and incubated at 37° C. for 1.5 h. Afterwards, the lysate was clarified at 2,000 RCF for 10 min and loaded onto a density step gradient containing OptiPrep (Cosmo Bio, AXS-1114542) at 60%, 40%, 25% and 15% at a volume of 5, 5, 6, and 6 ml, respectively in OptiSeal tubes (Beckman, 361625). The step gradients were spun in a Beckman Type 70ti rotor (Beckman, 337922) in a Sorvall WX+ ultracentrifuge (Thermo Fisher Scientific, 75000090) at 69,000 r.p.m. for 1 h at 18° C. Afterwards, 4-4.5 ml of the 40-60% interface was extracted using a 16-gauge needle, filtered through a 0.22-μm PES filter and then buffer exchanged with 100 K MWCO protein concentrators (Thermo Fisher Scientific, 88532) into PBS containing 0.001% Pluronic F-68 and concentrated down to a volume of 500 ul. The concentrated virus was then filtered through a 0.22-μm PES filter and stored at 4° C. or −80° C.

The AAV capsid library was produced similarly as above with following exceptions: each plate was triple transfected with 39.93 ug of total plasmid DNA encoding pHelper, RepStop encoding the AAV2 Rep genes, pUC19 at a ratio of 2:1:1, respectively, and with 10 ng of assembled library DNA; the media and cell lysates were harvested 60 hours post transfection and purified following the published protocol (Deverman et al. Nature Biotechnology 2016).

ddPCR Titering

Vector aliquots were treated with Turbonuclease (Cat #) at 37 C for 1 hour and then deactivated by adding 5 uL EDTA and heated at 70C for 10 minutes. Afterwards, the samples were treated with proteinase K for 2 hours and heated inactivated diluted to within the dynamic range of the ddPCR assay (1 to 104 vector genomes) per reaction.

### SDS Page

Briefly, proteins in HEK293 cell media pre-or post-pull down with ProA magnetic beads were separated on Bolt 4-12% Bis-Tris Plus gels and stained with SYPRO Ruby (Thermofisher).

### Animals

All procedures were performed as approved by the Broad Institute IACUC. BALB/cJ (000651), C57B1/6J (000664), and NSG (00557) were obtained from The Jackson Laboratory (JAX). Recombinant AAV vectors were administered intravenously via the retro-orbital sinus in young adult male or female mice. Mice were randomly assigned to groups based on predetermined sample sizes.

### Tissue Processing, Immunohistochemistry and Imaging

Mice were anesthetized with Euthasol and transcardially perfused with phosphate buffered saline (PBS) at room temperature followed by 4% paraformaldehyde (PFA) in ice cold PBS. Tissues were post-fixed overnight in 4% PFA in PBS and sectioned by vibratome. IHC was performed on floating sections with antibodies diluted in PBS containing 10% donkey serum, 0.1% Triton X-100, and 0.05% sodium azide. Primary antibodies were incubated at room temperature overnight. The sections were then washed and stained with secondary (Alexa-conjugated antibodies, 1:1000) for 4 hours or overnight.

### In Vivo Vector and Capsid Biodistribution

5- to 6-week-old C57B1/6J mice or BALB/cJ mice were injected intravenously with 10e11 vg of AAV vector packaged into the indicated capsid. One or two hours after injection, the mice were perfused with PBS and tissues were collected and frozen at −80° C. Samples were processed for AAV genome biodistribution analysis and normalized to the number of copies of mouse genomes using qPCR for the GFP element and mouse glucagon by qPCR as previously described (2). NSG mice were used for experiments involving sequential injection of AAV-BI30 carrying human TFRC or Ly6a (1e11 vg/mouse) followed by AAV-BI19: CAG-mScarlet or AAV-PHP.eB: CAG-mScarlet (3e11 vg/mouse) 1 week after the initial injection.

### Luciferase Transduction Assay

Human, mouse, or marmoset TFRC (0.1 ug/well) was transfected into the indicated cells (HEK293/17: 4×105/well; CHO: 2.5×104/well, BMVECs: 5×103/well) in 96-well plates (PerkinElmer, 6005680) in triplicate. 48 hours later, cells were transduced with AAV-CAG-GFP-2A-Luciferase-WPRE packaged into AAV9 or the indicated AAV-BI #capsid. Luciferase assays were performed with Britelite plus Reporter Gene Assay System (PerkinElmer, 6066766). Luciferase activity was reported as relative light units (RLU) as raw data or normalized to non-transfected control wells transduced with AAV9.

### Cell Binding Assays

TFRC family members or GFP control (0.5 ug/well) were transfected into HEK293T cells (3e5/well) using PEI or into CHO cells (1.5e5/well) with lipofectamine 3000 reagent (ThermoFisher, L3000001) in 24-well plates. 48 hours later, the cells were chilled to 4° C. and the media was exchanged with fresh cold media containing the indicated recombinant AAV (1e5 copies per cell). One hour later, cells were washed with cold PBS for 3 times, then fixed with 4% PFA for IHC or lysed for genomic DNA extraction and qPCR analyses or NGS sequencing. For mouse and human BMVECs, 2e4 cells/well were seeded in 12 well plates the day before exposure to the AAVs. The assay was performed as above except AAV vectors were added at 106 copies/cell. HEK293T/17 cells were seeded at 2E7 per T75 flask 12-24 hours prior to being transfected with 20 μg of cDNA encoding eGFP, human TFRC, mouse TFRC or marmoset TFRC. At 24-48 hours post transfection, the cells were incubated with an AAV9 K449R library (7-mer insertion between amino acids 588 and 589) at 1e11 vg/T75 at 4° C. for 2 hours. Afterwards, the media was exchanged with PBS for 3 times in order to wash away unbound viruses. The AAVs that remained bound to the cells were extracted with TRIzol (Invitrogen) or with whole genomic DNA isolation reagents (DNeasy, Qiagen) to isolate their viral genomes. The viral genomes were then prepared for NGS to quantify the enrichment of peptides that conferred increased capsid ability to bind cells expressing the target protein.

### Transduction Screening In Vitro

For in vitro screening on cells in 2D culture, 1e11 vg of the AAV capsid library was added to HEK293, CHO, or confluent BMVECs or hCMEC/D3 cells and cellular mRNA was collected 60 hours after administration. BBB spheroids were obtained by co-culture of hCMEC/D3, primary human astrocytes and human pericytes as described (Bergmann et al. Nature Protocols 2018). For in vitro screening on spheroids, 1e4 vg/cell was used for binding and 2e5 vg/cell was used for transduction in human serum free media.

mRNA from in vitro assays were recovered using TRIzol (Invitrogen, 15596026) followed by RNA cleanup with RNeasy Mini kit (QIAGEN, 74104). The recovered mRNA was next converted to complementary DNA using an oligonucleotide dT primer using Maxima H Minus Reverse Transcriptase (Thermo Fisher, EP0751).

### Fc-Protein Fusion Cloning and Protein Purification

The extracellular portion together with signal peptide of protein targets are cloned into Fc-tag backbone (Addgene plasmid #115773) using Xbal/EcoRV. To generate Fc-fusion proteins the N terminal cytoplasmic and transmembrane domains were replaced with an H7 signal peptide (Haryadi et al 2015, PLOS One. 2015; 10(2): e0116878; MEFGLSWVFLVALFRGVQC (SEQ ID NO: 42463). The C terminal stop codon was replaced with the Fc tag coding sequence containing intron sequences. Also, there is a HRV 3C site and a linker sequence between TFRC and Fc. The Fc-receptor plasmid was transfected into HEK293 cells (40 ug per 150 mm dish with PEI) in complete DMEM media with 5% FBS. 12-16 hours after transfection, the transfected plate was rinsed with PBS, and 15 ml of serum free media (Lonza, BEBP12-764Q) was added. Media containing the secreted Fc-fusion proteins was collected at 48 and 96 hours, filtered (Millipore SE1M003M00) and stored at 4C until use. 35 ul protein A beads (ThermoFisher, 10001D) and Tween 20 (0.05% final concentration) were added to 30 ml of media and incubated at 4C with end to end rotation. The next day, the beads were washed 3 times with cold PBS containing 0.05% Tween-20. Expression was assessed by running a 5 ul aliquot of protein bound beads on a 4-12% protein gel; the remaining fraction was used for pulldown assay.

Protein A-conjugated beads were used to pull down the LY6A-Fc, LY6C1-Fc, or Fc-only control, which were then washed and incubated with an AAV capsid library.

### Fc-Fusion Pulldown Assay

10 ul Fc-fusion protein bound protein A beads were mixed with 1e10 vg AAV capsid library in PBS with 0.05% Tween-20 and 1% BSA and incubated overnight at 4 C. The next day, beads were washed 3 times with PBS with 0.05% Tween-20 at the indicated pH and then treated with proteinase K to extract the viral genome for NGS preparation after purification with AMPure XP beads following the manufacturer's protocol.

### PCR Recovery of Library Sequences

The AAV genome samples (binding assays) or cDNA (transduction assays) were prepared for next-generation sequencing (NGS) with two rounds of polymerase chain reaction (PCR). In the first round of PCR (PCR1), a set of forward primers and reverse primers containing gene specific priming regions and an overhang sequence containing a portion of the Illumina Read 1 sequence (forward primers) or Illumina Read 2 sequence (reverse primers) were used to selectively amplify AAV genomes from the cDNA with Q5® High-Fidelity 2× Master Mix (NEB #M0492S), with 0.5 μM of each primer.

The forward and reverse primers contain zero or up to eight N nucleotides inserted in between the gene specific priming region and the partial Illumina Read 1 (forward primers) or Read 2 (reverse primers) overhang sequence. The forward and reverse primers were paired to produce amplicons of the same size.

The number of cycles performed in PCR1 using 1 uL of cDNA input was chosen to stop before the exponential amplification phase and was determined with qPCR using FastStart Universal SYBR Green Master (Millipore Sigma #4913850001) or Q5® High-Fidelity 2× Master Mix (NEB #M0492S) with SYBR® Green I nucleic acid stain (VWR #12001-798) diluted from 10,000× to 8× per reaction.

Following PCR1, the amplified DNA was cleaned up using Agencourt AMPure XP SPRI paramagnetic beads (Beckman Coulter #A63880) or column purified using a Zymo Research DNA Clean & Concentrator-5 kit (Zymo Research #D4013) following the manufacturer's protocol. PCR 1 samples were then barcoded for Illumina NGS with NEBNext Multiplex Oligos for Illumina Dual Index Primers Set 1 and 2 (NEB #E7600S and #E7780S) with 2 μL PCR1 input and amplified for 5 cycles to generate PCR2 products. The PCR2 products were again purified using Agencourt AMPure XP SPRI paramagnetic beads or column purified using a Zymo Research DNA Clean & Concentrator-5 kit (Zymo Research #D4013) following the manufacturer's protocol.

### Preparation of Amplified Sequences for NGS

The concentrations of purified PCR2 samples were determined using a Qubit™ dsDNA HS Assay Kit (Invitrogen™ #Q32854) then diluted and pooled according to the Illumina Nextseq System Denature and Dilute Libraries Guide or MiSeq System Denature and Dilute Libraries Guide along with 10-15% PhiX Control v3 (Illumina #FC-110-3001) spiked in. The pooled samples were quantified and checked for correct sizes using an Agilent High Sensitivity DNA Kit (Agilent #5067-4626) on an Agilent 2100 Electrophoresis Bioanalyzer.

Then samples were either sequenced on an Illumina NextSeq or Miseq machine using a NextSeq 500/550 High Output Kit v2.5 (150 Cycles) (Illumina #20024907), NextSeq 500/550 Mid Output Kit v2.5 (150 Cycles) (Illumina #20024904) or MiSeq Reagent Kit v3 (150-cycle) (Illumina #MS-102-3001) with the indexes read from both ends after 150 read cycles.

### NGS Analysis

Following NGS, sequences were aligned to an AAV9 template with 21 N nucleotides insertion between amino acid 588 and 589 to represent the 7-mer insertion using Bowtie 2. Further post processing was performed using SAMtools, Python 3, NumPy and Pandas. Briefly, the flanking regions up to the 7mer (prefix) and after the 7mer (suffix) region were clipped. The resulting sequence was checked to be 21 bp in length. The nucleotide sequences were converted to amino acid sequences and exported using Pandas. Read counts associated with each nucleotide sequence were converted to normalized read counts (reads per million) to adjust for sequencing depth differences between samples. Enrichment scores for each sequence are calculated by log 2 (normalized read count post screening/normalized read count in the initial virus library).

### Clustering Analysis

Capsid sequences were one-hot encoded into vectors of length 20×7=140, and projected with UMAP with the following parameters: n_neighbors=8, min_dist=0.01, metric=Euclidean. Capsid sequences were then clustered using their UMAP projection values (Xx1, Xx2) with the GaussianMixture model from scikit-learn with parameters n_components=30, random_state=1, n_init=10, max_iter=1000.

### References for Examples 1˜4 & Methods

Brown, T. D., Nowak, M., Bayles, A. V., Prabhakarpandian, B., Karande, P., Lahann, J., Helgeson, M. E., and Mitragotri, S. (2019). A microfluidic model of human brain (uHuB) for assessment of blood brain barrier. Bioeng Transl Med 4, e10126.
Bergmann, S. et al. Blood-brain-barrier organoids for investigating the permeability of CNS therapeutics. Nat Protoc 13, 2827-2843 (2018).
Challis, R. C. et al. Systemic AAV vectors for widespread and targeted gene delivery in rodents. Nat Protoc 14, 379-414 (2019).
Chan, K. Y., Jang, M. J., Yoo, B. B., Greenbaum, A., Ravi, N., Wu, W.-L., Sánchez-Guardado, L., Lois, C., Mazmanian, S. K., Deverman, B. E., et al. (2017). Engineered AAVs for efficient noninvasive gene delivery to the central and peripheral nervous systems. Nat. Neurosci. 20, 1172-1179.
Cho, C.-F., Wolfe, J. M., Fadzen, C. M., Calligaris, D., Hornburg, K., Chiocca, E. A., Agar, N. Y. R., Pentelute, B. L., and Lawler, S. E. (2017). Blood-brain-barrier spheroids as an in vitro screening platform for brain-penetrating agents. Nat. Commun. 8, 15623.
Dalkara, D., Byrne, L. C., Klimczak, R. R., Visel, M., Yin, L., Merigan, W. H., Flannery, J. G., and Schaffer, D. V. (2013). In vivo-directed evolution of a new adeno-associated virus for therapeutic outer retinal gene delivery from the vitreous. Sci. Transl. Med. 5, 189ra76.
Depla, J. A., Sogorb-Gonzalez, M., Mulder, L. A., Heine, V. M., Konstantinova, P., van Deventer, S., Wolthers, K. C., Pajkrt, D., Sridhar, A., and Evers, M. M. (2020). Cerebral Organoids: A Human Model for AAV Capsid Selection and Therapeutic Transgene Efficacy in the Brain. Mol Ther Methods Clin Dev 18, 167-175.
Deverman, B. E., Pravdo, P. L., Simpson, B. P., Kumar, S. R., Chan, K. Y., Banerjee, A., Wu, W.-L., Yang, B., Huber, N., Pasca, S. P., et al. (2016). Cre-dependent selection yields AAV variants for widespread gene transfer to the adult brain. Nat. Biotechnol. 34, 204-209.
Ferrero, S. et al. Antibody-Based Inhibition of Pathogenic New World Hemorrhagic Fever Mammarenaviruses by Steric Occlusion of the Human Transferrin Receptor 1 Apical Domain. J Virol 95, e01868-20 (2021).
Garita-Hernandez, M., Routet, F., Guibbal, L., Khabou, H., Toualbi, L., Riancho, L., Reichman, S., Duebel, J., Sahel, J.-A., Goureau, O., et al. (2020). AAV-Mediated Gene Delivery to 3D Retinal Organoids Derived from Human Induced Pluripotent Stem Cells. Int. J. Mol. Sci. 21.
Hordeaux, J., Wang, Q., Katz, N., Buza, E. L., Bell, P., and Wilson, J. M. (2018). The Neurotropic Properties of AAV-PHP.B Are Limited to C57BL/6J Mice. Mol. Ther. 26, 664-668.
Huang, Q., Chan, K. Y., Tobey, I. G., Chan, Y. A., Poterba, T., Boutros, C. L., Balazs, A. B., Daneman, R., Bloom, J. M., Seed, C., et al. (2019). Delivering genes across the blood-brain barrier: LY6A, a novel cellular receptor for AAV-PHP.B capsids. PLOS One 14, e0225206.
Körbelin, J., Dogbevia, G., Michelfelder, S., Ridder, D. A., Hunger, A., Wenzel, J., Seismann, H., Lampe, M., Bannach, J., Pasparakis, M., et al. (2016). A brain microvasculature endothelial cell-specific viral vector with the potential to treat neurovascular and neurological diseases. EMBO Mol. Med. 8, 609-625.
Krolak, T., Chan, K. Y., Kaplan, L., Huang, Q., Wu, J., Zheng, Q., Kozareva, V., Beddow, T., Tobey, I. G., Pacouret, S., et al. (2022). A high-efficiency AAV for endothelial cell transduction throughout the central nervous system. Nature Cardiovascular Research 1, 389-400.
Langmead, B., and Salzberg, S. L. (2012). Fast gapped-read alignment with Bowtie 2. Nat. Methods 9, 357-359.
Li, H., Handsaker, B., Wysoker, A., Fennell, T., Ruan, J., Homer, N., Marth, G., Abecasis, G., Durbin, R., and 1000 Genome Project Data Processing Subgroup (2009). The Sequence Alignment/Map format and SAMtools. Bioinformatics 25, 2078-2079.
Lisowski, L., Dane, A. P., Chu, K., Zhang, Y., Cunningham, S. C., Wilson, E. M., Nygaard, S., Grompe, M., Alexander, I. E., and Kay, M. A. (2013). Selection and evaluation of clinically relevant AAV variants in a xenograft liver model. Nature 506, 382-386.
McInnes, L., Healy, J., and Melville, J. (2018). UMAP: Uniform Manifold Approximation and Projection for Dimension Reduction.
Muik, A., Reul, J., Friedel, T., Muth, A., Hartmann, K. P., Schneider, I. C., Münch, R. C., and Buchholz, C. J. (2017). Covalent coupling of high-affinity ligands to the surface of viral vector particles by protein trans-splicing mediates cell type-specific gene transfer. Biomaterials 144, 84-94.
Münch, R. C., Janicki, H., Völker, I., Rasbach, A., Hallek, M., Büning, H., and Buchholz, C. J. (2013). Displaying high-affinity ligands on adeno-associated viral vectors enables tumor cell-specific and safe gene transfer. Mol. Ther. 21, 109-118.
Münch, R. C., Muth, A., Muik, A., Friedel, T., Schmatz, J., Dreier, B., Trkola, A., Plückthun, A., Büning, H., and Buchholz, C. J. (2015). Off-target-free gene delivery by affinity-purified receptor-targeted viral vectors. Nat. Commun. 6, 6246.
Nonnenmacher, M., Wang, W., Child, M. A., Ren, X.-Q., Huang, C., Ren, A. Z., Tocci, J., Chen, Q., Bittner, K., Tyson, K., et al. (2021). Rapid evolution of blood-brain-barrier-penetrating AAV capsids by RNA-driven biopanning. Mol Ther Methods Clin Dev 20, 366-378.
Ponnazhagan, S., Mahendra, G., Kumar, S., Thompson, J. A., and Castillas, M., Jr (2002). Conjugate-based targeting of recombinant adeno-associated virus type 2 vectors by using avidin-linked ligands. J. Virol. 76, 12900-12907.
Ravindra Kumar, S., Miles, T. F., Chen, X., Brown, D., Dobreva, T., Huang, Q., Ding, X., Luo, Y., Einarsson, P. H., Greenbaum, A., et al. (2020). Multiplexed Cre-dependent selection yields systemic AAVs for targeting distinct brain cell types. Nat. Methods 17, 541-550.
Reul, J., Muik, A., and Buchholz, C. J. (2019). Ligand Coupling to the AAV Capsid for Cell-Specific Gene Transfer. Methods Mol. Biol. 1950, 35-50.
Sherman, H., and Rossi, A. E. (2019). A Novel Three-Dimensional Glioma Blood-Brain Barrier Model for High-Throughput Testing of Tumoricidal Capability. Front. Oncol. 9, 351.
Tabebordbar, M., Lagerborg, K. A., Stanton, A., King, E. M., Ye, S., Tellez, L., Krunnfusz, A., Tavakoli, S., Widrick, J. J., Messemer, K. A., et al. (2021). Directed evolution of a family of AAV capsid variants enabling potent muscle-directed gene delivery across species. Cell 184, 4919-4938.e22.
Tervo, D. G. R., Hwang, B.-Y., Viswanathan, S., Gaj, T., Lavzin, M., Ritola, K. D., Lindo, S., Michael, S., Kuleshova, E., Ojala, D., et al. (2016). A Designer AAV Variant Permits Efficient Retrograde Access to Projection Neurons. Neuron 92, 372-382.
Zhang, Y., Chen, K., Sloan, S. A., Bennett, M. L., Scholze, A. R., O'Keeffe, S., Phatnani, H. P., Guarnieri, P., Caneda, C., Ruderisch, N., et al. (2014). An RNA-sequencing transcriptome and splicing database of glia, neurons, and vascular cells of the cerebral cortex. J. Neurosci. 34, 11929-11947.
(2021). In situ sensors for blood-brain barrier (BBB) on a chip. Sensors and Actuators Reports 3, 100031.
WO2021222636A1.
US20210403946A1.

### Example 5—Additional Examples

AAV capsids engineered to bind human TFRC transduce human brain endothelial cells more efficiently than AAV9, an effect that is further enhanced by increasing the surface expression of TFRC. Applicants also showed that transduction of human brain endothelial cells by one of the TFRC binding AAVs, BI-19, is blocked in a dose-dependent manner by an antibody against TFRC. Furthermore, in mice made to express human TFRC on the brain vasculature, Applicants observed increased targeting of BI-19 to the brain, indicating that BI-19 can target human TFRC in vivo and suggesting that the engagement of TFRC can increase both endothelial transduction and crossing of the BBB. Together, the data support further exploration of TFRC targeted AAVs as delivery vehicles for CNS gene therapy.

TFRC binding capsids and sequence space exploration by mutagenesis identifies capsids for individual evaluation.

| Capsid ID | Nucleic Acid Sequence | Amino Acid Sequence |
|---|---|---|
| BI-19 | TACTCACGCATCGGCCCCAAC | YSRIGPN |
| | (SEQ ID NO: 3681) | (SEQ ID NO: 14632) |

Individually validated human TFRC binding AAV 588 7-mer sequences identified through binding a Fc-TFRC fusion protein.

| Capsid ID | Nucleic Acid Sequence | Amino Acid Sequence |
|---|---|---|
| BI-98 | TATTCTCGCTTAAACATG | YSRLNMN (SEQ ID NO: |
| | AAT (SEQ ID NO: 5678) | 14301) |
| BI-99 | TACTCACGCCTTAATAA | YSRLNKD (SEQ ID NO: |
| | GGAT (SEQ ID NO: 6495) | 16577) |
| BI-100 | GTACATCGCCTGCAAGA | VHRLQDK (SEQ ID NO: |
| | TAAG (SEQ ID NO: 6626) | 16602) |
| BI-101 | TACCATCGTTTGTCCAAT | YHRLSNN (SEQ ID NO: |
| | AAC (SEQ ID NO: 5787) | 16636) |
| BI-102 | TTACACGCTTTGTCACAC | LHALSHN (SEQ ID NO: |
| | AAT (SEQ ID NO: 6528) | 16608) |

### Example 6—Conclusions

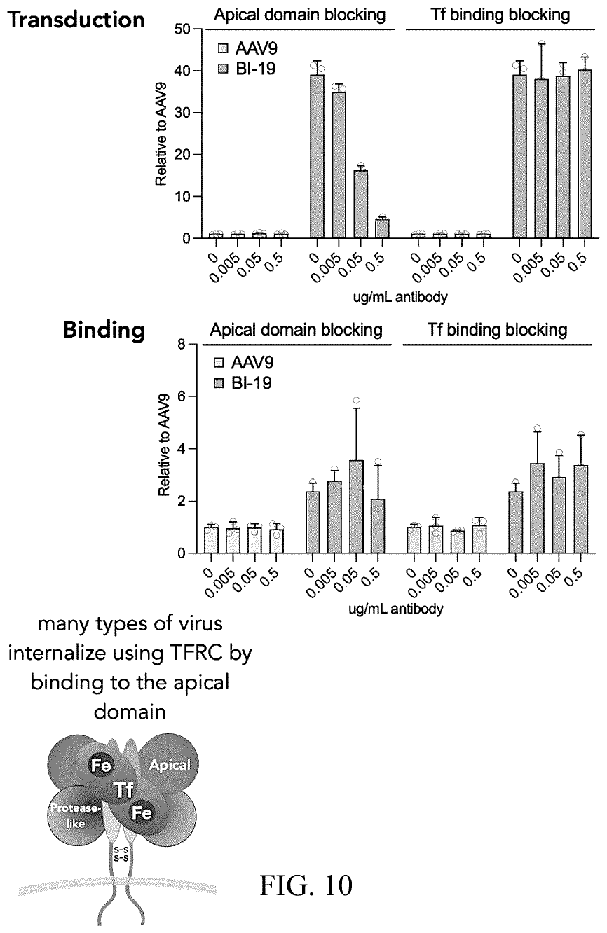

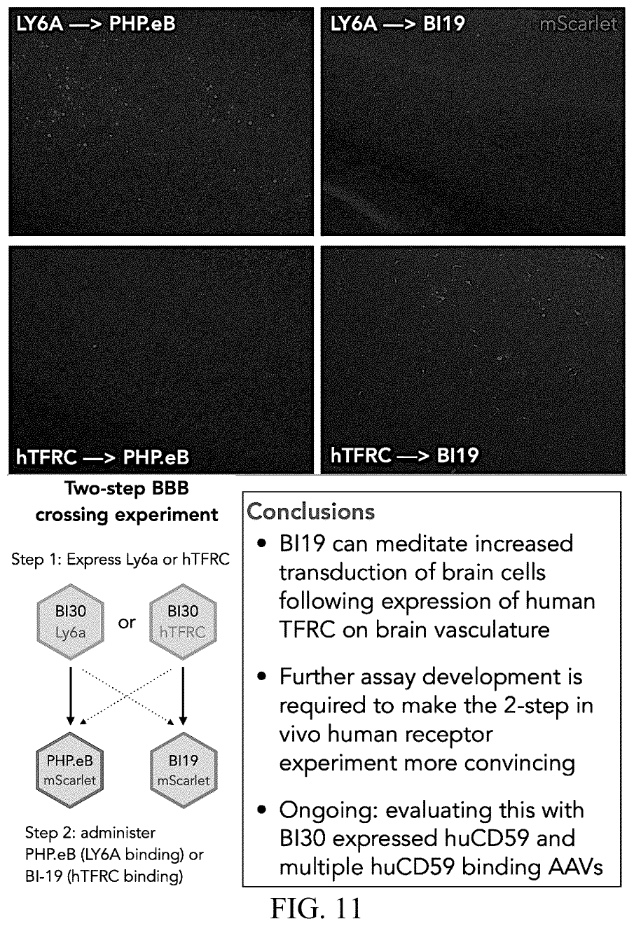

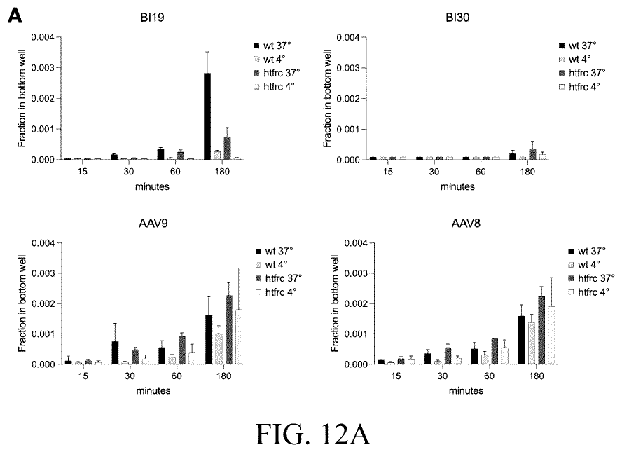

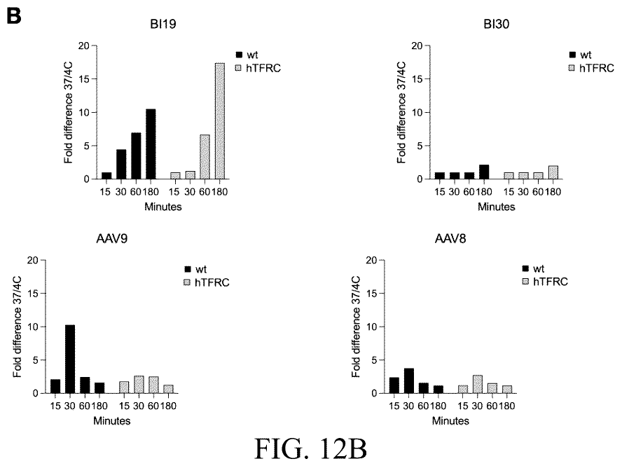

Antibody blocking experiments (FIG. 10) indicate that the enhanced transduction activity of BI-19 requires TFRC binding. BI-19 appears to bind TFRC through the apical domain, see FIG. 10. TFRC binding contributes minimally to BI-19 hCMEC binding. BI-19 can mediate increased transduction of brain cells following expression of human TFRC on brain vasculature, see FIG. 11.

### Example 7

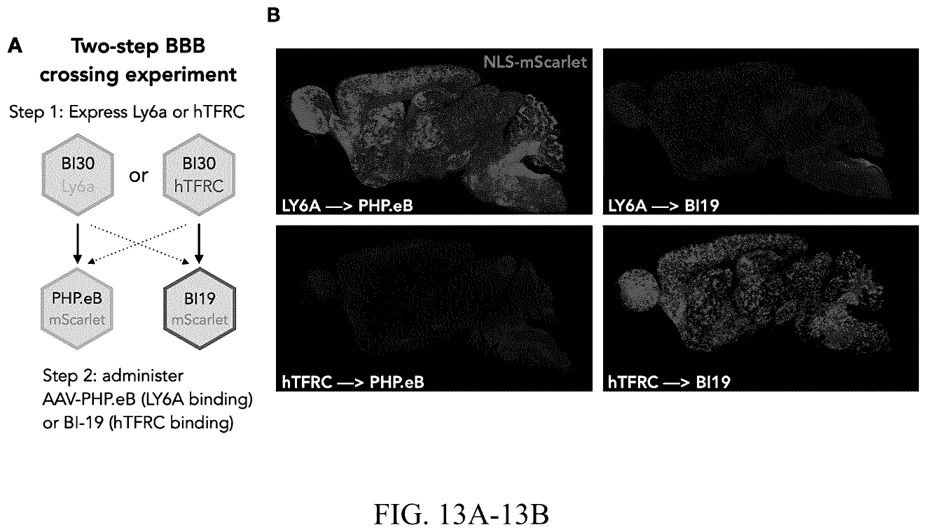

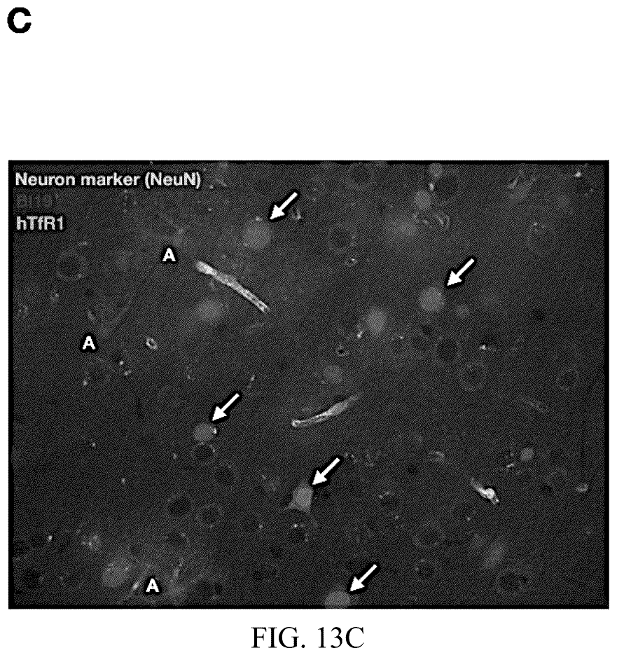

Here Applicants present new evidence that AAV-BI19 can efficiently cross a human brain vascular barrier model and the mouse blood brain barrier (BBB) and transduce cells throughout the mouse CNS when the mice are made to express human TFRC. In the first experiment, Applicants evaluated the ability of BI19 to cross a brain endothelial barrier made from a human brain endothelial cell line (hCMEC-D3) grown on a transwell insert. After establishing a monolayer of hCMEC-D3 cells, which express TFRC, AAV-BI19 was added to the upper well of the chamber (which models the luminal/blood side), and the fraction of virus that crossed the endothelial barrier and entered the lower chamber (brain side) was assessed. The assay was performed at 37° C. or at 4° C., which serves as a control to distinguish passive crossing (occurs at 37° C. and 4° C.) from active transport (occurs only at 37° C.). AAV-BI19 showed temperature- and time-dependent entry into the lower chamber (FIG. 13A). In contrast, other AAV capsids including AAV9, AAV8, and AAV-BI30 were detected at similar levels in the lower chamber at 37° C. and 4° C. suggesting that the crossing detected with these AAVs occurred primarily from passive leakage through the endothelial cell barrier. Taking the ratio of the fraction detected in the lower chamber at 37° C. vs 4° C. demonstrated that the crossing of AAV-BI19 through the endothelial barrier is more temperature-dependent than the other viruses (FIG. 13B).

Next Applicants sought to assess whether AAV-BI19 could cross the BBB in vivo. Because AAV-BI19 binds to human TFR1 but not the TFRC from mouse, marmosets, or macaques, on target in vivo validation necessitates expression of human TFRC on mouse brain vasculature. Applicants therefore performed a “two-step BBB crossing experiment” where Applicants used the brain endothelial targeted capsid AAV-BI30 (Krolack et al. Nature Cardiovascular Research 2022) to express the target receptor (human TFRC or mouse Ly6a) in mice prior to delivery of the capsid to be tested (AAV-BI19 or positive control AAV-PHP.eB) (FIG. 13A). Applicants performed the experiment in adult immunocompromised NGS mice to prevent an acquired immune response from blocking transduction by the second AAV. NSG mice were injected with 1e11 vg/mouse of AAV-BI30 expressing human TFRC, or mouse Ly6a cDNA as a control. Four weeks later the mice were injected with AAV-BI19 or AAV-PHP.eB carrying an mScarlet reporter driven by the strong, ubiquitous CAG promoter. mScarlet expression was observed throughout the CNS 3 weeks after IV administration of the second AAV (FIG. 13B). Applicants found that AAV-BI19 efficiently crosses the BBB and transduces cells in the mouse brain of animals that were previously injected with AAV-BI30 carrying human TFRC but not in animals that were previously injected with AAV-BI30 carrying Ly6a. In contrast, AAV-PHP.eB efficiently transduced the brains of mice that expressed AAV-BI30 carrying mouse Ly6a. Importantly, AAV-BI19 crossed the BBB and transduced neurons (FIG. 13C). These results demonstrate that AAV-BI19 is capable of crossing the huTFRC expressing BBB in vivo.

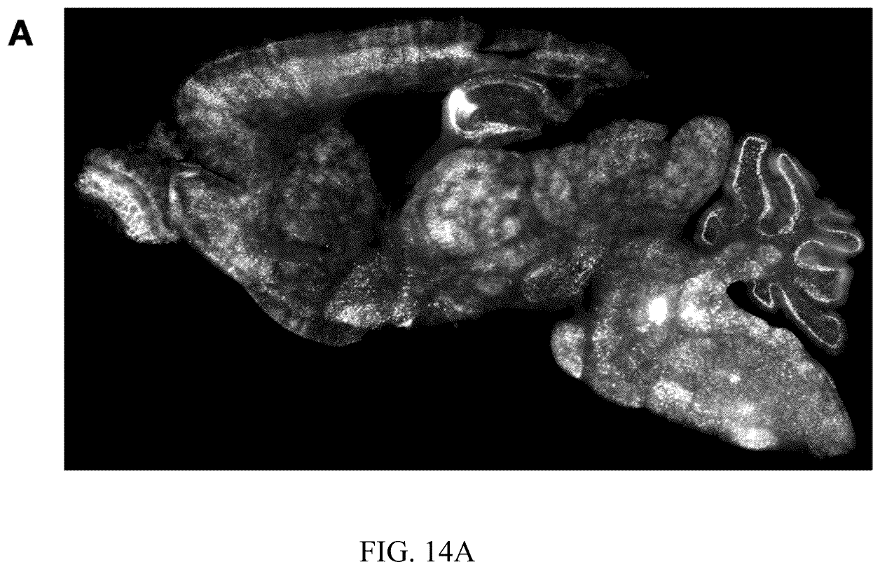

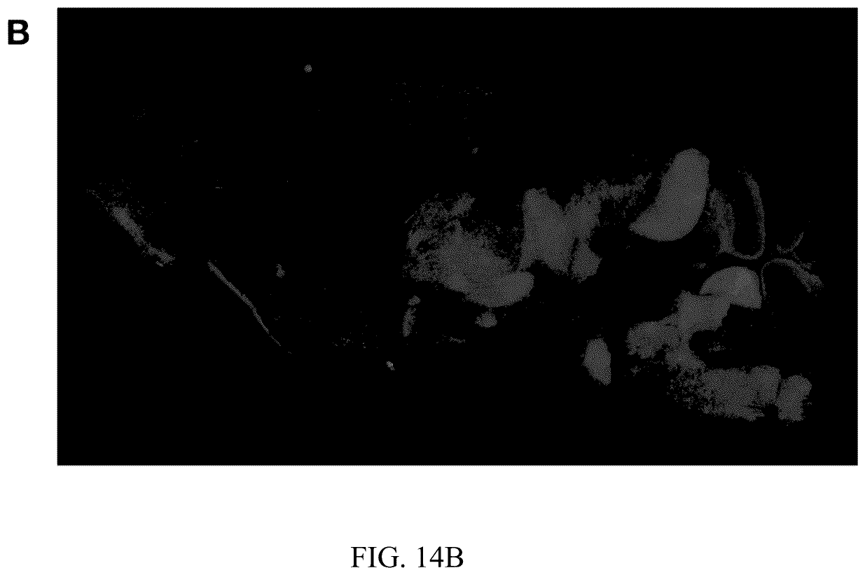

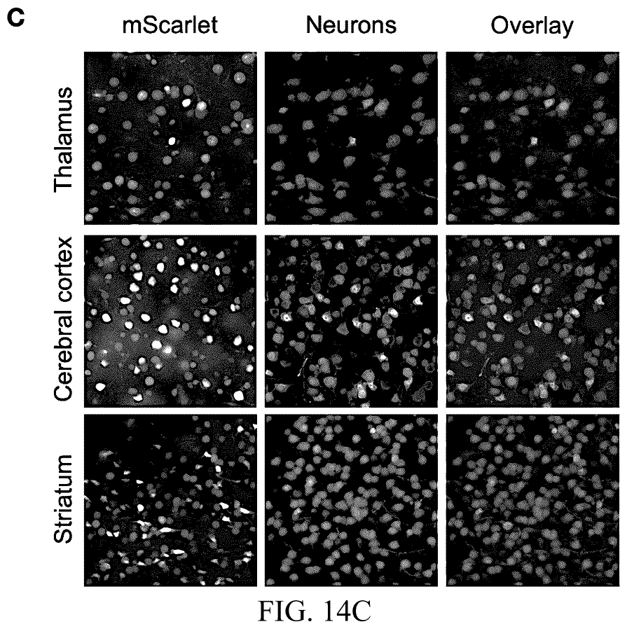

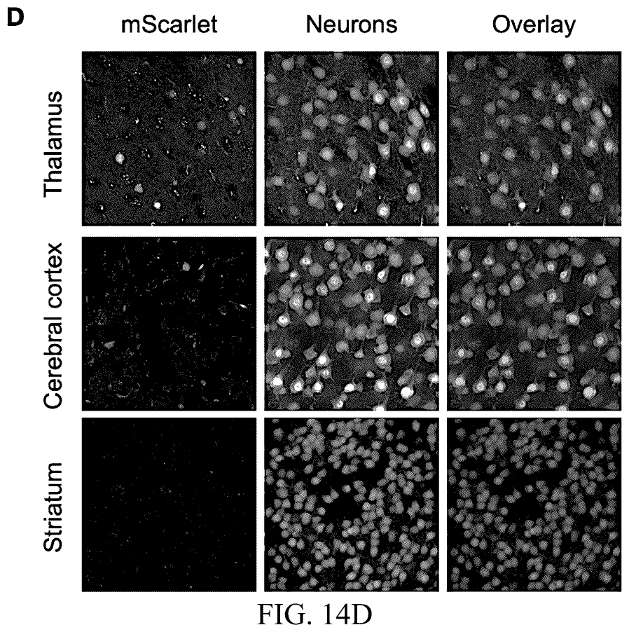

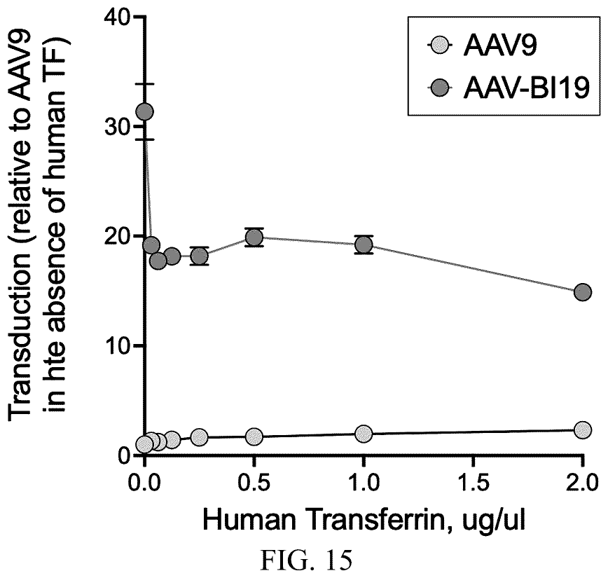

One possible caveat to the experiment above is that transduction by AAV-BI30 may cause huTFRC to be expressed at levels that exceed the normal expression of the protein and expression by AAV-BI30 may cause ectopic huTFRC expression in other cell types where TFRC is normally low. Therefore, Applicants next evaluated AAV-BI19 transduction in mice that have had the exons encoding the extracellular domain of mouse TFRC replaced with the corresponding exons encoding the ECD of human TFRC (referred to as huTFRC KI mice). In huTFRC KI mice, TFRC is expressed under control of the mouse TFRC gene regulatory elements at levels comparable with endogenous mouse Tfrc (BioCytogen). Confirming the results with the 2-step assay, Applicants find that AAV-BI19, but not AAV9, efficiently transduces the brain of huTFRC KI mice (FIG. 14), providing strong evidence in support of the use of AAV-BI19 for gene delivery across the human BBB. As Applicants observed in the two-step assay, AAV-BI19 efficiently transduced neurons throughout the CNS (FIG. 14C). Finally, Applicants also demonstrate that AAV-BI19 is capable of enhanced transduction of hCMEC cells as compared to AAV9 even in the presence of human Transferrin (FIG. 15), although the extent of transduction relative to AAV9 is slightly reduced. These data suggest that AAV-BI19 has the potential to transduce human brain endothelial cells by engaging human TFR1 or TFR1-Transferrin complexes.

### Example 8—AAV Capsids Reprogrammed to Bind Human Transferrin Receptor Mediate Brain-Wide Gene Delivery

### Abstract

Developing the means to efficiently deliver genes throughout the human central nervous system (CNS) could provide opportunities to treat a wide range of genetic diseases. Here, Applicants reprogrammed a suite of peptide-modified AAV9 capsids to bind to the human Transferrin Receptor (TfR1) protein, which is highly expressed on the brain endothelium, and demonstrated that this approach could drive blood-brain barrier (BBB)-crossing ability in vitro and in vivo. The strategy circumvented a key limitation of traditional in vivo screens that select for enhanced CNS-wide transduction without specifying a known mechanism of action and therefore identify capsids with unpredictable species specificity and translational potential. The huTfR1-binding capsids transduced human brain endothelial cells through interactions with TfR1. Applicants chose one capsid, AAV-BI19 for further characterization in mice that express a chimeric TFRC gene with a humanized extracellular domain. Relative to AAV9, AAV-BI19 efficiently transduced neurons and astrocytes across the brain and spinal cord after intravenous delivery and mediated a 50-fold increase in reporter gene expression in the brain; this enhanced tropism of AAV-BI19 was not observed in other organs evaluated. These results support the further assessment of AAVs programmed to target TfR1 to cross the BBB for human CNS gene therapy.

### Introduction

The field of gene therapy has benefitted from a range of AAV serotypes identified from nature. Most notably, the discovery of AAV9, which can transduce motor neurons as well as neurons and glia within the neonatal CNS, led to the development of an FDA-approved gene therapy for spinal muscular atrophy type 1 (SMA1) (Foust, K. D.; et al. Intravascular AAV9 Preferentially Targets Neonatal Neurons and Adult Astrocytes. Nature Biotechnology, 2008, 27, 59-65, Waldrop, M. A.; et al. Gene Therapy for Spinal Muscular Atrophy: Safety and Early Outcomes. Pediatrics, 2020, 146). However, numerous neurodevelopmental, neurodegenerative, and metabolic monogenic CNS diseases still have no available treatments. There is an urgent need for AAV capsids that are engineered to cross the blood-brain barrier (BBB), particularly in adult patients, and more effectively deliver genes to disease-relevant cells at a lower dose. Developing capsids that can effectively deliver gene therapies across the adult BBB will significantly broaden the scope of treatable indications.

Applicants recently demonstrated that AAV capsids can be reprogrammed to deliver genes across the mouse BBB through a mechanism-focused approach that selects for interactions between capsids and receptor proteins expressed on host cells (Huang, Q.; et al. Targeting AAV Vectors to the CNS viade NovoEngineered Capsid-Receptor Interactions, 2022). This receptor-targeting approach solves a key challenge faced in conventional AAV capsid screens, which can identify rare functional capsids without an understanding of their mechanism that determines their tropism and translatability across species. In this work, Applicants extended the receptor-targeting approach to a human target protein and directly engineered a suite of capsids that cross the blood-brain barrier (BBB) by leveraging the human Transferrin Receptor (huTfR1). TfR1 was chosen as a target because of its high expression on the human BBB; its ability to mediate constitutive, ligand-independent endocytosis and transcytosis across the CNS vasculature (Ajioka, R. S.; Kaplan, J. Intracellular Pools of Transferrin Receptors Result from Constitutive Internalization of Unoccupied Receptors. Proceedings of the National Academy of Sciences, 1986, 83, 6445-6449, Maxfield, F. R.; McGraw, T. E. Endocytic Recycling. Nature Reviews Molecular Cell Biology, 2004, 5, 121-132, Moos, T.; Morgan, E. H. Cellular and Molecular Neurobiology, 2000, 20, 77-95, Pardridge, W. M.; et al. Human Blood-Brain Barrier Transferrin Receptor. Metabolism, 1987, 36, 892-895, Jefferies, W. A.; et al. Transferrin Receptor on Endothelium of Brain Capillaries. Nature, 1984, 312, 162-163); its history as a receptor for viruses including arenaviruses and canine and feline parvoviruses (Goodman, L. B.; et al. Binding Site on the Transferrin Receptor for the Parvovirus Capsid and Effects of Altered Affinity on Cell Uptake and Infection. Journal of Virology, 2010, 84, 4969-4978, Flanagan, M. L.; et al. New World Clade B Arenaviruses Can Use Transferrin Receptor 1 (TfR1)-Dependent and -Independent Entry Pathways, and Glycoproteins from Human Pathogenic Strains Are Associated with the Use of TfR1. Journal of Virology, 2008, 82, 938-948); and its track record as a target to increase the delivery of biologics into the CNS of mice, nonhuman primates (NHPs) (Kariolis, M. S.; et al. Brain Delivery of Therapeutic Proteins Using an Fc Fragment Blood-Brain Barrier Transport Vehicle in Mice and Monkeys. Science Translational Medicine, 2020, 12), and humans as an approved antibody-based therapeutic for Mucopolysaccharidosis type II (MPS II) (Okuyama, T.; et al. A Phase 2/3 Trial of Pabinafusp Alfa, IDS Fused with Anti-Human Transferrin Receptor Antibody, Targeting Neurodegeneration in MPS-II. Molecular Therapy, 2021, 29, 671-679).

Applicants first screened peptide-modified AAV9 capsid libraries for their ability to bind to huTfR1 in vitro. The top-performing capsid, AAV-BI19, was then validated and found to exhibit enhanced gene delivery to human brain endothelial cells and to the majority of neurons and glia in human TFRC knock-in mice. From in vivo biodistribution and mRNA reporter transduction data, the enhanced tropism of AAV-BI19 was found to be specific to the CNS in the huTfR1 knock-in mice. This finding is consistent with the high level of expression of TFRC on CNS vasculature relative to the vasculature in other organs (Jefferies, W. A.; et al. Transferrin Receptor on Endothelium of Brain Capillaries. Nature, 1984, 312, 162-163). These huTfR1-binding capsids represent promising vectors for CNS gene therapy applications.

### Results

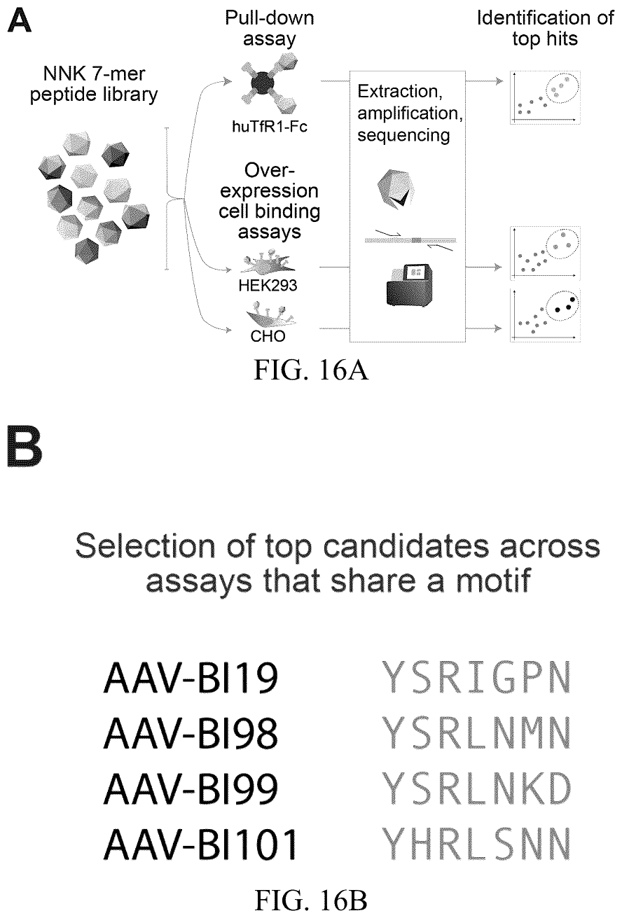

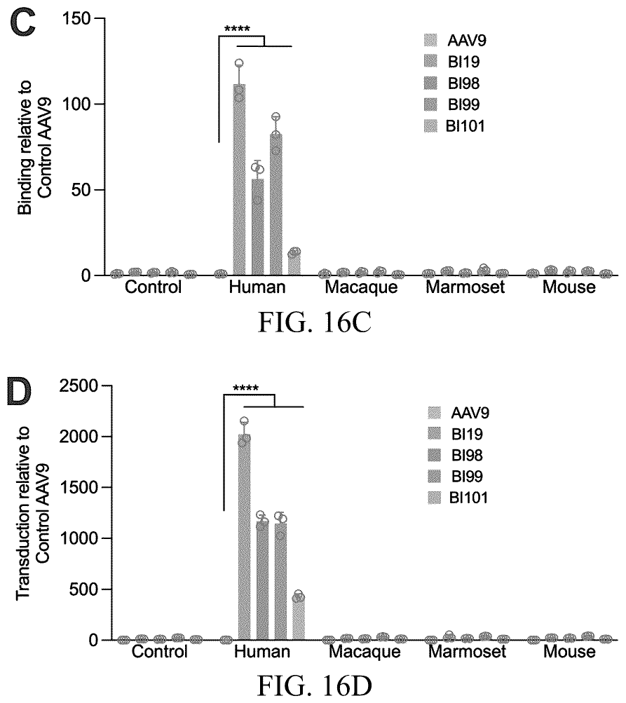

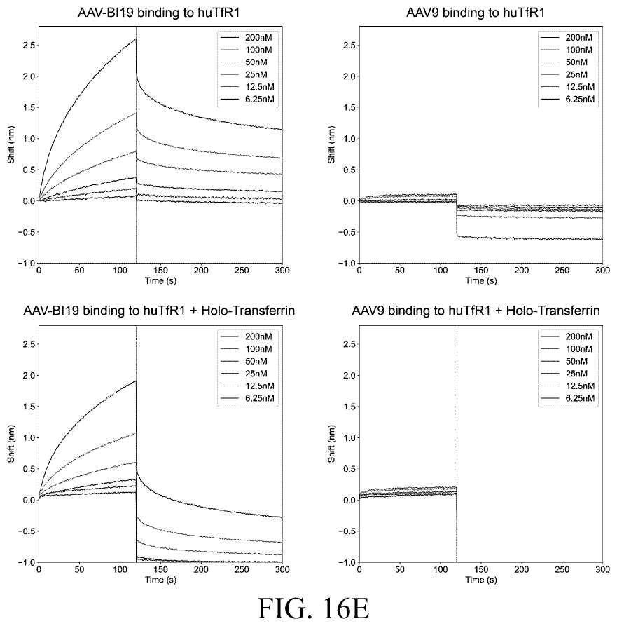

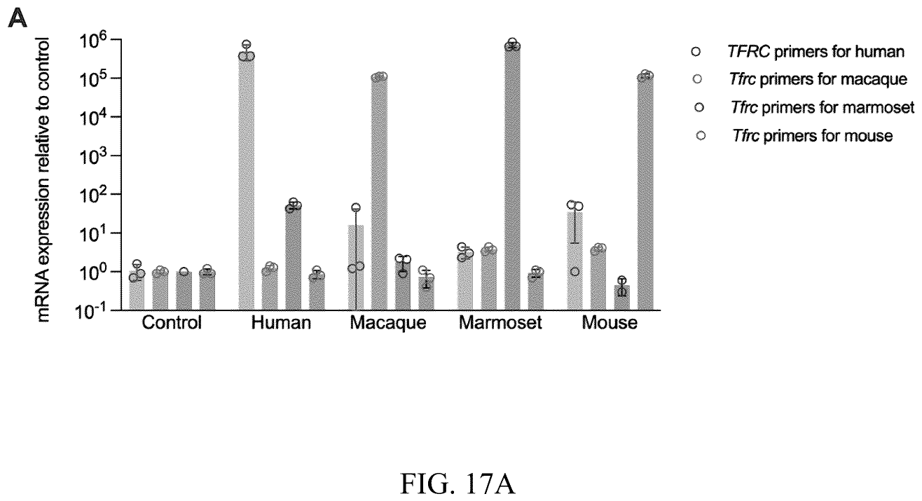

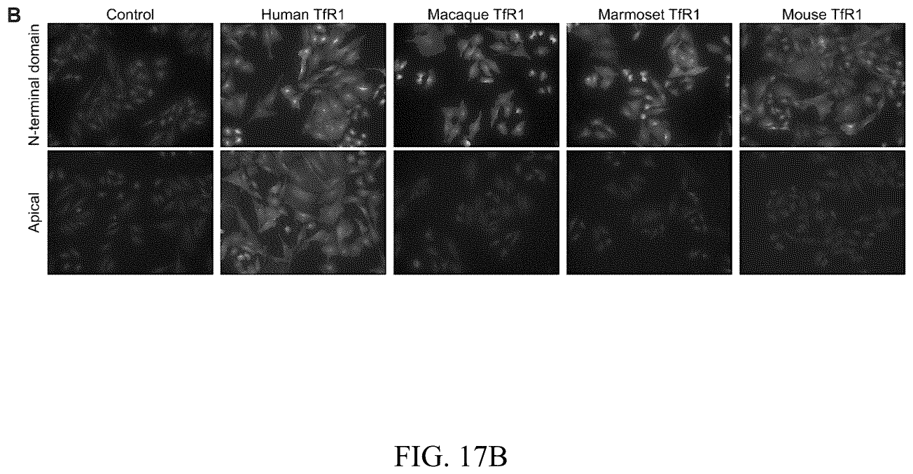

Applicants screened AAV9-based 7-mer NNK capsid libraries (random 7-mer insertion between residues 588-589 in VP1) for selective binding to huTfR1 as an Fc fusion protein and for selective binding to HEK293 and CHO cells overexpressing human TFRC as previously described (Huang, Q.; et al. Targeting AAV Vectors to the CNS viade NovoEngineered Capsid-Receptor Interactions, 2022) (FIG. 16A). Based on the screen results, Applicants focused on a set of four capsids, AAV-BI19, BI98, BI99, and BI101 that share a common sequence motif (FIG. 16B). These capsids, alongside AAV9, were individually tested for their ability to bind to TfR1 from human, rhesus macaque, marmoset, or mouse when stably expressed on CHO cells (FIG. 16C,D and FIG. 17). Each capsid packaged a genome that expresses nuclear localized mScarlet and Luciferase under the control of the ubiquitous CAG promoter (AAV-CAG-NLS-mScarlet-P2A-Luciferase-SV40-WPRE). The four capsids exhibited selectively elevated binding to CHO cells expressing human TFRC but not the control cells or those expressing mouse or NHP Tfrc (FIG. 16C,D).

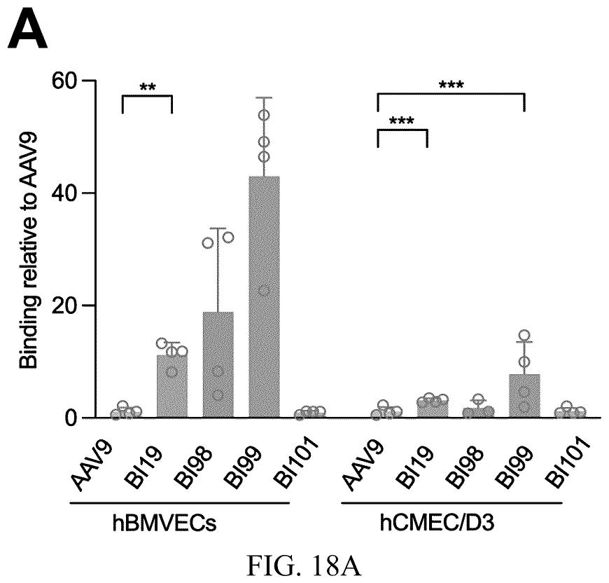

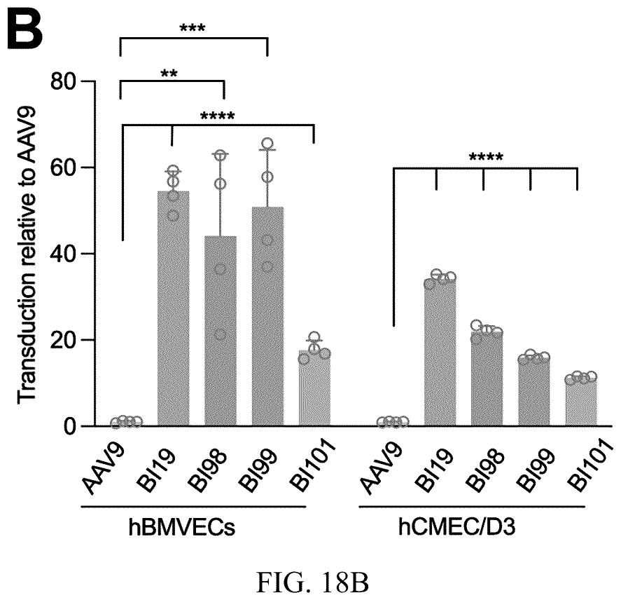

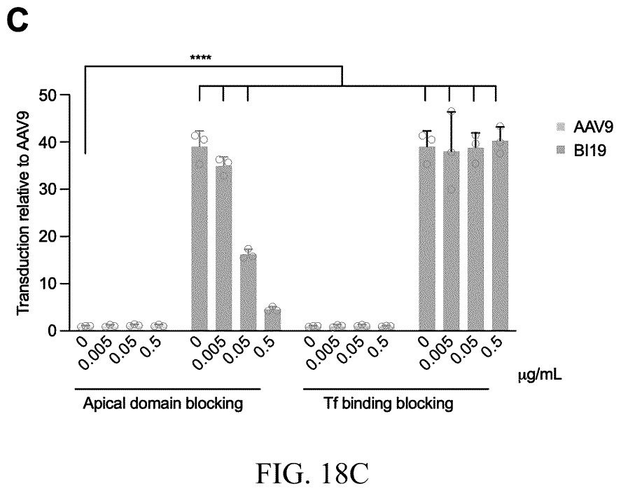

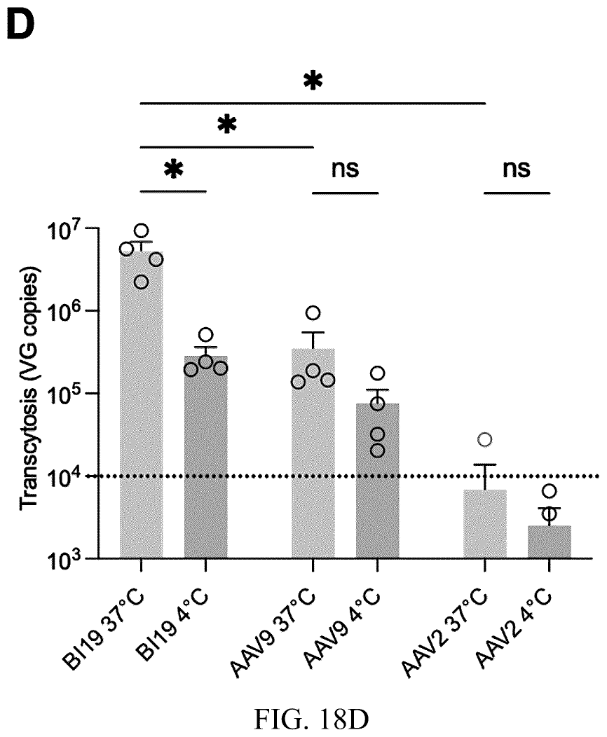

The four huTfR1-binding capsids exhibited enhanced binding and transduction of human brain endothelial cells (FIG. 18A,B). The best-performing capsid, AAV-BI19 was chosen for more in-depth analysis. It exhibited comparable post-purification production yields to AAV9. Its enhanced transduction of hCMEC/D3 cells was found to be inhibited by the OKT9 antibody that binds the apical domain of TfR1 but not a polyclonal antibody AF2474 that competes with Tf for binding to TfR1 (FIG. 18C). AAV-BI19 partially colocalized with huTfR1 as puncta on the surface of hCMEC/D3s following incubation at 4° C. The number of AAV-BI19 puncta and the colocalization of AAV-BI19 with huTfR1 was dramatically reduced in the presence of OKT9 (1 ug/mL) but not Transferrin (1 ug/mL) (FIG. 18D). In contrast to AAV-BI19, AAV2 puncta were not found to colocalize with huTfR1 nor were AAV2 puncta affected by OKT9 Transferrin. To assess internalization, AAV-BI19 or AAV2 were added to hCMEC/D3 cells at 25,000 vg/cell for one hour at 37° C. Immunostaining for the AAV capsid and endosomal markers showed partial overlap with both Rab5 and Rab7 proteins, which mark early and late endosomes, respectively. Minimal overlap between AAV2 capsids and Rab5 or Rab7 was observed.

TFRC is thought to be expressed in virtually all nucleated human cells. Therefore, Applicants next asked whether the introduction of binding to human TFRC rendered AAV-BI19 independent of AAVR for transduction. AAV9 or AAV-BI19 expressing a nuclear GFP was added to hCMEC/D3 or hCMEC/D3 AAVR KO cells at 20,000 vg/cell. 24 hours later the cells were fixed and stained with a pan-AAV antibody (B1). While AAV-BI19 capsids could be detected on or within the hCMEC/D3 AAVR KO cells at a level that was as high or higher than the hCMEC/D3 control cells, transduction in the AAVR KO cells was nearly undetectable for AAV-BI19. In contrast a large fraction of cells were marked with GFP+ nuclei in the control hCMEC/D3 cells. This result suggests that AAV-BI19 remains dependent on AAVR for transduction of hCMEC/D3 cells.

Applicants next assessed transport across a human endothelial cell barrier in a BBB transwell model established using the hCMEC/D3 cell line. To minimize variance between wells, Applicants mixed AAV-BI19, AAV9, and AAV2 carrying barcoded genomes that were individually identifiable by qPCR. AAV-BI19 D). Of the three capsids, only AAV-BI19 had a statistically significantly higher amount of virus that crossed the hCMEC/D3 cell barrier and transwell membrane at the biological temperature of 37° C. as compared to at 4° C., a temperature at which ATP-dependent transcytosis is suppressed, albeit passive crossing can occur (FIG. 18D). Applicants detected more AAV9 in the bottom chamber relative to AAV2, as previously reported in the literature (Merkel, S. F.; et al. Trafficking of Adeno-Associated Virus Vectors across a Model of the Blood-Brain Barrier; a Comparative Study of Transcytosis and Transduction Using Primary Human Brain Endothelial Cells. Journal of Neurochemistry, 2016, 140, 216-230, Di Pasquale, G.; Chiorini, J. A. AAV Transcytosis through Barrier Epithelia and Endothelium. Molecular Therapy, 2006, 13, 506-516, Song, R.; et al. Selection of rAAV Vectors That Cross the Human Blood-Brain Barrier and Target the Central Nervous System Using a Transwell Model. Molecular Therapy-Methods &amp; Clinical Development, 2022, 27, 73-88), although there was no statistically significant difference between the amount of virus that crossed the hCMEC/D3 cell barrier and transwell membrane at 37° C. versus 4° C. (FIG. 18D).

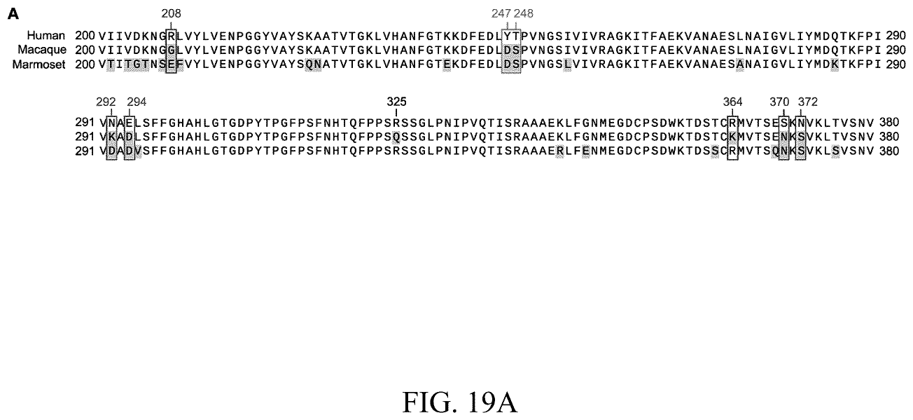

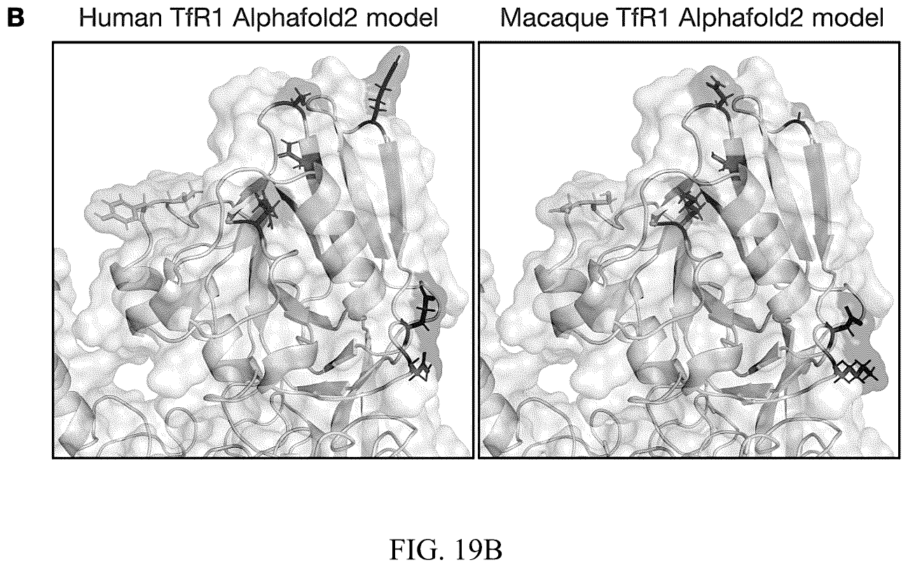

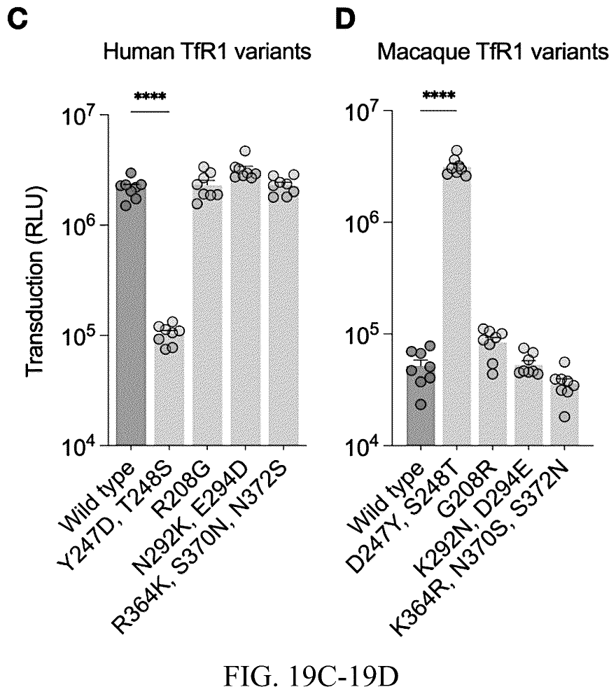

To determine key residues that facilitate the species-specific binding of AAV-BI19 to huTfR1, Applicants identified surface exposed residues that differ between the human versus macaque TfR1 apical domain and created four huTfR1 mutants each containing a different set of closely positioned substitutions such that macaque residues replaced the corresponding human residues at specific positions across the apical domain of huTfR1 (FIG. 19A). CHO cells were transiently transfected to express each mutant and the ability of AAV-BI19 to transduce these cells was assessed. The huTfR1 mutant carrying Y247D and T248S was no longer able to mediate the enhanced transduction phenotype by AAV-BI19 (FIG. 19A). Fitting with this finding, introducing the human amino acid substitutions D247Y and S248T into the macaque TfR1 was sufficient to mediate the enhanced transduction phenotype by AAV-BI19 (FIG. 19B).

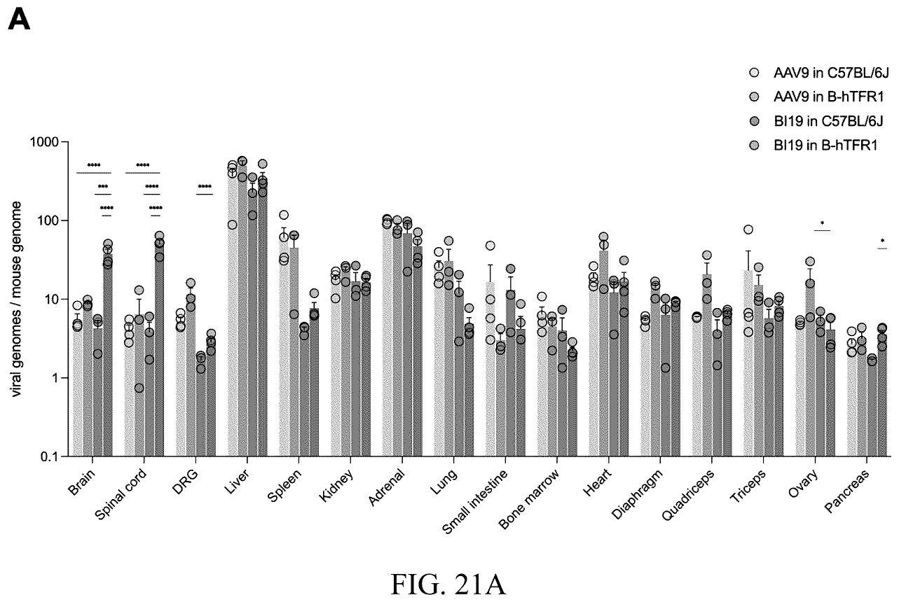

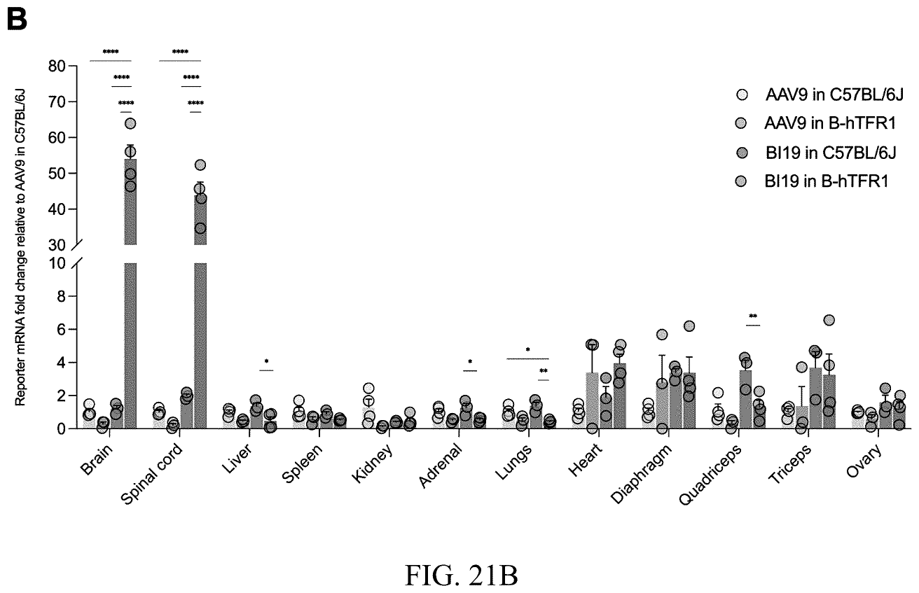

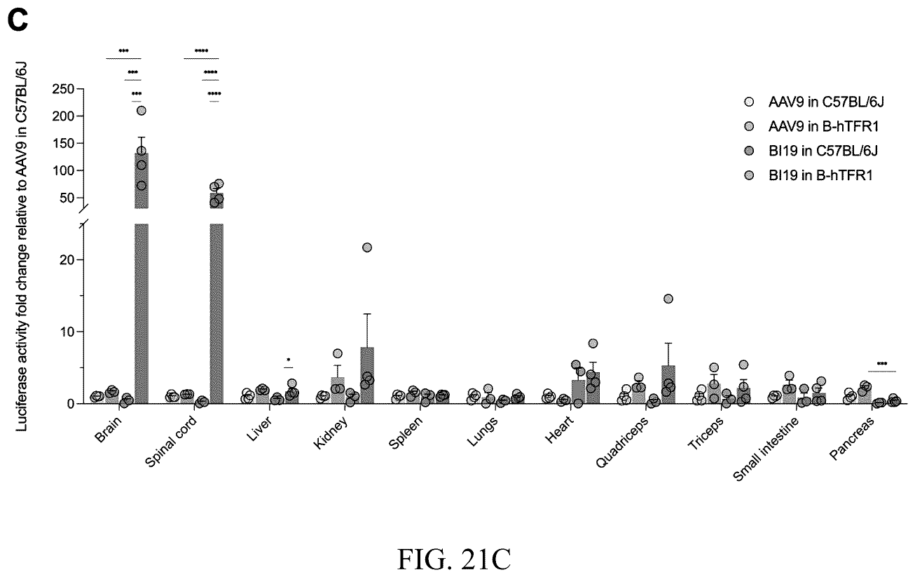

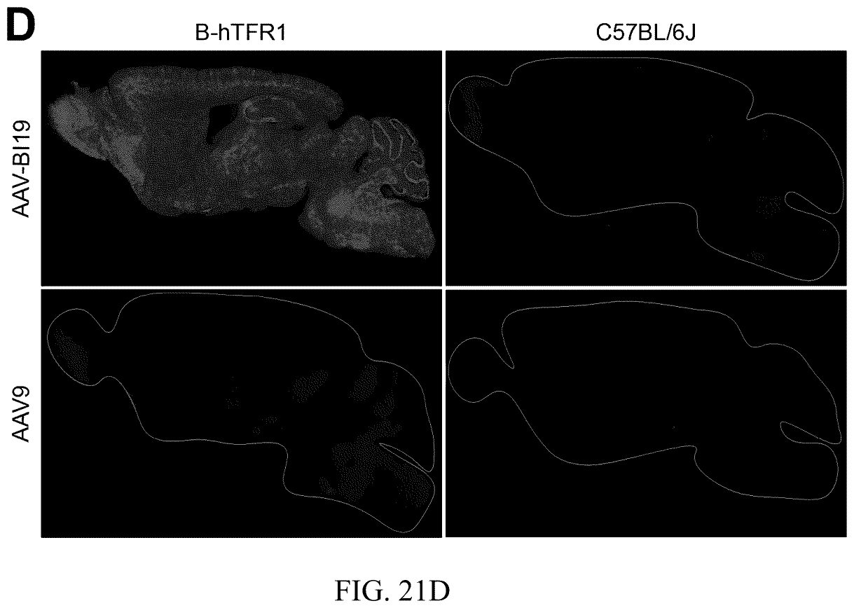

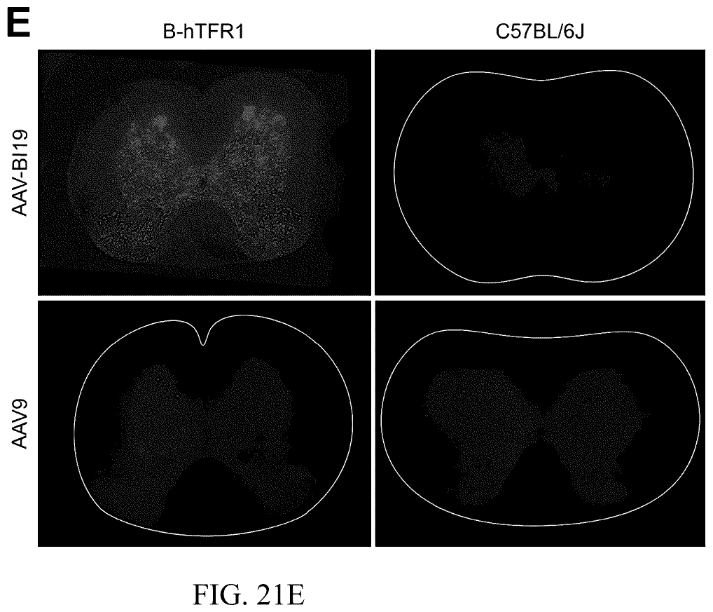

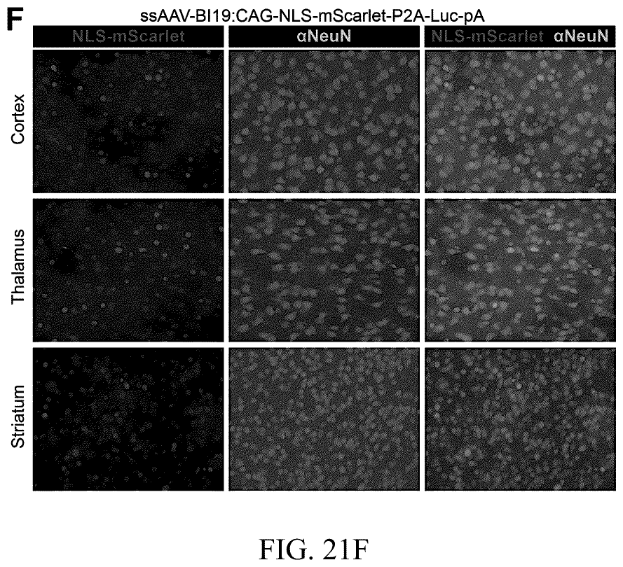

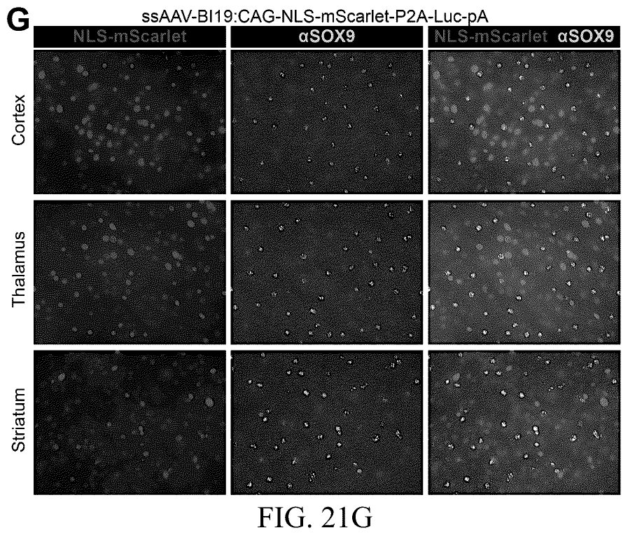

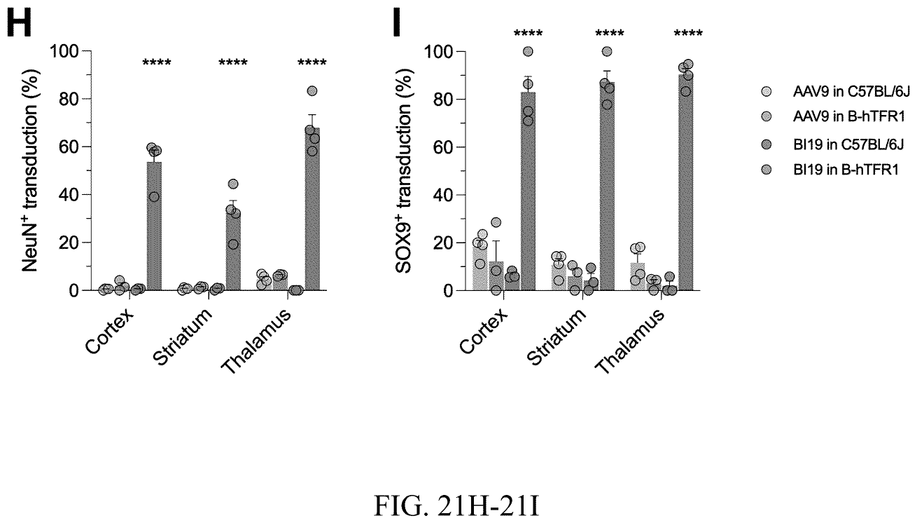

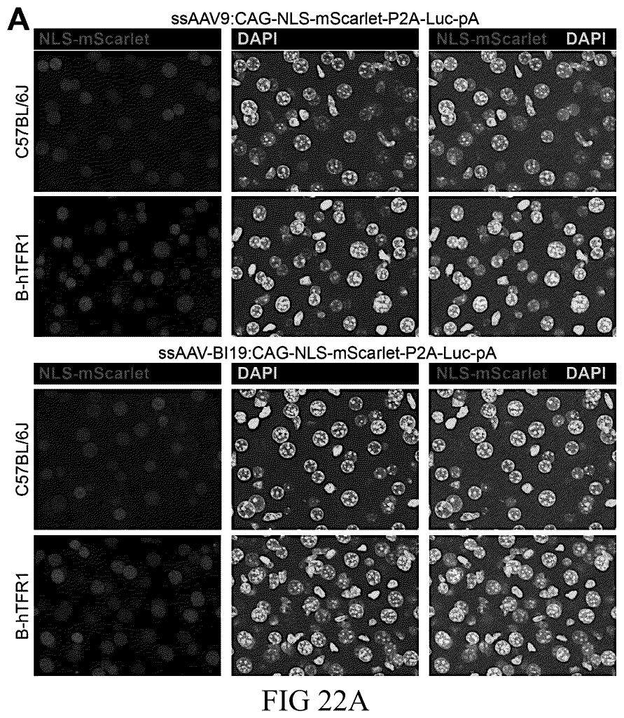

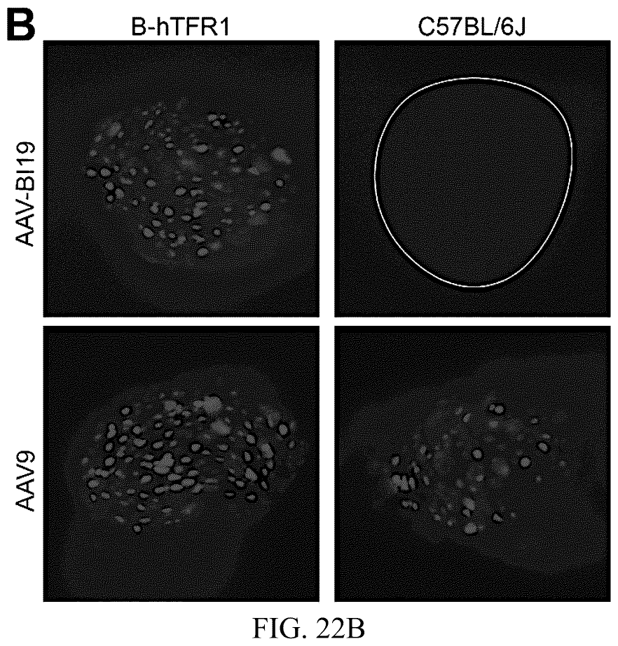

Due to its species-specific mechanism of action, Applicants tested AAV-BI19 in a knock-in mouse model where the huTfR1 extracellular domain had replaced the endogenous mouse TfR1 extracellular domain (FIG. 4) (Liu, C.; et al. Abstract 377: Evaluating Delivery of TFR1-Targeting Therapies to the CNS in a Novel Humanized TFR1 Mouse Model. Cancer Research, 2022, 82, 377-377). Either AAV-BI19 or AAV9 carrying a CAG-NLS-mScarlet-P2A-Luciferase-pA construct was intravenously injected (5e11 vg/mouse) into adult female C57BL/6J or C57BL/6-Tfr1tm1TFR1/Bcgen (B-hTFR1) mice. At three weeks post-injection, relative to AAV9, AAV-BI19 exhibited enhanced biodistribution to and transduction of the brain and spinal cord in B-hTFR1 but not C57BL/6J mice (FIG. 21A-E). This enhancement was not observed in other organs that were assessed. Compared to the C57BL/6J mice that received AAV9, B-hTFR1 mice that received AAV-BI19 showed a 6 and 12-fold increase in viral genomes recovered in the brain and spinal cord, respectively (FIG. 21A); a 54 and 43-fold increase in viral mRNA transcript levels in the brain and spinal cord, respectively (FIG. 19B); and a 132 and 58-fold in the luciferase reporter enzymatic activity in the brain and spinal cord, respectively (FIG. 19C). In addition, relative to AAV9, AAV-BI19 transduced a significant portion of the neurons and astrocytes in the cortex, striatum, and thalamus in B-hTFR1 but not C57BL/6J mice (FIG. 21F-I). In B-hTFR1 mice, AAV-BI19 transduced 54%, 32%, and 67% of NeuN⁺ cells, a marker for neurons, in the cortex, striatum, and thalamus, respectively (FIGS. 21F and 21H). In contrast, in C57BL/6J, AAV-BI19 transduced less than 1% of NeuN⁺ cells in the cortex and striatum and only 5% of NeuN⁺ cells in the thalamus. AAV9 transduced only 1-6% of NeuN⁺ cells in these same brain regions regardless of the mouse strain. More than 80% of SOX9⁺ cells were transduced by AAV-BI19 in B-hTFR1 mice, as compared to 20% or fewer SOX9⁺ cells transduced in the cortex, striatum, and thalamus by AAV9 in C57BL/6J mice (FIGS. 21G and 21H). The mScarlet reporter expression in the liver was comparable between AAV9 and AAV-BI19 in both C57BL/6J and B-hTFR1 mice (FIG. 22A) in alignment with the biodistribution and transduction data. In C57BL/6J mice, AAV-BI19 was less efficient at transducing the CNS and cells within the dorsal root ganglion (DRG) compared to AAV9; however, DRG cell transduction was comparable between AAV9 and AAV-BI19 in B-hTFR1 mice (FIG. 22B).

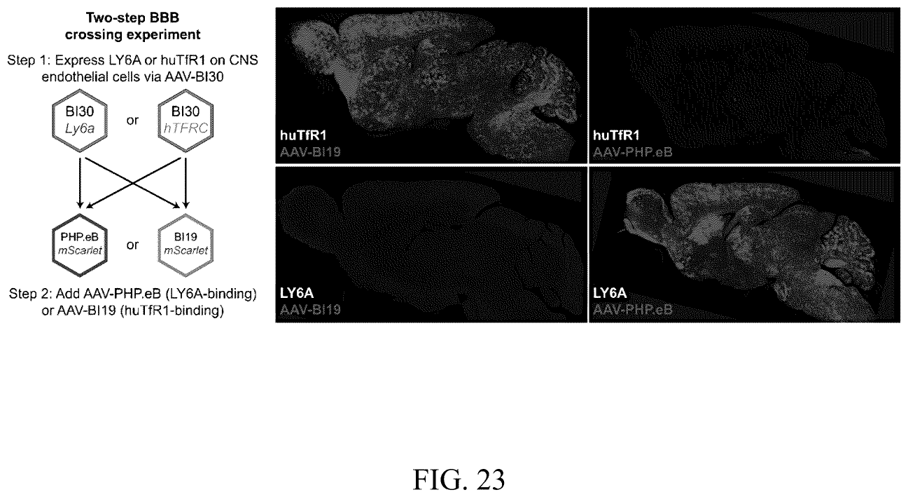

Applicants next sought to determine whether the improved uptake and transport across the brain endothelium mediated by huTfR1 is sufficient to explain the enhanced tropism of AAV-BI19. To test this, Applicants established a two-step BBB-crossing experiment (FIG. 23). In this experiment, NSG mice were first intravenously injected with AAV-BI30, a capsid that targets CNS endothelial cells (Krolak, T.; et al. A High-Efficiency AAV for Endothelial Cell Transduction throughout the Central Nervous System. Nature Cardiovascular Research, 2022, 1, 389-400), carrying either human TFRC or mouse Lyba; LY6A is the receptor that mediates the enhanced CNS tropism of AAV-PHP.eB in some strains of mice (Huang Q, et al. Delivering genes across the blood-brain barrier: LY6A, a novel cellular receptor for AAV-PHP.B capsids. PLOS One. 2019 Nov. 14; 14(11):e0225206) and serves as a positive control in this experiment (NSG wild type mice have the non-permissive Ly6a allele). Next, at 28 days post-injection, the mice were either intravenously injected with AAV-BI19 or AAV-PHP.eB carrying a CAG-mScarlet reporter. As predicted, AAV-BI19 effectively transduced the CNS of mice that had received AAV-BI30:CAG-hTFRC but not AAV-BI30:CAG-LY6A, and AAV-PHP.eB effectively transduced the CNS of mice that had received AAV-BI30:CAG-LY6A but not AAV-BI30:CAG-hTFRC (FIG. 23). These results show that endothelial expression of human TFRC is sufficient to explain much if not all of the enhanced CNS tropism of AAV-BI19. This experiment does rule out the possibility that interactions with TfR1 on brain parenchymal cells in the B-hTfR1 KI mice also impact the tropism of AAV-BI19.

### Discussion

In this work, Applicants demonstrated that AAV capsids can be reprogrammed to target a human cell surface protein in order to more efficiently cross the BBB in vivo. This builds on prior work by Applicants' group that successfully yielded suites of LY6A and LY6C1-targeting AAV capsids that cross the BBB in mice. Collectively, these efforts show that it is feasible to target AAV capsids to specific host proteins, including human proteins, to generate potent gene delivery vectors with known mechanisms of action and predictable tropisms. Furthermore, this approach which begins with in vitro receptor-binding screens produces reproducible and quantitative data that enables downstream saturation mutagenesis and machine learning-guided exploration of the capsid sequence space (Huang et al 2022).

While the approach results in greater confidence that the capsids are leveraging a mechanism that exists in humans, the capsids Applicants have reprogrammed to date including those that bind LY6A, LY6C1, and TFR1 have led to species-specific tropism enhancements. Therefore, to leverage in this study, Applicants validated the huTfR1-binding capsids in a knock-in mouse model, C57BL/6-Tfr1tm1TFR1/Bcgen (B-hTFR1) mice (FIG. 21), as well as in a two-step experiment where mice were first made to express hTfR1 on their CNS endothelium (FIG. 23). Given the highly specificity binding of AAV-BI19 for human TfR1, with minimal detected binding to the TfR1 from new world or old world monkeys, it may be worthwhile to establish new standards where gene therapy vectors do not need to be validated in NHP when they have a known mechanism of action that is human-specific.

In future work, it may be possible to reprogram capsids such that they bind to the same receptor across host species. However, enforcing cross-species receptor-targeting within a single capsid will require a focus on only the most highly conserved surface proteins, which may eliminate well characterized and validated candidate receptors like TfR1 thereby constraining the impact of this receptor-focused retargeting approach. Alternatively, dual efforts to identify human protein-targeted capsids and surrogates with matched affinities and tropism characteristics in one or more pre-clinical species can be undertaken, but this requires significant additional resources. In this context, gene therapy developers will need to weigh the importance of having a capsid that is highly optimized across human-specific assays versus capsids that can be used to demonstrate preclinical.

### Methods

### Plasmids

AAV9, AAV-BI19 and BI98-BI101 Rep-Cap plasmids were generated by gene synthesis (GenScript). The CAG-WPRE-hGH pA backbone was obtained from Viviana Gradinaru through Addgene (#99122). GFP, GFP-2A-luciferase, and mScarlet cDNAs were synthesized as gBlocks (IDT) or synthesized and cloned (GenScript).

Full length TFRC cDNA expression and lentiviral plasmids were cloned by inserting the open reading frames of TFRC (human: NM_003234.4; mouse: NM_011638.4; marmoset: NM_001301847.1, macaque: NM_001257303.1) into the lentiviral backbone pLenti-EF-FH-TAZ-ires-blast (Addgene #52083) with EcoRI/SalI sites.

For Fc fusion proteins, the coding sequence of the extracellular regions of TfR1 (human, marmoset, or macaque: 89-760aa; mouse: 89-763aa) were amplified by PCR and inserted into pCMV6-XL4 FLAG-NGRN-Fc (Addgene #115773) with EcoRV and Xbal sites. Signal peptide H7 (ATGGAGTTTGGGCTGAGCTGGGTTTTCCTCGTTGCTCTTTTTAGAGGTGTCCAGTGT (SEQ ID NO: 20493)) was introduced to N terminal of TFRC sequences to direct protein secretion.

### Cell Lines and Primary Cultures

HEK293T/17 (CRL-11268), Pro5 (CRL-1781) and CHO-K1 (CCL-61) were obtained from ATCC. mBMVEC cells (C57-6023) and hBMVECs cells (H-6023) were obtained from Cell Biologics and cultured as directed by the manufacturer. hCMEC/D3 cells were from Millipore-Sigma (SCC066).

Reconstitution of Full-Length TfR1 into Peptidiscs

Peptidisc peptide with the sequence N-FAEKFKEAVKDYFAKFWDPAAEKLKEAVKDYFAKLWD-C(SEQ ID NO: 42434) was purchased from PEPTIDISC LAB (peptidisc.com) and was dissolved at 2 mg/mL in buffer containing 20 mM TrisHCI, pH-8. TfR1 was reconstituted into Peptidiscs using the ‘on-bead’ method. Briefly, HEK293 cells expressing C-terminally tagged full length huTfR1 were harvested by centrifugation at 300×g. Cells were lysed by sonication and fractionated by centrifugation at 15,000×g to remove nuclei and cell debris and at 100,000×g to pellet the cell membrane. Membrane was resuspended in TrisHCI, pH=7.5, 50 mM NaCl, 10% glycerol to 10 mg/mL and solubilized with 0.8% DDM at 4° C. with gentle agitation. Anti-FLAG M2 magnetic beads (Sigma-Aldrich) were incubated with the solubilized membrane and washed with a buffer of TrisHCI, pH=7.5, 50 mM NaCl, 10% glycerol, 0.02% DDM. Beads were incubated for five minutes at room temperature in a solution of 1 mg/mL Peptidisc peptide and then thoroughly washed in buffer without detergent. Peptidisc-TfR1 was eluted with 0.1 mg/mL 3×FLAG peptide and analyzed by SDS-PAGE.

### Bio-Layer Interferometry

For characterization of the binding dynamics between huTfR1 and AAV capsids, a GatorPrime instrument was used (Gator Bio). Purified AAV-BI19 or AAV9 capsids were diluted in Q buffer (1× PBS, 0.02% Tween 20, and 0.2% BSA) and immobilized on AAVX probes. Probes without capsids were used as a control for nonspecific binding. Peptidisc-reconstituted huTfR1 was similarly prepared in a dilution range from 200 nM to 6.125 nM in Q buffer. Alternatively, this Peptidisc-huTfR 1 dilution range was prepared with 200 nM Holo-Tf in each dilution to ensure a complex of huTfR1 with Holo-Tf. Probes were incubated in Q buffer to establish a baseline for 120 seconds, then incubated with AAV-BI19 or AAV9 capsids to reach a loading height of approximately 10 nm before reestablishing a baseline for 120 seconds. Association with Peptidisc-huTfR1 or Peptidisc-huTfR1-Holo-Tf was performed over 120 seconds and dissociation into Q buffer occurred over 180 seconds.

### Lentivirus Production

Lentivirus was produced with a third-generation lentivirus system by co-transfection of three packaging plasmids (pMDLg-RRE, pRSV-Rev, and pVSV-G) and vector plasmid encoding human TFRC at a ratio of 4:1:1:6 in HEK293T/17 cells. Lentivirus was harvested three days after transfection and filtered with 0.45 μm PES to remove the cell debris.

### Exogenous TfR1 Expression in CHO Cell Lines

CHO cell lines overexpressing human, macaque, marmoset, or mouse TfR1 were established via random lentiviral integration of exogenous constructs under the control of an EF-1 alpha promoter. Briefly, CHO cells were seeded at 150,000 cells/well in 12 well plates and transduced with lentivirus-containing media for 48 hours, then transferred to 10 cm dishes for selection with 10 ug/mL blasticidin for one week. Once all cells had died in the control plate, the selected CHO cells were maintained in media with 1 ug/mL blasticidin. For validation, RNA was extracted from cells using the Qiagen RNeasy Kit. Maxima H Minus Reverse Transcriptase (ThermoFisher, EP0753) and oligo dT were used for cDNA synthesis. Species-specific primers were used for qPCR (Table 23). The CHO cells expressing TfR1 from each species were immunostained with two anti-TfR1 antibodies: Abcam ab84036, polyclonal, or eBioscience™ OKT9, monoclonal (FIG. 17).

### AAV Production and Titering

The AAV9 variant capsids carrying CAG-NLS-mScarlet-P2A-Luciferase-SV40-WPRE were either produced in suspension followed by iodixanol purification and titered as previously described (Eid, F.-E.; et al. Systematic Multi-Trait AAV Capsid Engineering for Efficient Gene Delivery, 2022) or produced using adherent cells as previously described (Challis, R. C., et al. Systemic AAV vectors for widespread and targeted gene delivery in rodents. Nat Protoc 14, 379-414 (2019)).

### Binding Assays

CHO cells stably expressing TfR1 from human, macaque, marmoset, or mouse were seeded at 30,000 cells per well; hCMEC/D3 cells were seeded at 30,000 cells per well; and hBMVEC cells were seeded at 15,000 cells per well. At 24 hours post-seeding, each well was subjected to a media change with the fresh cold media that contained each AAV9 variant carrying CAG-NLS-mScarlet-P2A-Luciferase-SV40-WPRE at 3,000, 6,000, or 12,000 vg/cell for CHO, hCMEC/D3, or hBMVEC cells, respectively. The plate was then maintained at 4° C. with gentle rocking for one hour, followed by five phosphate buffered saline (PBS) washes. Cells were treated with Proteinase K at 56° C. for one hour, followed by deactivation at 95° C. for 10 minutes. Total DNA extraction was performed using the DNeasy kit (Qiagen). The DNA was diluted 1:20 and subjected to qPCR with the following mScarlet primers: forward primer 5′-CCGTGACCCAGGACACCTC-3′ (SEQ ID NO: 42432) and reverse primer 5′-GCCATCTTAATGTCGCCCTTCAG-3′ (SEQ ID NO: 42433).

### Transduction Assays

CHO cells stably expressing TfR1 from human, macaque, marmoset, or mouse were seeded at 100,000 cells per well; hCMEC/D3 cells were seeded at 10,000 cells per well; and hBMVEC cells were seeded at 5,000 cells per well. At 24 hours post-seeding, each well was subjected to a media change with the fresh media that contained each AAV9 variant carrying CAG-NLS-mScarlet-P2A-Luciferase-SV40-WPRE at 3,000, 6,000, or 12,000 vg/cell for CHO, hCMEC/D3, or hBMVEC cells, respectively, and the plate was maintained at 37° C. with 5% CO₂ for 24 hours. Transduction was measured using the britelite plus reporter gene assay system (PerkinElmer, 6066766). Luciferase activity was reported as relative light units (RLU) as raw data or normalized to control wells transduced with AAV9.

### Antibody Inhibition Assay

The hCMEC/D3 cells were seeded at 7,500 cells per well. Two days later, 100 μL of media with 3e8 vg/mL virus and the specified concentration of OKT9 or R&D AF2474 antibody was transferred to each well. The plate was maintained at 37° C. with 5% CO₂ for 24 hours. Transduction was measured using the britelite Plus reporter gene assay system (Revvity).

### Transwell Assay

The hCMEC/D3 cell line (Millipore Sigma, Cat. No. SCC066), under 9 passages, was maintained in EGM2-MV media (CC4147 Lonza) to reach confluency. Falcon® Permeable Support for 24-well Plate with 1.0 um Transparent PET Membrane (353104 Corning) were prepared by coating with collagen type I (08-115 Millipore) diluted 1:50 in PBS and incubated at 37° C. for two hours. On Day 0, 3e4/cm² of cells were plated on the transmembrane and grown for two days in 200 μL of EGM2-MV media with VEGF on the top of the transmembrane and 600 μL of media at the bottom of the membrane. On Day 4, the media at both the top and bottom of the transwell was changed to EGM2-MV media without VEGF. Starting from Day 4, TEER values were measured using the EVOM2 meter (World Precision Instruments). TEER values were calculated as the ohmmeter readout in wells with cells minus the ohmmeter readout in wells without cells, multiplied by the surface area in cm² units. Transwell permeabilities using this process were previously validated by measuring the permeability of Dextran FITC 4k (46944 Sigma), 40k (76221-470 biotium), and 70k (76221-460 biotium). TEER peaked on Day 6, reaching values >10 Ω/cm², which is when AAVs were added to the transwells. AAV-BI19, AAV2, and AAV9 with serotype-specific barcoded transgenes were pooled and added to the top well of the transwell at 25,000 vg/cell (each AAV). After 180 minutes of incubation at 37° C. or 4° C., 22 μL of media was extracted from the bottom of the transwell. To detect the amount of virus in the media, extracts were treated with DNAseI (M0303S NEB) for 15 minutes at room temperature, then diluted in TE buffer (5 mM EDTA) and denatured at 70° C. for 10 minutes to deactivate DNAseI. DNA extracts were then diluted in 5% Tween 20 with random DNA filler (UltraPure™ Salmon Sperm DNA Solution, ThermoFisher, Cat. No. 15632011). The amount of virus that had passed through the transwell was measured by qPCR using a standard curve of known virus quantities.

### Immunofluorescence Staining and Colocalization

For huTfR1 colocalization, AAV-BI19 and AAV2 were added to monocultured HCMEC/D3 cells in optical plates (PhenoPlate 96-well, black, optically clear flat-bottom, tissue-culture treated plates, Perkinelmer 6055302) at 25,000 vg/cell, while Transferrin-647 (Transferrin From Human Serum, Alexa Fluor™ 647 Conjugate, T23366, ThermoFisher) and purified TfR1 antibody (Purified Anti-human CD71 Antibody OKT-9, 1:200 dilution, 10713000, AAT Bioquest) were added to wells to reach a final concentration of 1 μg/mL each. Cells were incubated for one hour at 37° C. or 4° C. accordingly, followed by rinse and fixation with 4% paraformaldehyde.

To visualize AAV particles, fixed cells were blocked by endogenous biotin (Avidin/Biotin Blocking Kit, SP-2001, Vector Laboratories) and then applying Anti-AAV9 (CaptureSelect™ Biotin Anti-AAV9 Conjugate, 1:200 dilution, 7103332100, ThermoFisher) to the wells treated with AAV-BI19 or wells with no virus, or Anti-AAVX (CaptureSelect™ Biotin Anti-AAVX Conjugate, 1:200 dilution, 7103522100, ThermoFisher) to the wells treated with AAV2. After 3 PBS washes to clean away unused primary antibodies, Neutravidin 488 (NeutrAvidin Protein, DyLight™ 488, 1:500 dilution, 22832, Thermofisher) was applied for visualization of the primary antibodies.

To visualize human TfR1, fixed cells were stained with huTfR1 antibody (Purified Anti-human CD71 Antibody OKT-9, 1:200 dilution, 10713000, AAT Bioquest), followed by Alexa-anti-mouse (CyTM3 AffiniPure Goat Anti-Mouse IgG (H+L), 1:500 dilution, 115-165-003, Jackson ImmunoResearch) were used for detection.

For organelle colocalization, the following stainings were applied: golgi (RCAS1 (D2B6N) XP® Rabbit mAb, 1:200 dilution, 12290, Cell Signaling Technology), early endosome (Rab 5 Antibody (D-11), 1:100 dilution, sc-46692, Santa Cruz Biotechnology), late endosome (Recombinant Alexa Fluor® 647 Anti-RAB7 antibody [EPR7589] (ab198337), 1:400 dilution, ab198337, abcam), and endoplasmic reticulum (Recombinant Alexa Fluor® 488 Anti-KDEL antibody, 1:500 dilution, ab184819, abcam). The following secondary antibodies were used: Streptavidin-555 nm (Streptavidin, AlexaFluor 555, 1:500 dilution, S21381, ThermoFisher), Goat anti mouse 647 nm (Alexa Fluor® 647 AffiniPure Goat Anti-Mouse IgG (H+L), 1:500 dilution, 115-605-062, Jackson ImmunoResearch), Goat anti mouse 488 nm (Alexa Fluor™ 488 Goat Anti-Mouse IgG (H+L) Cross-Adsorbed Secondary Antibody, 1:500 dilution, A-11001, ThermoFisher), Goat anti rabbit 488 nm (Alexa Fluor® 488 AffiniPure Goat Anti-Rabbit IgG (H+L), 1:500 dilution, 111-545-144, Jackson ImmunoResearch).

For wells requiring additional mouse antibodies, cells were blocked for 60 minutes with mouse-on-mouse blocking reagent (ReadyProbes™ Mouse-on-Mouse IgG Blocking Reagent, 1:30 dilution, R37621, ThermoFisher).

Images were obtained with a 60× oil objective on a Nikon Ti-e inverted spinning disc confocal microscope using predetermined, optimized and fixed exposure time to allow image comparisons.

### TfR1 Substitution Transduction Assay

CHO cells were transiently transfected with Mirus TransIT-LT1 (Mirus, MIR 2300) plasmids encoding a CAG promoter that drives expression of human TfR1, macaque TfR1, or variants with the indicated amino acid substitutions. Cells were maintained at 37° C. with 5% CO₂. At 24 hours post-transfection, AAV-BI19 or AAV9 encoding ssCAG-NLS-mScarlet-P2A-Lucierase-pA was applied at 10,000 vg/cell. After 24 hours, viral transduction was measured using the britelite Plus reporter gene assay system.

### Animals

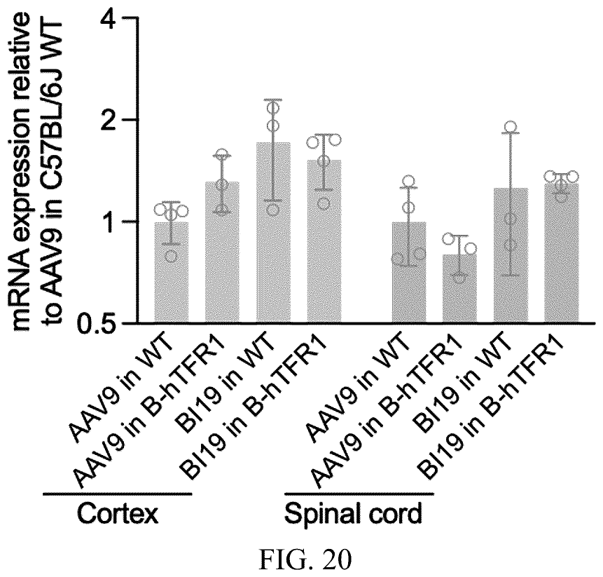

All procedures were performed as approved by the Broad Institute IACUC. C57BL/6J (000664) and NOD.Cg-Prkdcscid Il2rgtm1Wjl/SzJ (NSG) mice (005557) were obtained from The Jackson Laboratory (JAX). C57BL/6-Tfr1tm1TFR1/Bcgen (B-hTFR1, 110861) were obtained from Biocytogen. Recombinant AAV vectors were administered intravenously via the retro-orbital sinus in young adult male or female mice. Mice were randomly assigned to groups based on predetermined sample sizes. Transgenic animals were genotyped using DNeasy Blood & Tissue Kit (Qiagen, 69504) using primers listed in Table 23. Two mice were excluded from analysis in FIG. 21 and FIG. 20. The first mouse was assigned to the AAV9 in B-hTFR1 group but was determined to be negative for huTfR1 on Western blot and by genotyping. The second mouse in the AAV-BI19 in C57BL/6J group displayed no evidence of transduction in the biodistribution, transduction, or luciferase assays, and showed no fluorescent reporter signal in the liver or the brain.

Mice were anesthetized with Euthasol and transcardially perfused with PBS at room temperature followed by 4% paraformaldehyde (PFA) in ice cold PBS. Tissues were post-fixed overnight in 4% PFA in PBS and sectioned by vibratome. Immunohistochemistry (IHC) was performed on floating sections with antibodies diluted in PBS containing 5% donkey serum, 0.1% Triton X-100, and 0.05% sodium azide. Primary antibodies NeuN 1:500 (Invitrogen, MA5-33103), SOX9 1:250 (abcam, ab 185966) were incubated at room temperature overnight. The sections were then washed and stained with secondary Alexa-conjugated antibodies Alexa Fluor 647 (Invitrogen, A-31573) and Alexa Fluor 555 (Invitrogen, A-21427) at 1:1000 for four hours or overnight.

### In Vivo Capsid Biodistribution, Transduction, and Luciferase Measurement

AAV-BI19 or AAV9 encoding ssCAG-NLS-mScarlet-P2A-Luciferase-pA was intravenously injected into adult female C57BL/6J or C57BL/6-Tfr1tm1TFR1/Bcgen (B-hTFR1) mice at a dose of 5×10¹¹ vg/mouse. After three weeks, the mice were perfused with PBS. Parts of the mouse brain, liver, spinal cord, and dorsal root ganglions were dropped fixed into 4% PFA. Remaining tissues were collected and frozen at −80° C. For biodistribution, samples were processed for AAV genomes using a DNeasy 96 Blood & Tissue Kit (Qiagen, 69581) and qPCR was performed targeting mScarlet and mouse glucagon as previously described (see primer sequences in Table 23) (Deverman, B. E.; et al. Cre-Dependent Selection Yields AAV Variants for Widespread Gene Transfer to the Adult Brain. Nature Biotechnology, 2016, 34, 204-209). For the mRNA transduction assessment, RNA was isolated using TRIzol (Invitrogen, 15596026) and purified using an RNeasy 96 Kit (Qiagen, 74171); cDNA synthesis was performed using Maxima H Minus Reverse Transcriptase (Thermo Scientific, EP0753); qPCR targeting mScarlet and mouse GAPDH was performed. For the luciferase measurements, protein was extracted from the mice tissues using the lysis buffer T-Per with 1× Halt protease inhibitor (Invitrogen, 78430); 10 μg of total protein was used to assess the reporter gene using the britelite Plus reporter gene assay system (Perkin Elmer, 6066761).

For the two-step mouse BBB-crossing experiments, NSG mice were first intravenously injected with AAV-BI30 (Krolak, T.; et al. A High-Efficiency AAV for Endothelial Cell Transduction throughout the Central Nervous System. Nature Cardiovascular Research, 2022, 1, 389-400) at 1e11 vg/mouse to deliver AAV-CAG-hTFRC-WPRE-3x-miR122-pA or AAV-CAG-LY6A-WPRE-3x-miR122-pA. At 28 days post-injection, 5e11 vg of AAV-BI19: CAG-mScarlet or AAV-PHP.eB: CAG-mScarlet was systemically administered to each mouse. Transduction was assessed three weeks later.

Experiment 1: Screening for 7-Mer Modified AAV Capsids that Bind Selectively to Cells Expressing Human TFRC (hTFRC).

A library of capsids with a 7-mer insertion between AA588 and 589 of AAV9 K449R was screened for capsids that more efficiently bind to HEK293 or CHO cells transiently transfected with human TFRC (hTFRC) than cells expressing a control protein (GFP). Sequences that bind more efficiently to cells expressing hTFRC were identified by amplicon NGS. Analysis of these sequences identified several motifs present in a subset of sequences: [YFL][AS][RK]X[NG]X[N] and XXS[ST]NG[IVR], where X=any amino acid.

Applicants generated a second round library comprising sequences identified in the cell-based screen for hTFRC binding and all of the sequences conforming to the above two motifs using oligo pool synthesis. The oligo pool was used to generate a capsid library. The second round library was screened on HEK293 cells with or without transient expression of hTFRC (5,000 vg/cell) as performed in the first round (Table 1). In addition, the second round library was screened to identify capsids that more efficiently transduced (1) hCMEC/D3 cells, which express endogenous hTFRC, (2) mouse brain microvascular endothelial cells (BMVEC) s (Table 2) as well as (3) mixed culture spheroids consisting of hCMEC/D3 cells, and primary human astrocytes and pericytes (Cho et al, Nature Communications 2017).

Applicants chose several novel TFRC binding capsids for further study: AAV-BI19: YSRIGPN (SEQ ID NO: 14632); AAV-BI98: YSRLNMN (SEQ ID NO: 14301); AAV-BI99: YSRLNKD SEQ ID NO: 16577); AAV-BI100: VHRLQDK (SEQ ID NO: 16602); AAV-BI101: YHRLSNN (SEQ ID NO: 16636); and AAV-BI102: LHALSHN (SEQ ID NO: 16608). Applicants used these capsids to package a reporter transgene and transduced CHO cells or CHO cells expressing human TFRC (FIG. 2), as well as hCMEC cells or hCMEC cells overexpressing human TFRC (FIG. 3). In both cell types, each of the TFRC binding capsids showed enhanced ability to bind and transduce cells expressing human TFRC relative to AAV9. Notably, the enhanced transduction by these engineered capsids was absent in CHO cells, which lack expression of human TFRC. In contrast, the engineered AAVs each showed enhanced transduction of unmodified hCMEC cells relative to AAV9, which is fitting with the known expression of human TFRC in these cells. The role of TFRC for the enhanced hCMEC transduction of these capsid variants is supported by the finding that transduction was further enhanced in cells with greater cell surface exposed TFRC (FIGS. 3 and 4).

To assess whether the enhanced transduction of hCMEC cells by AAV-BI19 requires endogenous TFRC, applicants attempted to block the interaction of the AAV with TFRC using several anti-TFRC antibodies: OKT9 (Thermo) and AF2474 (R&D Systems). Applicants found that OKT9, which binds to the TFRC apical domain (Ferrero et al J. of Virology doi.org/10.1128/JVI.01868-20) blocked transduction by AAV-BI19 in a dose-dependent manner (FIG. 5). In contrast, AF2474, which blocks binding of Transferrin to TFRC, did not inhibit transduction by AAV-BI19. Together these results suggest that the enhanced tropism of AAV-BI19 for human brain endothelial cells is mediated through binding to the human TFRC apical domain.

Experiment 2: Screening for 7-Mer Modified AAV Capsids that Bind Selectively to hTFRC-Fc

In a second experiment, applicants screened a separately produced AAV9 K499R 7-mer 588 insertion library for variants capable of selectively binding hTFRC when produced as an Fc fusion protein. To perform this assay, applicants expressed the human or marmoset TFRC-Fc fusions in HEK293 cells, and pulled down the Fc fusion proteins from the cell culture media using ProA-magentic beads (FIG. 7).

The recovered 7-mers found to bind hTFRC-Fc were synthesized as an oligo pool and used to generate a second-round library. Applicants screened the second-round library for variants that bind hTFRC using the same hTFRC-Fc assay (Table 4), variants that bind to CHO cells stably expressing hTFRC (Table 5), variants that bind and transduce CHO cells expressing hTFRC (Table 6), and variants that bind HEK293 cells overexpressing hTFRC (Table 7). A subset of sequences bound HEK293 cells expressing either human TFRC or marmoset TFRC (Table 8).

### Experiment 3: Multiple Regions of the AAV Capsid can be Engineered to Enable TFRC Binding.

To determine whether other regions of the AAV capsid could be engineered to bind to TFRC, applicants generated libraries where a stretch of 10 AA in loop IV of AAV9 were substituted with a random string of AAs of seven or 10 AAs in length. Applicants also repeated the screen using a new loop VIII 7-mer insertion library as a positive control. Applicants screened these libraries for capsids that gained the ability to bind selectively to human TFRC (Table 9 and Table 10) or human TFRC where the apical domain was replaced with the corresponding domain from mouse TFRC (Table 11). Notably, several of the 10 AA sequences recovered in the loop IV substitution library (IPFSRVNPDT (SEQ ID NO: 20285), LGFARTGAAD (SEQ ID NO: 20274), LGFTKSSGSD (SEQ ID NO: 20270), LRYSKTQGES (SEQ ID NO: 20266), SPYARSSAGV (SEQ ID NO: 20271), and VGWSRLDLTT (SEQ ID NO: 20262)) contain the partial [YWFL][AST][RK] (SEQ ID NO: 20492) motif like that observed in human TFRC binding 7-mer insertions at loop VIII, suggesting that this binding motif is functional in multiple structural contexts.

Experiment 4: Toward a Systematic Mapping of the Relationship Between AAV 7-Mer Sequence and hTFRC Binding.

Applicants have generated a novel type of library (Fit4Function) composed of AAV capsids that uniformly samples only the high production fitness sequence space (cite WO2021222636A1). When used to screen for novel functions (e.g., new receptor binding), the Fit4Function libraries can generate highly reproducible quantitative data suitable for training ML models that predict the function of previously untested sequences within the theoretical sequence space. Applicants screened a Fit4Function library for capsids that selectively bind to TFRC-Fc. Binding was measured at both pH7.4 and pH5.5 and is reported in Table 12. Notably, even within this smaller library (240K unique sequences), several examples of sequences conforming to previously identified motifs were identified (e.g., YAKGGSN (SEQ ID NO: 11535) and YSKSGPG (SEQ ID NO: 20422), [YFL][AS][RK]X[NG]X[N] (SEQ ID NO: 20484); VKSSNGV (SEQ ID NO: 11671), XXS[ST]NG[IVR] (SEQ ID NO: 20485)).

Experiment 5: Secondary Evaluation of Recovered Variants from Previous Assays and Use of Those Sequences to Train Generative Models to Predict Additional High Performing Sequences.

To assess the function of AAV capsids discovered from Experiments 1-4 relative to each other and identify the top performing capsids across several relevant in vitro and in vivo assays, applicants generated a pooled oligo library containing loop VIII 7-mer insertion variants from Experiments 1-4 (>9K unique sequences). Applicants also used a subset of these 7-mer sequences with activity observed across multiple readouts to train a machine learning model (Wavenet that has been previously adapted for use in protein design) to generate additional sequences predicted to bind human TFR1 (>24K unique sequences). All sequences in the library were encoded by at least two unique nucleotide sequences, providing internal replicates for each 7-mer sequence. The pooled oligo library was then produced in two batches, one for in vitro studies. The library was then screened for binding to human, macaque, marmoset and mouse TFR1-Fc fusion proteins, for binding and transduction of control CHO cells or CHO cells stably expressing human, macaque, marmoset or mouse TFRC, for binding and transduction of hCMEC, for binding and transduction of hCMEC cells expressing exogenous human TFRC (hCMEC+hTFRC) in the presence or absence of holo-, total-, or apo-Transferrin (Tf), and for transduction in the brain, spinal cord, liver, muscle, kidney, and heart in mice expressing a chimeric form of TFRC where the extracellular domain of mouse Tfrc (Exons 4-19) have been replaced by the corresponding coding sequence for the extracellular domain of human TFRC (B-hTFR1 mice, Biocytogen). In addition, applicants also developed a two-step assay where AAV-BI30, a capsid that selectively transduces CNS endothelial cells (Krolak et al 2022) was first administered intravenously to immunodeficient NSG mice to express human TFRC, or mouse LY6A as a control, in the CNS vasculature. Subsequently, following expression (4 weeks) from AAV-BI30, the capsid library was administered intravenously and tissue samples were collected 4 weeks later. Capsid sequences are provided that were found to (1) bind TFR1-Fc fusion proteins with and without an additional 10 minute wash step in dPBS (pH5.5) (Table 14), (2) bind CHO cells expressing human TFRC (Table 15), (3) bind hCMEC and hCMEC+hTFRC (Table 16), (4) transduce hCMEC and hCMEC+hTFRC cells (Table 17), express Cap in the CNS of KI and/or 2-step TFRC mice (Table 18), bind hCMEC+hTFRC cells in the absence of Tf or in the presence of holo-, apo-, or total-Tf (Table 19).

Experiment 6: Screening for Capsids that Bind Human, Macaque, Marmoset TFR1-Fc Fusion Proteins

A new AAV9 K499R 7-mer 588 insertion library was screened for variants capable of selectively binding human, macaque, and mouse TFRC when produced as an Fc fusion protein. Top variants that were found to be enriched through binding human TFR1-Fc are provided (Table 20).

### Selection of Top Functional Variants Based on Convergence Across Multiple Functional Assays

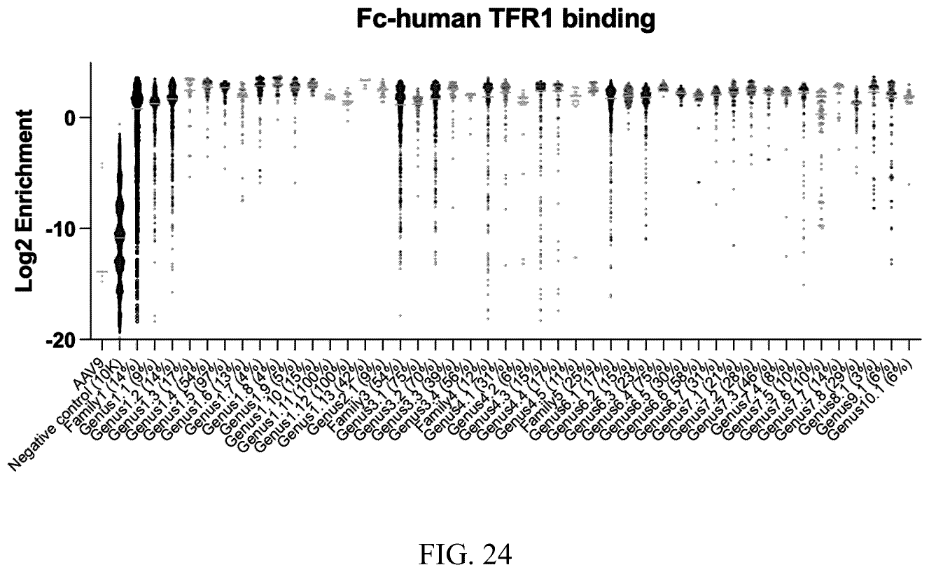

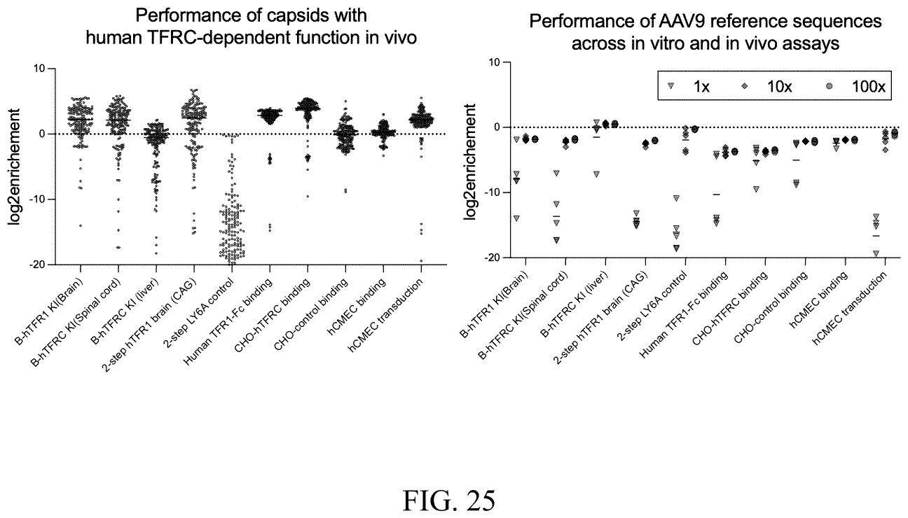

To identify top candidate capsids that engage TFR1 through specific motifs, applicants grouped the recovered sequences from Experiment 5 into 10 families and genera based on common sequence elements (Table 21). The motif definitions were used to extract all of the capsid sequences within the library that conformed to each family or genus definition regardless of activity. Values are reported as the log 2 enrichment (human TFR1-Fc binding assay RPM/AAV library RPM) for all sequences within the library for each motif (FIG. 24). Data for AAV9 and a set of negative control sequences (previously determined not to bind human TFRC-Fc) were included to facilitate interpretation of the enrichment scores relative to the parental vector and non-binding capsid sequences. Individual 7-mer AA sequences were encoded by two or more nucleotide references. Note that for each family and genus definition, the vast majority of the tested sequences were found to bind human TFR1-Fc better than AAV9 or the control capsid set (FIG. 24).

Next, top candidate capsids representing many of the families described above were chosen based on several functional attributes including production fitness (manufacturability), binding enrichment at pH 7 and following a low pH wash (pH 5.5), binding and transduction of hCMEC cells, binding in the presence of Tf (the preferred states are moderate or insensitive), CNS transduction in the humanized mouse models (B-hTFR1 KI mice and 2-step TFRC mice), and liver transduction in the B-hTFR1 mice (Table 22). Exemplary choices from several families include, but are not limited to, LHRLGPN (SEQ ID NO: 36834), YSRIGPN (SEQ ID NO: 14632) or YARTGPN (SEQ ID NO: 14269) in Family 1; LHAKLPN (SEQ ID NO: 36316) in Family 2; VESTNGR (SEQ ID NO: 36431), VQSTNGV (SEQ ID NO: 36423), and TESTNGR (SEQ ID NO: 17558) in Family 3; RMEDAYP (SEQ ID NO: 36895), RGEDVYP (SEQ ID NO: 36864), and RTYDSFP (SEQ ID NO: 37806) in Family 4; LCKPCLN (SEQ ID NO: 36936) or LCKPCLD (SEQ ID NO: 36437) in Family 5; KDDFTHF (SEQ ID NO: 36464), KDDFTTY (SEQ ID NO: 36336), and REDYVKW (SEQ ID NO: 37652) in Family 7; and NALEGRD (SEQ ID NO: 36407) and IALKGWD (SEQ ID NO: 36248) in Family 8.

### Tables Cont.

Table 14. Capsid that selectively bind to TFR1-Fc fusion proteins. A second round 7-mer 588 insertion library was screened for capsids that bind human, macaque, marmoset, or mouse TFR1-Fc fusion proteins in a pull-down assay. The binding assays were performed at pH7. In some assays the pulled down fusion protein-capsid complexes were washed with pH 5.5 buffer for 10 minutes (+pH5.5 wash). The table provides an assessment of the enrichment of capsids as assessed by Illumina sequencing where the enrichment is the log 2 assay reads per million (RPM)/RPM in the virus library before screening. Enrichment scores are listed within the following ranges: “−”<=0; “+”>0 to <=2; “++”>2 to <=3; “+++”>3.

Table 15. Capsids that selectively bind human TFR1-Fc fusion protein and CHO cells expressing TFRC. A second round 7-mer 588 insertion library was screened for capsids that bind (b) and transduce (t) control CHO cells or CHO cells stabling expressing human, macaque, marmoset, or mouse TFRC. The table provides an assessment of the enrichment of capsids as assessed by Illumina sequencing where the enrichment is the log 2 assay reads per million (RPM)/RPM of each variant in the virus library. Enrichment scores are listed within the following ranges: “−”<=0; “+”>0 to <=2; “++”>2 to <=3; “+++”>3.

Table 16. Capsids that bind hCMEC/D3s. A second round 7-mer 588 insertion library was screened for capsids that bind the hCMEC/D3 human brain endothelial cell line, which endogenously expresses TFRC (hCMEC binding) or hCMEC/D3 cells made to stably express human TFRC via transduction with a human TFRC expressing lentivirus (hCMEC-hTFRC binding). The table provides an assessment of the enrichment of capsids as assessed by Illumina sequencing where the enrichment is the log 2 assay reads per million (RPM)/RPM of each variant in the virus library. Enrichment scores are listed within the following ranges: “−”<=0; “+”>0 to <=1; “++”>1 to <=2; “+++”>2.

Table 17. Capsids that transduce hCMEC/D3s. A second round 7-mer 588 insertion library was screened for capsids that transduce the hCMEC/D3 human brain endothelial cell line, which endogenously expresses TFRC (hCMEC binding) or hCMEC/D3 cells made to stably express human TFRC via transduction with a human TFRC expressing lentivirus (hCMEC-hTFRC binding). The table provides an assessment of the enrichment of capsids as assessed by RT-PCR followed by Illumina sequencing where the enrichment is the log 2 assay reads per million (RPM)/RPM of each variant in the virus library. Enrichment scores are listed within the following ranges: “−”<=0; “+”>0 to <=1; “++”>1 to <=3; “+++”>3.

Table 18. Capsids that transduced cells in vivo following intravenous administration of a second round AAV library to mice engineered to express human TFRC or Lyba as a control. A second round 7-mer 588 insertion library was screened for capsids that transduce cells in the CNS of B-hTFR1mice. The same library was also administered to NGS mice made to express human TFRC (2-step assay) via an AAV-BI30 packaging AAV-CAG-hTFRC or AAV-CMV-CBA-hTFRC. As controls, the same library was also administered to separate cohorts of mice injected with AAV-CAG-Ly6a or AAV-CMV-CBA-Ly6a. The table provides an assessment of the enrichment of capsids as assessed by RT-PCR in the indicated in vivo assays followed by Illumina sequencing. Enrichment is the log 2 assay reads per million (RPM)/RPM of each variant in the virus library. Enrichment scores are listed within the following ranges: “−”<=0; “+”>0 to <=1; “+”>1 to <=3; “+”>3.

Table 19. Capsid binding to hCMEC-hTFRC cells in the presence or absence of human Transferrin (Tf). A second round 7-mer 588 insertion library was screened for capsids that bind hCMEC/D3 human brain endothelial cells made to stably express human TFRC via transduction with a human TFRC expressing lentivirus (hCMEC-hTFRC binding) in the absence of Tf (no Tf) or in the presence of total-Tf, holo-Tf, or apo-Tf (2 ug/ul). The table provides an assessment of the enrichment of capsids as assessed by PCR followed by Illumina sequencing where the enrichment is the log 2 assay reads per million (RPM)/RPM of each variant in the virus library. Enrichment scores are listed within the following ranges: “−”<=0; “+”>0 to <=1; “++”>1 to <=3; “+++”>3.

Table 20. Capsid binding to human or macaque TFR1-Fc fusion proteins and transduction in vivo following intravenous administration to mice expressing human or macaque TFRC. A new second round 7-mer 588 insertion library was screened for capsids that bind human, macaque, or mouse TFR1-Fc fusion proteins. The same library was also administered to NGS mice made to express human TFRC (2-step assay) via AAV-BI30 packaging AAV-CAG-hTFRC or AAV-CAG-macaque-TFRC. As controls, a separate group of mice was left uninjected with a receptor encoding AAV (2-step, control). The table provides an assessment of the enrichment of capsids as assessed by RT-PCR followed by Illumina sequencing where the enrichment is the log 2 assay reads per million (RPM)/RPM of each variant in the virus library. Enrichment scores are listed within the following ranges: “−”<=0; “+”>0 to <=1; “++”>1 to <=3; “+++”>3.

Table 21. Sequence motif families. The table provides sequence motifs that define specific capsid families and genera, the total number of possible sequence species within the motif, the number of unique 7-mer sequences within each family or genus, and number and fraction of possible sequences from each motif that were tested in the library. Amino acids presented in brackets indicate amino acids acceptable at the specific position. For example, Genus 1.11 FSRLG[AHNLVSPT]N (SEQ ID NO: 42435) has the sequence F-S-R-L-G-X1-N(SEQ ID NO: 42436), where X₁ can be any amino acid with the brackets (AHNL VSPT (SEQ ID NO: 42437)).

| | | | Sequences | | |
|---|---|---|---|---|---|
| | | | with | | |
| | | Defining sequence motif | the motif | | Fraction |
| Family | Genus | (SEQ ID NOS: 42384-42428) | family/genus | Assayed | assayed |
| Family 1 | | [YMFL][SHTA][KR][AGILMNQSTV][NG] | 11520 | 1608 | 13.96% |
| | | [ATHNFIPLY][DN] | | | |
| Family 1 | Genus1.1 | [YML][SHTA]\|KR][AGILMNQSTV][N][ATHNFIPLY] | 4320 | 399 | 9.24% |
| | | [DN] | | | |
| Family 1 | Genus 1.2 | [YML][SHTA][KR][AGILMNQSTV][G] | 5760 | 825 | 14.32% |
| | | [AGFHILNPSTVY][DN] | | | |
| Family 1 | Genus 1.3 | [LY][AHS][KR][AGHILMNQSTV]GPD | 132 | 23 | 17.42% |
| Family 1 | Genus 1.4 | [LY][AHS][KR][AGHILMNQSTV]GPN | 132 | 71 | 53.79% |
| Family 1 | Genus 1.5 | YSK[AILMNQSTV]G[YPTQVFLHSAEDIM]N | 126 | 122 | 96.83% |
| Family 1 | Genus 1.6 | [LY]S[RK][VITLA][SA][PRYFHIKW]D | 320 | 42 | 13.13% |
| Family 1 | Genus1.7 | [FLMY]H[KR][ALM][AGLMNQST] | 4224 | 172 | 4.07% |
| | | [ADEFHILMNQPSTVY][DN] | | | |
| Family 1 | Genus 1.8 | [LMY]H[KR][ALM][AGLMNQST] | 1584 | 61 | 3.85% |
| | | [ADEFHILMNQPSTVY]N | | | |
| Family 1 | Genus1.9 | [LMY]H[KR][ALM][AGLMNQST] | 1584 | 87 | 5.49% |
| | | [ADEFHILMNQPSTVY]D | | | |
| Family 1 | Genus1.10 | [LMY]H[KR]L[SQGTNL][PTVIQLA]D | 252 | 38 | 15.08% |
| Family 1 | Genus1.11 | FSRLG[AHNLVSPT]N | 8 | 8 | 100.00% |
| Family 1 | Genus1.12 | FAR[TSN]G[YFHPA]N | 15 | 15 | 100.00% |
| Family 1 | Genus1.13 | FH[KR]LG[IPS][ND] | 12 | 5 | 41.67% |
| Family2 | Genus2.1 | [YL]HA[KRNA][GQLS][PLINDT]N | 192 | 17 | 8.85% |
| Family3 | | [AFHILNPRSTVY][ADEFGHIKLMNQRSTWY]S | 1224 | 657 | 53.68% |
| | | [STING]IRV] | | | |
| Family3 | Genus3.1 | [FLY][DEHNQST]S[STING[IV] | 84 | 63 | 75.00% |
| Family3 | Genus3.2 | [VPISTHA][ADEFGHIKLMNQRSTY]S[ST]NG[IV] | 448 | 315 | 70.31% |
| Family3 | Genus3.3 | [AFILPSTVY][DENQST]S[TS]NGR | 90 | 35 | 38.89% |
| Family3 | Genus3.4 | R[EDQTIS[ST]NG[IV] | 16 | 9 | 56.25% |
| Family4 | | R[TLMSGDNE][GEDIFHSAMPVY]D[AVST][YF][AP] | 1536 | 184 | 11.98% |
| Family4 | Genus4.1 | R[TMLS][YSAMIFP]D[AVST][YF]P | 224 | 69 | 30.80% |
| Family4 | Genus4.2 | R[TMLS][YSAMIFP]D[AVST][YF]A | 224 | 13 | 5.80% |
| Family4 | Genus4.3 | R[GTDSNE][EDPSG][DTEHN][VAT][YF]P | 900 | 132 | 14.67% |
| Family4 | Genus4.4 | R[GLTDS][DPSG][DEHN][VT][YF]P | 320 | 53 | 16.56% |
| Family4 | Genus4.5 | T[RKYW][EWYQST]GD[PA][AP] | 96 | 11 | 11.46% |
| Family5 | | LC[KR]PC[LSDANQHPV][ETGADNS] | 126 | 32 | 25.40% |
| Family6 | Genus6.1 | [YF][WFY][TS]G[ITVQMH][IPHLMAQTV][ASDE] | 2592 | 445 | 17.17% |
| Family6 | Genus6.2 | [YF][WFY][TS]G[ITVQMH][KR][ASDE] | 576 | 89 | 15.45% |
| Family6 | Genus6.3 | [YF][WFY][TS]G[KR][IPHLMAQTV][ASDE] | 864 | 201 | 23.26% |
| Family6 | Genus6.4 | YFTG[KRQMHI][TRHKVL]E | 36 | 27 | 75.00% |
| Family6 | Genus6.5 | YF[TSHA]G[KRT][IPHLMAQT][DN] | 192 | 58 | 30.21% |
| Family6 | Genus6.6 | YWTG[KMVT][PVIHQTML][ED] | 64 | 37 | 57.81% |
| Family6 | Genus6.7 | [YF]F[SHAQ]G[KQR][IVLKHRQM]E | 192 | 60 | 31.25% |
| Family7 | Genus7.1 | [KR][DSN]EF[TKSRV][TSGV][FWY] | 360 | 77 | 21.39% |
| Family7 | Genus7.2 | [KR]DD[FY][TSVH][TSGVA][FWY] | 240 | 68 | 28.33% |
| Family7 | Genus7.3 | [SRGNTMQ]DG[TVSNY]S[KR]W | 70 | 32 | 45.71% |
| Family7 | Genus7.4 | [RVDIQK][MLG][SEARQ]DR[TASGKN]W | 540 | 44 | 8.15% |
| Family7 | Genus7.5 | [DIEQVSK][LMGP][EASDQT][SA]R[DSETGA]W | 2016 | 194 | 9.62% |
| Family7 | Genus7.6 | G[ESPGA][DEPN][GHTSN]V[RKS][WY] | 600 | 60 | 10.00% |
| Family7 | Genus7.7 | RE[DEPN][GHTSN]V[RKS][WY] | 120 | 17 | 14.17% |
| Family7 | Genus7.8 | G[GS][GESAPD][TGS]S[STHKRAN]W | 252 | 74 | 29.37% |
| Family8 | | [VIRND][AGS][LTSHG][KRE]G[WRAI][DG] | 1800 | 50 | 2.78% |
| Family9 | | [LMW][FRWKTY][DS]GT[PGSNAR][APSY] | 864 | 56 | 6.48% |
| Family10 | | [PNK][YF]A[RK]S[PVARILSE][EDML] | 384 | 23 | 5.99% |

Table 22. Top performing capsids across multiple measures relevant to traits important for a CNS gene therapy vector. Data from a second-round library was used to identify capsid sequences with in vivo BBB crossing activity in either or both B-hTFR1 KI mice or in NSG mice made to express human TFRC in brain vascular endothelial cells using AAV-BI30: CAG-human TFRC. Production fitness was assessed as log 2 enrichment for each sequence in the AAV library AAV RPM/plasmid library DNA RPM in two separate library batches, one used for in vivo studies and one used for in vitro studies. pH sensitive binding was assessed as log 2 enrichment (Fc-hTFR1 binding and washing at pH 7) minus log 2 enrichment (Fc-hTFR1 binding pH 7 and washing at pH 7 plus a final pH 5.5 rinse (dPBS) and incubation for 10 min). Capsids were screened to assess whether they are sensitive to competition for binding to TFR1 from human Transferrin (Tf) by assessing binding to hCMEC-hTFRC expressing cells at 4C in the presence of holo-Tf or no-Tf. Insensitive, moderate, and sensitive scores were defined as log 2 (enrichment score, hCMEC-hTFR+holo-Tf) minus log 2 (enrichment score, hCMEC-hTFR noTf >−0.4, <=−0.4 to −1, and <−1, respectively).

TABLE 23
Primers used for qPCR in this study.

| Primer name | Primer sequence |
|---|---|
| huTfR1-qPCR-F | GCTATGGGACTATTGCTGTGATCGTC (SEQ ID NO: |
| | 42438) |
| huTfR1-qPCR-R | CCAATATAAGCGACGTGCTGCAG (SEQ ID NO: 42439) |
| msTfR1-qPCR-F | TGTGGAGATGAAACTGGCTGC (SEQ ID NO: 42440) |
| msTfR1-qPCR-R | AGTGCAATAGCTGCAAAGCAGAG (SEQ ID NO: 42441) |
| marTfR1-qPCR-F | AACAAGAGCGTGAAGCTCTCTG (SEQ ID NO: 42442) |
| marTfR1-qPCR-R | CTCCAGGACCCCAGGAGTC (SEQ ID NO: 42443) |
| macTfR1-qPCR-F | GGAAAACAAGAGTGTGAAGCTCACG (SEQ ID NO: |
| | 42444) |
| macTfR1-qPCR-R | GGAGAGCTGTCCCCACACTG (SEQ ID NO: 42445) |
| MSCARLET-QPCR-F | CCGTGACCCAGGACACCTC (SEQ ID NO: 42432) |
| mScarlet-qPCR-R | CTTCTTCTGCATTACGGGGC (SEQ ID NO: 42446) |
| GAPDH-qPCR-F | ACCACAGTCCATGCCATCAC (SEQ ID NO: 42447) |
| GAPDH-qPCR-R | TCCACCACCCTGTTGCTGTA (SEQ ID NO: 42448) |
| mGlucagon-qPCR-F | AAGGGACCTTTACCAGTGATGTG (SEQ ID NO: 42449) |
| mGlucagon-qPCR-R | ACTTACTCTCGCCTTCCTCGG (SEQ ID NO: 42450) |
| msTfR1-exonland2-F | CTAACTTGTTTGGTGGGGAAC (SEQ ID NO: 42451) |
| msTfR1-exonland2-R | CTTCATCTGCAGCCAGTTTCAT (SEQ ID NO: 42452) |
| genotyping-h&mTFRC-F | CTCGTGAGGCTGGATCTCAAAAAGATG (SEQ ID NO: |
| | 42453) |
| genotyping-hTFRC-R | CACCAGGTAAACAAGTCTACCGTTCTTATC (SEQ ID |
| | NO: 42454) |
| genotyping-mTFRC-R | ACTGGGTCTAAGTTACCATTTGACTGC (SEQ ID NO: |
| | 42455) |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the

1. A composition comprising a targeting moiety effective to increase transduction of central nervous system (CNS) tissues via binding to a transferrin receptor (TFRC), optionally further comprising a cargo coupled to or otherwise associated with the targeting moiety.

2. The composition of claim 1, wherein the targeting moiety binds to an extracellular domain of the TFRC,
optionally, wherein the targeting moiety binds to one or more of an apical, helical, and/or protease-like domain of the extracellular domain, and
optionally, wherein the targeting moiety binds to the apical domain.

3-4. (canceled)

5. The composition of claim 1,
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from Y, M, F, and L;
X₂ comprises an amino acid chosen from S, H, T, and A;
X₃ comprises an amino acid chosen from K and R;
X₄ comprises an amino acid chosen from A, G, I, L, M, N, Q, S, T, V and H;
X₅ comprises an amino acid chosen from N, G, A, L, M, Q, S, and T;
X₆ comprises an amino acid chosen from A, T, H, N, F, I, P, L, Y, G, S, V, D, E, M, and Q; and
X₇ comprises an amino acid chosen from D and N;
or
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from Y or L;
X₂ comprises amino acid H;
X₃ comprises amino acid A;
X₄ comprises an amino acid chosen from K, R, N, and A;
X₅ comprises an amino acid chosen from G, Q, L, and S;
X₆ comprises an amino acid chosen from P, L, I, N, D, and T; and
X₇ comprises an amino acid chosen from N,
or
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from A, F, H, I, L, N, P, R, S, T, V, and Y;
X₂ comprises an amino acid chosen from A, D, E, F, G, H, I, K, L, M, N, Q, R, S, T, W, and Y;
X₃ comprises amino acid S;
X₄ comprises an amino acid chosen from S and T;
X₅ comprises amino acid N;
X₆ comprises amino acid G; and
X₇ comprises an amino acid chosen from I, R, and V,
or
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from R and T;
X₂ comprises an amino acid chosen from T, L, M, S, G, D, N, E, R, K, Y, and W;
X₃ comprises an amino acid chosen from G, E, D, I, F, H, S, A, M, P, V, Y, W, Q, and T;
X₄ comprises an amino acid chosen from D, T, E, H, N, and G;
X₅ comprises an amino acid chosen from A, V, S, T, and D;
X₆ comprises an amino acid chosen from Y, F, P and A; and
X₇ comprises an amino acid chosen from A and P,
or
wherein the targeting moiety comprises a n-mer motif, the n-mer motif comprising or consisting of an amino acid sequence of X₁-X₂-X₃-X₄-X₅-X₆-X₇,
wherein:
X₁ comprises amino acid L;
X₂ comprises amino acid C;
X₃ comprises an amino acid chosen from K and R;
X₄ comprises amino acid P;
X₅ comprises amino acid C;
X₆ comprises an amino acid chosen from L, S, D, A, N, Q, H, P, and V; and
X₇ comprises an amino acid chosen from E, T, G, A, D, N, and S,
or
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from Y, and F;
X₂ comprises an amino acid chosen from W, F, and Y;
X₃ comprises an amino acid chosen from S, T, H, A and Q;
X₄ comprises amino acid G;
X₅ comprises an amino acid chosen from I, T, V, Q, M, H, K, and R;
X₆ comprises an amino acid chosen from I, P, H, L, M, A, Q, T, V, K, and R; and
X₇ comprises an amino acid chosen from A, S, D, E, and N,
or
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from K, R, S, G, N, T, M, Q, V, D, I, and E;
X₂ comprises an amino acid chosen from D, S, N, M, L, G, P, E, and A;
X₃ comprises an amino acid chosen from E, D, G, S, A, R, Q, T, P, and N;
X₄ comprises an amino acid chosen from F, Y, T, V, S, N, A, G, and H;
X₅ comprises an amino acid chosen from T, K, S, R, V, and H;
X₆ comprises an amino acid chosen from T, S, G, V, A, K, R, N, D, E, and H; and
X₇ comprises an amino acid chosen from F, W, and Y,
or
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from V, I, R, N and D;
X₂ comprises an amino acid chosen from A, G, and S;
X₃ comprises an amino acid chosen from L, T, S, H, and G;
X₄ comprises an amino acid chosen from K, R, and E;
X₅ comprises amino acid G;
X₆ comprises an amino acid chosen from W, R, A, and I; and
X₇ comprises an amino acid chosen from D and G,
or
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from L, M, and W;
X₂ comprises an amino acid chosen from F, R, W, K, T, and Y;
X₃ comprises an amino acid chosen from D and S;
X₄ comprises amino acid G;
X₅ comprises amino acid T;
X₆ comprises an amino acid chosen from P, G, S, N, A, and R; and
X₇ comprises an amino acid chosen from A, P, S, and Y,
or
wherein the targeting moiety comprises a n-mer motif, the n-mer motif comprising or consisting of an amino acid sequence of X₁-X₂-X₃-X₄-X₅-X₆-X₇,
wherein:
X₁ comprises an amino acid chosen from P, N, and K;
X₂ comprises an amino acid chosen from Y and F;
X₃ comprises amino acid A;
X₄ comprises an amino acid chosen from R and K;
X₅ comprises amino acid S;
X₆ comprises an amino acid chosen from P, V, A, R, I, L, S, E; and
X₇ comprises an amino acid chosen from E, D, M, and L,
or
wherein the targeting moiety comprises a n-mer motif, the n-mer motif comprising or consisting of an amino acid sequence of Z₁-X₁-Z₂-X₂-X₃-X₄-X₅
wherein
Z₁ is selected from the group consisting of Y, F, and L,
Z₂ is selected from the group consisting of S, R, and K, and
X₁-X₅ are independently selected amino acids, or
wherein the targeting moiety comprises a n-mer motif, the n-mer motif comprising or consisting of an amino acid sequence of X₁-H-X₂-L-X₃-X₄-X₅,
wherein
X₁-X₅ are independently selected amino acids,
or
wherein the targeting n-mer motif is a 7-mer peptide having an amino acid sequence chosen from any one of SEQ ID NOs: 10952-20481 and 36241-42428.

6. The composition of claim 0,
wherein the n-mer motif is comprising or consisting of the amino acid sequence of X₁-X₂-X₃-X₄-X₅-X₆-X₇ and:
wherein
X₁ comprises an amino acid chosen from Y, M, and L;
X₂ comprises an amino acid chosen from S, H, T, and A;
X₃ comprises an amino acid chosen from K and R;
X₄ comprises an amino acid chosen from A, G, I, L, M, N, Q, S, T, and V;
X₅ comprises amino acid N;
X₆ comprises an amino acid chosen from A, T, H, N, F, I, P, L, and Y; and
X₇ comprises an amino acid chosen from D and N;
or
X₁ comprises an amino acid chosen from Y, M, and L;
X₂ comprises an amino acid chosen from S, H, T, and A;
X₃ comprises an amino acid chosen from K and R;
X₄ comprises an amino acid chosen from A, G, I, L, M, N, Q, S, T, and V;
X₅ comprises amino acid G;
X₆ comprises an amino acid chosen from A, G, F, H, I, L, N, P, S, T, V, and Y; and
X₇ comprises an amino acid chosen from D and N;
or
X₁ comprises an amino acid chosen from L and Y;
X₂ comprises an amino acid chosen from A, H, and S;
X₃ comprises an amino acid chosen from K and R;
X₄ comprises an amino acid chosen from A, G, H, I, L, M, N, Q, S, T, and V;
X₅ comprises amino acid G;
X₆ comprises amino acid P; and
X₇ comprises amino acid D;
or
X₁ comprises an amino acid chosen from L and Y;
X₂ comprises an amino acid chosen from A, H, and S;
X₃ comprises an amino acid chosen from K and R;
X₄ comprises an amino acid chosen from A, G, H, I, L, M, N, Q, S, T, and V;
X₅ comprises amino acid G;
X₆ comprises amino acid P; and
X₇ comprises amino acid N;
or
X₁ comprises amino acid Y;
X₂ comprises amino acid S;
X₃ comprises amino acid K;
X₄ comprises an amino acid chosen from A, I, L, M, N, Q, S, T, and V;
X₅ comprises amino acid G;
X₆ comprises amino acid X; and
X₇ comprises an amino acid chosen from Y, P, T, Q, V, F, L, H, S, A, E, D, I, and M;
or
X₁ comprises an amino acid chosen from L and Y;
X₂ comprises amino acid S;
X₃ comprises an amino acid chosen from R and K;
X₄ comprises an amino acid chosen from V, I, T, L, and A;
X₅ comprises an amino acid chosen from S and A;
X₆ comprises an amino acid chosen from P, R, Y, F, H, I, K, and W; and
X₇ comprises amino acid D;
or
X₁ comprises an amino acid chosen from F, L, M, and Y;
X₂ comprises amino acid H;
X₃ comprises an amino acid chosen from K and R;
X₄ comprises an amino acid chosen from A, L, and M;
X₅ comprises an amino acid chosen from A, G, L, M, N, Q, S, and T;
X₆ comprises an amino acid chosen from A, D, E, F, H, I, L, M, N, Q, P, S, T, V, and Y; and
X₇ comprises an amino acid chosen from D and N;
or
X₁ comprises an amino acid chosen from L, M, and Y;
X₂ comprises amino acid H;
X₃ comprises an amino acid chosen from K and R;
X₄ comprises an amino acid chosen from A, L, and M;
X₅ comprises an amino acid chosen from A, G, L, M, N, Q, S, and T;
X₆ comprises an amino acid chosen from A, D, E, F, H, I, L, M, N, Q, P, S, T, V, and Y; and
X₇ comprises amino acid N;
or
X₁ comprises an amino acid chosen from L, M, and Y;
X₂ comprises amino acid H;
X₃ comprises an amino acid chosen from K and R;
X₄ comprises an amino acid chosen from A, L, and M;
X₅ comprises an amino acid chosen from A, G, L, M, N, Q, S, and T;
X₆ comprises an amino acid chosen from A, D, E, F, H, I, L, M, N, Q, P, S, T, V, and Y; and
X₇ comprises amino acid D;
or
X₁ comprises an amino acid chosen from L, M, and Y;
X₂ comprises amino acid H;
X₃ comprises an amino acid chosen from K and R;
X₄ comprises amino acid L;
X₅ comprises an amino acid chosen from S, Q, G, T, N, and L;
X₆ comprises an amino acid chosen from P, T, V, I, Q, L, and A; and
X₇ comprises amino acid D;
or
X₁ comprises amino acid F;
X₂ comprises amino acid S;
X₃ comprises amino acid R;
X₄ comprises amino acid L;
X₅ comprises amino acid G;
X₆ comprises an amino acid chosen from A, H, N, L, V, S, P, and T; and
X₇ comprises amino acid N;
or
X₁ comprises amino acid F;
X₂ comprises amino acid A;
X₃ comprises amino acid R;
X₄ comprises an amino acid chosen from T, S, and N;
X₅ comprises amino acid G;
X₆ comprises an amino acid chosen from Y, F, H, P, and A; and
X₇ comprises amino acid N;
or
X₁ comprises amino acid F;
X₂ comprises amino acid H;
X₃ comprises an amino acid chosen from K and R;
X₄ comprises amino acid L;
X₅ comprises amino acid G;
X₆ comprises an amino acid chosen from I, P, and S; and
X₇ comprises an amino acid chosen from N and D;
or
X₁ comprises an amino acid chosen from F, L, and Y;
X₂ comprises an amino acid chosen from D, E, H, N, Q, S, and T;
X₃ comprises amino acid S;
X₄ comprises an amino acid chosen from S and T;
X₅ comprises amino acid N;
X₆ comprises amino acid G; and
X₇ comprises an amino acid chosen from I and V;
or
X₁ comprises an amino acid chosen from V, P, I, S, T, H, and A;
X₂ comprises an amino acid chosen from A, D, E, F, G, H, I, K, L, M, N, Q, R, S, T, and Y;
X₃ comprises amino acid S;
X₄ comprises an amino acid chosen from S and T;
X₅ comprises amino acid N;
X₆ comprises amino acid G; and
X₇ comprises an amino acid chosen from I and V;
or
X₁ comprises an amino acid chosen from A, F, I, L, P, S, T, V, and Y;
X₂ comprises an amino acid chosen from D, E, N, Q, S, and T;
X₃ comprises amino acid S;
X₄ comprises an amino acid chosen from T and S;
X₅ comprises amino acid N;
X₆ comprises amino acid G; and
X₇ comprises amino acid R;
or
X₁ comprises amino acid R;
X₂ comprises an amino acid chosen from E, D, Q, and T;
X₃ comprises amino acid S;
X₄ comprises an amino acid chosen from S and T;
X₅ comprises amino acid N;
X₆ comprises amino acid G; and
X₇ comprises an amino acid chosen from I and V;
or
X₁ comprises amino acid R;
X₂ comprises an amino acid chosen from T, M, L, and S;
X₃ comprises an amino acid chosen from Y, S, A, M, I, F, and P;
X₄ comprises amino acid D;
X₅ comprises an amino acid chosen from A, V, S, and T;
X₆ comprises an amino acid chosen from Y and F; and
X₇ comprises amino acid P;
or
X₁ comprises amino acid R;
X₂ comprises an amino acid chosen from T, M, L, and S;
X₃ comprises an amino acid chosen from Y, S, A, M, I, F, and P;
X₄ comprises amino acid D;
X₅ comprises an amino acid chosen from A, V, S, and T;
X₆ comprises an amino acid chosen from Y and F; and
X₇ comprises amino acid A:
or X₁ comprises amino acid R;
X₂ comprises an amino acid chosen from G, T, D, S, N, and E;
X₃ comprises an amino acid chosen from E, D, P, S, and G;
X₄ comprises an amino acid chosen from D, T, E, H, and N;
X₅ comprises an amino acid chosen from V, A, and T;
X₆ comprises an amino acid chosen from Y and F; and
X₇ comprises amino acid P;
or
X₁ comprises amino acid R;
X₂ comprises an amino acid chosen from G, L, T, D, and S;
X₃ comprises an amino acid chosen from D, P, S, and G;
X₄ comprises an amino acid chosen from D, E, H, and N;
X₅ comprises an amino acid chosen from V and T;
X₆ comprises an amino acid chosen from Y and F; and
X₇ comprises amino acid P;
or
X₁ comprises amino acid T;
X₂ comprises an amino acid chosen from R, K, Y, and W;
X₃ comprises an amino acid chosen from E, W, Y, Q, S, and T;
X₄ comprises amino acid G;
X₅ comprises amino acid D;
X₆ comprises an amino acid chosen from P and A; and
X₇ comprises an amino acid chosen from A and P;
or X₁ comprises an amino acid chosen from Y and F;
X₂ comprises an amino acid chosen from W, F, and Y;
X₃ comprises an amino acid chosen from T and S;
X₄ comprises amino acid G;
X₅ comprises an amino acid chosen from I, T, V, Q, M, and H;
X₆ comprises an amino acid chosen from I, P, H, L, M, A, Q, T, and V; and
X₇ comprises an amino acid chosen from A, S, D, and E;
or
X₁ comprises an amino acid chosen from Y and F;
X₂ comprises an amino acid chosen from W, F, and Y;
X₃ comprises an amino acid chosen from T and S;
X₄ comprises amino acid G;
X₅ comprises an amino acid chosen from I, T, V, Q, M, and H;
X₆ comprises an amino acid chosen from K and R; and
X₇ comprises an amino acid chosen from A, S, D, and E;
or
X₁ comprises an amino acid chosen from Y and F;
X₂ comprises an amino acid chosen from W, F, and Y;
X₃ comprises an amino acid chosen from T and S;
X₄ comprises amino acid G;
X₅ comprises an amino acid chosen from K and R;
X₆ comprises an amino acid chosen from I, P, H, L, M, A, Q, T, and V; and
X₇ comprises an amino acid chosen from A, S, D, and E;
or
X₁ comprises amino acid Y;
X₂ comprises amino acid F;
X₃ comprises amino acid T;
X₄ comprises amino acid G;
X₅ comprises an amino acid chosen from K, R, Q, M, H, and I;
X₆ comprises an amino acid chosen from T, R, H, K, V, and L; and
X₇ comprises amino acid E;
or
X₁ comprises amino acid Y;
X₂ comprises amino acid F;
X₃ comprises an amino acid chosen from T, S, H, and A;
X₄ comprises amino acid G;
X₅ comprises an amino acid chosen from K, R, and T;
X₆ comprises an amino acid chosen from I, P, H, L, M, A, Q, and T; and
X₇ comprises an amino acid chosen from D and N;
or
X₁ comprises amino acid Y;
X₂ comprises amino acid W;
X₃ comprises amino acid T;
X₄ comprises amino acid G;
X₅ comprises an amino acid chosen from K, M, V, and T;
X₆ comprises an amino acid chosen from P, V, I, H, Q, T, M, and L; and
X₇ comprises an amino acid chosen from E and D;
or
X₁ comprises an amino acid chosen from Y and F;
X₂ comprises amino acid F;
X₃ comprises an amino acid chosen from S, H, A, and Q;
X₄ comprises amino acid G;
X₅ comprises an amino acid chosen from K, Q, and R;
X₆ comprises an amino acid chosen from I, V, L, K, H, R, Q, and M; and
X₇ comprises amino acid E
or
X₁ comprises an amino acid chosen from K and R;
X₂ comprises an amino acid chosen from D, S, and N;
X₃ comprises amino acid E;
X₄ comprises amino acid F;
X₅ comprises an amino acid chosen from T, K, S, R, and V;
X₆ comprises an amino acid chosen from T, S, G, and V; and
X₇ comprises an amino acid chosen from F, W, and Y;
or
X₁ comprises an amino acid chosen from K and R;
X₂ comprises amino acid D;
X₃ comprises amino acid D;
X₄ comprises an amino acid chosen from F and Y;
X₅ comprises an amino acid chosen from T, S, V, and H;
X₆ comprises an amino acid chosen from T, S, G, V, and A; and
X₇ comprises an amino acid chosen from F, W, and Y;
or
X₁ comprises an amino acid chosen from S, R, G, N, T, M, and Q;
X₂ comprises amino acid D;
X₃ comprises amino acid G;
X₄ comprises an amino acid chosen from T, V, S, N, and Y;
X₅ comprises amino acid S;
X₆ comprises an amino acid chosen from K and R; and
X₇ comprises amino acid W;
or
X₁ comprises an amino acid chosen from R, V, D, I, Q, and K;
X₂ comprises an amino acid chosen from M, L, and G;
X₃ comprises an amino acid chosen from S, E, A, R, and Q;
X₄ comprises amino acid D;
X₅ comprises amino acid R;
X₆ comprises an amino acid chosen from T, A, S, G, K, and N; and
X₇ comprises amino acid W;
or
X₁ comprises an amino acid chosen from D, I, E, Q, V, S, and K;
X₂ comprises an amino acid chosen from L, M, G, and P;
X₃ comprises an amino acid chosen from E, A, S, D, Q, and T;
X₄ comprises an amino acid chosen from S and A;
X₅ comprises amino acid R;
X₆ comprises an amino acid chosen from D, S, E, T, G, and A; and
X₇ comprises amino acid W;
or
X₁ comprises amino acid G;
X₂ comprises an amino acid chosen from E, S, P, G, and A;
X₃ comprises an amino acid chosen from D, E, P, and N;
X₄ comprises an amino acid chosen from G, H, T, S, and N;
X₅ comprises amino acid V;
X₆ comprises an amino acid chosen from R, K, and S; and
X₇ comprises an amino acid chosen from W and Y;
or
X₁ comprises amino acid R;
X₂ comprises amino acid E;
X₃ comprises an amino acid chosen from D, E, P, and N;
X₄ comprises an amino acid chosen from G, H, T, S, and N;
X₅ comprises amino acid V;
X₆ comprises an amino acid chosen from R, K, and S; and
X₇ comprises an amino acid chosen from W and Y;
or
X₁ comprises amino acid G;
X₂ comprises an amino acid chosen from G and S;
X₃ comprises an amino acid chosen from G, E, S, A, P, and D;
X₄ comprises an amino acid chosen from T, G, and S;
X₅ comprises amino acid S;
X₆ comprises an amino acid chosen from S, T, H, K, R, A, and N; and
X₇ comprises amino acid W,
or
wherein the n-mer motif is comprising or consisting the amino acid sequence Z₁-X₁-Z₂-X₂-X₃-X₄-X₅ and;
X₁ comprises an amino acid chosen from A, S, or H;
X₂ comprises an amino acid chosen from S, T, L, or I;
X₃ comprises an amino acid chosen from N or G;
X₄ comprises amino acid G;
X₅ comprises an amino acid chosen from N, D, I, V, or R;
or any combination thereof.

7. The composition of claim 0, wherein the n-mer motif is selected from the group consisting of LHRLGPN (SEQ ID NO: 36834), YSRIGPN (SEQ ID NO: 14632), LHRLGPN (SEQ ID NO: 36834), LHRLGPD (SEQ ID NO: 36413), LHRAGPD (SEQ ID NO: 36894), YSRIGPD (SEQ ID NO: 38223), LSRIGPD (SEQ ID NO: 36274), LARSGPD (SEQ ID NO: 18035), YSRNSDN (SEQ ID NO: 16626), LHKAGPN (SEQ ID NO: 36305), LSRIGPN (SEQ ID NO: 36347), LAKSGPN (SEQ ID NO: 36287), YARNGPN (SEQ ID NO: 14048) and YSRNSDN (SEQ ID NO: 16626).

8-11. (canceled)

12. The composition of claim 6, wherein the n-mer motif is selected from the group consisting of FRSTNGV (SEQ ID NO: 16070), VESTNGR (SEQ ID NO: 36431), VDSTNGV (SEQ ID NO: 12206), VQSTNGV (SEQ ID NO: 36423), VSSTNGV (SEQ ID NO: 12333), TESTNGR (SEQ ID NO: 17558), VQSTNGI (SEQ ID NO: 11292) and FVSTNGV (SEQ ID NO: 11162).

13-14. (canceled)

15. The composition of claim 6, wherein the n-mer motif is selected from the group consisting of RGEDVYP (SEQ ID NO: 36864), RLEDVFP (SEQ ID NO: 36264), RTYDSYP (SEQ ID NO: 37938), RTYDAYP (SEQ ID NO: 38571), RTYDSFP (SEQ ID NO: 37806), RTETVYP (SEQ ID NO: 36486), RTETVFP (SEQ ID NO: 36389), and RTEHVFP (SEQ ID NO: 36603).

16. (canceled)

17. The composition of claim 5, wherein the n-mer motif comprises of LCKPCLD (SEQ ID NO: 36437) or LCKPCPT (SEQ ID NO: 36438).

18-21. (canceled)

22. The composition of claim 6, wherein the n-mer motif is selected from the group consisting of KDEFTTF (SEQ ID NO: 36308), KDDFTTY (SEQ ID NO: 36336), RDEFTTY (SEQ ID NO: 36615), KDEFSTY (SEQ ID NO: 36390), RDEFTSF (SEQ ID NO: 36701), and REDHVSW (SEQ ID NO: 37067).

23. (canceled)

24. The composition of claim 5, wherein the n-mer motif is selected from the group consisting of IALKGWD (SEQ ID NO: 36248), NALEGRD (SEQ ID NO: 36407), VALEGRD (SEQ ID NO: 36604), and VALKGWD (SEQ ID NO: 17701).

25-32. (canceled)

33. The composition of claim 5, wherein the n-mer motif is selected from the group consisting of YSRIGPN (SEQ ID NO: 14632), YSRLNMN (SEQ ID NO: 14301), YSRLNKD (SEQ ID NO: 16577), and YHRLSNN (SEQ ID NO: 16636).

34. (canceled)

35. The composition of claim 5, wherein the n-mer motif is VHRLQDK (SEQ ID NO: 16602) or LHALSHN (SEQ ID NO: 16608).

36. The composition of claim 5, wherein the n-mer motif comprises PSATNGV (SEQ ID NO: 20486), QVSTNGI (SEQ ID NO: 16021), SYSSNGV (SEQ ID NO: 16234), HQSSNGV (SEQ ID NO: 15978), VGSINGI (SEQ ID NO: 16199), AMSTNGR (SEQ ID NO: 16000), SASTNGV (SEQ ID NO: 16127), YMSTNGV (SEQ ID NO: 16042), YYSSNGV (SEQ ID NO: 16206), VHSTNGI (SEQ ID NO: 16134), PLSTNGV (SEQ ID NO: 16233), VYSTNGI (SEQ ID NO: 16059), IISTNGV (SEQ ID NO: 16054), RSVSSNGV (SEQ ID NO: 20502), YKSSNGV (SEQ ID NO: 16123), FRSTNGV (SEQ ID NO: 16070), and/or FVSTNGV (SEQ ID NO: 11162).

37. The composition of claim 5, wherein the n-mer is selected from any one of the amino acid sequences as listed in Tables 1-22, SEQ ID NO: 10952-20481 and 36241-42428, or any combination thereof.

38. (canceled)

39. The composition of claim 1, wherein the targeting moiety is part of a viral capsid protein,
optionally, wherein the targeting moiety is inserted or substituted in loop IV, loop VIII, or both of an AAV capsid protein, and
optionally, wherein the targeting moiety is IPFSRVNPDT (SEQ ID NO: 20285), LGFARTGAAD (SEQ ID NO: 20274), LGFTKSSGSD (SEQ ID NO: 20270), LRYSKTQGES (SEQ ID NO: 20266), SPYARSSAGV (SEQ ID NO: 20271), VGWSRLDLTT (SEQ ID NO: 20262).

40. The composition of claim 5, wherein the targeting moiety is inserted or substituted in loop IV, loop VIII, or both of an AAV capsid protein, optionally wherein the targeting moiety is IPFSRVNPDT (SEQ ID NO: 20285), LGFARTGAAD (SEQ ID NO: 20274), LGFTKSSGSD (SEQ ID NO: 20270), LRYSKTQGES (SEQ ID NO: 20266), SPYARSSAGV (SEQ ID NO: 20271), VGWSRLDLTT (SEQ ID NO: 20262).

41. (canceled)

42. The composition of claim 39, wherein targeting moiety is inserted between two amino acids of one or more capsid proteins such that the targeting moiety is external to an AAV capsid.

43. The composition of claim 39, wherein the viral capsid protein is an AAV viral capsid protein, optionally wherein the capsid protein is VP1, VP2, VP3, or a combination thereof.

44. The composition of claim 1, wherein the targeting moiety is inserted between amino acids 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10 or wherein the targeting moiety is inserted between two consecutive amino acids within amino acid 451-460 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10.

45-46. (canceled)

47. The composition of claim 1, wherein the cargo is a polynucleotide, a recombinant AAV genome comprising a transgene, one or more polypeptides, a ribonucleoprotein complex,
optionally, wherein the polynucleotide is operably linked to a regulatory sequence that promotes expression in the CNS, and
optionally, wherein the polynucleotide encodes one or more polypeptides and/or a RNAi oligonucleotide,
optionally, wherein the one or more polypeptides comprise enzymes or antibodies, and
optionally, wherein the polynucleotide encodes a CRISPR-Cas system.

48-51. (canceled)

52. The composition of claim 47, wherein the polynucleotide is operably linked to a regulatory sequence that promotes expression in the CNS.

53. A viral capsid or viral particle, comprising the composition of claim 1,
optionally, wherein the viral capsid or viral particle is an AAV viral capsid or AAV viral particle, and
optionally, wherein the targeting moiety is inserted between amino acids of 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10.

54. The viral capsid or viral particle of claim 53, further comprising a recombinant viral genome, wherein the recombinant viral genome encodes a therapeutic protein or nucleic acid, control polypeptide or nucleic acid, and/or selectable marker polypeptide or nucleic acid,
optionally, wherein the therapeutic protein or nucleic acid, control polypeptide or nucleic acid, and/or selectable marker polypeptide or nucleic acid is operably linked to a regulatory sequence that promotes expression in the CNS, and
optionally, wherein the recombinant viral genome is a recombinant AAV viral genome.

55-58. (canceled)

59. A vector system comprising one or more vectors, wherein at least one of the one or more vectors encodes a targeting moiety effective to increase transduction of central nervous system tissues (CNS) via binding to a transferrin receptor (TFRC), and optionally wherein at least one of the one or more vectors encodes a recombinant AAV genome comprising a transgene encoding a protein or a polypeptide, optionally further comprising a cargo.

60. The vector system of claim 59, wherein the targeting moiety binds to an extracellular domain of the TFRC,
optionally, wherein the targeting moiety binds to one or more of an apical, helical, and/or protease-like domain of the extracellular domain.

61-62. (canceled)

63. The vector system of claim 59, wherein the targeting moiety comprises a n-mer motif,
wherein the targeting n-mer motif peptide is a 7-mer peptide having an amino acid sequence of X₁-X₂-X₃-X₄-X₅-X₆-X₇,
wherein:
X₁ comprises an amino acid chosen from Y, M, F, and L;
X₂ comprises an amino acid chosen from S, H, T, and A;
X₃ comprises an amino acid chosen from K and R;
X₄ comprises an amino acid chosen from A, G, I, L, M, N, Q, S, T, V and H;
X₅ comprises an amino acid chosen from N, G, A, L, M, Q, S, and T;
X₆ comprises an amino acid chosen from A, T, H, N, F, I, P, L, Y, G, S, V, D, E, M, and Q; and
X₇ comprises an amino acid chosen from D and N,
or
wherein the targeting n-mer motif peptide is a 7-mer peptide having an amino acid sequence of X₁-X₂-X₃-X₄-X₅-X₆-X₇,
wherein:
X₁ comprises an amino acid chosen from Y or L;
X₂ comprises amino acid H;
X₃ comprises amino acid A;
X₄ comprises an amino acid chosen from K, R, N, and A;
X₅ comprises an amino acid chosen from G, Q, L, and S;
X₆ comprises an amino acid chosen from P, L, I, N, D, and T; and
X₇ comprises amino acid N,
or
wherein the targeting n-mer motif peptide is a 7-mer peptide having an amino acid sequence of X₁-X₂-X₃-X₄-X₅-X₆-X₇,
wherein:
X₁ comprises an amino acid chosen from A, F, H, I, L, N, P, R, S, T, V, and Y;
X₂ comprises an amino acid chosen from A, D, E, F, G, H, I, K, L, M, N, Q, R, S, T, W, and Y;
X₃ comprises amino acid S;
X₄ comprises an amino acid chosen from S and T;
X₅ comprises amino acid N;
X₆ comprises an amino acid chosen from G; and
X₇ comprises an amino acid chosen from I, R, and V,
or
wherein the targeting n-mer motif peptide is a 7-mer peptide having an amino acid sequence of X₁-X₂-X₃-X₄-X₅-X₆-X₇,
wherein:
X₁ comprises an amino acid chosen from R and T;
X₂ comprises an amino acid chosen from T, L, M, S, G, D, N, E, R, K, Y, and W;
X₃ comprises an amino acid chosen from G, E, D, I, F, H, S, A, M, P, V, Y, W, Q, and T;
X₄ comprises an amino acid chosen from D, T, E, H, N, and G;
X₅ comprises an amino acid chosen from A, V, S, T, and D;
X₆ comprises an amino acid chosen from Y, F, P and A; and
X₇ comprises an amino acid chosen from A and P,
or
wherein the targeting n-mer motif peptide is a 7-mer peptide having an amino acid sequence of X₁-X₂-X₃-X₄-X₅-X₆-X₇,
wherein:
X₁ comprises amino acid L;
X₂ comprises amino acid C;
X₃ comprises an amino acid chosen from K and R;
X₄ comprises amino acid P;
X₅ comprises amino acid C;
X₆ comprises an amino acid chosen from L, S, D, A, N, Q, H, P, and V; and
X₇ comprises an amino acid chosen from E, T, G, A, D, N, and S,
or
wherein the targeting n-mer motif peptide is a 7-mer peptide having an amino acid sequence of X₁-X₂-X₃-X₄-X₅-X₆-X₇,
wherein:
X₁ comprises an amino acid chosen from Y, and F;
X₂ comprises an amino acid chosen from W, F, and Y;
X₃ comprises an amino acid chosen from S, T, H, A and Q;
X₄ comprises amino acid G;
X₅ comprises an amino acid chosen from I, T, V, Q, M, H, K, and R;
X₆ comprises an amino acid chosen from I, P, H, L, M, A, Q, T, V, K, and R; and
X₇ comprises an amino acid chosen from A, S, D, E, and N,
or
wherein the targeting n-mer motif peptide is a 7-mer peptide having an amino acid sequence of X₁-X₂-X₃-X₄-X₅-X₆-X₇,
wherein:
X₁ comprises an amino acid chosen from K, R, S, G, N, T, M, Q, V, D, I, and E;
X₂ comprises an amino acid chosen from D, S, N, M, L, G, P, E, and A;
X₃ comprises an amino acid chosen from E, D, G, S, A, R, Q, T, P, and N;
X₄ comprises an amino acid chosen from F, Y, T, V, S, N, A, G, and H;
X₅ comprises an amino acid chosen from T, K, S, R, V, and H;
X₆ comprises an amino acid chosen from T, S, G, V, A, K, R, N, D, E, and H; and
X₇ comprises an amino acid chosen from F, W, and Y,
or
wherein the targeting n-mer motif peptide is a 7-mer peptide having an amino acid sequence of X₁-X₂-X₃-X₄-X₅-X₆-X₇,
wherein:
X₁ comprises an amino acid chosen from V, I, R, N and D;
X₂ comprises an amino acid chosen from A, G, and S;
X₃ comprises an amino acid chosen from L, T, S, H, and G;
X₄ comprises an amino acid chosen from K, R, and E;
X₅ comprises amino acid G;
X₆ comprises an amino acid chosen from W, R, A, and I; and
X₇ comprises an amino acid chosen from D and G,
or
wherein the targeting n-mer motif peptide is a 7-mer peptide having an amino acid sequence of X₁-X₂-X₃-X₄-X₅-X₆-X₇,
wherein:
X₁ comprises an amino acid chosen from L, M, and W;
X₂ comprises an amino acid chosen from F, R, W, K, T, and Y;
X₃ comprises an amino acid chosen from D and S;
X₄ comprises amino acid G;
X₅ comprises amino acid T;
X₆ comprises an amino acid chosen from P, G, S, N, A, and R; and
X₇ comprises an amino acid chosen from A, P, S, and Y,
or
wherein the targeting n-mer motif peptide is a 7-mer peptide having an amino acid sequence of X₁-X₂-X₃-X₄-X₅-X₆-X₇,
wherein:
X₁ comprises an amino acid chosen from P, N, and K;
X₂ comprises an amino acid chosen from Y and F;
X₃ comprises amino acid A;
X₄ comprises an amino acid chosen from R and K;
X₅ comprises amino acid S;
X₆ comprises an amino acid chosen from P, V, A, R, I, L, S, E; and
X₇ comprises an amino acid chosen from E, D, M, and L, or
wherein the targeting n-mer motif peptide is a 7-mer peptide having an amino acid sequence of Z₁-X₁-Z₂-X₂-X₃-X₄-X₅
wherein
Z₁ is selected from the group consisting of Y, F, and L,
Z₂ is selected to the group consisting of S, R, and K, and
X₁-X₅ are independently selected amino acids,
or
wherein the targeting n-mer motif peptide is a 7-mer peptide having an amino acid sequence of X₁-H-X₂-L-X₃-X₄-X₅,
wherein X₁-X₅ are independently selected amino acids,
or
wherein the targeting n-mer motif peptide is a 7-mer peptide having an amino acid sequence of any one of SEQ ID NOs: 10952-20481 and 36241-42428.

64-73. (canceled)

74. The vector system of claim 63, wherein the n-mer is selected from any one as listed in Tables 1-22, or any combination thereof.

75. (canceled)

76. The vector system of claim 59, wherein the targeting moiety is part of a viral capsid protein;
optionally, wherein the viral capsid protein is an AAV viral capsid protein,
optionally, wherein the capsid protein is VP1, VP2, VP3, or a combination thereof;
optionally, wherein the targeting moiety is inserted or substituted in loop IV and/or loop VIII; and
optionally, wherein targeting moiety is inserted between two amino acids of one or more capsid proteins such that the targeting moiety is external to the AAV capsid,
optionally, wherein the targeting moiety is inserted between amino acids 588 and 589 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10, and
optionally, wherein the targeting moiety is inserted between amino acids 451-460 of a capsid protein of AAV9, or in an analogous position of a capsid protein from AAV1, AAV2, AAV3, AAV4, AAV5, AAV6, AAV7, AAV8, AAVrh8, AAV rh.74, or AAV rh. 10.

77. (canceled)

78. The vector system of claim 59, wherein the targeting moiety is IPFSRVNPDT (SEQ ID NO: 20285), LGFARTGAAD (SEQ ID NO: 20274), LGFTKSSGSD (SEQ ID NO: 20270), LRYSKTQGES (SEQ ID NO: 20266), SPYARSSAGV (SEQ ID NO: 20271), VGWSRLDLTT (SEQ ID NO: 20262).

79-83. (canceled)

84. The vector system of claim 59, wherein the cargo is a polynucleotide, a recombinant AAV genome comprising a transgene, one or more polypeptides, a ribonucleoprotein complex,
optionally, wherein the polynucleotide is operably linked to a regulatory sequence that promotes expression in the CNS, and
optionally, wherein the polynucleotide encodes one or more polypeptides and/or a RNAi oligonucleotide,
optionally, wherein the one or more polypeptides comprise enzymes or antibodies, and
optionally, wherein the polynucleotide encodes a CRISPR-Cas system.

85-89. (canceled)

90. A polypeptide encoded or produced by the vector system of claim 59, optionally wherein the polypeptide is a capsid protein, optionally an AAV capsid polypeptide.

91. (canceled)

92. A particle produced by the vector system of claim 59, optionally wherein the particle is a viral particle, optionally an AAV particle.

93. (canceled)

94. A cell comprising the composition of claim 1, a vector system comprising one or more vectors wherein at least one of the one or more vectors encodes the composition, a polypeptide encoded or produced by the vector system, or particle produced by the vector system.

95. A method of delivering one or more cargos to the CNS, comprising:
administering, in vivo or in vitro, the capsid and/or viral particle of claim 53;
optionally wherein the cargo is recombinant AAV genome comprising a transgene, a polynucleotide which encodes an RNAi oligonucleotide, a polynucleotide encoding a polypeptide, or a polypeptide, optionally wherein the polypeptide includes an enzyme or antibody;
optionally wherein the cargo is a Cas polypeptide, a guide molecule, or both;
optionally wherein the cargo encodes a nuclease or a nucleic acid component of a RNA-guided nuclease; and
optionally wherein the cargo is one or more polynucleotides encoding the nuclease and nucleic acid component of a RNA-guided nuclease.

96-100. (canceled)

101. A method of creating humanized transgenic non-human animals comprising:
delivering to one or more cells of a non-human animal a vector system or a recombinant viral particle comprising a recombinant viral genome, wherein;
the vector system or recombinant viral genome encodes a human transferrin polypeptide, wherein the encoded human transferrin polypeptide is under control of a tissue-specific promoter or miRNA binding element that has selective activity in a desired cell, tissue, or organ,
optionally wherein the recombinant viral particle, optionally an AAV viral particle, comprises a capsid polypeptide, optionally an AAV capsid polypeptide, wherein the capsid polypeptide comprises a CNS-specific n-mer motif,
optionally wherein the CNS-specific n-mer motif comprises X1-N-X3-X4-X5-X6-X7, wherein X5 is independently selected from K or R, and X1, X3, X4, X6 and X7 are independently selected from any amino acid, optionally wherein the overall charge of the n-mer motif at neutral pH is between 0 and +2,
optionally, wherein the CNS-specific n-mer motif comprises or consists of NNSTRGG (SEQ ID NO: 42429), GNSARNI (SEQ ID NO: 42430), and GNSVRDF (SEQ ID NO: 42431), and
optionally, wherein the transgenic non-human animal is a rodent, optionally a mouse.

102. The method of claim 0, wherein the one or more cells are endothelial cells, CNS cells, or endothelial cells are of the CNS vasculature,
optionally, wherein the one or more cells are of the CNS vasculature, lungs, kidneys, liver, or any combination thereof.

103-109. (canceled)

110. A humanized transgenic non-human animal comprising:
one or more cells expressing a human transferrin polypeptide, optionally wherein the one or more cells are CNS cells,
optionally, wherein the transgenic non-human animal is a rodent, optionally a mouse.

111. (canceled)

112. A humanized transgenic non-human animal produced by the method of claim 101,
optionally, wherein the humanized non-human animal has a suppressed immune system.

113. (canceled)

114. A method of screening n-mer motifs capable of conferring transduction of central nervous system (CNS) tissues via binding to a transferrin receptor (TFRC) in humanized transgenic non-human animals comprising:
introducing one or more compositions comprising a candidate n-mer motif to humanized non-human transgenic animal of claim 101; and
detecting binding of the compositions that bind to a transferrin receptor (TFRC) and/or detecting transduction or uptake by one or more CNS cells of the humanized transgenic non-human animal,
optionally, wherein the candidate n-mer motif comprises or consists of X1-N-X3-X4-X5-X6-X7,
wherein X5 is independently selected from K or R, and X1, X3, X4, X6 and X7 are independently selected from any amino acid, optionally, wherein the overall charge of the n-mer motif at neutral pH is between 0 and +2.

115. (canceled)

116. The method of claim 114, wherein the composition is a viral particle comprising one or more capsid proteins each comprising the candidate n-mer motif,
optionally, wherein the viral particle is an AAV viral particle, and the one or more capsid proteins is an AAV capsid protein,
optionally, wherein the candidate n-mer motif is inserted in between amino acids 588-589 of an AAV9 capsid polypeptide or in an analogous position of a capsid protein from AAV1, AAV2, AAV3, AAV4, AAV5, AAV6, AAV7, AAV8, AAVrh8, AAV rh.74, or AAV rh. 10, and
optionally, wherein at least one of the one or more compositions furthers comprises a cargo,
optionally, wherein the cargo is or encodes a therapeutic nucleic acid or polypeptide, a selectable marker, or a control polypeptide or nucleic acid.

117-119. (canceled)

120. A recombinant AAV particle comprising
(1) a recombinant capsid protein having a TFR1-targeting n-mer motif peptide inserted within the capsid protein such that the peptide is displayed on the surface when incorporated into the AAV particle; and
(2) a recombinant AAV genome comprising a transgene encoding a therapeutic polypeptide or polynucleotide operably linked to one or more regulatory sequences which promote expression of the transgene in CNS cells, flanked by AAV ITR sequences,
wherein the AAV particle has enhanced binding, transduction or transgene expression in CNS cells relative to an AAV particle having a reference capsid that does not contain the TFR1-targeting n-mer motif;
optionally, wherein the recombinant AAV particle is an AAV9 capsid protein (SEQ ID NO: 20506), an AAV9 K449R capsid protein (SEQ ID NO: 20507), or a capsid protein having at least 90%, 90% or 99% sequence identity to the AAV9 capsid protein and forms a capsid that transduces CNS cells; and
optionally, wherein the peptide is inserted between amino acids 588 and 589 of the AAV9 capsid protein or at a corresponding position of another AAV capsid protein or the peptide is inserted between two consecutive amino acids within amino acids 451-460 of the AAV9 capsid protein or at a corresponding position within another AAV capsid protein.

121. The recombinant AAV particle of claim 0,
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from Y, M, F, and L;
X₂ comprises an amino acid chosen from S, H, T, and A;
X₃ comprises an amino acid chosen from K and R;
X₄ comprises an amino acid chosen from A, G, I, L, M, N, Q, S, T, V and H;
X₅ comprises an amino acid chosen from N, G, A, L, M, Q, S, and T;
X₆ comprises an amino acid chosen from A, T, H, N, F, I, P, L, Y, G, S, V, D, E, M, and Q; and
X₇ comprises an amino acid chosen from D and N,
or
wherein the TFR1-targeting n-mer motif peptide is a 7-mer peptide having an amino acid sequence of X₁-X₂-X₃-X₄-X₅-X₆-X₇
wherein:
X₁ comprises an amino acid chosen from Y or L;
X₂ comprises amino acid H;
X₃ comprises amino acid A;
X₄ comprises an amino acid chosen from K, R, N, and A;
X₅ comprises an amino acid chosen from G, Q, L, and S;
X₆ comprises an amino acid chosen from P, L, I, N, D, and T; and
X₇ comprises amino acid N,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from A, F, H, I, L, N, P, R, S, T, V, and Y;
X₂ comprises an amino acid chosen from A, D, E, F, G, H, I, K, L, M, N, Q, R, S, T, W, and Y;
X₃ comprises amino acid S;
X₄ comprises an amino acid chosen from S and T;
X₅ comprises amino acid N;
X₆ comprises amino acid G; and
X₇ comprises an amino acid chosen from I, R, and V,
or
wherein the TFR1-targeting n-mer motif peptide is a 7-mer peptide having an amino acid sequence of X₁-X₂-X₃-X₄-X₅-X₆-X₇
wherein:
X₁ comprises an amino acid chosen from R and T;
X₂ comprises an amino acid chosen from T, L, M, S, G, D, N, E, R, K, Y, and W;
X₃ comprises an amino acid chosen from G, E, D, I, F, H, S, A, M, P, V, Y, W, Q, and T;
X₄ comprises an amino acid chosen from D, T, E, H, N, and G;
X₅ comprises an amino acid chosen from A, V, S, T, and D;
X₆ comprises an amino acid chosen from Y, F, P and A; and
X₇ comprises an amino acid chosen from A and P,
or
wherein the TFR1-targeting n-mer motif peptide is a 7-mer peptide having an amino acid sequence of X₁-X₂-X₃-X₄-X₅-X₆-X₇,
wherein:
X₁ comprises amino acid L;
X₂ comprises amino acid C;
X₃ comprises an amino acid chosen from K and R;
X₄ comprises amino acid P;
X₅ comprises amino acid C;
X₆ comprises an amino acid chosen from L, S, D, A, N, Q, H, P, and V; and
X₇ comprises an amino acid chosen from E, T, G, A, D, N, and S,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from Y, and F;
X₂ comprises an amino acid chosen from W, F, and Y;
X₃ comprises an amino acid chosen from S, T, H, A and Q;
X₄ comprises amino acid G;
X₅ comprises an amino acid chosen from I, T, V, Q, M, H, K, and R;
X₆ comprises an amino acid chosen from I, P, H, L, M, A, Q, T, V, K, and R; and
X₇ comprises an amino acid chosen from A, S, D, E, and N,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from K, R, S, G, N, T, M, Q, V, D, I, and E;
X₂ comprises an amino acid chosen from D, S, N, M, L, G, P, E, and A;
X₃ comprises an amino acid chosen from E, D, G, S, A, R, Q, T, P, and N;
X₄ comprises an amino acid chosen from F, Y, T, V, S, N, A, G, and H;
X₅ comprises an amino acid chosen from T, K, S, R, V, and H;
X₆ comprises an amino acid chosen from T, S, G, V, A, K, R, N, D, E, and H; and
X₇ comprises an amino acid chosen from F, W, and Y,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from V, I, R, N and D;
X₂ comprises an amino acid chosen from A, G, and S;
X₃ comprises an amino acid chosen from L, T, S, H, and G;
X₄ comprises an amino acid chosen from K, R, and E;
X₅ comprises amino acid G;
X₆ comprises an amino acid chosen from W, R, A, and I; and
X₇ comprises an amino acid chosen from D and G,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from L, M, and W;
X₂ comprises an amino acid chosen from F, R, W, K, T, and Y;
X₃ comprises an amino acid chosen from D and S;
X₄ comprises amino acid G;
X₅ comprises amino acid T;
X₆ comprises an amino acid chosen from P, G, S, N, A, and R; and
X₇ comprises an amino acid chosen from A, P, S, and Y,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from P, N, and K;
X₂ comprises an amino acid chosen from Y and F;
X₃ comprises amino acid A;
X₄ comprises an amino acid chosen from R and K;
X₅ comprises amino acid S;
X₆ comprises an amino acid chosen from P, V, A, R, I, L, S, E; and
X₇ comprises an amino acid chosen from E, D, M, and L,
or
wherein the TFR1-targeting n-mer motif peptide is a 7-mer peptide having an amino acid sequence of Z₁-X₁-Z₂-X₂-X₃-X₄-X₅,
wherein
Z₁ is selected from the group consisting of Y, F, and L,
Z₂ is selected the from the group consisting of S, R, and K, and
X₁-X₅ are independently selected amino acids,
or
wherein the TFR1-targeting n-mer motif peptide is a 7-mer peptide having an amino acid sequence of X₁-H-X₂-L-X₃-X₄-X₅,
wherein X₁-X₅ are independently selected amino acids,
or
wherein the TFR1-targeting n-mer motif peptide is a 7-mer peptide having an amino an amino acid sequence of any one of SEQ ID NOs: 10952-20481 and 36241-42428.

122-126. (canceled)

127. A method of delivering a therapeutic polypeptide or polynucleotide to the CNS of a subject in need thereof, said method comprising administering to said subject the recombinant AAV particle of claim 120.

128. A recombinant engineered AAV capsid protein having a TFR1-targeting n-mer motif peptide inserted within the capsid protein such that the peptide is displayed on the surface when incorporated into the AAV capsid protein;
optionally wherein the recombinant capsid protein is an AAV9 capsid protein (SEQ ID NO: 20506), an AAV9 K449R capsid protein (SEQ ID NO: 20507), or a capsid protein having at least 90%, 90% or 99% sequence identity to the AAV9 capsid protein and forms a capsid that transduces CNS cells; and
optionally wherein the peptide is inserted between amino acids 588 and 589 of the AAV9 capsid protein or at a corresponding position of another AAV capsid protein.

129. The recombinant engineered AAV capsid protein of claim 0,
wherein the TFR1-targeting n-mer motif peptide is a 7-mer peptide having an amino acid sequence of X₁-X₂-X₃-X₄-X₅-X₆-X₇
wherein:
X₁ comprises an amino acid chosen from Y, M, F, and L;
X₂ comprises an amino acid chosen from S, H, T, and A;
X₃ comprises an amino acid chosen from K and R;
X₄ comprises an amino acid chosen from A, G, I, L, M, N, Q, S, T, V and H;
X₅ comprises an amino acid chosen from N, G, A, L, M, Q, S, and T;
X₆ comprises an amino acid chosen from A, T, H, N, F, I, P, L, Y, G, S, V, D, E, M, and Q; and
X₇ comprises an amino acid chosen from D and N,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from Y or L;
X₂ comprises amino acid H;
X₃ comprises amino acid A;
X₄ comprises an amino acid chosen from K, R, N, and A;
X₅ comprises an amino acid chosen from G, Q, L, and S;
X₆ comprises an amino acid chosen from P, L, I, N, D, and T; and
X₇ comprises amino acid N,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from A, F, H, I, L, N, P, R, S, T, V, and Y;
X₂ comprises an amino acid chosen from A, D, E, F, G, H, I, K, L, M, N, Q, R, S, T, W, and Y;
X₃ comprises amino acid S;
X₄ comprises an amino acid chosen from S and T;
X₅ comprises amino acid N;
X₆ comprises amino acid G; and
X₇ comprises an amino acid chosen from I, R, and V,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from R and T;
X₂ comprises an amino acid chosen from T, L, M, S, G, D, N, E, R, K, Y, and W;
X₃ comprises an amino acid chosen from G, E, D, I, F, H, S, A, M, P, V, Y, W, Q, and T;
X₄ comprises an amino acid chosen from D, T, E, H, N, and G;
X₅ comprises an amino acid chosen from A, V, S, T, and D;
X₆ comprises an amino acid chosen from Y, F, P and A; and
X₇ comprises an amino acid chosen from A and P,
or
wherein the TFR1-targeting n-mer motif peptide is a 7-mer peptide having an amino acid sequence of X₁-X₂-X₃-X₄-X₅-X₆-X₇,
wherein:
X₁ comprises amino acid L;
X₂ comprises amino acid C;
X₃ comprises an amino acid chosen from K and R;
X₄ comprises amino acid P;
X₅ comprises amino acid C;
X₆ comprises an amino acid chosen from L, S, D, A, N, Q, H, P, and V; and
X₇ comprises an amino acid chosen from E, T, G, A, D, N, and S,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from Y, and F;
X₂ comprises an amino acid chosen from W, F, and Y;
X₃ comprises an amino acid chosen from S, T, H, A and Q;
X₄ comprises amino acid G;
X₅ comprises an amino acid chosen from I, T, V, Q, M, H, K, and R;
X₆ comprises an amino acid chosen from I, P, H, L, M, A, Q, T, V, K, and R; and
X₇ comprises an amino acid chosen from A, S, D, E, and N,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from K, R, S, G, N, T, M, Q, V, D, I, and E;
X₂ comprises an amino acid chosen from D, S, N, M, L, G, P, E, and A;
X₃ comprises an amino acid chosen from E, D, G, S, A, R, Q, T, P, and N;
X₄ comprises an amino acid chosen from F, Y, T, V, S, N, A, G, and H;
X₅ comprises an amino acid chosen from T, K, S, R, V, and H;
X₆ comprises an amino acid chosen from T, S, G, V, A, K, R, N, D, E, and H; and
X₇ comprises an amino acid chosen from F, W, and Y,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from V, I, R, N and D;
X₂ comprises an amino acid chosen from A, G, and S;
X₃ comprises an amino acid chosen from L, T, S, H, and G;
X₄ comprises an amino acid chosen from K, R, and E;
X₅ comprises amino acid G;
X₆ comprises an amino acid chosen from W, R, A, and I; and
X₇ comprises an amino acid chosen from D and G,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from L, M, and W;
X₂ comprises an amino acid chosen from F, R, W, K, T, and Y;
X₃ comprises an amino acid chosen from D and S;
X₄ comprises amino acid G;
X₅ comprises amino acid T;
X₆ comprises an amino acid chosen from P, G, S, N, A, and R; and
X₇ comprises an amino acid chosen from A, P, S, and Y,
or
wherein the TFR1-targeting n-mer motif peptide is a 7-mer peptide having an amino acid sequence of X₁-X₂-X₃-X₄-X₅-X₆-X₇,
wherein:
X₁ comprises an amino acid chosen from P, N, and K;
X₂ comprises an amino acid chosen from Y and F;
X₃ comprises amino acid A;
X₄ comprises an amino acid chosen from R and K;
X₅ comprises amino acid S;
X₆ comprises an amino acid chosen from P, V, A, R, I, L, S, E; and
X₇ comprises an amino acid chosen from E, D, M, and L,
or
wherein the TFR1-targeting n-mer motif peptide is a 7-mer peptide having an amino acid sequence of Z₁-X₁-Z₂-X₂-X₃-X₄-X₅,
wherein
Z₁ is selected from the group consisting of Y, F, and L,
Z₂ is selected from the group consisting of S, R, and K, and
X₁-X₅ are independently selected amino acids,
or
wherein the TFR1-targeting n-mer motif peptide is a 7-mer peptide having an amino acid sequence of X₁-H-X₂-L-X₃-X₄-X₅
wherein X₁-X₅ are independently selected amino acids,
or
wherein the TFR1-targeting n-mer motif peptide is a 7-mer peptide having an amino acid sequence of any one of SEQ ID NOs: 10952-20481 and 36241-42428.

130-133. (canceled)

134. A host cell for producing the recombinant AAV particles of claim 120, said host cell comprising a first construct comprising a nucleic acid encoding the recombinant AAV capsid protein and a second construct comprising a nucleic acid encoding the recombinant AAV genome.

135. A method of producing the recombinant AAV particle of claim 120, said method comprising culturing a host cell under conditions sufficient for production of the recombinant AAV particle and harvesting of said recombinant AAV particle.
